(19) United States
(12) Patent Application Publication
(10) Pub. No.: US 2026/0053950 A1
(43) Pub. Date: Feb. 26, 2026

(54) EPIGENETIC MODULATION OF GENOMIC TARGETS TO CONTROL EXPRESSION OF PWS-ASSOCIATED GENES

(71) Applicant: Duke University, Durham, NC (US)

(72) Inventors: Charles A. Gersbach, Chapel Hill, NC (US); Dahlia Rohm, Durham, NC (US)

(21) Appl. No.: 19/104,624

(22) PCT Filed: Aug. 18, 2023
(86) PCT No.: PCT/US2023/072524

§ 371 Date: Feb. 18, 2025

Related U.S. Application Data
(60) Provisional application No. 63/399,121, filed on Aug. 18, 2022.
(60) Provisional application No. 63/418,910, filed on Oct. 24, 2022.

(51) Int. Cl. A61K 48/00; A61K 38/17; A61P 21/00; C12N 9/02; C12N 9/22; C12N 15/11; C12N 15/85

(52) U.S. Cl. CPC A61K 48/005; A61K 38/1709; A61P 21/00; C12N 9/0071; C12N 9/226; C12N 15/111; C12N 15/85; C07K 2319/71; C12N 2310/20; C12Y 114/11

(57) ABSTRACT

Disclosed herein are DNA targeting systems that target a regulatory element of a gene within the 15q11-13 locus. Further provided are DNA targeting systems including at least one gRNA and a Cas9 protein, as well as compositions comprising the same. The compositions may be used in methods for treating Prader-Willi Syndrome (PWS) in a subject. The method may include administering to a subject the DNA targeting system.

### CROSS-REFERENCE TO RELATED APPLICATIONS

This application claims priority to U.S. Provisional Patent Application No. 63/399,121, filed Aug. 18, 2022, and U.S. Provisional Patent Application No. 63/418,910, filed Oct. 24, 2022, each of which is incorporated herein by reference in its entirety.

### STATEMENT REGARDING FEDERALLY SPONSORED RESEARCH

This invention was made with government support under grants R01DA036865, U01AI146356, UM1HG013053, and RM1HG011123 awarded by the National Institutes of Health. The government has certain rights in the invention.

### SEQUENCE LISTING

This application includes a Sequence Listing which has been submitted electronically in XML format and is hereby incorporated by reference in its entirety. Said XML copy created on Aug. 18, 2023, is named “028193-9495-WO01 Sequence Listing.xmi” and is 1,629,585 bytes in size.

### FIELD

This disclosure relates to compositions and methods for the treatment of genetic and epigenetic disorders by modulating expression of genes in the region of the Prader-Willi Syndrome (PWS) imprinted locus and stable expression of a gene within the 15q11-13 PWS-associated locus.

### INTRODUCTION

Complex mechanisms of epigenetic regulation, such as imprinting and X-inactivation, are part of mammalian development, and dysregulation of these processes is the basis for several human disorders. Although the identity and dynamic control of the epigenetic markers involved in these processes have been investigated extensively, the mechanisms of heritable and allele-specific changes in gene expression at specific loci are still relatively poorly understood. Altering specific epigenetic modifications at particular imprinted loci could provide insight into the contributions of these marks to the stability of gene expression patterns. The advent of epigenome editing with DNA-targeting technologies such as CRISPR-Cas9 has revolutionized the ability to manipulate gene expression states in living cells and organisms. For example, nuclease-deactivated Cas9 (dCas9) fused to transcriptional regulators or epigenome modifiers can recruit transcription factors to promoters or enhancers, directly alter histone marks or DNA methylation, or sterically block transcription or transcription factor binding.

dCas9-based epigenome editing technologies can deposit chromatin modifications in a highly specific fashion. Recent success using CRISPR/Cas9 for targeted epigenome editing has enabled a more precise evaluation of the link between chromatin modification and gene expression. These technologies have also been applied for the unbiased identification of distal gene regulatory elements using pooled gRNA screens. Screens with dCas9-based epigenetic editors can elucidate the roles of particular epigenetic markers and define key regulatory elements that would otherwise be challenging to predict computationally. In addition to mapping the regulatory landscape in the human genome, these studies may identify potential drug targets for next-generation epigenetic therapies.

PWS is a neuroendocrine and neurobehavioral disorder linked to genetic aberrations at the 15q11-13 imprinted locus. PWS is characterized clinically by hyperphagia, early-onset obesity, and intellectual disability. While the exact genetic basis that causes PWS remains unclear, patient mutation profiles have implicated a small nucleolar RNA (snoRNA) gene cluster SNORD116 downstream of the SNURF-SNRPN open reading frame within 15q11-13 as a likely primary contributor to the disease etiology. Because 15q11-13 is an imprinted locus, PWS arises from mutations or large deletions on the paternal allele, while the corresponding maternal copy remains intact but epigenetically silenced. Consequently, activation of the maternal allele provides a therapeutic opportunity to restore expression of PWS-associated genes.

Activation of PWS-associated genes from the maternal allele has been realized through knockdown and broad inhibition of epigenetic modifying enzymes and transcription factors. While small molecule-based inhibition of epigenetic modifiers could serve as a viable therapeutic strategy for PWS, it also carries the added risk of off-target activity resulting from the global loss of an enzyme critical to gene regulation. Epigenetic modifying enzymes are often expressed across a diversity of cell types and can associate with thousands of genomic loci. It can be challenging to predict the consequences of generally inhibiting such an enzyme in vivo. A more targeted approach utilizing recently developed DNA-targeting platforms could achieve similar epigenetic modification at the intended locus with minimal off-target activity. Currently there is no cure for PWS and no effective treatment for its symptoms of hyperphagia and anxiety. There remains a need for improved and/or additional therapies for treating PWS.

### SUMMARY

In an aspect, the disclosure relates to method of stably activating a gene or gene product within the imprinted 15q11-13 locus in a subject having Prader Willi Syndrome (PWS) or Prader-Willi-like disorder. The method may include non-virally administering to the subject a DNA targeting system that targets a target region in the imprinted 15q11-13 locus, the DNA targeting system comprising: a Cas protein or a fusion protein, wherein the fusion protein comprises two heterologous polypeptide domains, wherein the first polypeptide domain comprises a DNA-binding protein and wherein the second polypeptide domain has an activity selected from transcription activation activity, transcription repression activity, transcription release factor activity, histone modification activity, nuclease activity, nucleic acid association activity, methylase activity, demethylase activity, acetylation activity, and deacetylation activity, wherein the Cas protein or fusion protein is targeted to the target region in the imprinted 15q11-13 locus. In some embodiments, at least one component of the DNA targeting system is transiently expressed in a cell from the subject or transiently delivered to a cell from the subject. In some embodiments, expression of a gene within the imprinted 15q11-13 locus is maintained in a cell from the subject for at least 10, at least 15, at least 20, at least 25, at least 26, at least 30, at least 35, at least 40, at least 45, at least 48, at least 50, or at least 55 days post-administration. In some embodiments, the DNA-binding protein comprises a Cas protein, a zinc finger protein, or a transcription activator-like effector (TALE) protein. In some embodiments, the DNA-binding protein comprises a Cas protein and the DNA targeting system further comprises one or more guide RNAs (gRNA) that binds to the target region in the imprinted 15q11-13 locus. In some embodiments, the Cas protein comprises a Cas9 protein. In some embodiments, the second polypeptide domain comprises VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSDI/BHC110 and homologs (SpLsd1/Swml/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1. In some embodiments, the second polypeptide domain comprises KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARID1B/PLU-1, JARIDIC/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hdal, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A. Laminin B, or CTCF. In some embodiments, the second polypeptide domain comprises Tet1c or Tetiv4. In some embodiments, the second polypeptide domain comprises the amino acid sequence of SEQ ID NO: 1139 or SEQ ID NO: 1166, or is encoded by a polynucleotide comprising the sequence of SEQ ID NO: 1138 or SEQ ID NO: 1167. In some embodiments, the fusion protein comprises VP64-dCas9-VP64, dCas9-KRAB, Tet1c-dCas9, or Tetiv4-dCas9. In some embodiments, the fusion protein comprises the amino acid sequence of SEQ ID NO: 1168 or SEQ ID NO: 1169, or is encoded by a polynucleotide comprising the sequence of SEQ ID NO: 1169 or SEQ ID NO: 1171. In some embodiments, the target region in the imprinted 15q11-13 PWS-associated locus is on the maternal copy. In some embodiments, the target region in the imprinted 15q11-13 PWS-associated locus is on the paternal copy. In some embodiments, the expression of a gene or gene product within the imprinted 15q11-13 locus is increased. In some embodiments, the gene within the imprinted 15q11-13 locus comprises SNRPN, MAGEL2, MKRN3, NDN, C150RF2, SNURF-SNRPN, SNHG14, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, SNG14, or a snoRNA in the SNORD116 cluster, or a combination thereof. In some embodiments, the gene within the imprinted 15q11-13 locus comprises SNRPN, SNORD116, MAGEL2, SNORD115, SPA1, and/or SPA2. In some embodiments, the expression of MAGEL2 or its products is increased. In some embodiments, the expression of SNORD116 or its products is increased. In some embodiments, the expression of the SNRPN gene or its products is increased. In some embodiments, expression of the SNRPN gene is maintained in a cell from the subject for at least 10, at least 15, at least 20, at least 25, at least 26, at least 30, at least 35, at least 40, at least 45, at least 48, at least 50, or at least 55 days post-administration. In some embodiments, the gRNA is encoded by a polynucleotide comprising a sequence selected from SEQ ID NOs: 1148-1156 or binds to a polynucleotide comprising a sequence selected from SEQ ID NOs: 1148-1156 or comprises a sequence selected from SEQ ID NOs: 1157-1165. In some embodiments, the DNA targeting system comprises two or more gRNAs. In some embodiments, the subject is administered a vector comprising a polynucleotide encoding the DNA targeting system. In some embodiments, the vector is a plasmid or a synthetic vector. In some embodiments, the vector comprises RNA. In some embodiments, the vector comprises ribonucleoprotein (RNP). In some embodiments, the vector is a vector within a nanoparticle. In some embodiments, the nanoparticle is a lipid nanoparticle or a polymeric nanoparticle.

In a further aspect, the disclosure relates to DNA targeting system that targets the imprinted 15q11-13 locus. The DNA targeting system may include (a) a Cas9 fusion protein, wherein the fusion protein comprises two heterologous polypeptide domains, wherein the first polypeptide domain comprises a Cas protein and the second polypeptide domain comprises Tet1, Tet1c, or Tet1v4; and (b) one or more guide RNAs (gRNA) that bind to a target region in the imprinted 15q11-13 locus. In some embodiments, the DNA targeting system is for use in stably activating expression of a gene or gene product within the imprinted 15q11-13 locus in a subject having Prader Willi Syndrome (PWS) or Prader-Willi-like disorder.

Another aspect of the disclosure provides isolated polynucleotide sequence encoding a DNA targeting system as detailed herein.

Another aspect of the disclosure provides a vector comprising an isolated polynucleotide sequence as detailed herein.

Another aspect of the disclosure provides a nanoparticle. The nanoparticle may include a DNA targeting system as detailed herein, or an isolated polynucleotide sequence as detailed herein, or a vector as detailed herein, or a combination thereof. In some embodiments, the nanoparticle is a lipid nanoparticle or a polymeric nanoparticle.

Another aspect of the disclosure provides a pharmaceutical composition. The pharmaceutical composition may include a DNA targeting system as detailed herein, or an isolated polynucleotide sequence as detailed herein, or a vector as detailed herein, or a nanoparticle as detailed herein, or a combination thereof.

The disclosure provides for other aspects and embodiments that will be apparent in light of the following detailed description and accompanying figures.

### BRIEF DESCRIPTION OF THE DRAWINGS

FIGS. 1A-1H. High-throughput screens reveal regulatory elements of maternal and paternal SNRPN alleles. (FIG. 1A) Schematic of the PWS locus on chr15 with common PWS deletions and the PWS gRNA library. Each thin vertical line represents a single gRNA. Genes colored blue are maternally imprinted, those that are pink are paternally imprinted, and those that are grey are not imprinted. (FIG. 1B) Summary of the PWS gRNA library. (FIG. 1C) Schematic of experimental protocol for CRISPRa/CRISPRi screens. (FIG. 1D) CRISPR screen results (zoomed in, see FIG. 2C) displayed as −log₁₀(p_{adj}), where p_{adj} is the multiple-hypothesis-corrected p-value from DESeq2. Notable regions are highlighted in beige. (FIG. 1E) Summary of the PWS gRNA sub-library. (FIG. 1F) qPCR of SNRPN-GFP for validations of pools of individual gRNAs of the matSNRPN-2A-GFP CRISPRa VP64-dCas9-VP64 screen. (FIG. 1G) Plot of −log₁₀(p_{adj}) values of each gRNA in the VP64-dCas9-VP64 full library screen vs. Tetlc-dCas9 sublibrary screen, plotting only the gRNAs present in both screens. (Significant p_{adj}<0.05.) (FIG. 1H) qPCR of SNRPN-GFP for validations of individual gRNAs of the matSNRPN-2A-GFP CRISPRa Tet1c-dCas9 screen.

FIGS. 2A-2H. CRISPRa/i screens. (FIG. 2A) Schematic of derivation of maternally or paternally tagged SNRPN-2A-GFP iPSCs. (FIG. 2B) Flow cytometry validation of differential GFP fluorescence of maternally and paternally-tagged lines. (FIG. 2C) CRISPR screen results shown in FIG. 1D, view zoomed out to cover the entire span of the human PWS gRNA library. (FIG. 2D) CRISPRi dCas9-KRAB patSNRPN-2A-GFP screen results (shown in FIG. 1D), separated by strand on which the gRNA is located. (FIG. 2E) Plot of −log₁₀(p_{adj}) values of each gRNA in the VP64-dCas9-VP64 full library screen vs. dCas9-KRAB screen. (Significant p_{adj}<0.05.) (FIG. 2F) qPCR of SNRPN-GFP from individual gRNA validations of each of the gRNAs in the mat1 and mat2 pools shown in FIG. 1F. (FIG. 2G) qPCR of SNRPN-GFP in patSNRPN-2A-GFP dCas9-KRAB iPSCs with gRNAs from the pat4, mat1, and mat2 regions. (FIG. 2H) qPCR of SNRPN-GFP in matSNRPN-2A-GFP VP64-dCas9-VP64 iPSCs with gRNAs from the CRISPRi screen hits. For qPCR in (FIG. 2F), (FIG. 2G), and (FIG. 2H), fold change values are plotted mean+/−SD, but statistics were calculated on ddCt values (normalized to GAPDH and empty vector sample); one-way ANOVA followed by Dunnett's test vs. empty vector. **p<0.001, ***p<0.0001. Unmarked comparisons are not significant.

FIGS. 3A-3I. KRAB can activate paternal SNRPN expression. (FIG. 3A) Flow cytometry of SNRPN-GFP MFI for validations of individual gRNAs of the patSNRPN-2A-GFP CRISPRi dCas9-KRAB screen. MFI values normalized to Empty vector. One-way ANOVA followed by Dunnett's multiple comparisons test vs. Empty. *p<0.01, **p<0.001, ***p<0.0001. (FIG. 3B) CRISPRi screen results (shown in FIG. 1D), plotted as log₂(fold change) gRNA enrichment between low and high GFP sorted bins. (FIG. 3C) qPCR of SNRPN-GFP from total mRNA in patSNRPN-2A-GFP dCas9-KRAB lines. (FIG. 3D) qPCR of SNRPN-GFP from polyadenylated (poly-A) mRNA in patSNRPN-2A-GFP dCas9-KRAB lines. (FIG. 3E) qPCR of the indicated genes from total mRNA in patSNRPN-2A-GFP dCas9-KRAB lines. In (FIG. 3C), (FIG. 3D), and (FIG. 3E), qPCR results are plotted as fold change values mean+/−SD, but statistics were calculated on ddCt values (normalized to GAPDH and empty vector control); one-way ANOVA followed by Dunnett's test vs. ΔPWS NT gRNA *p<0.05, ***p<0.001, ****p<0.0001. (FIG. 3F) Schematic of 3′ RACE-seq of SNRPN transcript. (FIG. 3G, FIG. 3H) Comparison of SNRPN 3′ UTR sequence variants in control cells with dCas9-KRAB and an empty gRNA vector and cells treated with either (FIG. 3G) a pat6 gRNA or (FIG. 3H) a pat8 gRNA (FIG. 3I) Sequences of the four most predominant 3′ UTR variants detected in all conditions. Number labels in (FIG. 3G) and (FIG. 3H) match the corresponding numbered sequences in (FIG. 3I).

FIGS. 4A-4D. Dual gRNA screen with VP64-dCas9-VP64 reveals additional regulatory regions of SNRPN (FIG. 4A) Genome browser track depicting results from both the single and dual gRNA screens. (FIG. 4B) Comparison of the significant hits (p_{adj}<0.05) between the single and dual gRNA CRISPRa screens upstream of the SNRPN promoter. (FIG. 4C) Plot of −log₁₀(p_{adj}) values of each gRNA in the VP64-dCas9-VP64 full library screen vs. sublibrary screen. (Significant p_{adj}<0.05.) (FIG. 4D) Plot of −log₁₀(p_{adj}) values of each gRNA in the Tet1c-dCas9 sublibrary screen vs. dCas9-KRAB full library screen. (Significant pd, <0.05.)

FIGS. 5A-5G. Tet1c and VP64 activate maternally imprinted PWS genes in ΔPWS iPSCs. (FIG. 5A) Schematic of chr15 in isogenic wildtype (WT) and PW Type II deletion (ΔPWS) iPSCs. (FIG. 5B) qPCR of SNRPN in WT or ΔPWS iPSCs with VP64-dCas9-VP64 14 days after transduction with the indicated gRNA or gRNA pool. (FIG. 5C) qPCR of SNRPN in WT or ΔPWS iPSCs with Tet1c-dCas9 14 days after transduction with the indicated gRNA or gRNA pool. For both qPCR plots, fold change values are plotted mean+/−SD, but statistics were calculated on ddCt values (normalized to GAPDH and WT ctrl sample); one-way ANOVA followed by Dunnett's test vs. ΔPWS NT gRNA ****p<0.0001. (FIG. 5D) Differential expression analysis of total RNA sequencing of VP64-dCas9-VP64 ΔPWS iPSCs, comparing mat1 g3 to NT gRNA (FIG. 5E) Differential expression analysis of total RNA sequencing of Tetlc-dCas9 ΔPWS iPSCs, comparing IC g5 to NT gRNA. (FIG. 5F) HCR FlowFISH of VP64-dCas9-VP64 iPSCs (WT or ΔPWS) with the indicated gRNA. SNHG14 signal on X axis, with TBP as a control for cell size and staining. (FIG. 5G) HCR FlowFISH of Tet1c-dCas9 iPSCs (WT or ΔPWS) with the indicated gRNA. SNRPN (transcript variant 1) signal on X axis, with TBP as a control for cell size and staining.

FIGS. 6A-6G. PWS gene expression in VP64-dCas9-VP64 or Tet1c-dCas9 ΔPWS iPSCs. (FIG. GA, FIG. 6C): qPCR of WT or ΔPWS VP64-dCas9-VP64 iPSCs with NT or mat1 g3 gRNA for either (FIG. 6A) SNORD116 or (FIG. 6C) sets of SNRPN transcript variants. (FIG. 6B, FIG. 6D) qPCR of WT or ΔPWS Tet1c-dCas9 iPSCs with NT or IC g5 gRNA for either (FIG. 6B) SNORD116 or (FIG. 6D) sets of SNRPN transcript variants. Fold change values (relative to GAPDH) are plotted mean+/−SD, but statistics were calculated on dCt values (normalized to GAPDH): one-way ANOVA followed by Sidak's multiple comparisons test for select groups WT+targeting gRNA vs. NT gRNA or ΔPWS+targeting gRNA vs. NT gRNA. *p<0.05, ***p<0.001,***p<0.0001. (FIG. 6E, FIG. 6F) Two replicates of HCR FlowFISH (Rep. 1 of each was shown in FIG. 3F and FIG. 3G, respectively) of WT or ΔPWS iPSCs with either (FIG. 6E) VP64-dCas9-VP64 and NT or mat1/2 gRNAs, or (FIG. 6F) Tetlc-dCas9 and NT or IC gRNAs. (FIG. 6G) Browser tracks of ATAC sequencing (rpkm-normalized BigWig) of ΔPWS or WT iPSCs with VP64-dCas9-VP64 and NT or mat1 g3 gRNA at the PWAR1 gene.

FIGS. 7A-7D. Additional sequencing of VP64-dCas9-VP64 or Tet1c-dCas9 ΔPWS iPSC conditions. (FIG. 7A) Genome browser visualization of RNA sequencing (rpkm-normalized BigWig) of VP64-dCas9-VP64 WT or ΔPWS iPSCs with NT or mat1 g3 gRNA, zoomed in on SNRPN upstream exons. (FIG. 7B) Browser tracks of ATAC sequencing (rpkm-normalized BigWig) of ΔPWS or WT iPSCs with Tet1c-dCas9 and NT or IC g5 gRNA. (FIG. 7C) Quantification of ATAC-seq reads (counts per million) at each of the two peaks at the PWS-IC (mat3 g5 is located within the first of the two peaks, see S4B). ΔPWS+NT vs. mat3 gRNA not significant, Tukey's test following one-way ANOVA. (FIG. 7D) qPCR of SNRPN expression in ΔPWS iPSCs with NT or mat3 g5 gRNA, comparing 3 different Tet1c-dCas9 constructs, all delivered by lentivirus.

FIGS. 8A-8D. Tet1c and VP64 alter chromatin accessibility and/or DNA methylation at the PWS locus. (FIG. 8A) Targeted bisulphite sequencing of ΔPWS iPSCs with VP64-dCas9-VP64 covering 24 CpG sites within the PWS locus (hg19 chr15: 25200353-25200693) (FIG. 8B) Targeted bisulphite sequencing of ΔPWS iPSCs with Tet1c-dCas9 covering 24 CpG sites within the PWS locus (hg19 chr15: 25200353-25200693). Data for (FIG. 8A) and (FIG. 8B) are shown as the range of the data, with the plotted point being the median. (FIG. 8C) Browser tracks of ATAC sequencing (rpkm-normalized BigWig) of ΔPWS or WT iPSCs with VP64-dCas9-VP6′ and NT or mat1 g3 gRNA. (FIG. 8D) Quantification of ATAC-seq reads (counts per million) at the peak at the mat1 g3 guide binding site (dashed line in (FIG. 8A)). **p<0.001, Tukey's test following one-way ANOVA.

FIGS. 9A-9D. Transient expression of Tetiv4-dCas9 in ΔPWS iPSCs stably activates maternal PWS genes. (FIG. 9A) Schematic of experimental protocol for transient delivery of Tetiv4-dCas9 plasmid and PWS gene expression analysis. (FIG. 9B) qPCR of dCas9 or SNRPN in WT or ΔPWS iPSCs after transient delivery of Tetiv4-dCas9 on Day 0. (FIG. 9C) qPCR of PWS genes in iPSC-derived neurons. Data plotted as mean fold change+/−SD, but statistics computed on ddCt (normalized to GAPDH and WT+NT). Two-way ANOVA followed by Dunnett's test, compared to ΔPWS+NT gRNA: **p<0.01, ***p<0.001, ****p<0.0001. (FIG. 9D) Targeted bisulphite sequencing of ΔPWS iPSC-derived neurons approximately 21 days post-differentiation, covering 24 CpG sites within the PWS locus (hg19 chr15: 25200353-25200693). Data shown as median+/−range.

### DETAILED DESCRIPTION

As detailed herein, a CRISPR/dCas9-based screening approach was used to identify genomic regulatory elements at the 15q11-13 locus controlling expression of the SNURF-SNRPN host transcript in human induced pluripotent stem cells (iPSCs). Through independent screens against either the paternal or maternal allele, regulatory elements controlling expression of PWS-associated genes were identified. The successful activation of the maternal host transcript and other PWS genes, such as SNRPN and the transcript containing SNORD115 and SNORD116, was demonstrated using targeted epigenetic editing with different dCas9-based effectors, including both DNA methylation-dependent and DNA methylation-independent mechanisms. These discoveries provide a new avenue for targeted epigenetic therapy.

The present invention is directed to methods of treating Prader-Willi Syndrome (PWS), Prader-Willi-like syndrome, or disorders that would benefit from activation of the genes within the PWS locus, wherein activation of the genes within one allele of the 15q11-13 locus reintroduces lost functional gene expression. The compositions and methods detailed herein may stably activate a gene or gene product within the imprinted 15q11-13 locus. As further detailed herein, non-viral administration of the DNA targeting systems resulted in stable expression of genes within the 15q11-13 imprinted region, even though expression of the DNA targeting system was transient.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and,” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

The term “about” as used herein as applied to one or more values of interest, refers to a value that is similar to a stated reference value. In certain aspects, the term “about” refers to a range of values that fall within 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Adeno-associated virus” or “AAV” as used interchangeably herein refers to a small virus belonging to the genus Dependovirus of the Parvoviridae family that infects humans and some other primate species, including variants thereof. AAV is not currently known to cause disease and consequently the virus causes a very mild immune response.

“Amino acid” as used herein refers to naturally occurring and non-natural synthetic amino acids, as well as amino acid analogs and amino acid mimetics that function in a manner similar to the naturally occurring amino acids. Naturally occurring amino acids are those encoded by the genetic code. Amino acids can be referred to herein by either their commonly known three-letter symbols or by the one-letter symbols recommended by the IUPAC-IUB Biochemical Nomenclature Commission. Amino acids include the side chain and polypeptide backbone portions.

“Binding region” as used herein refers to the region within a target region that is recognized and bound by the DNA binding portion of a DNA Targeting System including a Targeted Activator System or a Targeted Repressor System, such as a nuclease or DNA binding domain fused to an activator or repressor.

“Coding sequence” means a nucleotide sequence (RNA or DNA) which encodes a protein. The coding sequence can further include initiation and termination signals operably linked to regulatory elements including a promoter and polyadenylation signal capable of directing expression in the cells of an individual or mammal to which the nucleic acid is administered. The coding sequence may be codon optimized.

“Complement” or “complementary” as used herein with respect to a nucleic acid means Watson-Crick (such as, A-T/U and C-G) or Hoogsteen base pairing between nucleotides or nucleotide analogs of nucleic acids. “Complementarity” refers to a property shared between two nucleic acid sequences, such that when they are aligned antiparallel to each other, the nucleotide bases at each position will be complementary.

The terms “control,” “reference level,” and “reference” are used herein interchangeably. The reference level may be a predetermined value or range, which is employed as a benchmark against which to assess the measured result. “Control group” as used herein refers to a group of control subjects. The predetermined level may be a cutoff value from a control group. The predetermined level may be an average from a control group. Cutoff values (or predetermined cutoff values) may be determined by Adaptive Index Model (AIM) methodology. Cutoff values (or predetermined cutoff values) may be determined by a receiver operating curve (ROC) analysis from biological samples of the patient group. ROC analysis, as generally known in the biological arts, is a determination of the ability of a test to discriminate one condition from another, such as, to determine the performance of each marker in identifying a patient having CRC. A description of ROC analysis is provided in P. J. Heagerty et al. (Biometrics 2000, 56, 337-44), the disclosure of which is hereby incorporated by reference in its entirety. Alternatively, cutoff values may be determined by a quartile analysis of biological samples of a patient group. For example, a cutoff value may be determined by selecting a value that corresponds to any value in the 25th-75th percentile range, preferably a value that corresponds to the 25th percentile, the 50th percentile or the 75th percentile, and more preferably the 75th percentile. Such statistical analyses may be performed using any method known in the art and can be implemented through any number of commercially available software packages (such as, from Analyse-it Software Ltd., Leeds, UK; StataCorp LP, College Station, TX; SAS Institute Inc., Cary, NC.). The healthy or normal levels or ranges for a target or for a protein activity may be defined in accordance with standard practice. A control may be an subject or cell without an agonist as detailed herein. A control may be a subject, or a sample therefrom, whose disease state is known. The subject, or sample therefrom, may be healthy, diseased, diseased prior to treatment, diseased during treatment, or diseased after treatment, or a combination thereof.

“Donor DNA”, “donor template,” and “repair template” as used interchangeably herein refers to a double-stranded DNA fragment that includes at least a portion of the gene of interest.

“Frameshift” or “frameshift mutation” as used interchangeably herein refers to a type of gene mutation wherein the addition or deletion of one or more nucleotides causes a shift in the reading frame of the codons in the mRNA. The shift in reading frame may lead to the alteration in the amino acid sequence at protein translation, such as a missense mutation or a premature stop codon.

“Fusion protein” as used herein refers to a chimeric protein created through the covalent or non-covalent joining of two or more separate proteins. In some embodiments, translation of a fusion gene created through joining of two or more genes that originally coded for separate proteins results in a single polypeptide with functional properties derived from each of the original proteins.

“Genetic construct” as used herein refers to the DNA or RNA that comprise a polynucleotide that encodes a protein or RNA. The coding sequence includes initiation and termination signals operably linked to regulatory elements including a promoter and polyadenylation signal capable of directing expression in the cells of the individual to whom the nucleic acid is administered. As used herein, the term “expressible form” refers to gene constructs that contain the necessary regulatory elements operable linked to a coding sequence that encodes a protein or RNA such that when present in the cell of the individual, the coding sequence will be expressed.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the CRISPR/Cas9-based gene editing system, then the cellular machinery will repair the break by homologous recombination, which is enhanced several orders of magnitude in the presence of DNA cleavage. When the homologous DNA piece is absent, non-homologous end joining may take place instead.

“Genome editing” as used herein refers to changing a gene. Genome editing may include correcting or restoring a mutant gene or adding additional mutations. Genome editing may include knocking out a gene, such as a mutant gene or a normal gene.

“Identical” or “identity” as used herein in the context of two or more nucleic acids or polypeptide sequences means that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation. When comparing DNA and RNA, thymine (T) and uracil (U) may be considered equivalent. Identity may be performed manually or by using a computer sequence algorithm such as BLAST or BLAST 2.0. Identity of related peptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press. New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al, SIAM J. Applied Math. 48, 1073 (1988), herein incorporated by reference in their entirety.

As used herein, the term “imprinting” refers to the differential expression of alleles of the same gene in a parent-of-origin-specific manner, or to the biological process by which such a pattern is established. An “imprinted gene” is a gene that is subject to imprinting. Mammalian somatic cells are normally diploid, i.e., they contain two homologous sets of autosomes (chromosomes that are not sex chromosomes)—one set inherited from each parent, and a pair of sex chromosomes. Thus, mammalian somatic cells normally contain two copies of each autosomal gene—a maternal copy and a paternal copy. The two copies (often referred to as “alleles”) may be identical or may differ at one or more nucleotide positions. For most genes, the alleles inherited from the mother and father exhibit similar expression levels. In contrast, imprinted genes are normally expressed in a parent-of-origin specific manner-either the maternal allele (the allele on the chromosome inherited from the mother) is expressed and the paternal allele (the allele present on the chromosome inherited from the father) is not, or the paternal allele is expressed and the maternal allele is not. The allele that is not expressed may be referred to as the “imprinted allele” or “imprinted copy”. Imprinted genes can occur in large, coordinately regulated clusters or small domains composed of only one or two genes. Imprinting has generally been found to be conserved between mice and humans, i.e., if a gene is imprinted in mice, the orthologous gene is typically imprinted in humans as well, and vice versa. Parental allele-specific expression of imprinted genes is generally due to an imprinting control region.

As used herein, an “imprinting center” is a DNA region that controls the imprinting of at least one gene (typically a cluster of genes). In other words, the imprinting center controls the mono-allelic expression of the at least one gene in a manner that depends on the parental origin of the alleles. An imprinting center must be on the same chromosome as the imprinted gene(s) whose expression it affects but can be located a considerable distance away (such as, up to several megabases away).

The term “imprinting disorder” refers to any disorder caused by alterations in the normal imprinting pattern, any disorder caused by changes in expression or gene dosage of an imprinted gene, and/or any disorder caused by the mutation or deletion of an imprinted gene. Non-limiting examples of imprinting disorders include Angelman syndrome. Prader-Willi syndrome.

“Mutant gene” or “mutated gene” as used interchangeably herein refers to a gene that has undergone a detectable mutation. A mutant gene has undergone a change, such as the loss, gain, or exchange of genetic material, which affects the normal transmission and expression of the gene. A “disrupted gene” may refer to a mutant gene that has a mutation that causes a premature stop codon. The disrupted gene product is truncated relative to a full-length undisrupted gene product.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or after the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur but is much more common when the overhangs are not compatible.

“Normal gene” as used herein refers to a gene that has not undergone a change, such as a loss, gain, or exchange of genetic material. The normal gene undergoes normal gene transmission and gene expression. For example, a normal gene may be a wild-type gene.

“Nuclease mediated NHEJ” as used herein refers to NHEJ that is initiated after a nuclease cuts double stranded DNA.

“Nucleic acid” or “oligonucleotide” or “polynucleotide” as used herein means at least two nucleotides covalently linked together. The depiction of a single strand also defines the sequence of the complementary strand. Thus, a polynucleotide also encompasses the complementary strand of a depicted single strand. Many variants of a polynucleotide may be used for the same purpose as a given polynucleotide. Polynucleotides may be single stranded or double stranded, or may contain portions of both double stranded and single stranded sequence. The polynucleotide can be nucleic acid, natural or synthetic, DNA, genomic DNA, cDNA, RNA, or a hybrid, where the polynucleotide can contain combinations of deoxyribo- and ribo-nucleotides, and combinations of bases including uracil, adenine, thymine, cytosine, guanine, inosine, xanthine hypoxanthine, isocytosine, and isoguanine. Polynucleotides can be obtained by chemical synthesis methods or by recombinant methods.

“Open reading frame” refers to a stretch of codons that begins with a start codon and ends at a stop codon. In eukaryotic genes with multiple exons, introns are removed, and exons are then joined together after transcription to yield the final mRNA for protein translation. An open reading frame may be a continuous stretch of codons. In some embodiments, the open reading frame only applies to spliced mRNAs, not genomic DNA, for expression of a protein.

“Operably linked” as used herein means that expression of a gene is under the functional control of a regulatory element, such as a promoter. For instance, a promoter or enhancer is operably linked to a coding sequence if it regulates, or contributes to the modulation of, the transcription of the coding sequence. A promoter may be positioned 5′ (upstream) or 3′ (downstream) of a gene under its control. As is known in the art, variation in the distance between the promoter and the gene it controls may be accommodated without loss of promoter function. Enhancers may function when separated from the promoter by up to several kilobases or more. Thus, regulatory elements may be operably linked without being contiguous.

“Partially-functional” as used herein describes a protein that is encoded by a mutant gene and has less biological activity than a functional protein but more than a non-functional protein.

A “peptide” or “polypeptide” is a linked sequence of two or more amino acids linked by peptide bonds. The polypeptide can be natural, synthetic, or a modification or combination of natural and synthetic. The terms “polypeptide”, “protein.” and “peptide” are used interchangeably herein. “Primary structure” refers to the amino acid sequence of a particular peptide. “Secondary structure” refers to locally ordered, three dimensional structures within a polypeptide. These structures are commonly known as domains, such as, enzymatic domains, extracellular domains, transmembrane domains, pore domains, and cytoplasmic tail domains. “Domains” are portions of a polypeptide that form a compact unit of the polypeptide and are typically 15 to 350 amino acids long. Exemplary domains include domains with enzymatic activity or ligand binding activity. Typical domains are made up of sections of lesser organization such as stretches of beta-sheet and alpha-helices. “Tertiary structure” refers to the complete three-dimensional structure of a polypeptide monomer. “Quaternary structure” refers to the three-dimensional structure formed by the noncovalent association of independent tertiary units. A “motif” is a portion of a polypeptide sequence and includes at least two amino acids. A motif may be 2 to 20, 2 to 15, or 2 to 10 amino acids in length. In some embodiments, a motif includes 3, 4, 5, 6, or 7 sequential amino acids. A domain may be comprised of a series of the same type of motif.

“Premature stop codon” or “out-of-frame stop codon” as used interchangeably herein refers to nonsense mutation in a sequence of DNA, which results in a stop codon at location not normally found in the wild-type gene. A premature stop codon may cause a protein to be truncated or shorter compared to the full-length version of the protein.

The term “recombinant” when used with reference to, for example, a cell, nucleic acid, protein, or vector, indicates that the cell, nucleic acid, protein, or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed, or not expressed at all.

“Transcriptional regulatory elements” or “regulatory elements” refers to a genetic element which can control the expression of nucleic acid sequences, such as activate, enhancer, or decrease expression, or alter the spatial and/or temporal expression of a nucleic acid sequence. Examples of regulatory elements include promoters, enhancers, splicing signals, polyadenylation signals, and termination signals. “Promoter” as used herein means a synthetic or naturally-derived nucleotide sequence which is capable of conferring, activating or enhancing expression of a nucleic acid in a cell. A promoter may also comprise distal enhancer or repressor elements, which may be located as much as several thousand base pairs from the start site of transcription. A promoter or other regulatory element may be derived from sources including viral, bacterial, fungal, plants, insects, and animals. A promoter may regulate the expression of a gene component constitutively, or differentially with respect to cell, the tissue or organ in which expression occurs or, with respect to the developmental stage at which expression occurs, or in response to external stimuli such as physiological stresses, pathogens, metal ions, or inducing agents. The selection of a particular promoter and enhancer depends on the recipient cell type. Some eukaryotic promoters and enhancers have a broad host range while others are functional in a limited subset of cell types. Representative examples of promoters include the bacteriophage T7 promoter, bacteriophage T3 promoter, SP6 promoter, lac operator-promoter, tac promoter, SV40 late promoter, SV40 early promoter, RSV-LTR promoter, CMV IE promoter, SV40 early promoter or SV40 late promoter, human U6 (hU6) promoter, and CMV IE promoter. A promoter and/or enhancer can be “endogenous,” “exogenous,” or “heterologous” with respect to the gene to which it is operably linked. An “endogenous” promoter/enhancer is one which is naturally linked with a given gene in the genome. An “exogenous” or “heterologous” enhancer or promoter is one which is not normally linked with a given gene but is placed in operable linkage with a gene by genetic manipulation.

As used herein, the term “heterologous” refers to a nucleic acid or polypeptide comprising two or more subsequences that are not found in the same relationship to each other in nature. For instance, a nucleic acid that is recombinantly produced typically has two or more sequences from unrelated genes synthetically arranged to make a new functional nucleic acid, such as, a promoter from one source and a coding region from another source. The two nucleic acids are thus heterologous to each other in this context. When added to a cell, the recombinant nucleic acids would also be heterologous to the endogenous genes of the cell.

“Sample” or “test sample” as used herein can mean any sample in which the presence and/or level of a target is to be detected or determined or any sample comprising a DNA targeting system or component thereof as detailed herein. Samples may include liquids, solutions, emulsions, or suspensions. Samples may include a medical sample. Samples may include any biological fluid or tissue, such as blood, whole blood, fractions of blood such as plasma and serum, muscle, interstitial fluid, sweat, saliva, urine, tears, synovial fluid, bone marrow, cerebrospinal fluid, nasal secretions, sputum, amniotic fluid, bronchoalveolar lavage fluid, gastric lavage, emesis, fecal matter, lung tissue, peripheral blood mononuclear cells, total white blood cells, lymph node cells, spleen cells, tonsil cells, cancer cells, tumor cells, bile, digestive fluid, skin, or combinations thereof. In some embodiments, the sample comprises an aliquot. In other embodiments, the sample comprises a biological fluid. Samples can be obtained by any means known in the art. The sample can be used directly as obtained from a patient or can be pre-treated, such as by filtration, distillation, extraction, concentration, centrifugation, inactivation of interfering components, addition of reagents, and the like, to modify the character of the sample in some manner as discussed herein or otherwise as is known in the art.

“Subject” as used herein can mean a mammal that is in need of the herein described compositions or methods. The subject may be a patient. The subject may be a human or a non-human. The subject may be any vertebrate. The subject may be a mammal. The mammal may be a primate or a non-primate. The mammal can be a non-primate such as, for example, dog, cat, horse, cow, pig, mouse, rat, mouse, camel, llama, goat, rabbit, sheep, hamster, and guinea pig. The mammal can be a primate such as a human. The mammal can be a non-human primate such as, for example, monkey, cynomolgous monkey, rhesus monkey, chimpanzee, gorilla, orangutan, and gibbon. The subject may be of any age or stage of development, such as, for example, an adult, an adolescent, a child, such as age 0-2, 2-4, 2-6, or 6-12, or an infant, such as age 0-1. The subject may be male. The subject may be female. In some embodiments, the subject has a specific genetic marker. The subject may be undergoing other forms of treatment.

“Substantially identical” can mean that a first and second amino acid or polynucleotide sequence are at least 60%, 65%, 70%, 75%, 80%, 85%, 90%, 95%, 96%, 97%, 98%, or 99% over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids or nucleotides, respectively.

“Target gene” as used herein refers to any nucleotide sequence encoding a known or putative gene product that is intended to be corrected or for which its expression is intended to be modulated. The target gene may be a mutated gene involved in a genetic disease. In certain embodiments, the target gene is within or near the 15q11-q13 locus.

“Target region” as used herein refers to the region of the chromosome to which the DNA Targeting System, Targeted Activator System or Targeted Repressor System is is designed to bind and modulate.

“Transgene” as used herein refers to a gene or genetic material containing a gene sequence that has been isolated from one organism and is introduced into a different organism. This non-native segment of DNA may retain the ability to produce RNA or protein in the transgenic organism, or it may alter the normal function of the transgenic organism's genetic code. The introduction of a transgene has the potential to change the phenotype of an organism.

“Treatment” or “therapy” or “treating,” when referring to protection of a subject from a disease, means suppressing, repressing, ameliorating, or completely eliminating the disease. Preventing the disease involves administering a composition of the present invention to a subject at risk of having the disease, prior to onset of the disease. Suppressing the disease involves administering a composition of the present invention to a subject after induction of the disease but before its clinical appearance. Repressing or ameliorating the disease involves administering a composition of the present invention to a subject after clinical appearance of the disease. Such treatment will result in a reduction in the incidence, frequency, severity or duration of symptoms of the disease.

As used herein, the term “gene therapy” refers to a method of treating a patient wherein polypeptides or nucleic acid sequences are transferred into cells of a patient such that activity and/or the expression of a particular gene is modulated. In certain embodiments, the expression of the gene is suppressed. In certain embodiments, the expression of the gene is enhanced. In certain embodiments, the temporal or spatial pattern of the expression of the gene is modulated.

“Variant” used herein with respect to a polynucleotide means (i) a portion or fragment of a referenced nucleotide sequence; (ii) the complement of a referenced nucleotide sequence or portion thereof; (iii) a nucleic acid that is at least 60%, 65%, 70%, 75%, 80%, 85%, 90%, 95%, 96%, 97%, 98%, or 99%, or less than 100% identical to a referenced nucleic acid or the complement thereof over its full length or over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 nucleotides; or (iv) a nucleic acid that hybridizes under stringent conditions to the referenced nucleic acid, or complement thereof.

“Variant” with respect to a peptide or polypeptide means a polypeptide that differs in amino acid sequence from a referenced amino acid sequence by the insertion, deletion, and/or conservative substitution of amino acids, such as, at least 60%, 65%, 70%, 75%, 80%, 85%, 90%, 95%, 96%, 97%, 98%, or 99%, or less than 100% identical over its full length or over a region of 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100 amino acids, but which retains at least one biological activity. Representative examples of “biological activity” include the ability to be bound by a specific antibody or polypeptide or to promote a physiological response. Variant can mean a functional fragment thereof. Variant can also mean multiple copies of a polypeptide. The multiple copies can be in tandem or separated by a linker. A conservative substitution of an amino acid, i.e., replacing an amino acid with a different amino acid of similar properties (such as, hydrophilicity, degree and distribution of charged regions) is recognized in the art as typically involving a minor change. These minor changes may be identified, in part, by considering the hydropathic index of amino acids, as understood in the art. Kyte et al., J. Mol. Biol. 157:105-132 (1982). The hydropathic index of an amino acid is based on a consideration of its hydrophobicity and charge. It is known in the art that amino acids of similar hydropathic indexes may be substituted and still retain protein function. In one aspect, amino acids having hydropathic indexes of ±2 are substituted. The hydrophilicity of amino acids may also be used to reveal substitutions that would result in proteins retaining biological function. A consideration of the hydrophilicity of amino acids in the context of a peptide permits calculation of the greatest local average hydrophilicity of that peptide. Substitutions may be performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties.

“Vector” as used herein means a nucleic acid construct capable of directing the delivery or transfer of a polynucleotide sequence to target cells, where it can be replicated or expressed. A vector may contain an origin of replication, one or more regulatory elements, and/or one or more coding sequences. A vector can be integrating or non-integrating. Major types of vectors include, but are not limited to, a plasmids, episomal vectors, viral vectors, cosmids, and artificial chromosomes. A vector may be a DNA or RNA vector. A vector may be a self-replicating extrachromosomal vector. A vector may be a DNA plasmid. The vector may be non-viral. The vector may be viral. Viral vectors include, but are not limited to, adenovirus vector, adeno-associated virus vector, retrovirus vector, or lentivirus vector. A vector may be delivered within a nanoparticle, such as a lipid nanoparticle or polymeric nanoparticle.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear: in the event however of any latent ambiguity, definitions provided herein take precedent over any dictionary or extrinsic definition. Further, unless otherwise required by context, singular terms shall include pluralities and plural terms shall include the singular.

### 2. PRADER-WILLI SYNDROME (PWS)

Prader-Willi Syndrome (PWS) is a rare genetic disease with a prevalence ranging from approximately one in 8,000 to one in 25,000 patients in the U.S. Prader-Willi Syndrome (PWS) is a neuroendocrine and neurobehavioral disorder associated with genetic and epigenetic abnormalities. It is believed that the genetics underlying PWS involve a loss of function of one or more genes on chromosome 15 in humans, in particular, within the PWS region 15q11-13 (Schaaf et al. Nat. Genet. 2013, 45, 1405-09, incorporated herein by reference). Individuals with PWS display mild cognitive impairment and develop a false mental state of starvation that causes hyperphagia beginning in childhood, often resulting in extreme obesity unless strict environmental controls are enforced by caregivers to physically limit access to food. Other symptoms include neonatal hypotonia (weak muscles at birth), growth hormone deficiency, behavioral disturbances such as tantrums, outbursts and self-harm, anxiety, and compulsivity.

While the exact genetic basis of PWS remains unclear, patient mutation profiles have implicated a snoRNA cluster SNORD116 downstream of the SNURF-SNRPN open reading frame controlled by a CpG island imprinting center as a likely contributor to the disease etiology. The genes implicated in PWS are typically expressed only from the paternal copy of chromosome 15, while the PWS genes present on the maternal chromosome are epigenetically silenced. Thus, for example, a patient with paternal deletions or mutations within 15q11.2-13 can present with PWS while retaining functional copies of these genes on the maternal allele. Seventy percent of PWS cases are caused by a large 4-5 Mb deletion on the paternal allele. Twenty-five percent of PWS cases are caused by uniparental maternal disomy (UPD) 15, in which two copies of the maternal chromosome are inherited instead of one copy from each parent. Infrequently, PWS is caused by mutations or microdeletions of the PWS imprinting center (imprinting defects). Exceedingly rare cases of PWS are caused by paternal microdeletions of PWS critical region genes, including SNORD116. Microdeletions or epimutations of the imprinting center account for approximately 2-5% of cases (Bittel and Butler, Expert Rev. Mot. Med. 2005, 7, 1-20; Cassidy and Driscoll, Eur. J. Hum. Genet. 2009, 17, 3-13; each incorporated herein by reference). Thus, the vast majority of individuals with PWS have at least one “good” (unmutated from a DNA sequence perspective) copy of the PWS chromosomal region in every cell. The vast majority of individuals with UPD have two good copies.

There are several imprinted genes within the 15q11-13 locus (the PWS-associated locus), including the paternally-expressed coding genes MAGEL2, NDN and SNURF-SNRPN, and MKRN3, along with numerous noncoding RNAs (ncRNAs), including the snoRNA clusters SNORD115 and SNORD116. As noted above, PWS patient genotypes most commonly consist of deletions within 15q11-13 that encompass both coding and noncoding genes, although a rare subset of genotypes emphasize the snoRNA clusters as having particular influence in the etiology of PWS (Bieth et al., Eur. J. Hum. Genet. 2015, 23, 252-255; de Smith et al., Hum. Mot. Genet. 2009, 18, 3257-3265; Duker et al., Eur. J. Hum. Genet. 2010, 18, 1196-1201; Sahoo et al., Nat. Genet. 2008, 40, 719-721; each incorporated herein by reference). Further evidence suggests that SNURF-SNRPN and downstream ncRNAs, including SPA RNAs and snoRNAs, are processed from a single host transcript that initiates at the imprinting center located in upstream exon 1 of SNRPN (Wu et al., Mol. Cell 2016, 64, 534-548, incorporated herein by reference).

Further known genes and gene products of the PWS-associated locus are as follows: NPAP1 (NCBI gene ID: 23742), SNORD107 (snoRNA) (NCBI gene ID: 91380), SNORD64 (snoRNA cluster) (NCBI gene ID: 347686), SNORD109A (snoRNA) (NCBI gene ID: 338428), SNORD116 or SNORD116@ (snoRNA gene cluster) (NCBI gene ID: 692236), SPA1 (long noncoding RNA transcribed from the SNORD116 gene cluster), SPA2 (long noncoding RNA transcribed from the SNORD116 gene cluster) (for SPA1 and SPA2, see V W et al., Mol. Cell 2016, 64, 534-548, incorporated herein by reference), 116HG (long non-coding RNA transcribed from SNORD116 gene cluster) (Kocher et al., Genes 2017, 8, 358, incorporated herein by reference), SNORD116-1 to 30 (snoRNAs or processed snoRNA derivatives transcribed from the SNORD116 cluster) (SNORD116 1-30 NCBI gene ID Nos: 100033413, 100033414, 100033415, 00033416, 100033417, 100033418, 100033419, 100033420, 100033421, 100033422, 100033423, 100033424, 100033425, 100033426, 100033427, 100033428, 100033429, 100033430, 727708, 100033431, 100033432, 100033433, 100033434, 100033435, 100033436, 100033438, 100033439, 100033820, and 100033821, respectively), SNORD116-30: 100873856, Sno-Inc RNA 1 to 5 (long non coding RNA with snoRNA ends transcribed from the SNORD116 cluster) (Yin et al., Mol. Cell 2012, 48, 219-230, incorporated herein by reference), IPW (long noncoding RNA) (NCBI gene ID: 3653), SNORD115 or SNORD115@ (noncoding snoRNA cluster) (NCBI gene ID: 493919), 115HG (long noncoding snoRNA transcribed from SNORD115 cluster) (Powell et al., Hum. Molec. Genet. 2013, 22, 4318-4328, incorporated herein by reference), SNORD115-1 to 48 (snoRNAs or processed snoRNA derivates transcribed from SNORD115 cluster) (SNORD115 1-48 NCBI gene ID Nos: 338433, 100033437, 100033440, 100033441, 100033442, 100033443, 100033444, 100033445, 100033446, 100033447, 100033448, 100033449, 100033450, 100033451, 100033453, 100033454, 100033455, 100033456, 100033458, 100033460, 100033603, 100033799, 100033800, 100036563, 100033801, 100033802, 100036564, 100036565, 100033803, 100033804, 100033805, 100033806, 100033807, 100033808, 100033809, 100033810, 100033811, 100033812, 100033813, 100033814, 100033815, 100033816, 100033817, 100033818, 100036566, 100873857, 100036567, 100033822, or SNORD109B (snoRNA) (NCBI gene ID: 338429), or SNHG14 (PWS region long transcript) (NCBI gene ID: 104472715). In some embodiments, the gene within the 15q11-13 locus is selected from, for example, SNRPN, SNORD115, SNORD116, SNORD109A, IPW and/or MAGEL2.

Prader-Willi-like syndromes and disorders may include but are not limited to PWS-like syndrome, PWS Type 1 large deletion, PWS Type 2 large deletion, PWS imprinting center mutation or PWS uniparental disomy, PWS microdeletion, atypical deletion encompassing MAGEL2, Scaaf Yang Syndrome (SYS), Chitayat-Hall Syndrome, MAGEL2 disorder, MAGEL2 related disorders, and deletions encompassing Mage12 but not SNORD116. Schaaf-Yang Syndrome (SYS) or MAGEL2-related disorder is a disorder caused by paternally inherited truncating mutations in the MAGEL2 gene (McCarthy et al. Am. J. Med. Genet. A. 2018, 176, 2564-2574, incorporated herein by reference). Chitayat-Hall syndrome can also be cause by paternally inherited truncating mutations in the MAGEL2 gene (Jobling et al. J. Med. Genet. 2018, 55, 316-321, incorporated herein by reference).

MAGEL2 is a maternally imprinted gene in the PWS region. Patients with Schaaf-Yang syndrome (SYS) display many overlapping symptoms as patients with PWS, including neonatal hypotonia, feeding difficulties during infancy, global developmental delay, and intellectual disabilities (McCarthy et al. Am. J. Med. Genet. A. 2018, 176, 2564-2574, incorporated herein by reference). However, there are several features that do not overlap with PWS. Individuals with SYS very commonly present with arthrogryposis or joint contractures which have never been reported in PWS. Additionally, people with SYS have a higher prevalence of Autism spectrum disorder than is observed in people with PWS. MAGEL2 is a monoexonic gene and therefore missense mutations are not subject to nonsense mediated decay. This, along with the additional phenotypes observed in SYS that are not seen in PWS, or in paternal deletions encompassing MAGEL2 but not SNORD116, suggests that the truncated forms of MAGEL2 present in SYS may have dominant negative activity. Although SYS does not completely overlap with PWS, it demonstrates the importance of the loss of function of the MAGEL2 gene to the PWS phenotype.

The DNA targeting systems detailed herein, such as one or more Targeted Activator Systems or one or more Targeted Repressor Systems as described herein, may be used to treat a subject with, for example, any of the following disorders: PWS, PWS-like syndrome, PWS Type 1 large deletion, PWS Type 2 large deletion, PWS imprinting center mutation or PWS uniparental disomy; PWS microdeletion, atypical deletion encompassing MAGEL2, Heterozygous Schaaf-Yang syndrome. Chitayat-Hall syndrome, MAGEL2 disorder, MAGEL2-related disorder.

DNA targeting system(s), including one or more Targeted Activator Systems or one or more Targeted Repressor Systems, can be delivered, such as via gene therapy, to cells of the patients to be treated. The Targeted Activator Systems are designed to target the target regions identified herein as amenable to increasing PWS gene expression through administration of activators. The Targeted Repressor Systems are designed to target the target regions identified herein as amenable to increasing PWS gene expression through administration of repressors. Alternatively, the gene therapy methods of the disclosure can be accomplished by CRISPR/Cas9 based gene editing to incorporate an insertion, deletion and/or substitution in any of the target regions identified herein that eliminates the imprinting (silencing) of the PWS region genes.

The disclosure also contemplates that expression of one or more of the following genes or gene products (including noncoding RNAs) or clusters is upregulated, i.e., increased, in the subject by administration of the DNA targeting system(s) described herein: MKRN3 (gene), MAGEL2 (gene), NDN (gene), C150RF2, SNURF-SNRPN (gene), SNORD107 (snoRNA), SNORD64 (snoRNA cluster), SNORD109A (snoRNA), SNORD116 or SNORD116@ (snoRNA gene cluster), SPA1 (long noncoding RNA transcribed from the SNORD116 gene cluster), SPA2 (long noncoding RNA transcribed from the SNORD118 gene cluster), 116HG (long non-coding RNA transcribed from SNORD116 gene cluster), SNORD116-1 to 30 (snoRNAs transcribed from the SNORD116 cluster), Sno-Inc RNA 1 to 5 (long non coding RNA with snoRNA ends transcribed from the SNORD116 cluster), IPW (long noncoding RNA), SNORD115 or SNORD115@ (noncoding snoRNA cluster), 115HG (long noncoding snoRNA transcribed from SNORD115 cluster), SNORD115-1 to 48 (snoRNAs transcribed from SNORD115 cluster), SNORD109B (snoRNA), or SNHG14 (PWS region long transcript).

In some embodiments, the DNA targeting system as described herein targets a region that results in increased expression of SNORD118, or increased expression of MAGEL2, or both. In some embodiments, a first Targeted Activator System targets a region that results in increased expression of SNORD116 and a second Targeted Activator System targets a region that results in increased expression of MAGEL2. The disclosure contemplates that additional Targeted Activator Systems may be utilized concurrently. In some embodiments, a first Targeted Repressor System targets a region that results in increased expression of SNORD116 and a second Targeted Repressor System targets a region that results in increased expression of MAGEL2. The disclosure contemplates that additional Targeted Repressor Systems may be utilized concurrently. Multiple DNA targeting systems may be utilized concurrently.

The treatment methods as described herein, for PWS, may result in amelioration/reduction of symptoms including, for example, hypotonia, growth hormone deficiency, infantile failure to thrive, global developmental delay, neonatal hypophagia, anxiety, obsessive compulsive disorder, obsessive compulsive-like disorder, intellectual impairment, intellectual disability, hyperphagia, obesity due to hyperphagia, metabolic syndrome secondary to obesity, type 2 diabetes in PWS, behavioral disturbances such as tantrums, outbursts and self-harm, anxiety and compulsivity, and/or skin picking. Other characteristics or symptoms may include small hands, small feet, straight ulnar borders on hands, characteristic facial features: almond shaped eyes, thin upper lip, temperature instability, chronic constipation, decreased gut/intestinal motility, scoliosis, hyperghrelinemia, and/or hypoinsulinemia.

The treatment methods as described herein, for SYS, may result in amelioration of symptoms including neonatal hypotonia, growth hormone deficiency, infantile failure to thrive, global developmental delay, hyperghrelinemia, autism spectrum disorder, infantile respiratory distress, gastroesophageal reflux, chronic constipation, skeletal abnormalities, sleep apnea, temperature instability, and/or arthrogryposis.

### 3. DNA TARGETING SYSTEMS

A “DNA targeting system” as used herein is a system capable of specifically targeting a particular region of DNA and modulating gene expression by binding to that region. Non-limiting examples of these systems are CRISPR-Cas-based systems, zinc finger (ZF)-based systems, and/or transcription activator-like effector (TALE)-based systems. The DNA targeting system may be a nuclease system that acts through mutating or editing the target region (such as by insertion, deletion or substitution) or it may be a system that delivers a functional second polypeptide domain, such as an activator or repressor, to the target region. The DNA targeting system may comprise a Cas protein or a fusion protein, wherein the fusion protein comprises two heterologous polypeptide domains, and wherein the first polypeptide domain comprises a DNA-binding protein. The DNA-binding protein may comprise a Cas protein, a zinc finger protein, or a transcription activator-like effector (TALE) protein. DNA targeting systems are also described in International Patent Application No. PCT/US2021/054292, published as WO/2022/076901, and International Patent Application No. PCT/US2020/54160, published as WO/2021/067878, each of which is incorporated herein by reference.

A “DNA targeting system” may be a Targeted Activator System, or a Targeted Repressor System, or a combination thereof. A “Targeted Activator System” as used herein is a system capable of specifically targeting a particular region of DNA and activating gene expression by binding to that target region. A “Targeted Repressor System” as used herein is a system capable of specifically targeting a particular region of DNA and repressing gene expression by binding to that target region.

As indicated above, each of these systems may comprise a DNA-binding portion or domain, such as a Cas protein with guide RNA, a zinc finger protein (ZF), or a transcription activator-like effector (TALE) protein, that specifically recognizes and binds to a particular target region of a target DNA. The DNA-binding portion (for example, Cas9 protein, ZF, or TALE) can be linked to a second protein domain, such as a polypeptide with transcription activation activity, transcription repression activity, transcription release factor activity, histone modification activity, nuclease activity, nucleic acid association activity, methylase activity, demethylase activity, acetylation activity, or deacetylation activity. For example, the DNA-binding portion can be linked to an activator and thus guide the activator to a specific target region of the target DNA. Similarly, the DNA-binding portion can be linked to a repressor and thus guide the repressor to a specific target region of the target DNA.

Some CRISPR-Cas-based systems can operate to activate or repress expression using the Cas protein alone, not linked to an activator or repressor. For example, a nuclease-null Cas9 can act as a repressor on its own, or a nuclease-active Cas9 can act as an activator when paired with an inactive (dead) guide RNA. In addition, RNA or DNA that hybridizes to a particular target region of the target DNA can be directly linked (covalently or non-covalently) to an activator or repressor.

### 4. CRISPR/CAS9-BASED GENE EDITING SYSTEM

The gene therapy methods of the disclosure can be accomplished by administering a DNA targeting system, such as Targeted Activator System or Targeted Repressor System, that comprises a CRISPR-Cas-based system, which may comprise (a) one or more guide RNAs and (b) one or more Cas polypeptides. In some embodiments, the Cas polypeptides are fusion proteins comprising a Cas protein or fragment or variant thereof, and a second heterologous polypeptide domain. Administration of the DNA targeting system may upregulate expression of one or more genes within the 15q11-13 locus. Alternatively, the gene therapy methods of the disclosure can be accomplished by CRISPR/Cas9 based gene editing to incorporate an insertion, deletion, and/or substitution that eliminates the imprinting (silencing) of the PWS region genes.

CRISPR (Clustered Regularly Interspaced Short Palindromic Repeats)-Cas, refers to RNA-guided endonuclease systems that comprise (a) an RNA portion that guides the endonuclease system to target DNA by hybridizing to a DNA sequence within the target region of the target DNA, and (b) a nuclease portion that binds to and cleaves the target DNA at or near that location. The most commonly used CRISPR-Cas systems are the Type II CRISPR systems, such as CRISPR-Cas9 or CRISPR-Cpf1, in which the nuclease portion is a single enzyme. However, multi-protein nuclease systems, such as the Type I system, can be harnessed for the same purpose. One example of such a Type I multi-protein nuclease complex is described in U.S. Patent Appl. Pub. No. 2018/0334688, incorporated by reference herein in its entirety. Another example of a Cpf1-based complex is described in U.S. Patent Appl. Pub. No. 2019/0151476, incorporated by reference herein in its entirety

Provided herein are genetic constructs for genome editing, genomic alteration, or altering gene expression of a gene, for example, on chromosome 15, for the treatment of PWS, PWS-like syndrome, PWS Type 1 large deletion, PWS Type 2 large deletion, PWS imprinting center mutation or PWS uniparental disomy; PWS microdeletion, atypical deletion encompassing MAGEL2, Heterozygous Schaaf-Yang syndrome, Chitayat-Hall syndrome, MAGEL2 disorder, or MAGEL2-related disorder. The genetic constructs may include at least one gRNA that targets a target region, such as a gene sequence or a regulatory element thereof. The disclosed gRNAs can be included in a CRISPR/Cas9-based gene editing system to target regions in the 15q11-13 imprinted locus, or a regulatory element of a gene within the 15q11-13 locus, causing activation of imprinted genes within the 15q11-13 locus in cells from patients such as PWS patients.

In some embodiments, the at least one gRNA targets an activating regulatory element of a gene within the 15q11-13 locus. In some embodiments, the gRNA may be combined with a Cas9 protein that introduces a mutation in the regulatory element such as an insertion, deletion, and/or substitution, as detailed below, such that the activity of the activating regulatory element is increased, thereby activating expression of the maternal gene within the 15q11-13 locus for the treatment of PWS. In other embodiments, the at least one gRNA targets an inhibitory regulatory element of a gene within the 15q11-13 locus. In some embodiments, the gRNA may be combined with a Cas9 protein that introduces a mutation in the regulatory element such as an insertion, deletion, and/or substitution, as detailed below, such that the activity of the inhibitory regulatory element is decreased, thereby activating expression of the maternal gene within the 15q11-13 locus for the treatment of PWS.

In some embodiments, the gRNA may be combined with a fusion protein that activates transcription, as detailed below, such that the activity of the activating regulatory element is increased, thereby activating expression of an imprinted gene within the 15q11-13 locus for the treatment of PWS.

In other embodiments, the gRNA may be combined with a fusion protein that represses transcription, as detailed below, such that the activity of the inhibitory regulatory element is decreased, thereby activating expression of the maternal gene within the 15q11-13 locus for the treatment of PWS.

A CRISPR/Cas9-based system may be specific for a gene within the 15q11-13 locus or a regulatory element thereof.

“Clustered Regularly Interspaced Short Palindromic Repeats” and “CRISPRs”, as used interchangeably herein, refers to loci containing multiple short direct repeats that are found in the genomes of approximately 40% of sequenced bacteria and 90% of sequenced archaea. The CRISPR system in nature is a microbial nuclease system involved in defense against invading phages and plasmids that provides a form of acquired immunity. The CRISPR loci in microbial hosts contain a combination of CRISPR-associated (Cas) genes as well as non-coding RNA elements capable of programming the specificity of the CRISPR-mediated nucleic acid cleavage. Short segments of foreign DNA, called spacers, are incorporated into the genome between CRISPR repeats, and serve as a “memory” of past exposures.

CRISPR (Clustered Regularly Interspaced Short Palindromic Repeats)-Cas, refers to RNA-guided endonuclease systems that comprise (a) an RNA portion that guides the endonuclease system to target DNA by hybridizing to a DNA sequence within the target region of the target DNA, and (b) a nuclease portion that binds to and cleaves the target DNA at or near that location.

Three classes of CRISPR systems (Types I, II, and III effector systems) are known. The natural Type II effector system carries out targeted DNA double-strand breaks using a complex comprising a single effector enzyme, Cas9, together with a duplex of two RNAs, a crRNA and a tracrRNA. Collectively, the duplex of two RNAs is called the “guide RNA.” A predefined 20 bp portion at the 5′ end of the natural crRNA recognizes its target by complementary base pairing to a DNA sequence of the target DNA. This 20 bp portion may be swapped for a different portion of similar nucleotide length to change the target recognition of the Type II effector system. The CRISPR-Cas systems can target multiple distinct genomic loci by co-expressing a single Cas9 or Cpf1 protein with two or more guide RNA.

An engineered improvement to this system links the two RNAs together, either via chemical covalent linkage, or with a nucleotide linker (such as GAAA), to form a single guide RNA (sgRNA). As explained below, the RNA(s) can be chemically modified to improve stability and reduce degradation in the cellular environment. Type II Cas9 systems and their use in Targeted Activator Systems are described, such as in Perez-Pinera et al., Nat. Methods 2013, 10, 973-976, incorporated herein by reference. Type II Cpf1 systems and their use in Targeted Activator Systems are described, such as in Zhang et al., Protein Cell 2018, 9, 380-383, incorporated herein by reference.

For DNA cleavage, the DNA sequence recognized by the crRNA may also be immediately followed by the protospacer-adjacent motif (PAM), a short sequence recognized by the Cas9. Different Cas9 from different bacteria have differing PAM requirements. For example, the PAM for Streptococcus pyogenes (S. pyogenes) Cas9 (SpCas9) as 5′-NRG-3′, where R is either A or G. Thus, the DNA-targeting mechanism of the type II CRISPR-Cas9 system involves a guide RNA which directs the Cas9 endonuclease to cleave the target DNA in a sequence-specific manner, dependent on the presence of a Protospacer Adjacent Motif (PAM) on the target DNA.

For example, the S. pyogenes Type II system naturally prefers to use an “NGG” sequence, where “N” can be any nucleotide, but also accepts other PAM sequences, such as “NAG” in engineered systems (Hsu et al., Nature Biotechnology 2013 doi:10.1038/nbt.2647, incorporated by reference). Similarly, the Cas9 derived from Neisseria meningitidis (NmCas9) normally has a native PAM of NNNNGATT, but has activity across a variety of PAMs, including a highly degenerate NNNNGNNN PAM (Esvelt et al. Nature Methods 2013 doi:10.1038/nmeth.2681, incorporated by reference).

A Cas9 protein of Staphylococcus aureus (S. aureus) recognizes the sequence motif NNGRR (R=A or G) (SEQ ID NO: 19) and directs cleavage of a target nucleic acid sequence 1 to 10, such as, 3 to 5, bp upstream from that sequence. In certain embodiments, a Cas9 protein of S. aureus recognizes the sequence motif NNGRRN (R=A or G) (SEQ ID NO: 20) and directs cleavage of a target nucleic acid sequence 1 to 10, such as, 3 to 5, bp upstream from that sequence. In certain embodiments, a Cas9 protein of S. aureus recognizes the sequence motif NNGRRT (R=A or G) (SEQ ID NO: 21) and directs cleavage of a target nucleic acid sequence 1 to 10, such as, 3 to 5, bp upstream from that sequence. In certain embodiments, a Cas9 protein of S. aureus recognizes the sequence motif NNGRRV (R=A or G) (SEQ ID NO: 22) and directs cleavage of a target nucleic acid sequence 1 to 10, such as, 3 to 5, bp upstream from that sequence. In the aforementioned embodiments, N can be any nucleotide residue, such as, any of A, G, C, or T. Cas9 proteins can be engineered to alter the PAM specificity of the Cas9 protein.

a. Cas Protein

The CRISPR/Cas9-based gene editing system can include a Cas protein or a Cas fusion protein. The Cas9 protein can be from any bacterial or archaea species, including, but not limited to, Streptococcus pyogenes (also in U.S. Patent Appl. Pub. No. 2019/0127713, incorporated by reference herein in its entirety), Staphylococcus aureus (S. aureus) (also in U.S. 2019/0127713, incorporated by reference herein in its entirety), Streptococcus thermophilus (LMD-9, YP_820832.1), L. innocua (Clip11262, NP_472073.1), Acidovorax avenae, Actinobacillus pleuropneumoniae, Actinobacillus succinogenes, Actinobacillus suis, Actinomyces sp., cycliphilus denitrinfcans, Aminomonas paucivorans, Azospirillum, Bacillus cereus, Bacillus smithii, Bacillus thunngiensis, Bacteroides sp., Blastopirellula manna, Bradyrhizobium sp., Brevibacillus laterosporus, Campylobacter coli, Campylobacterjejuni (subsp. jejuni NCTC 11168, YP_002344900.1), Campylobacter lari, Candidatus puniceispirillum, Clostridium cellulolyticum, Clostrdium perfringens, Corynebacterium accolens, Corynebacterium diphtheria, Corynebactenum matruchotii, Dinoroseobacter shibae, Eubacterium dolichum, Eubacterium ventriosum, gamma proteobacterium, Gluconacetobacter diazotrophicus. Haemophilus parainfluenzae, Haemophilus sputorum, Helicobacter canadensis, Helicobacter cinaedi. Helicobacter mustelae, Ilyobacter polytropus, Kingella kingae, Lactobacillus crispatus, Lactobacillus farciminis, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, N. meningitidis (Z2491, YP_002342100.1), Neisseria sp., Neisseria wadsworthii, Nitratifractor salsuginis, Nitrosomonas sp., Parvibaculum lavamentivorans, Pasteurela multocida, Phascolarctobacterium succinatutens, Ralstonia syzygii, Rhodopseudomonas palustris, Rhodovulum sp., Roseburia intestinalis, Simonsiella muelleri, Sphaerochaeta, Sphingomonas sp., Sporolactobacillus vineae, Staphylococcus lugdunensis, Streptococcus sp., Streptococcus pasteurianus, Subdoligranulum sp., Tistrella mobilis, Treponema sp., or Verminephrobacter eiseniae. CRISPR loci have been identified in more than 40 prokaryotes (See such as, Jansen et al., Mol. Microbiol. 2002, 43, 1565-1575; and Mojica et al., J. Molec. Evolution 2005, 60, 174-182; each incorporated herein by reference) including, but not limited to Aeropyrum, Pyrobaculum, Sulfolobus, Archaeoglobus, Halocarcula, Methanobacterium, Methanococcus, Methanosarcina, Methanopyrus, Pyrococcus, Picrophilus, Thermoplasma, Corynebacterium, Mycobacterium, Streptomyces, Aquifex, Porphyromonas, Chlorobium, Thermus, Bacillus, Listeria, Staphylococcus, Clostridium, Thermoanaerobacter, Mycoplasma, Fusobacterium, Azarcus, Chromobacterium, Neisseria, Nitrosomonas, Desulfovibrio, Geobacter, Myxococcus, Campylobacter, Wolinella, Acinetobacter, Erwinia, Escherichia, Legionella, Methylococcus, Pasteurella, Photobacterum, Salmonella, Xanthomonas, Yersinia, Treponema, and Thermotoga.

In certain embodiments, the Cas9 protein is a Streptococcus pyogenes Cas9 protein (also referred herein as “SpCas9”) or a variant thereof. In certain embodiments, the Cas9 protein is a Staphylococcus aureus Cas9 protein (also referred herein as “SaCas9”) or a variant thereof.

The Cas polypeptide can also comprise a modified form of the Cas polypeptide that retains DNA-targeting activity and is at least 65% identical, preferably at least 70%, 75%, 80%, 85%, 90%, 95%, 96%, 97%, 98%, or at least 99% identical to a naturally occurring Cas protein amino acid sequence. The modified form of the Cas polypeptide can include an amino acid change (such as, deletion, insertion, and/or substitution) that reduces the naturally occurring nuclease activity of the Cas protein. For example, in some instances, the modified form of the Cas protein has less than 50%, less than 40%, less than 30%, less than 20%, less than 10%, less than 5%, or less than 1% of the nuclease activity of the corresponding wild-type Cas polypeptide (US20140068797, incorporated by reference). In some cases, the modified form of the Cas polypeptide has no substantial nuclease activity and is referred to as “nuclease null” or “deactivated” Cas (dCas). For Cas9, this can be accomplished, such as, by mutating one or both of the nuclease domains of Cas9, the HNH domain or the RuvC domain. Each of these nuclease domains is responsible for cleaving one of the two strands of the target DNA. For S. pyogenes cas9, mutations at positions 10 and 840 (such as D10A, H840A) render it nuclease null. For S. aureus Cas9, mutations at positions 10 and 580 (such as D10A, N580A) render it nuclease null.

The ability of a Cas9 protein or a Cas9 fusion protein to recognize a PAM sequence can be determined, such as, using a transformation assay as known in the art. In certain embodiments, the ability of a Cas9 protein or a Cas9 fusion protein to interact with and cleave a target nucleic acid is PAM sequence dependent. A PAM sequence is a sequence in the target nucleic acid. In certain embodiments, cleavage of the target nucleic acid occurs upstream from the PAM sequence. Cas9 proteins from different bacterial species can recognize different sequence motifs (such as, PAM sequences). In certain embodiments, a Cas9 protein of S. pyogenes recognizes the sequence motif NGG and directs cleavage of a target nucleic acid sequence 1 to 10, such as, 3 to 5, bp upstream from that sequence. In certain embodiments, a Cas9 protein of S. thermophilus recognizes the sequence motif NGGNG (SEQ ID NO: 33) and/or NNAGAAW (W=A or T) (SEQ ID NO: 17) and directs cleavage of a target nucleic acid sequence 1 to 10, such as, 3 to 5, bp upstream from these sequences. In certain embodiments, a Cas9 protein of S. mutans recognizes the sequence motif NGG and/or NAAR (R=A or G) (SEQ ID NO: 18) and directs cleavage of a target nucleic acid sequence 1 to 10, such as, 3 to 5 bp, upstream from this sequence. In certain embodiments, a Cas9 protein of S. aureus recognizes the sequence motif NNGRR (R=A or G) (SEQ ID NO: 19) and directs cleavage of a target nucleic acid sequence 1 to 10, such as, 3 to 5, bp upstream from that sequence. In certain embodiments, a Cas9 protein of S. aureus recognizes the sequence motif NNGRRN (R=A or G) (SEQ ID NO: 20) and directs cleavage of a target nucleic acid sequence 1 to 10, such as, 3 to 5, bp upstream from that sequence. In certain embodiments, a Cas9 protein of S. aureus recognizes the sequence motif NNGRRT (R=A or G) (SEQ ID NO: 21) and directs cleavage of a target nucleic acid sequence 1 to 10, such as, 3 to 5, bp upstream from that sequence. In certain embodiments, a Cas9 protein of S. aureus recognizes the sequence motif NNGRRV (R=A or G; V=A or C or G) (SEQ ID NO: 22) and directs cleavage of a target nucleic acid sequence 1 to 10, such as, 3 to 5, bp upstream from that sequence. In the aforementioned embodiments, N can be any nucleotide residue, such as, any of A, G, C, or T. Cas9 proteins can be engineered to alter the PAM specificity of the Cas9 protein.

In certain embodiments, the vector encodes at least one Cas9 protein that recognizes a Protospacer Adjacent Motif (PAM) of either NNGRRT (SEQ ID NO: 21) or NNGRRV (SEQ ID NO: 22). In certain embodiments, the at least one Cas9 protein is an S. aureus Cas9 protein. In certain embodiments, the at least one Cas9 protein is a mutant S. aureus Cas9 protein.

The Cas9 protein can be mutated so that the nuclease activity is inactivated. An inactivated Cas9 protein (“iCas9”, also referred to as “dCas9”) with no endonuclease activity has been targeted to genes in bacteria, yeast, and human cells by gRNAs to silence gene expression through steric hindrance. Exemplary mutations with reference to the S. pyogenes Cas9 sequence to inactivate the nuclease activity include D10A, E762A, H840A, N854A, N863A, and/or D986A. Exemplary mutations with reference to the S. aureus Cas9 sequence to inactivate the nuclease activity include D10A and N580A. In certain embodiments, the Cas9 protein is a mutant S. aureus Cas9 protein.

In certain embodiments, the mutant S. aureus Cas9 protein comprises a D10A mutation. The nucleotide sequence encoding this mutant S. aureus Cas9 protein is set forth in SEQ ID NO: 31.

In certain embodiments, the mutant S. aureus Cas9 protein comprises a N580A mutation. The nucleotide sequence encoding this mutant S. aureus Cas9 protein is set forth in SEQ ID NO: 32.

In some embodiments, the Cas9 protein is a VQR variant. The VQR variant of Cas9 is a mutant with a different PAM recognition, as detailed in Kleinstiver, et al. (Nature 2015, 523, 481-485, incorporated herein by reference).

A polynucleotide encoding a Cas9 protein can be a synthetic polynucleotide. For example, the synthetic polynucleotide can be chemically modified. The synthetic polynucleotide can be codon optimized, such as, at least one non-common codon or less-common codon has been replaced by a common codon. For example, the synthetic polynucleotide can direct the synthesis of an optimized messenger mRNA, such as, optimized for expression in a mammalian expression system, such as, described herein.

Additionally or alternatively, a nucleic acid encoding a Cas9 polypeptide may comprise a nuclear localization sequence (NLS). Nuclear localization sequences are known in the art. An exemplary codon optimized nucleic acid sequence encoding a Cas9 protein of S. pyogenes is set forth in SEQ ID NO: 23. The corresponding amino acid sequence of an S. pyogenes Cas9 protein is set forth in SEQ ID NO: 24.

Exemplary codon optimized nucleic acid sequences encoding a Cas9 protein of S. aureus, and optionally containing nuclear localization sequences (NLSs), are set forth in SEQ ID NOs: 25-29, 34, and 35. Another exemplary codon optimized nucleic acid sequence encoding a Cas9 protein of S. aureus comprises the nucleotides 1293-4451 of SEQ ID NO: 37. An amino acid sequence of an S. aureus Cas9 protein is set forth in SEQ ID NO: 30. An amino acid sequence of an S. aureus Cas9 protein is set forth in SEQ ID NO: 36.

Alternatively or additionally, the CRISPR/Cas9-based gene editing system can include a fusion protein as described herein.

b. Guide RNA (gRNA)

In embodiments wherein the DNA-binding protein comprises a Cas protein, the DNA targeting system may further comprise one or more guide RNAs (gRNA). The CRISPR/Cas-based gene editing system includes at least one gRNA molecule. The at least one gRNA molecule can bind and recognize a target region. The guide RNA is the part of the CRISPR-Cas system that provides DNA targeting specificity to the system. The guide RNA comprises at its 5′ end a DNA-targeting domain that is sufficiently complementary to the target region to be able to hybridize to 10 to 20 nucleotides of the target region of the target DNA, when it is followed by an appropriate Protospacer Adjacent Motif (PAM). It has been reported that the specificity of the CRISPR system can be improved by reducing the length of the DNA-targeting domain in the guide RNA to 17-18 nucleotides (Fu et al. Nat. Biotechnol. 2014, 32, 279-284, incorporated herein by reference). The “target region” or “target sequence” or“protospacer” refers to the region of the target gene to which the CRISPR/Cas9-based gene editing system targets and binds. The portion of the gRNA that targets the target sequence in the genome may be referred to as the “targeting sequence” or “targeting portion” or “targeting domain.” “Protospacer” or “gRNA spacer” may refer to the region of the target gene to which the CRISPR/Cas9-based gene editing system targets and binds: “protospacer” or “gRNA spacer” may also refer to the portion of the gRNA that is complementary to the targeted sequence in the genome. The gRNA may include a gRNA scaffold. A gRNA scaffold facilitates Cas9 binding to the gRNA and may facilitate endonuclease activity. The gRNA scaffold is a polynucleotide sequence that follows the portion of the gRNA corresponding to sequence that the gRNA targets. Together, the gRNA targeting portion and gRNA scaffold form one polynucleotide. The constant region of the gRNA may include the sequence of SEQ ID NO: 1141 (RNA), which is encoded by a sequence comprising SEQ ID NO: 1140 (DNA). For example, the sequence of the full gRNA corresponding to SEQ ID NO: 588 (defined below) may be SEQ ID NO: 1142.

The DNA-targeting domain of the guide RNA does not need to be perfectly complementary to the target region of the target DNA. In example embodiments, the DNA-targeting domain of the guide RNA sequence is at least 80% complementary, preferably at least 85%, 90%, 95%, 96%, 97%, 98%, or at least 99% complementary to (or has 1, 2 or 3 mismatches compared to) the target region over a length of, such as, 10, 11, 12, 13, 14, 15, 16, 17, 18, 19 or 20 nucleotides. For example, the DNA-targeting domain of the guide RNA sequence is at least 80% complementary over at least 18 nucleotides of the target region. The target region may be on either strand of the target DNA.

The portion of the guide RNA that corresponds to the tracrRNA can be variably truncated and a range of lengths has been shown to function in both a system comprising separate RNAs and a system comprising a single-guide RNA. For example, in some embodiments, tracrRNA may be truncated from its 3′ end by at least 1, 2, 3, 4, 5, 6, 7, 8, 9, 10, 15, 20, 25, 30, 35, or 40 nucleotides. In some embodiments, the tracrRNA may be truncated from its 5′ end by at least 1, 2, 3, 4, 5, 6, 7, 8, 9, 10, 15, 20, 21, 22, 23, 24, or 25 nucleotides. Alternatively, the tracrRNA may be truncated from both the 5′ and 3′ end, such as, by at least 1, 2, 3, 4, 5, 6, 7, 8, 9, 10, 15, or 20 nucleotides on the 5′ end and at least 1, 2, 3, 4, 5, 6, 7, 8, 9, 10, 15, 20, 25, 30, 35, or 40 nucleotides on the 3′ end. See, such as, Jinek et al., Science 2012, 337, 816-821; Mali et al., Science 2013, 339, 823-826; Cong et al., Science 2013, 339, 819-823; and Hwang and Fu et al., Nat. Biotechnol. 2013, 31, 227-229; Jinek et al. Elife 2, 2013, e00471, each incorporated by reference).

The guide RNAs are complementary to a target region of the genomic target DNA. For example, they are complementary to a target region within about 100-1000, about 100-900, about 100-800, about 100-700, about 100-600, about 100-500, about 100-400, about 100-300 or about 100-200 bp upstream, or downstream, of the target region identified herein. In some embodiments, the gRNAs target the sense strand. In some embodiments, the gRNAs target the antisense strand.

The guide RNA can be designed to target known transcription response elements (such as, promoters, enhancers, etc.), known upstream activating sequences (UAS), sequences of unknown or known function that are suspected of being able to control expression of the target DNA, etc. In some such cases, the CRISPR-Cas-based DNA targeting system, including Targeted Activator System or Targeted Repressor System, is targeted by the guide RNA to a specific location (i.e., sequence) in the target region of the DNA and exerts locus-specific regulation such as blocking RNA polymerase binding to a promoter (which selectively inhibits transcription activator function), and/or modifying the local chromatin status or epigenetic status (such as modifying the target DNA or proteins associated with the target DNA, such as, nucleosomal histones. In some cases, the changes are transient (such as, transcription repression or activation). In some cases, the changes are inheritable by daughter cells.

The CRISPR/Cas9-based gene editing system includes at least one gRNA. In any of the embodiments, more than one target region can be targeted with 2, 3, 4, 5, or more gRNAs directed to different sites in the same locus of the target DNA. The at least one gRNA may target an activating regulatory element of a gene within the 15q11-13 locus. The at least one gRNA may target an inhibitory regulatory element of a gene within the 15q11-13 locus. The number of gRNAs encoded by a genetic construct (such as a vector) can be at least 1 gRNA, at least 2 different gRNA, at least 3 different gRNA at least 4 different gRNA, at least 5 different gRNA, at least 6 different gRNA, at least 7 different gRNA, at least 8 different gRNA, at least 9 different gRNA, at least 10 different gRNAs, at least 11 different gRNAs, at least 12 different gRNAs, at least 13 different gRNAs, at least 14 different gRNAs, at least 15 different gRNAs, at least 16 different gRNAs, at least 17 different gRNAs, at least 18 different gRNAs, at least 18 different gRNAs, at least 20 different gRNAs, at least 25 different gRNAs, at least 30 different gRNAs, at least 35 different gRNAs, at least 40 different gRNAs, at least 45 different gRNAs, or at least 50 different gRNAs. The number of gRNAs encoded by a presently disclosed vector can be between at least 1 gRNA to at least 50 different gRNAs, at least 1 gRNA to at least 45 different gRNAs, at least 1 gRNA to at least 40 different gRNAs, at least 1 gRNA to at least 35 different gRNAs, at least 1 gRNA to at least 30 different gRNAs, at least 1 gRNA to at least 25 different gRNAs, at least 1 gRNA to at least 20 different gRNAs, at least 1 gRNA to at least 16 different gRNAs, at least 1 gRNA to at least 12 different gRNAs, at least 1 gRNA to at least 8 different gRNAs, at least 1 gRNA to at least 4 different gRNAs, at least 4 gRNAs to at least 50 different gRNAs, at least 4 different gRNAs to at least 45 different gRNAs, at least 4 different gRNAs to at least 40 different gRNAs, at least 4 different gRNAs to at least 35 different gRNAs, at least 4 different gRNAs to at least 30 different gRNAs, at least 4 different gRNAs to at least 25 different gRNAs, at least 4 different gRNAs to at least 20 different gRNAs, at least 4 different gRNAs to at least 16 different gRNAs, at least 4 different gRNAs to at least 12 different gRNAs, at least 4 different gRNAs to at least 8 different gRNAs, at least 8 different gRNAs to at least 50 different gRNAs, at least 8 different gRNAs to at least 45 different gRNAs, at least 8 different gRNAs to at least 40 different gRNAs, at least 8 different gRNAs to at least 35 different gRNAs, 8 different gRNAs to at least 30 different gRNAs, at least 8 different gRNAs to at least 25 different gRNAs, 8 different gRNAs to at least 20 different gRNAs, at least 8 different gRNAs to at least 16 different gRNAs, or 8 different gRNAs to at least 12 different gRNAs. In certain embodiments, the genetic construct (such as, a vector) encodes one gRNA, i.e., a first gRNA, and optionally a Cas9 protein. In certain embodiments, a first genetic construct (such as, a first vector) encodes one gRNA, i.e., a first gRNA, and optionally a Cas9 protein, and a second genetic construct (such as, a second vector) encodes one gRNA, i.e., a second gRNA, and optionally a Cas9 protein.

The gRNA may comprise a “G” at the 5′ end of the targeting domain or complementary polynucleotide sequence, such as as a result of in vitro transcription by a T7 RNA polymerase. The DNA-targeting domain of a gRNA may comprise at least a 10 base pair, at least a 11 base pair, at least a 12 base pair, at least a 13 base pair, at least a 14 base pair, at least a 15 base pair, at least a 16 base pair, at least a 17 base pair, at least a 18 base pair, at least a 19 base pair, at least a 20 base pair, at least a 21 base pair, at least a 22 base pair, at least a 23 base pair, at least a 24 base pair, at least a 25 base pair, at least a 30 base pair, or at least a 35 base pair polynucleotide sequence complementary to the target region DNA sequence followed by a PAM sequence. In certain embodiments, the targeting domain of a gRNA has 19-25 nucleotides in length. In certain embodiments, the targeting domain of a gRNA is 20 nucleotides in length. In certain embodiments, the targeting domain of a gRNA is 21 nucleotides in length. In certain embodiments, the targeting domain of a gRNA is 22 nucleotides in length. In certain embodiments, the targeting domain of a gRNA is 23 nucleotides in length.

The gRNA may target a region within or near the 15q11-q13 locus or a regulatory element thereof. In certain embodiments, the gRNA can target at least one of exons, introns, the promoter region, the enhancer region, or the transcribed region of the gene. In some embodiments, the gRNA targets a gene selected from SNRPN, SNORD115, SNORD116, SPA1, SPA2, and MAGEL2, or a combination thereof. In some embodiments, the gRNA targets a SNRPN activating regulatory element, SNORD115 activating regulatory element, SNORD116 activating regulatory element, or a combination thereof. In some embodiments, the gRNA targets a SNRPN promoter, SNORD115 promoter, SNORD116 promoter, or a combination thereof. The gRNA may include a targeting domain that comprises a polynucleotide sequence corresponding to at least 10, 11, 12, 13, 14, 15, 16, 17, 18, 19, or 20 consecutive bases of any one of SEQ ID NOs: 1-12, or of any one of SEQ ID NOs: 47-86, 91-1122, 591, 585, 685, 697, 750, 752, 763, 771, 196, 812, 861, and 1069 (which are also detailed in International Patent Application No. PCT/US2020/54160, published as WO/2021/067878, incorporated herein by reference; and in TABLE 1, TABLE 2, TABLE 3, TABLE 4) or a complement thereof or an allelic variant thereof. The protospacers and guides represented in TABLE 1 may be useful for targeted delivery of polypeptides that have activator activity. The protospacers and guides represented in TABLE 2 and TABLE 4 may be useful for targeted delivery of polypeptides that have demethylase activity. SEQ ID NOs: 96-516 may be especially useful for targeted delivery of polypeptides that have repressor activity. SEQ ID NOs: 519-580 may be especially useful for targeted delivery of polypeptides that have activator activity when targeted with a pair of gRNAs. The DNA Targeting system may include at least one gRNA corresponding to any one of SEQ ID NOs: 1-12, 47-86, 91-1122, 96-516, 519-580, 591, 585, 685, 697, 750, 752, 763, 771, 196, 812, 861, and 1069, or a truncation, complement, and/or variation thereof. The DNA Targeting system may include at least one gRNA that includes a targeting domain that comprises a polynucleotide sequence corresponding to at least 10, 11, 12, 13, 14, 15, 16, 17, 18, 19, or 20 consecutive bases of any one of SEQ ID NOs: 1-12, 47-86, 91-1122, 591, 585, 685, 697, 750, 752, 763, 771, 196, 812, 861, and 1069. In some embodiments, the at least one gRNA includes a targeting domain that comprises a polynucleotide sequence corresponding to at least 10, 11, 12, 13, 14, 15, 16, 17, 18, 19, or 20 consecutive bases of any one of SEQ ID NOs: 685, 697, 750, 752, 763, 771, 196, 812, and 861, or a truncation, complement, and/or variation thereof. In some embodiments, the at least one gRNA is complementary to a polynucleotide sequence corresponding to at least 10, 11, 12, 13, 14, 15, 16, 17, 18, 19, or 20 consecutive bases of any one of SEQ ID NOs: 53, 55, 64, 65, 68, 70, 196, 291, or 335, or a truncation, complement, and/or variation thereof.

The protospacer sequence (also referred to as target sequence of the gRNA) and the gRNAs in TABLE 1 and TABLE 2 and TABLE 4 bind to (are complementary to) the same sequence (on the opposite strand) of the target DNA. For example, a guide RNA that corresponds to the target region identified by AAAGCATGCGCTACAATAAC (SEQ ID NO: 47) may comprise at least 18 consecutive bases of the sequence AAAGCAUGCGCUACAAUAAC (SEQ ID NO: 591).

TABLE 1
Examples of gRNAs targeting a
gene within or near the 15q11-q13 locus.

| | SEQ | | SEQ | |
|---|---|---|---|---|
| | ID | protospacer | ID | gRNA |
| gRNA name | NO | sequence | NO | sequence |
| PWS_G.3511 | 1 | CTAAGCAGTG | 1052 | CUAAGCAGUG |
| | | GACTAAGGAT | | GACUAAGGAU |
| PWS_G.3549 | 2 | CTACTTTAGA | 1122 | CUACUUUAGA |
| | | TTCTATTGTA | | UUCUAUUGUA |
| PWS_G.4662 | 3 | GCAGCTCAGA | 1054 | GCAGCUCAGA |
| | | AAGTGATGCA | | AAGUGAUGCA |
| PWS_G.4738 | 4 | GCCAGGTTAC | 1058 | GCCAGGUUAC |
| | | TGATTCATAG | | UGAUUCAUAG |
| PWS_G.4862 | 5 | GCGCACTGCA | 1057 | GCGCACUGCA |
| | | GCGCAGACCA | | GCGCAGACCA |
| PWS_G.5117 | 6 | GGAACCAGTC | 588 | GGAACCAGUC |
| | | AGAACAGGTG | | AGAACAGGUG |
| PWS_G.5706 | 7 | GGTCGGCTTC | 1051 | GGUCGGCUUC |
| | | AGGAGGGAAG | | AGGAGGGAAG |
| PWS_G.6131 | 8 | GTGGATAGGT | 1059 | GUGGAUAGGU |
| | | GCGTTCAAGG | | GCGUUCAAGG |
| PWS_G.6318 | 9 | GTTTCAGGCC | 1053 | GUUUCAGGCC |
| | | TGGACTGGGT | | UGGACUGGGU |
| PWS_G.8738 | 10 | ACAGTTCAGG | 1055 | ACAGUUCAGG |
| | | GCATGAGATA | | GCAUGAGAUA |
| PWS_G.9414 | 11 | CAGTTCAGGG | 586 | CAGUUCAGGG |
| | | CATGAGATAA | | CAUGAGAUAA |
| PWS_G.10218 | 12 | GGGCATGAGA | 1056 | GGGCAUGAGA |
| | | TAAGGGCAGT | | UAAGGGCAGU |
| PWS_G.717 | 91 | ACCCACCCAG | 581 | ACCCACCCAG |
| (pat1) | | CAACCACCAG | | CAACCACCAG |
| PWS_G.9876 | 92 | GACAATGACA | 582 | GACAAUGACA |
| (pat3) | | AGGGTTGTGG | | AGGGUUGUGG |
| PWS_G.7517 | 93 | TGCTTGTTTG | 584 | GGUUUUGAGC |
| (pat5) | | CCGCAGTGCA | | AAGCCCUCCU |
| PWS_G.7097 | 94 | TCGTTGTGAC | 586 | UCGUUGUGAC |
| (pat7) | | ACTACTGAGT | | ACUACUGAGU |
| PWS_G.3780 | 95 | CTGCCACGCT | 587 | CUGCCACGCU |
| (pat8) | | GTGACTCAGA | | GUGACUCAGA |

TABLE 2
Examples of target regions
within or near the 15q11-q13 locus.

| | SEQ | | SEQ | |
|---|---|---|---|---|
| | ID | | ID | |
| gRNA name | NO | Protospacer sequence | NO | gRNA sequence |
| PWS_G.81 | 47 | AAAGCATGCGCTACAATAAC | 591 | AAAGCAUGCGCUACAAUAAC |
| PWS_G.595 | 48 | ACAGATGCGTCAGGCATCTC | 606 | ACAGAUGCGUCAGGCAUCUC |
| PWS_G.648 | 49 | ACATCCTCTATTCTGATCAT | 585 | ACAUCCUCUAUUCUGAUCAU |
| PWS_G.1288 | 50 | AGGAGCGGTCAGTGACGCGA | 634 | AGGAGCGGUCAGUGACGCGA |
| PWS_G.1867 | 51 | ATGCCTGACGCATCTGTCTG | 657 | AUGCCUGACGCAUCUGUCUG |
| PWS_G.2532 | 52 | CATAAGCAACCTGGGATCAA | 679 | CAUAAGCAACCUGGGAUCAA |
| PWS_G.2815 | 53 | CCAGGTCATTCCGGTGAGGG | 685 | CCAGGUCAUUCCGGUGAGGG |
| PWS_G.2977 | 54 | CCCTCACCGGAATGACCTGG | 696 | CCCUCACCGGAAUGACCUGG |
| PWS_G.2979 | 55 | CCCTCAGGTCTTCCTATGTG | 697 | CCCUCAGGUCUUCCUAUGUG |
| PWS_G.3026 | 56 | CCGCACATAGGAAGACCTGA | 698 | CCGCACAUAGGAAGACCUGA |
| PWS_G.3107 | 57 | CCTATTGCGGGTGTCTGCGG | 704 | CCUAUUGCGGGUGUCUGCGG |
| PWS_G.3193 | 58 | CCTGTCGCGACACCACAGTT | 709 | CCUGUCGCGACACCACAGUU |
| PWS_G.3268 | 59 | CGAGCGGACAGGATACCATC | 712 | CGAGCGGACAGGAUACCAUC |
| PWS_G.3437 | 60 | CGGTCAGTGACGCGATGGAG | 717 | CGGUCAGUGACGCGAUGGAG |
| PWS_G.3477 | 61 | CGTGGGGGGACCAGTGCATA | 719 | CGUGGGGGGACCAGUGCAUA |
| PWS_G.3482 | 62 | CGTGTGGCGAGGGTACGTGG | 720 | CGUGUGGCGAGGGUACGUGG |
| PWS_G.3858 | 63 | CTGTCGCGACACCACAGTTG | 734 | CUGUCGCGACACCACAGUUG |
| PWS_G.4363 | 64 | GAGCGGACAGGATACCATCG | 750 | GAGCGGACAGGAUACCAUCG |
| PWS_G.4521 | 65 | GATGCGTCAGGCATCTCCGG | 752 | GAUGCGUCAGGCAUCUCCGG |
| PWS_G.4627 | 66 | GCACGCCTGCGCGGCCGCAG | 759 | GCACGCCUGCGCGGCCGCAG |
| PWS_G.4658 | 67 | GCAGCGAGTCTGGCGCAGAG | 761 | GCAGCGAGUCUGGCGCAGAG |
| PWS_G.4670 | 68 | GCAGGCTGGCGCGCATGCTC | 763 | GCAGGCUGGCGCGCAUGCUC |
| PWS_G.4871 | 69 | GCGCCCCAATGCGAGCGGAC | 770 | GCGCCCCAAUGCGAGCGGAC |
| PWS_G.4903 | 70 | GCGGCGACAGTGGGTATTGG | 771 | GCGGCGACAGUGGGUAUUGG |
| PWS_G.5274 | 71 | GGCAAACAAGCACGCCTGCG | 785 | GGCAAACAAGCACGCCUGCG |
| PWS_G.5478 | 72 | GGGACGCGCCCCAATGCGAG | 792 | GGGACGCGCCCCAAUGCGAG |
| PWS_G.5691 | 73 | GGTCAGTGACGCGATGGAGC | 798 | GGUCAGUGACGCGAUGGAGC |
| PWS_G.6001 | 74 | GTCGCGACAGGTCCTATTGC | 808 | GUCGCGACAGGUCCUAUUGC |
| PWS_G.6065 | 75 | GTGACGCGATGGAGCGGGCA | 813 | GUGACGCGAUGGAGGGGGCA |
| PWS_G.6298 | 76 | GTTGTGCCGTTCTGCCCCGA | 819 | GUUGUGCCGUUCUGCCCCGA |
| PWS_G.7011 | 77 | TCCGTGTGGCGAGGGTACGT | 847 | UCCGUGUGGCGAGGGUACGU |
| PWS_G.7296 | 78 | TGACCGAGGCGAGGAGGCTA | 858 | UGACCGAGGCGAGGAGGCUA |
| PWS_G.7302 | 79 | TGACGCATCTGTCTGAGGAG | 859 | UGACGCAUCUGUCUGAGGAG |
| PWS_G.7440 | 80 | TGCCGCTGCTGCAGCGAGTC | 862 | UGCCGCUGCUGCAGCGAGUC |
| PWS_G.7517 | 81 | TGCTTGTTTGCCGCAGTGCA | 583 | UGCUUGUUUGCCGCAGUGCA |
| PWS_G.7751 | 82 | TGTCGCGACACCACAGTTGG | 870 | UGUCGCGACACCACAGUUGG |
| PWS_G.7924 | 83 | TTAGGCGGAGGTAGGTATAT | 876 | UUAGGCGGAGGUAGGUAUAU |
| PWS_G.9680 | 84 | CTCAAATCAATCTCCCTGTA | 941 | CUCAAAUCAAUCUCCCUGUA |
| PWS_G.9721 | 85 | CTGCAATTCGTTCCTTCCAG | 943 | CUGCAAUUCGUUCCUUCCAG |
| PWS_G.10360 | 86 | GTGAGTGGAGGTTGACTAAA | 973 | GUGAGUGGAGGUUGACUAAA |

TABLE 3 identifies the regions of DNA targeted in paternal screens (CRISPRi screens) with a repressor in which gRNA hits were enriched relative to other targeted DNA regions (pat1-pat8), and the regions of DNA targeted in maternal screens (CRISPRa screens) with an activator or demethylase in which gRNA hits were enriched relative to other targeted DNA regions (mat1-mat2 or mat3-mat4, respectively). Guide RNAs within two regions, pat6 and pat8, increased observed levels of SNRPN expression when paired with a Cas9-repressor protein. Guide RNAs within four regions, mat1-mat4, increased observed levels of SNRPN expression when paired with an activator or epigenetic modifier protein.

TABLE 3
Regions of DNA targeted in paternal and maternal screens.

| | | | | | | SNRPN |
|---|---|---|---|---|---|---|
| | | | | | Chr. coordinate | coordinate of |
| | | SEQ | Starting | Ending | of example | example |
| | Chromosomal | ID | SNRPN | SNRPN | validated single | validated single |
| Region | coordinates | NO: | coordinate | coordinate | gRNA | gRNA |
| pat1 | chr15: 25099211- | 1123 | −100858 | −94723 | 25101568 | −98501 |
| | 25105346 | | | | | |
| pat2 | chr15: 25142337- | 1124 | −57732 | −52414 | | |
| | 25147655 | | | | | |
| pat3 | chr15: 25195722- | 1125 | −4347 | −3547 | 25196467 | −3602 |
| | 25196522 | | | | | |
| pat4 | chr15: 25198794- | 1126 | −1275 | +1921 | 25200029 | −40 |
| | 25201990 | | | | | |
| pat5 | chr15: 25203015- | 1127 | +2720 | +23516 | 25211664 | 11595 |
| | 25223585 | | | | | |
| pat6 | chr15: 25223912- | 1128 | +23843 | +24582 | 25224155 | 24086 |
| | 25224651 | | | | | |
| pat7 | chr15: 25224909- | 1129 | +24840 | +35218 | 25227019 | 26950 |
| | 25235287 | | | | | |
| pat8 | chr15: 25235775- | 1130 | +35706 | +36168 | 25235867 | 35798 |
| | 25236237 | | | | | |
| mat1 | chr15: 25074022- | 1131 | −126047 | −125228 | 25074809 | −125260 |
| | 25074874 | | | | | |
| mat1-A | chr15: 25068632- | 1132 | −131437 | −131080 | | |
| | 25068989 | | | | | |
| mat1-B | chr15: 25071154- | 1133 | −128915 | −128215 | | |
| | 25071854 | | | | | |
| mat1-C | chr15: 25076771- | 1134 | −124298 | −122940 | | |
| | 25077129 | | | | | |
| mat2 | chr15: 25108001- | 1135 | −92068 | −91960 | 25108002 | −92067 |
| | 25108109 | | | | | |
| mat3 | chr15: 25199722- | 1136 | −347 | +876 | | |
| | 25200915 | | | | | |
| mat4 | chr15: 25213427- | 1137 | +13358 | +13526 | | |
| | 25213595 | | | | | |

TABLE 4
Examples of regions of DNA targeted in a demethylase screen.

| | | | relative | |
|---|---|---|---|---|
| | | | position | |
| | | | to SNRPN | |
| | | | exon 1 | |
| | | | start | |
| gRNA name | Protospacer sequence | chr15 | (25200069) | gRNA sequence |
| PWS_G.2979 | CCCTCAGGTCTTCCTATGTG | 25199775 | −294 | CCCUCAGGUCUUCCUAUGUG |
| | (SEQ ID NO: 55) | | | (SEQ ID NO: 697) |
| PWS_G.4670 | GCAGGCTGGCGCGCATGCTC | 25200068 | −1 | GCAGGCUGGCGCGCAUGCUC |
| | (SEQ ID NO: 68) | | | (SEQ ID NO: 763) |
| PWS_G.4521 | GATGCGTCAGGCATCTCCGG | 25200151 | 82 | GAUGCGUCAGGCAUCUCCGG |
| | (SEQ ID NO: 65) | | | (SEQ ID NO: 752) |
| PWS_G.4363 | GAGCGGACAGGATACCATCG | 25200391 | 322 | GAGCGGACAGGAUACCAUCG |
| | (SEQ ID NO: 64) | | | (SEQ ID NO: 750) |
| PWS_G.4903 | GCGGCGACAGTGGGTATTGG | 25200896 | 827 | GCGGCGACAGUGGGUAUUGG |
| | (SEQ ID NO: 70) | | | (SEQ ID NO: 771) |
| PWS_G.81 | AAAGCATGCGCTACAATAAC | 25213427 | 13358 | AAAGCAUGCGCUACAAUAAC |
| | (SEQ ID NO: 47) | | | (SEQ ID NO: 591) |
| PWS_G.648 | ACATCCTCTATTCTGATCAT | 25224155 | 24086 | ACAUCCUCUAUUCUGAUCAU |
| | (SEQ ID NO: 49) | | | (SEQ ID NO: 585) |
| PWS_G.306 | AAGCAATATGAAATGTTACC | 25068970 | −131099 | AAGCAAUAUGAAAUGUUACC |
| | (SEQ ID NO: 528) | | | (SEQ ID NO: 1069) |
| PWS_G.6064 | GTGACGCAACACAGACCCCC | 25201415 | 1346 | GUGACGCAACACAGACCCCC |
| | (SEQ ID NO: 291) | | | (SEQ ID NO: 812) |
| PWS_G.2815 | CCAGGTCATTCCGGTGAGGG | 25199993 | −76 | CCAGGUCAUUCCGGUGAGGG |
| | (SEQ ID NO: 53) | | | (SEQ ID NO: 685) |
| PWS_G.7410 | TGCATAGGGATTTTAGGCGG | 25200602 | 533 | UGCAUAGGGAUUUUAGGCGG |
| | (SEQ ID NO: 335) | | | (SEQ ID NO: 861) |
| PWS_G.3052 | CCGGACAGCGACAGGCCCCG | 25201020 | 951 | CCGGACAGCGACAGGCCCCG |
| | (SEQ ID NO: 196) | | | (SEQ ID NO: 196) |

Additional examples of gRNAs are shown in TABLE. The gRNA may be encoded by a polynucleotide comprising a sequence selected from SEQ ID NOs: 1148-1156 or a truncation, complement, and/or variation thereof, or bind to a polynucleotide comprising a sequence selected from SEQ ID NOs: 1148-1156 or a truncation, complement, and/or variation thereof, or comprise a sequence selected from SEQ ID NOs: 1157-1165 or a truncation, complement, and/or variation thereof.

TABLE 5
Targeting portion of
gRNAs targeting the 15q11-13 locus.

| gRNA (DNA) | gRNA (RNA) |
|---|---|
| GAGCGGACAGGATACCATCG | GAGCGGACAGGAUACCAUCG |
| (SEQ ID NO: 1148) | (SEQ ID NO: 1157) |
| TGCATAGGGATTTTAGGCGG | UGCAUAGGGAUUUUAGGCGG |
| (SEQ ID NO: 1149) | (SEQ ID NO: 1158) |
| GCCAGGTCATTCCGGTGAGGG | GCCAGGUCAUUCCGGUGAGGG |
| (SEQ ID NO: 1150) | (SEQ ID NO: 1159) |
| GCCCTCAGGTCTTCCTATGTG | GCCCUCAGGUCUUCCUAUGUG |
| (SEQ ID NO: 1151) | (SEQ ID NO: 1160) |
| GCCGGACAGCGACAGGCCCCG | GCCGGACAGCGACAGGCCCCG |
| (SEQ ID NO: 1152) | (SEQ ID NO: 1161) |
| GGATGCGTCAGGCATCTCCGG | GGAUGCGUCAGGCAUCUCCGG |
| (SEQ ID NO: 1153) | (SEQ ID NO: 1162) |
| GGCAGGCTGGCGCGCATGCTC | GGCAGGCUGGCGCGCAUGCUC |
| (SEQ ID NO: 1154) | (SEQ ID NO: 1163) |
| GGCGGCGACAGTGGGTATTGG | GGCGGCGACAGUGGGUAUUGG |
| (SEQ ID NO: 1155) | (SEQ ID NO: 1164) |
| GGTGACGCAACACAGACCCCC | GGUGACGCAACACAGACCCCC |
| (SEQ ID NO: 1156) | (SEQ ID NO: 1165) |

Single or multiplexed gRNAs can be designed to restore expression of imprinted genes within the 15q11-13 locus. Following treatment with a construct or system as detailed herein, expression of imprinted genes within the 15q11-13 locus can be restored in PWVS patient cells ex vivo. Genetically corrected patient cells may be transplanted into a subject.

i) Dead gRNAs

It has been reported that “dead” guide RNA can be used to guide catalytically active Cas9 to activate transcription without cleaving DNA. Dead guide RNA can be prepared by reducing the length of the DNA-targeting domain to 14-15 nucleotides (nt), and by adding MS2 binding loops into the sgRNA backbone. Guides having a DNA-targeting domain of from 20 bases to 16 bases resulted in indel formation (indicating DNA cleavage), whereas shorter guides (11 bases to 15 bases) did not show detectable levels of indel formation and were able to increase gene expression by as much as 10,000 fold (Dahlman et al., “Orthogonal gene knockout and activation with a catalytically active Cas9 nuclease,” Nat. Biotechnol. 2015, 33, 1159-1 161; correction in Nat. Biotechnol. 2016, 34, 441, each incorporated herein by reference).

Thus, the disclosure contemplates administering a Cas polypeptide with dead guide RNA comprising a DNA-targeting domain about 11-15, or 14-15 bases in length, or a DNA-targeting domain complementary to about 11-15 or 14-15 bases of the target region of the target DNA. The guide RNA may comprise mismatches at the 5′ end of the DNA-targeting domain.

One example system utilizes dead guide RNAs (comprising 14 or 15 bases of DNA-targeting domain) in conjunction with an engineered hairpin aptamer that contains two MS2 domains, which can recruit the MS2:P65:HSF1 (MPH) transcriptional activation complex to the target locus (Liao et al., Cell 2017, 171, 1495-1507, incorporated by reference).

ii) Modifications to gRNA

The activity, stability, or other characteristics of gRNAs can be altered through the incorporation of certain modifications. As one example, transiently expressed or delivered nucleic acids can be prone to degradation by, such as, cellular nucleases. Accordingly, the gRNAs described herein can contain one or more modified nucleosides or nucleotides which introduce stability toward nucleases. In addition, certain modified gRNAs described herein can exhibit a reduced innate immune response when introduced into cells. Such modifications include, without limitation, (a) alteration of the backbone linkage, such as, replacement, of one or both of the non-linking phosphate oxygens and/or of one or more of the linking phosphate oxygens in the phosphodiester backbone linkage; (b) alteration, such as, replacement, of the sugar, or a constituent of the ribose sugar, such as, of the 2′ hydroxyl on the ribose sugar; (c) replacement of the phosphate moiety with “dephospho” linkers; (d) modification or replacement of a naturally occurring nucleobase; (e) replacement or modification of the ribose-phosphate backbone; (f) modification of the 3′ end or 5′ end of the oligonucleotide, such as, removal, modification or replacement of a terminal phosphate group, capping, or conjugation of a moiety, and any combinations thereof. For example, a modified guide RNA may comprise one or more modified sugars and one or more modified backbone linkage. In other embodiments, a modified guide RNA may comprise one or more modified sugars and one or more modified nucleobases. Modifications, such as base, sugar or backbone linkages discussed in this section, can be included at every position or just some positions within a gRNA sequence including, without limitation at or near the 5′ end (such as, within 1-10, 1-5, 1-4, 1-3, or 1-2 nucleotides of the 5′ end) and/or at or near the 3′ end (such as, within 1-10, 1-5, 1-4, 1-3, or 1-2 nucleotides of the 3′ end). For example, three positions at the 5′ end and three positions at the 3′ end may be modified. In some cases, modifications are positioned within functional motifs, such as the repeat-anti-repeat duplex of a Cas9 gRNA, a stem loop structure of a Cas9 or Cpfl gRNA, and/or a targeting domain of a gRNA.

As one example, the 5′ end of a gRNA can include a eukaryotic mRNA cap structure or cap analog (such as, a G(5)ppp(5′)G cap analog, a m7G(5)ppp(5)G cap analog, or a 3′-O-Me-m7G(5)ppp(5′)G anti reverse cap analog (ARCA)). The 5′ end of the gRNA can lack a 5′ triphosphate. The 3′ terminal U ribose can be modified by oxidizing the two terminal hydroxyl groups of the U ribose to aldehyde groups with a concomitant opening of the ribose ring to afford a modified nucleoside. The 3′terminal U ribose can be modified with a 2′3′ cyclic phosphate.

Guide RNAs can contain modified nucleotides such as modified uridines, such as, 5-(2-amino)propyl uridine, and 5-bromo uridine, modified adenosines and guanosines, such as, with modifications at the 8-position, such as, 8-bromo guanosine, a deaza nucleotide, such as, 7-deaza-adenosine, or O- and N-alkylated nucleotides, such as, N6-methyl adenosine, or a modified nucleotide which is multicyclic (such as, tricyclo; and “unlocked” forms, such as glycol nucleic acid (GNA) (such as, R-GNA or S-GNA, where ribose is replaced by glycol units attached to phosphodiester bonds), orthreose nucleic acid (TNA, where ribose is replaced with α-L-threofuranosyl-(3′→2)). In example embodiments, one or more or all of the nucleotides in a gRNA are deoxynucleotides. In such embodiments, guide RNAs that comprise RNA-DNA-combinations are still referred to as guide RNA.

Sugar-modified ribonucleotides can be incorporated into the gRNA, such as, wherein the 2′ OH-group is replaced by another group. Example groups include H, —OR, —R (wherein R can be, such as, alkyl, cycloalkyl, aryl, aralkyl, heteroaryl or sugar), halo, —F, —Br, —Cl or —I, —SH, —SR (wherein R can be, such as, alkyl, cycloalkyl, aryl, aralkyl, heteroaryl or sugar), -arabino, F-arabino, amino (wherein amino can be, such as, NH2; alkylamino, dialkylamino, heterocyclyl, arylamino, diarylamino, heteroarylamino, diheteroarylamino, or amino acid); or cyano (—CN). One or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, such as, 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2-O-methoxyethyl-5-methylcytidine (m5Ceo), and any combinations thereof. Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

The phosphate backbone can be modified, such as, with a phosphothioate (PhTx) group or phosphonoacetate, thiophosphonoacetate, methylphosphonate, boranophosphate, or phosphorodithioate.

Guide RNAs can also include “locked” nucleic acids (LNA) in which the 2′ OH-group can be connected, such as, by a C1-8 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, such as, NH2; alkylamino, dialkylamino, heterocyclyl, arylamino, diarylamino, heteroarylamino, or diheteroarylamino, ethylenediamine, or polyamino) and aminoalkoxy or O(CH2)n-amino (wherein amino can be, such as, NH2; alkylamino, dialkylamino, heterocyclyl, arylamino, diarylamino, heteroarylamino, or diheteroarylamino, ethylenediamine, or polyamino).

As one example, gRNA having 2′-O-methyl (M) incorporated at three terminal nucleotides at both the 5′ and 3′ ends, generally exhibits improved stability against nucleases and also improved base pairing thermostability. As another example, gRNA having 2′-O-methyl-3′-phosphorothioate (MS) or 2′-O-methyl-3′-thioPACE (MSP) incorporated at three terminal nucleotides at both the 5′ and 3′ ends exhibits improved stability against nucleases.

### 5. ZINC FINGER (ZF)-BASED SYSTEMS

A DNA targeting system, such as a Targeted Activator System or Targeted Repressor System, can comprise a zinc finger (ZF)-based system.

By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but are not limited to, C2H2 zinc fingers, C3H zinc fingers, and C4 zinc fingers. Each finger typically contacts and selectively binds to three base pairs of DNA. Combining different zinc fingers together allows production of sequence-specific ZFBD. A “designed” zinc finger domain is a domain not occurring in nature whose design/composition results principally from rational criteria, such as, application of substitution rules and computerized algorithms for processing information in a database storing information of existing ZFP designs and binding data. See, for example, Kim and Kini, “Engineering and Application of Zinc Finger Proteins and TALEs for Biomedical Research,” Mot. Cells 2017, 40, 533-541; U.S. Pat. Nos. 6,140,081; 6,453,242; and 6,534,261; see also WO 98/53058; WO 98/53059; WO 98/53060; WO 02/016536 and WO03/016496, each incorporated herein by reference. A “selected” zinc finger domain is a domain not found in nature whose production results primarily from an empirical process such as phage display, interaction trap or hybrid selection. ZFBD can be fused to a nuclease, such as the Fokl nuclease, to form a zinc finger nuclease (ZFN). ZFBD can also be fused to an activator or repressor.

Thus, a ZF-Based System may comprise a ZFN or a fusion protein comprising (a) a ZF targeting a target region, or a variant thereof, and (b) an activator, or a variant thereof. A ZF-Based System may comprise a fusion protein comprising (a) a ZF targeting a target region, or a variant thereof and (b) a repressor, or a variant thereof. Alternatively, the ZF can be linked to the activator or repressor through reversible or irreversible covalent linkage or through a non-covalent linkage.

### 6. TRANSCRIPTION ACTIVATOR-LIKE EFFECTOR (TALE)-BASED SYSTEMS

A DNA targeting system, such as a Targeted Activator System or Targeted Repressor System, can comprise a transcription activator-like effector-(TALE)-based system.

A TALE is a “transcription activator-like effector DNA binding domain,” “TAL effector DNA binding domain,” or “TALE DNA binding domain” that is the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on modules consisting of repetitive sequences of 33-35 amino acid repeats, which comprise polymorphisms at select repeat positions, usually the 12th and 13th position, called repeat variable-diresidues (RVD). These RVD may determine the nucleotide specificity of each module. Combining these modules allows production of sequence-specific TALEs. TALEs are described in greater detail, for example, in U.S. Patent Application No. 2011/0145940, Kim and Kini (Mol. Cells 2017, 40, 533-541), and Moore et al. (ACS Synth Biol. 2014, 3, 708-716), each incorporated herein by reference. A TALE can be fused to a nuclease, such as the FokI nuclease, to form a TALE nuclease (TALEN). A TALE can also be fused to an activator or repressor.

Thus, a TALE-Based System may comprise a TALEN or a fusion protein comprising (a) a TALE targeting a target region, or a variant thereof and (b) an activator, or a variant thereof. A TALE-Based System may comprise a fusion protein comprising (a) a TALE targeting a target region, or a variant thereof and (b) a repressor, or a variant thereof. Alternatively, the TALE can be linked to the activator or repressor through reversible or irreversible covalent linkage or through a non-covalent linkage.

### 7. FUSION PROTEIN

Polypeptides may be linked to a second polypeptide domain such as, for example, an activator or repressor, to form a fusion protein. The fusion protein can comprise two heterologous polypeptide domains. The first polypeptide domain comprises a DNA-binding protein such as a ZFBD, TALE, or Cas polypeptide, as detailed above. The first polypeptide domain is fused to at least one second polypeptide domain. The second heterologous polypeptide domain may have an activity such as transcription activation activity, transcription repression activity, transcription release factor activity, histone modification activity, nuclease activity, nucleic acid association activity, methylase activity, demethylase activity, acetylation activity, or deacetylation activity.

The linkage to the second polypeptide domain can be through reversible or irreversible covalent linkage or through a non-covalent linkage, as long as the linker does not interfere with the function of the second polypeptide domain. For example, a ZFBD, TALE or Cas polypeptide can be linked to a second polypeptide domain as part of a fusion protein. As another example, they can be linked through reversible non-covalent interactions such as avidin (or streptavidin)-biotin interaction, histidine-divalent metal ion interaction (such as, Ni, Co, Cu, Fe), interactions between multimerization (such as, dimerization) domains, or glutathione S-transferase (GST)-glutathione interaction. As yet another example, they can be linked covalently but reversibly with linkers such as dibromomaleimide (DBM) or amino-thiol conjugation.

The second polypeptide domain may be at the C-terminal end of the first polypeptide domain, or at the N-terminal end of the first polypeptide domain, or a combination thereof. The fusion protein may include one second polypeptide domain, or two of the second polypeptide domains. For example, the fusion protein may include a second polypeptide domain at the N-terminal end as well as a second polypeptide domain at the C-terminal end of the first polypeptide domain. The two second polypeptide domains may be the same or different. In other embodiments, the fusion protein may include more than one (for example, two or three) second polypeptide domains in tandem.

In example embodiments, a fusion protein comprising (a) nuclease-active Cas9, a nuclease null Cas9 (dCas9), or a ZFBD, or a TALE linked to (b) an activator or repressor can be used to modulate gene expression (see examples of activators or repressors below). The fusion proteins optionally include a linker between the dCas9 (or ZF DNA-binding domain or TALE DNA-binding domain) and the activator or repressor. Suitable linkers include short stretches of amino acids (such as 2-20 amino acids) and are typically flexible (i.e. comprising amino acids with high degree of freedom such as glycine, alanine and serine). In example embodiments, the linker comprises one or more units consisting of GGGS or GGGGS, such as two, three, four or more repeats of the GGGS or GGGGS unit. Other linker sequences known in the art can also be used.

In some embodiments, the fusion protein includes a Cas9 protein or a mutated Cas9 protein, fused to a second polypeptide domain that has an activity such as transcription activation activity, transcription repression activity, transcription release factor activity, histone modification activity, nuclease activity, nucleic acid association activity, methylase activity, demethylase activity, acetylation activity, or deacetylation activity.

a. Transcription Activation Activity

The second polypeptide domain can have transcription activation activity, i.e., a transactivation domain. For example, gene expression of endogenous mammalian genes, such as human genes, can be achieved by targeting a fusion protein of dCas9 and a transactivation domain to mammalian promoters via combinations of gRNAs. The transactivation domain can include a VP 16 protein, multiple VP 16 proteins, such as a VP48 domain or VP64 domain, or p65 domain of NF kappa B transcription activator activity. For example, the fusion protein may be dCas9-VP64. In some embodiments, the fusion protein may be VP64-dCas9-VP64 (SEQ ID NO: 43 or a polypeptide at least 70%, 75%, 80%, 85%, 90%, 95%, 96%, 97%, 98%, or at least 99% identical thereto, encoded by the polynucleotide of SEQ ID NO: 44 or a polynucleotide at least 70%, 75%, 80%, 85%, 90%, 95%, 96%, 97%, 98%, or at least 99% identical thereto). A transcription activation domain may include p300, such as p300-core. A fusion protein that activates transcription may also include dCas9-p300, such as the polypeptide of SEQ ID NO: 45 or SEQ ID NO: 46, or a polypeptide at least 70%, 75%, 80%, 85%, 90%, 95%, 96%, 97%, 98%, or at least 99% identical thereto.

Non-limiting examples of activators may include: VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2): KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

In some embodiments, the second polypeptide domain comprises Tet1 or a variant thereof. One variant of Tet1 is known as Tet1CD or Tet1c (Ten-eleven translocation methylcytosine dioxygenase 1; polynucleotide sequence SEQ ID NO: 1138: amino acid sequence SEQ ID NO: 1139). Another variant of Tet1 is known as Tetv4. Tetv4 is detailed in, for example, Nuflez et al. Cell 2021, 184, 2503-2519, incorporated herein by reference. In some embodiments, the second polypeptide domain comprises Tet1, Tet1c, or Tet1v4. Tet1c may comprise a polypeptide having an amino acid sequence of SEQ ID NO: 1139, encoded by a polynucleotide comprising the sequence of SEQ ID NO: 1138, or a polypeptide or polynucleotide at least 70%, 75%, 80%, 85%, 90%, 95%, 96%, 97%, 98%, or at least 99% identical thereto. Tet1v4 may comprise a polypeptide having an amino acid sequence of SEQ ID NO: 1166, encoded by a polynucleotide comprising the sequence of SEQ ID NO: 1167, or a polypeptide or a polynucleotide at least 70%, 75%, 80%, 85%, 90%, 95%, 96%, 97%, 98%, or at least 99% identical thereto. A Tet1c-dCas9 fusion protein may comprise a polypeptide having an amino acid sequence of SEQ ID NO: 1168 or 1172, encoded by a polynucleotide comprising the sequence of SEQ ID NO: 1169 or 1173, respectively, or a polypeptide or a polynucleotide at least 70%, 75%, 80%, 85%, 90%, 95%, 96%, 97%, 98%, or at least 99% identical thereto. A Tet1v4-dCas9 fusion protein may comprise a polypeptide having an amino acid sequence of SEQ ID NO: 1170 or 1174, encoded by a polynucleotide comprising the sequence of SEQ ID NO: 1171 or 1175, respectively, or a polypeptide or a polynucleotide at least 70%, 75%, 80%, 85%, 90%, 95%, 96%, 97%, 98%, or at least 99% identical thereto.

b. Transcription Repression Activity

The second polypeptide domain can have transcription repression activity. The second polypeptide domain can have a Kruppel associated box activity, such as a KRAB domain, ERF repressor domain activity, MxII repressor domain activity, SID4X repressor domain activity, Mad-SID repressor domain activity, or TATA box binding protein activity. For example, the fusion protein may be dCas9-KRAB (polynucleotide sequence SEQ ID NO: 87; protein sequence SEQ ID NO: 88). KRAB may comprise a polypeptide having the amino acid sequence of SEQ ID NO: 1176, encoded by a polynucleotide comprising the sequence of SEQ ID NO: 1177, or a polypeptide or a polynucleotide at least 70%, 75%, 80%, 85%, 90%, 95%, 96%, 97%, 98%, or at least 99% identical thereto.

Non-limiting examples of repressors may include: KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARIDIB/PLU-1, JARIDIC/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hdal, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

c. Transcription Release Factor Activity

The second polypeptide domain can have transcription release factor activity. The second polypeptide domain can have eukaryotic release factor 1 (ERF1) activity or eukaryotic release factor 3 (ERF3) activity.

d. Histone Modification Activity

The second polypeptide domain can have histone modification activity. The second polypeptide domain can have histone deacetylase, histone acetyltransferase, histone demethylase, or histone methyltransferase activity. The histone acetyltransferase may be p300 or CREB-binding protein (CBP) protein, or fragments thereof, such as p300-core. For example, the fusion protein may be dCas9-p300.

e. Nuclease Activity

The second polypeptide domain can have nuclease activity that is different from the nuclease activity of the Cas9 protein. A nuclease, or a protein having nuclease activity, is an enzyme capable of cleaving the phosphodiester bonds between the nucleotide subunits of nucleic acids. Nucleases are usually further divided into endonucleases and exonucleases, although some of the enzymes may fall in both categories. Well known nucleases include deoxyribonuclease and ribonuclease.

f. Nucleic Acid Association Activity

The second polypeptide domain can have nucleic acid association activity or nucleic acid binding protein-DNA-binding domain (DBD). A DBD is an independently folded protein domain that contains at least one motif that recognizes double- or single-stranded DNA. A DBD can recognize a specific DNA sequence (a recognition sequence) or have a general affinity to DNA. A nucleic acid association region may be selected from helix-turn-helix region, leucine zipper region, winged helix region, winged helix-turn-helix region, helix-loop-helix region, immunoglobulin fold, B3 domain, Zinc finger, HMG-box, Wor3 domain, TAL effector DNA-binding domain.

g. Methylase Activity

The second polypeptide domain can have methylase activity, which involves transferring a methyl group to DNA, RNA, protein, small molecule, cytosine or adenine. In some embodiments, the second polypeptide domain includes a DNA methyltransferase.

h. Demethylase Activity

The second polypeptide domain can have demethylase activity. The second polypeptide domain can include an enzyme that removes methyl (CH3-) groups from nucleic acids, proteins (in particular histones), and other molecules. Alternatively, the second polypeptide can convert the methyl group to hydroxymethylcytosine in a mechanism for demethylating DNA. The second polypeptide can catalyze this reaction. For example, the second polypeptide that catalyzes this reaction can be Tet1. In some embodiments, the second polypeptide domain comprises Tet1 or a variant thereof. One variant of Tet1 is known as Tet1CD or Tet1c (Ten-eleven translocation methylcytosine dioxygenase 1; polynucleotide sequence SEQ ID NO: 1138; amino acid sequence SEQ ID NO: 1139). Another variant of Tet1 is known as Tetv4. Tetv4 is detailed in, for example, Nunez et al. Cell 2021, 184, 2503-2519, incorporated herein by reference. In some embodiments, the second polypeptide domain comprises Tet1, Tet1c, or Tet1v4. Tet1c may comprise a polypeptide having an amino acid sequence of SEQ ID NO: 1139, encoded by a polynucleotide comprising the sequence of SEQ ID NO: 1138, or a polypeptide or polynucleotide at least 70%, 75%, 80%, 85%, 90%, 95%, 96%, 97%, 98%, or at least 99% identical thereto. Tet1v4 may comprise a polypeptide having an amino acid sequence of SEQ ID NO: 1166, encoded by a polynucleotide comprising the sequence of SEQ ID NO: 1167, or a polypeptide or a polynucleotide at least 70%, 75%, 80%, 85%, 90%, 95%, 96%, 97%, 98%, or at least 99% identical thereto. A Tet1c-dCas9 fusion protein may comprise a polypeptide having an amino acid sequence of SEQ ID NO: 1168 or 1172, encoded by a polynucleotide comprising the sequence of SEQ ID NO: 1169 or 1173, respectively, or a polypeptide or a polynucleotide at least 70%, 75%, 80%, 85%, 90%, 95%, 96%, 97%, 98%, or at least 99% identical thereto. A Tet1v4-dCas9 fusion protein may comprise a polypeptide having an amino acid sequence of SEQ ID NO: 1170 or 1174, encoded by a polynucleotide comprising the sequence of SEQ ID NO: 1171 or 1175, respectively, or a polypeptide or a polynucleotide at least 70%, 75%, 80%, 85%, 90%, 95%, 96%, 97%, 98%, or at least 99% identical thereto.

i. Regulation of Gene Expression Using Cas Polypeptides Alone

A Targeted Activator System or Targeted Repressor System can comprise a Cas polypeptide alone, not linked to an activator or repressor. Catalytically active Cas polypeptides or nuclease null Cas polypeptides can be administered with a dead guide RNA to activate gene expression as described above. Cas polypeptides with reduced nuclease activity can also be administered with normal guide RNA to repress gene expression as described in Qi et al. Cell 2013, 152, 1173-1183, incorporated herein by reference.

### 8. REPAIR PATHWAYS

The DNA Targeting System may target a regulatory element of a gene in the PWS-associated locus and alter its activity by introducing a mutation in the regulatory element. In some embodiments, the at least one gRNA targets an inhibitory regulatory element of a gene within the 15q11-13 locus. In such embodiments, the gRNA may be combined with a Cas9 protein that introduces a mutation in the regulatory element such as an insertion, deletion and/or substitution, such that activity of the inhibitory regulatory element is decreased, thereby activating expression of the gene within the 15q11-13 locus for the treatment of PWS. In other embodiments, the at least one gRNA targets an activating regulatory element of a gene within the 15q11-13 locus. In such embodiments, the gRNA may be combined with a Cas9 protein that introduces a mutation in the regulatory element such as an insertion, deletion and/or substitution, such that activity of the activating regulatory element is increased, thereby activating expression of the gene within the 15q11-13 locus for the treatment of PWS.

A nuclease system, such as a CRISPR/Cas9-based gene editing system, may be used to introduce site-specific single or double strand breaks at targeted regions of genomic loci, such as a regulatory element of a gene within the 15q11-13 locus. Site-specific breaks are created when any of the nuclease-based gene editing systems described herein binds to a target DNA sequences, thereby permitting cleavage of the target DNA. This DNA cleavage may stimulate the natural DNA-repair machinery, leading to one of two possible repair pathways: homology-directed repair (HDR) or the non-homologous end joining (NHEJ) pathway.

a. Homology-Directed Repair (HDR)

Restoration of protein expression from a gene within the 15q11-13 locus may involve homology-directed repair. A donor template may be administered to a cell that has been treated with a nuclease system to induce a single- or double-stranded DNA break. The donor template may include a nucleotide sequence encoding a mutated version of a regulatory element (an inhibitory regulatory element or an activating regulatory element) of a gene within the 15q11-13 locus. Mutations may include, for example, nucleotide substitutions, insertions, deletions, or a combination thereof. For example, introduced mutation(s) into the inhibitory regulatory element of a gene within the 15q11-13 locus may reduce the transcription of or binding to the inhibitory regulatory element, thereby activating expression of the gene within the 15q11-13 locus for the treatment of PWS.

b. Non-Homologous End Joining (NHEJ)

Restoration of protein expression from a gene within the 15q11-13 locus may be through template-free NHEJ-mediated DNA repair. In certain embodiments, NHEJ is a nuclease mediated NHEJ, which in certain embodiments, refers to NHEJ that is initiated by a Cas9 protein that cuts double stranded DNA. The method comprises administering a nuclease system disclosed herein, such as a CRISPR/Cas9-based gene editing system, or a composition comprising thereof to a subject for genome editing.

Nuclease mediated NHEJ may mutate a regulatory element (an inhibitory regulatory element or an activating regulatory element) of a gene within the 15q11-13 locus. Nuclease mediated NHEJ may offer several potential advantages over the HDR pathway. For example, NHEJ does not require a donor template, which may cause nonspecific insertional mutagenesis. In contrast to HDR, NHEJ operates efficiently in all stages of the cell cycle and therefore may be effectively exploited in both cycling and post-mitotic cells, such as muscle fibers. This provides a robust, permanent gene restoration alternative to oligonucleotide-based exon skipping or pharmacologic forced read-through of stop codons and could theoretically require as few as one drug treatment. NHEJ-based mutation of a regulatory element using a CRISPR/Cas9-based gene editing system, as well as other engineered nucleases including meganucleases and zinc finger nucleases, may be combined with other existing ex vivo and in vivo platforms for cell- and gene-based therapies, in addition to the plasmid electroporation approach described here. For example, delivery of a CRISPR/Cas9-based gene editing system by mRNA-based gene transfer or as purified cell permeable proteins could enable a DNA-free genome editing approach that would circumvent any possibility of insertional mutagenesis.

### 9. GENETIC CONSTRUCTS

The DNA targeting system may be encoded by or comprised within one or more genetic constructs. The DNA targeting system may comprise one or more genetic constructs. Further provided herein is an isolated polynucleotide sequence encoding the DNA targeting system detailed herein. The polynucleotide may be DNA or RNA or a combination thereof. The genetic construct may be non-viral. The genetic construct, such as a plasmid or expression vector, may comprise a nucleic acid that encodes the DNA targeting system or at least one component thereof. In certain embodiments, a genetic construct encodes one gRNA molecule, i.e., a first gRNA molecule, and optionally a Cas9 molecule or fusion protein. In some embodiments, a genetic construct encodes two gRNA molecules, i.e., a first gRNA molecule and a second gRNA molecule, and optionally a Cas9 molecule or fusion protein. In some embodiments, a first genetic construct encodes one gRNA molecule, i.e., a first gRNA molecule, and optionally a Cas9 molecule or fusion protein, and a second genetic construct encodes one gRNA molecule, i.e., a second gRNA molecule, and optionally a Cas9 molecule or fusion protein. In some embodiments, a first genetic construct encodes one gRNA molecule and one donor sequence, and a second genetic construct encodes a Cas9 molecule or fusion protein. In some embodiments, a first genetic construct encodes one gRNA molecule and a Cas9 molecule or fusion protein, and a second genetic construct encodes one donor sequence.

Genetic constructs may include polynucleotides such as vectors and plasmids. The genetic construct may be a linear minichromosome including centromere, telomeres, or plasmids or cosmids. The vector may be an expression vectors or system to produce protein by routine techniques and readily available starting materials including Sambrook et al., Molecular Cloning and Laboratory Manual. Second Ed., Cold Spring Harbor (1989), which is incorporated fully by reference. The construct may be recombinant. The genetic construct may be part of a genome of a recombinant viral vector, including recombinant lentivirus, recombinant adenovirus, and recombinant adenovirus associated virus. The genetic construct may comprise regulatory elements for gene expression of the coding sequences of the nucleic acid. The regulatory elements may be a promoter, an enhancer, an initiation codon, a stop codon, or a polyadenylation signal.

The genetic construct may comprise heterologous nucleic acid encoding the DNA targeting system such as a CRISPR/Cas-based gene editing system and may further comprise an initiation codon, which may be upstream of the DNA targeting system coding sequence, and a stop codon, which may be downstream of the DNA targeting system coding sequence. The genetic construct may include more than one stop codon, which may be downstream of the DNA targeting system coding sequence. In some embodiments, the genetic construct includes 1, 2, 3, 4, or 5 stop codons. In some embodiments, the genetic construct includes 1, 2, 3, 4, or 5 stop codons downstream of the sequence encoding the donor sequence. A stop codon may be in-frame with a coding sequence in the DNA targeting system. For example, one or more stop codons may be in-frame with the donor sequence of a CRISPR/Cas-based gene editing system. The genetic construct may include one or more stop codons that are out of frame of a coding sequence in the DNA targeting system. For example, one stop codon may be in-frame with the donor sequence, and two other stop codons may be included that are in the other two possible reading frames. A genetic construct may include a stop codon for all three potential reading frames. The initiation and termination codon may be in frame with the DNA targeting system coding sequence.

The vector may also comprise a promoter that is operably linked to the DNA targeting system coding sequence. In some embodiments, the promoter is operably linked to a polynucleotide encoding a CRISPR/Cas-based genome editing system. The promoter may be a constitutive promoter, an inducible promoter, a repressible promoter, or a regulatable promoter. The promoter may be a ubiquitous promoter. The promoter may be a tissue-specific promoter. The tissue specific promoter may be a muscle specific promoter. The tissue specific promoter may be a skin specific promoter. The DNA targeting system may be under the light-inducible or chemically inducible control to enable the dynamic control of gene/genome editing in space and time. The promoter operably linked to the DNA targeting system coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Barr virus (EBV) promoter, or a Rous sarcoma virus (RSV) promoter. The promoter may also be a promoter from a human gene such as human ubiquitin C (hUbC), human actin, human myosin, human hemoglobin, human muscle creatine, or human metalothionein. Examples of a tissue specific promoter, such as a muscle or skin specific promoter, natural or synthetic, are described in U.S. Patent Application Publication No. US20040175727, the contents of which are incorporated herein in its entirety. The promoter may be a CK8 promoter, a Spc512 promoter, a MHCK7 promoter, for example.

The genetic construct may also comprise a polyadenylation signal, which may be downstream of the DNA targeting system. The polyadenylation signal may be a SV40 polyadenylation signal, LTR polyadenylation signal, bovine growth hormone (bGH) polyadenylation signal, human growth hormone (hGH) polyadenylation signal, or human (3-globin polyadenylation signal. The SV40 polyadenylation signal may be a polyadenylation signal from a pCEP4 vector (Invitrogen, San Diego, CA).

Coding sequences in the genetic construct may be optimized for stability and high levels of expression. In some instances, codons are selected to reduce secondary structure formation of the RNA such as that formed due to intramolecular bonding.

The genetic construct may also comprise an enhancer upstream of the DNA targeting system or the CRISPR/Cas-based gene editing system or gRNAs. The enhancer may be necessary for DNA expression. The enhancer may be human actin, human myosin, human hemoglobin, human muscle creatine or a viral enhancer such as one from CMV, HA, RSV, or EBV. Polynucleotide function enhancers are described in U.S. Pat. Nos. 5,593,972, 5,962,428, and WO94/016737, the contents of each are fully incorporated by reference. The genetic construct may also comprise a mammalian origin of replication in order to maintain the vector extrachromosomally and produce multiple copies of the vector in a cell. The genetic construct may also comprise a regulatory sequence, which may be well suited for gene expression in a mammalian or human cell into which the vector is administered. The genetic construct may also comprise a reporter gene, such as green fluorescent protein (“GFP”) and/or a selectable marker, such as hygromycin (“Hygro”).

The genetic construct may be useful for transfecting cells with nucleic acid encoding the DNA targeting system or CRISPR/Cas-based gene editing system, which the transformed host cell is cultured and maintained under conditions wherein expression of the DNA targeting system or CRISPR/Cas-based gene editing system takes place. The genetic construct may be transformed or transduced into a cell. The genetic construct may be formulated into any suitable type of delivery vehicle including, for example, a viral vector, lentiviral expression, mRNA electroporation, and lipid-mediated transfection for delivery into a cell. The genetic construct may be part of the genetic material in attenuated live microorganisms or recombinant microbial vectors which live in cells. The genetic construct may be present in the cell as a functioning extrachromosomal molecule.

Further provided herein is a cell transformed or transduced with a system or component thereof as detailed herein. Suitable cell types are detailed herein. In some embodiments, the cell is a stem cell. The stem cell may be a human stem cell. In some embodiments, the cell is an embryonic stem cell. The stem cell may be a human pluripotent stem cell (iPSCs). Further provided are stem cell-derived neurons, such as neurons derived from iPSCs transformed or transduced with a DNA targeting system or component thereof as detailed herein.

### 10. PHARMACEUTICAL COMPOSITIONS

Further provided herein are pharmaceutical compositions comprising the above-described genetic constructs or DNA targeting systems, including Targeted Activator System(s), Targeted Repressor System(s) or nuclease systems. Such systems, or at least one component thereof, as detailed herein may be formulated into pharmaceutical compositions in accordance with standard techniques well known to those skilled in the pharmaceutical art. The pharmaceutical compositions can be formulated according to the mode of administration to be used. In cases where pharmaceutical compositions are injectable pharmaceutical compositions, they are sterile, pyrogen free, and particulate free. An isotonic formulation is preferably used. Generally, additives for isotonicity may include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation.

The pharmaceutical composition may comprise about 1 ng to about 10 mg of DNA encoding the DNA targeting system(s), including the Targeted Activator System(s), Targeted Repressor System(s) or nuclease systems described herein, such as in the form of a DNA construct, an AAV vector or a lentivector. The pharmaceutical composition may comprise about 1 ng to about 10 mg of the gRNA described herein.

The composition may further comprise a pharmaceutically acceptable excipient. The pharmaceutically acceptable excipient may be functional molecules as vehicles, adjuvants, carriers, or diluents. The pharmaceutically acceptable excipient may be a transfection facilitating agent, which may include surface active agents, quinone analogs, vesicles such as squalene and squalene, hyaluronic acid, lipids, liposomes, calcium ions, viral proteins, polyanions, polycations, including poly-L-glutamate, or nanoparticles, or other known transfection facilitating agents. The carrier may be a non-toxic, inert solid, semi-solid or liquid filler, diluent, encapsulating material or formulation auxiliary of any type. Pharmaceutically acceptable carriers include, for example, diluents, antioxidants, preservatives, solvents, suspending agents, wetting agents, surfactants, propellants, humectants, powders, pH adjusting agents, and combinations thereof. The pharmaceutically acceptable excipient may be a transfection facilitating agent, which may include surface active agents, such as immune-stimulating complexes (ISCOMS), Freunds incomplete adjuvant, LPS analog including monophosphoryl lipid A, muramyl peptides, quinone analogs, vesicles such as squalene and squalene, hyaluronic acid, lipids, liposomes, calcium ions, viral proteins, polyanions, polycations, including poly-L-glutamate, or nanoparticles, or other known transfection facilitating agents.

The transfection facilitating agent may be a polyanion, polycation, including poly-L-glutamate (LGS), or lipid. The transfection facilitating agent is poly-L-glutamate, and more preferably, the poly-L-glutamate is present in the composition for genome editing in skeletal muscle or cardiac muscle at a concentration less than 6 mg/mL. The transfection facilitating agent may also include surface active agents such as immune-stimulating complexes (ISCOMS), Freunds incomplete adjuvant, LPS analog including monophosphoryl lipid A, muramyl peptides, quinone analogs and vesicles such as squalene and squalene, and hyaluronic acid may also be used administered in conjunction with the genetic construct. In some embodiments, the DNA vector encoding the composition may also include a transfection facilitating agent such as lipids, liposomes, including lecithin liposomes or other liposomes known in the art, as a DNA-liposome mixture (see for example International Patent Publication No. WO9324640), calcium ions, viral proteins, polyanions, polycations, or nanoparticles, or other known transfection facilitating agents. In some embodiments, the transfection facilitating agent is a polyanion, polycation, including poly-L-glutamate (LGS), or lipid.

### 11. ADMINISTRATION

The DNA targeting systems, or at least one component thereof, as detailed herein, or the pharmaceutical compositions comprising the same, may be administered to a subject. Such compositions can be administered in dosages and by techniques well known to those skilled in the medical arts taking into consideration such factors as the age, sex, weight, and condition of the particular subject, and the route of administration. The presently disclosed DNA targeting systems, or at least one component thereof, genetic constructs, or compositions comprising the same, may be administered to a subject by different routes including orally, parenterally, sublingually, transdermally, rectally, transmucosally, topically, intranasal, intravaginal, via inhalation, via buccal administration, intrapleurally, intravenous, intraarterial, intraperitoneal, subcutaneous, intradermally, epidermally, intramuscular, intranasal, intrathecal, intracranial, and intraarticular or combinations thereof. In certain embodiments, the DNA targeting system, genetic construct, or composition comprising the same, is administered to a subject intramuscularly, intravenously, or a combination thereof. For veterinary use, the DNA targeting systems, genetic constructs, or compositions comprising the same may be administered as a suitably acceptable formulation in accordance with normal veterinary practice. The veterinarian may readily determine the dosing regimen and route of administration that is most appropriate for a particular animal. The DNA targeting systems, genetic constructs, or compositions comprising the same may be administered by traditional syringes, needleless injection devices, “microprojectile bombardment gone guns,” or other physical methods such as electroporation (“EP”), “hydrodynamic method”, or ultrasound.

The DNA targeting systems, genetic constructs, or compositions comprising the same may be delivered to a subject by several technologies including DNA injection (also referred to as DNA vaccination) with and without in vivo electroporation, liposome mediated, nanoparticle facilitated, recombinant vectors such as recombinant lentivirus, recombinant adenovirus, and recombinant adenovirus associated virus. The composition may be injected into the skeletal muscle or cardiac muscle. For example, the composition may be injected into the tibialis anterior muscle or tail. The DNA Targeting Systems or at least one component thereof, genetic constructs, or compositions comprising the same, may be delivered to a subject non-virally. The DNA Targeting Systems or at least one component thereof, genetic constructs, or compositions comprising the same, may be delivered to a subject via a lipid nanoparticle.

a. Delivery of Protein and Nucleic Acids

DNA targeting systems, or at least one component thereof, such as fusion proteins, DNA binding proteins, guide RNA(s), and Cas polypeptides (including Cas fusion proteins) can be delivered to cells as DNA, RNA, or as pre-formed ribonucleoprotein complexes (RNPs) formats. The DNA format for the guide RNA will be transcribed into RNA. The DNA and RNA formats for Cas polypeptides or Cas fusion proteins require transcription and/or translation after being introduced into the cell, and the Cas polypeptide preferably also includes one, two or more nuclear localization sequences (NLS) to enhance entry into nucleus. Transfection methods include transfection into the cytoplasm (electroporation, lipofection) or the nucleus (nucleofection, microinjection), all well known in the art.

The pre-formed RNP format does not require any transcription or translation. If using RNPs, then delivery to the nucleus (nucleofection, microinjection) requires fewer steps. Other techniques to deliver RNPs into cells include induction of transmembrane internalization assisted by membrane filtration (TRIAMF) and induced transduction by osmocytosis and propane betaine (iTOP).

Similarly, ZF fusion proteins or TALE fusion proteins, comprising a nuclease, an activator or a repressor, can be delivered in a DNA or RNA format. These proteins preferably also include one, two or more NLS to enhance entry into nucleus.

DNA constructs comprising DNA encoding the guide RNA and/or Cas polypeptides and/or ZF fusion protein and/or TALE fusion protein described herein may comprise, such as, heterologous regulatory elements or transcriptional control signals as described herein for expression of the coding sequences of the nucleic acid. The regulatory elements may include, for example, a promoter, an enhancer, an initiation codon, a stop codon, or a polyadenylation signal.

The DNA targeting system, such as, nuclease system or Targeted Activator System or Targeted Repressor System, or one or more components thereof, may be encoded by or comprised within a genetic construct. Genetic constructs may include polynucleotides such as vectors and plasmids. The construct may be recombinant. In some embodiments, the genetic construct may comprise a promoter that is operably linked to the polynucleotide encoding at least one gRNA and/or a Cas9 protein. In some embodiments, the promoter is operably linked to the polynucleotide encoding a first gRNA, a second gRNA, and/or a Cas9 protein. The genetic construct may be present in the cell as a functioning extrachromosomal molecule that is not integrated into the chromosome. The genetic construct may be integrated into the chromosome. The genetic construct may be a linear minichromosome including centromere, telomeres, or plasmids or cosmids. The genetic construct may be transformed or transduced into a cell. Further provided herein is a cell transformed or transduced with a DNA targeting system or component thereof as detailed herein. In some embodiments, the cell is a stem cell. The stem cell may be a human stem cell. The stem cell may be a human pluripotent stem cell (iPSCs). Further provided are stem cell-derived neurons, such as neurons derived from iPSCs transformed or transduced with a DNA targeting system or component thereof as detailed herein.

### i) Viral Vectors

Viral vectors can also be used to transfer DNA or RNA into cells via transduction. Further provided herein is a viral delivery system. The nucleic acid encoding the DNA Targeting system, such as. Targeted Activator System, Targeted Repressor System or nuclease system, is packaged into viral particles which are then introduced into cells. For example, nucleic acid encoding the gRNA and/or Cas sequence is packaged into viral particles. To make the viral particles, generally the plasmid containing the gRNA or Cas9 encoding sequence and plasmids containing viral genes are introduced into a packaging cell line, and viral particles are harvested. Suitable viral vectors include lentivirus, adenovirus, adeno-associated virus (AAV), and herpes viruses. Platforms such as adeno-associated viral vectors (AAVs) are commonly used and can provide sustained expression without integration into the genome. AAV vectors possess significantly lower packaging capability than LVs (<5 kb). Lentivirus are effective in a variety of cells including non-dividing cells and can integrate into the genome or can be non-integrating. Viral vectors can be used to deliver DNA and RNA in vivo to subjects or ex vivo to their cells.

In some embodiments, the vector is an adeno-associated virus (AAV) vector. The AAV vector is a small virus belonging to the genus Dependovirus of the Parvoviridae family that infects humans and some other primate species. As used herein, the term “adenoviral associated virus (AAV) vector” refers to a vector having functional or partly functional ITR sequences and transgenes. As used herein, the term “ITR” refers to inverted terminal repeats (ITR). The ITR sequences may be derived from an adeno-associated virus serotype, including without limitation, AAV-1, AAV-2, AAV-3, AAV-4, AAV-5, AAV-6, AAV-7, AAV-8, AAV-9, AAV-10, AAV-11, AAV-12. The ITRs, however, need not be the wild-type nucleotide sequences, and may be altered (such as, by the insertion, deletion and/or substitution of nucleotides), so long as the sequences retain function to provide for functional rescue, replication and packaging. AAV vectors may have one or more of the AAV wild-type genes deleted in whole or part, preferably the rep and/or cap genes but retain functional flanking ITR sequences. Functional ITR sequences function to, for example, rescue, replicate and package the AAV virion or particle. Thus, an “AAV vector” is defined herein to include at least those sequences required for insertion of the transgene into a subject's cells. Optionally included are those sequences necessary in cis for replication and packaging (such as, functional ITRs) of the virus.

AAV vectors may be used to deliver DNA targeting systems using various construct configurations. For example, AAV vectors may deliver Cas9 and gRNA expression cassettes on separate vectors or on the same vector. Alternatively, if the small Cas9 proteins, derived from species such as Staphylococcus aureus or Neisseria meningitidis, are used then both the Cas9 and up to two gRNA expression cassettes may be combined in a single AAV vector within the 4.7 kb packaging limit.

In some embodiments, the AAV vector is a modified AAV vector. The modified AAV vector may have enhanced cardiac and/or skeletal muscle tissue tropism. The modified AAV vector may be capable of delivering and expressing the CRISPR/Cas9-based gene editing system in the cell of a mammal. For example, the modified AAV vector may be an AAV-SASTG vector (Piacentino et al. Human Gene Therapy 2012, 23, 635-846). The modified AAV vector may be based on one or more of several capsid types, including AAV1, AAV2, AAVS, AAV6, AAV8, and AAV9. The modified AAV vector may be based on AAV2 pseudotype with alternative muscle-tropic AAV capsids, such as AAV2/1, AAV2/6, AAV2/7, AAV2/8, AAV2/9, AAV2.5, and AAV/SASTG vectors that efficiently transduce skeletal muscle or cardiac muscle by systemic and local delivery (Seto et al. Current Gene Therapy 2012, 12, 139-151). The modified AAV vector may be AAV2i8G9 (Shen et al. J. Biot. Chem. 2013, 288, 28814-28823).

As used herein, the term “lentiviral vector” refers to a vector derived from lentivirus, a family of retroviruses characterized by long incubation periods. To be used safely as a vector, the lentivirus has been modified extensively to delete virulence and replication genes. In addition, the integrase of lentivirus can be deleted or mutated, resulting in a non-replicating and non-integrating lentivector. Integrase-deficient lentiviral vectors (IDLV) can be used to deliver CRISPR-cas systems. Lentivectors carrying Cas polypeptides and guide RNAs are described in U.S. Pub. App. No. 20180201951, incorporated herein by reference in its entirety.

### ii) Nanoparticles

Lipid materials have been used to create lipid nanoparticles (LNPs) based on ionizable cationic lipids, which exhibit a cationic charge in the lowered pH of late endosomes to induce endosomal escape, because of the tertiary amines in their structure. These LNPs have been used, for example, to deliver RNA interference (RNAi) components, as well as genetic constructs or CRISPR-Cas systems. See, such as, Wlbie et al. (Acc. Chem. Res. 2019, 52, 1555-1564, incorporated by reference) and Wang et al. (Proc. Natl. Acad. Sci. U.S.A. 2016, 113, 2868-2873, incorporated by reference) describe use of biodegradable cationic LNPs. Chang et al. (Acc. Chem. Res. 2019 52, 665-875, incorporated by reference) describe use of ionizable lipid along with cholesterol, DSPC, and a PEGylated lipid to create LNPs.

Polymer based particles can be used for genetic construct delivery in a similar manner as lipids. A number of materials have been used for delivery of nucleic acids. For example, cationic polymers such as polyethylenimine (PEI) can be complexed to nucleic acids and can induce endosomal uptake and release, similarly to cationic lipids. Dendrimeric structures of poly(amido-amine) (PAMAM) can also be used for transfection. These particles consist of a core from which the polymer branches. They exhibit cationic primary amines on their surface, which can complex to nucleic acids. Networks based on zinc to aid cross-linking of imidazole have been used as delivery methods, relying on the low pH of late endosomes which, upon uptake, results in cationic charges due to dissolution of the zeolitic imidazole frameworks (ZIF), after which the components are released into the cytosol. Colloidal gold nanoparticles have also been used (see, for example, Wilbie et al. Acc. Chem. Res. 2019, 52, 1555-1564, incorporated by reference).

In some embodiments, the DNA targeting system, or at least one component thereof, is delivered non-virally. Non-viral administration may include administration of the DNA targeting system, or at least one component thereof, without a viral vector. For example, the DNA targeting system, or at least one component thereof, or a polynucleotide encoding the DNA targeting system or at least one component thereof, may be delivered via a nanoparticle. The DNA targeting system, or at least one component thereof, or a polynucleotide encoding the DNA targeting system or at least one component thereof, may be encapsulated within a nanoparticle, such as a lipid nanoparticle or a polymeric nanoparticle.

### 12. METHODS

a. Methods for Stably Activating a Gene or Gene Product

Provided herein are methods of stably activating a gene or gene product within the imprinted 15q11-13 locus in a subject having Prader Willi Syndrome (PWS) or Prader-Willi-like disorder. The method may include non-virally administering to the subject a DNA targeting system that targets a target region in the imprinted 15q11-13 PWS-associated locus as detailed herein.

Further provided herein are methods of stably expressing a gene or gene product within the imprinted 15q11-13 locus in a subject having Prader Willi Syndrome (PWS) or Prader-Willi-like disorder. The method may include non-virally administering to the subject a DNA targeting system that targets a target region in the imprinted 15q11-13 PWS-associated locus as detailed herein.

In some embodiments, at least one component of the DNA targeting system is transiently expressed in a cell from the subject or transiently delivered to a cell from the subject. Transient expression may include expression for a short period of time, such as, for example, less than 24 hours, less than 1 day, less than 2 days, less than 3 days, less than 4 days, less than 5 days, less than 6 days, less than 7 days, or less than 8 days post-administration, relative to a control. Stable expression of a gene or gene product may include maintained and/or consistent expression of the gene or gene product for an extended period of time, relative to a control. Expression of a gene within the imprinted 15q11-13 locus may be maintained in a cell from the subject for, for example, at least 10 days, at least 15 days, at least 20 days, at least 25 days, at least 26 days, at least 30 days, at least 35 days, at least 40 days, at least 45 days, at least 48 days, at least 50 days, or at least 55 days post-administration, relative to a control. Expression of a gene within the imprinted 15q11-13 locus may be maintained in a cell from the subject for, for example, at least 1 week, at least 2 weeks, at least 3 weeks, at least 4 weeks, at least 5 weeks, at least 6 weeks, at least 7 weeks, at least 8 weeks, at least 9 weeks, or at least 10 weeks post-administration, relative to a control.

The expression of a gene or gene product within the imprinted 15q11-13 PWS-associated locus may be increased. The expression of a gene or gene product within the imprinted 15q11-13 PWS-associated locus may be increased by at least about 5%, 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene or gene product within the imprinted 15q11-13 PWS-associated locus may be increased by less than about 10%, 15%, 20%, 25%, 30%, 35%, 40%, 45%, 50%, 55%, 60%, 65%, 70%, 75%, 80%, 85%, 90%, 95%, 1.5-fold, 2-fold, 3-fold, 4-fold, 5-fold, 6-fold, 7-fold, 8-fold, 9-fold, or 10-fold, relative to a control. The expression of the gene or gene product within the imprinted 15q11-13 PWS-associated locus may be increased by about 5-95%, 10-90%, 15-85%, 20-80%, or 1.5-fold to 10-fold, relative to a control. A control may be, for example, an expression level pre-administration.

b. Methods for Treating Prader-Willi Syndrome (PWS)

Provided herein are methods for treating Prader-Willi Syndrome (PWS) in a subject in need thereof. The method may include administering to the subject a DNA targeting system as detailed herein, an isolated polynucleotide sequence as detailed herein, a vector or genetic construct as detailed herein, a modified cell as detailed herein, or a combination thereof. In some embodiments, the expression of at least one gene within the 15q11-q13 locus is increased in the subject or in a sample therefrom. In some embodiments, the expression of at least one gene within the 15q11-q13 locus is increased in the subject or in a sample therefrom, such as from activation of the maternal copy of the gene. In some embodiments, the expression of at least one RNA transcript selected from SNRPN, SNORD115, and SNORD116, or a combination thereof, is increased in the subject or in a sample therefrom. In some embodiments, the initiation of transcription from the SNRPN promoter, SNORD115 promoter, SNORD116 promoter, or a combination thereof, is increased in the subject or in a sample therefrom.

The disclosure herein identifies target regions within the PWS associated locus of chromosome 15q11-13 that can be targeted by the DNA targeting systems, such as, Targeted Activator Systems, for activation, to increase expression of genes and gene products to treat PWS. Relative to position 1 being the start sire of the SNRPN gene exon 1 of the PWS imprinting center on chromosome 15, the target regions of interest for activation are at nucleotide positions −127023 to −125023; nucleotide positions −93065 to −91065; and/or nucleotide positions −1104 to +896. More specifically, the target regions are at nucleotide positions −126523 to 125523; nucleotide positions −92565 to −91565; and/or nucleotide positions −604 to +396. Even more specifically, the target region encompasses nucleotide positions −126023, −92065, and/or −104, and may be within about 100-1000, about 100-900, about 100-800, about 100-700, about 100-600, about 100-500, about 100-400, about 100-300 or about 100-200 bp upstream, or downstream, of the target region of the DNA identified herein.

The disclosure herein also identifies target regions within the PWS associated locus of chromosome 15q11-13 that can be targeted by the DNA targeting systems, such as Targeted Repressor Systems, for repression, to increase expression of genes and gene products to treat PWS. Relative to position 1 being the start site of the SNRPN gene exon 1 of the PWS imprinting center on chromosome 15, the target regions of interest for activation are at nucleotide positions +23022 to +25022 and/or +34734 to +36734. More specifically, the target regions are at nucleotide positions +23522 to +24522 and/or +35234 to +36234. Even more specifically, the target region encompasses nucleotide positions +24022 and/or +35734, and may be within about 100-1000, about 100-900, about 100-800, about 100-700, about 100-600, about 100-500, about 100-400, about 100-300 or about 100-200 bp upstream, or downstream, of the target region of the DNA identified herein.

Relative to position 1 being the start site of the SNRPN gene exon 1 of the PWS imprinting center on chromosome 15, additional target regions of interest for activation and/or demethylation include regions at positions −126547 to −124695 [mat1]; −131937 to −130580 [mat1A]; −129415 to −127715 [mat1B]; −123798 to −122440 [mat1C]; −92568 to −91460 [mat2]; −797 to +1346 [mat3]; and/or +12858 to +14026 [mat4]. More specifically, within these regions, further subregions include target regions at positions: −126047 to −125195 [mat1]; −131437 to −131080 [mat1A]; −128915 to −128215 [mat1B]; −123298 to −122940 [mat1C]; −92068 to −91960 [mat2]; −297 to +846 [mat3]; and/or +13358 to +13526 [mat4]. Even more specifically, within these subregions, further subregions include target regions at positions: −126047 to −125947; −125997 to −125897; −125947 to −125847; −125897 to −125797; −125847 to −125747; −125797 to −125697; −125747 to −125647; −125697 to −125597; −125647 to −125547; −125597 to −125497; −125547 to −125447; −125497 to −125397; −125447 to −125347; −125397 to −125297; −125347 to −125247; −125297 to −125195; and/or −125247 to −125195 [mat1]; −131437 to −131337; −131387 to −131287; −131337 to −131237; −131287 to −131187; −131237 to −131137; −131187 to −131087; −131137 to −131037; and/or −131087 to −131080 [mat1A]; −128915 to −128815; −128865 to −128765; −128815 to −128715; −128765 to −128665; −128715 to −128615; −128665 to −128656; −128615 to −128515; −128565 to −128465; −128515 to −128415; −128465 to −128365; −128415 to −128315; −128365 to −128265; and/or −128315 to −128215 [mat1B]; −123298 to −123198; −123248 to −123148; −123198 to −123098; −123148 to −123048; −123098 to −122998; −123048 to −122948; and/or −122998 to −122940 [mat1C] −92068 to −91968; −92018 to −91918; and/or −91968 to −91960 [mat2]; −297 to −187; −247 to −147; −197 to −97; −147 to −47; −97 to +3; −47 to +53; +3 to +100; +53 to +153; +103 to +203; +153 to +253; +203 to +303; +253 to +353; +303 to +403; +353 to +453; +403 to +503; +453 to +553; +503 to +603; +553 to +653; +603 to +703; +653 to +753; +703 to +803; +753 to +846; and/or +803 to +848 [mat3] and/or +13358 to +13458; +13408 to +13508; +13458 to +13526; and/or +13508 to +13526 [mat4].

Relative to position 1 being the start site of the SNRPN gene exon 1 of the PWS imprinting center on chromosome 15, additional target regions of interest for repression include regions at positions −101358 to −94223 [pat1]; −58232 to −51914 [pat2]; −4847 to −3047 [pat3]; −1774 to +2421 [pat4]; +2446 to +24016 [pat5]; +23346 to +25082 [pat6]; +24340 to +35718 [pat7]; and/or +35206 to +36668 [pat8]. More specifically, within these regions, further subregions include target regions at positions: −100858 to −94723[pat1]; −57732 to −52414 [pat2]; −4347 to −3547 [pat3]; −1275 to +1921 [pat4]; +2946 to +23516 [pat5]; +23846 to +24582 [pat6]; +24840 to +35218 [pat7]; and/or +35706 to +36168 [pat8]. Even more specifically, within these subregions, further subregions include target regions at positions: +23846 to +23946; +23896 to +23996; +23946 to +24046; +23996 to +24096; +24046 to +24146; +24096 to +24196; +24146 to +24246; +24196 to +24296; +24246 to +24346; +24296 to +24396; +24346 to +24446; +24396 to +24496; +24446 to +24546; and/or +24496 to +24582; [pat6]; +35706 to +35806; +35756 to +35856; +35806 to +35906;+35856 to +35956; +35906 to +36006; +35956 to +36056; +36006 to +36106; +36056 to +36156; and/or +36106 to +36168 [pat8].

### 13. EXAMPLES

### Example 1

### Materials and Methods

Generation of SNRPN-2A-GFP Plurpotent Stem Cel Lines. A human iPS cell line (RVR-iPSCs) was used to construct the maternal and paternal SNRPN-2A-GFP reporter lines. RVR-iPSCs were retrovirally reprogrammed from BJ fibroblasts and characterized previously (Lee, et al. Cell, 2012, 151, 547-58, incorporated by reference). To generate the SNRPN-2A-GFP reporter lines, 3×10⁶ cells were dissociated with Accutase (Stemcell Tech, 7920) and electroporated with 6 μg of gRNA-Cas9 expression vector and 3 μg of SNRPN targeting vector using the P3 Primary Cell 4D-Nucleofector Kit (Lonza, V4XP-3032). The transfected cells were plated into a 10 cm dish coated with Matrigel (Corning, 354230) in complete mTesR1 (Stemcell Tech, 85850) supplemented with 10 μM Rock Inhibitor (Y-27632, Stemcell Tech, 72304). Twenty-four hours after transfection, positive selection began with 1 μg/mL puromycin for 7 days. Following selection, cells were transfected with a CMV-CRE recombinase expression vector to remove the foxed puromycin selection cassette. The transfected cells were expanded and plated at low density of 180 cells/cm² for clonal isolation. The resulting clones were mechanically picked and expanded, and gDNA was extracted using QuickExtract DNA Extraction Solution (Lucigen, QE09050) for PCR screening of targeting vector integration. A polyclonal cell line expressing lentivirally transduced dCas9-KRAB, VP64-dCas9-VP64, or Tet1c-dCas9 was used for the CRISPR screens and validations.

Cell Culture. Human iPSCs were maintained in mTeSR1 (StemCell Tech, 85850) or mTeSR Plus (StemCell Tech, 100-0276) on Matrigel-coated tissue culture plastic in a 37° C. incubator with 5% CO₂. Prior to use, plates were coated with Matrigel (Corning, 354230) at a concentration of 1 mg per 24 mL DMEM/F12 (Gibco, 11320033) and incubated for at least 1 hour at 37° C. The cell culture medium was supplemented with 10 μM ROCK inhibitor (ROCKi) (Y-27632, Stemcell Tech, 72304) for 16-48 hours before cells were passaged with Accutase (StemCell Tech, 07920; Innovative Cell Technologies, AT104; Gibco A1110501) and otherwise omitted.

IPSC Nucleofection and Cell Sorting. For plasmid nucleofection, iPSCs were detached from the plate with Accutase diluted 1:1 in divalent cation-free PBS. After cells were removed from the plate, the cells were counted, spun down for 10 minutes at 100×g, and resuspended in Lonza P3 Primary Cell Nucleofector Solution (Lonza, VXP-3024) at approximately 8 million cells per 100 μL reaction. The desired plasmid was prepared at concentration of 10 μg of plasmid per 100 μL reaction. After preparing the plasmid, cells were nucleofected with the Nucleofector X Unit (Lonza) using program CB-150. The samples were immediately resuspended in pre-warmed mTeSR Plus media with 10 μM ROCKi and transferred to 6-well plates at a cell density of 4-8 million cells per well.

After an 18-24 hour incubation, cells were washed with PBS, and adherent cells were detached from the plate with Accutase. Samples were stained in PBS with eBioscience Fixable Viability Dye eFluor 780 (Invitrogen, 65-0865-18) and Anti-Rat CD90 Antibody FITC (referred to in the text as mouse Thy1) (StemCell, 60024FI.1, Lot no. 1000072622). The remaining stained and un-transfected cells were used as a negative control for the anti-CD90 antibody. The live, CD90-positive cells were sorted with the Sony SH800Z cell sorter into tubes containing DMEM/F12, 10 μM ROCKi, and 1% antibiotic-antimycotic (Gibco, 15240062).

Approximately 20% of the sorted cells were pelleted and flash-frozen for RNA extractions. The remaining approximately 80% of the cells were pelleted for 5 minutes at 300×g, re-suspended in mTeSR Plus, 10 μM ROCKi, and 1% antibiotic-antimycotic, and plated in 24-well plates. One or two days later, the media was changed to mTeSR Plus without antibiotic-antimycotic. After approximately 2-3 days, colonies of cells appeared well-established and ROCKi was omitted from the media. The media was changed daily or once every two days.

Neuronal Differentiation. Wild-type (WT) and ΔPWS iPSCs were differentiated via Ngn2 over-expression as described in a published protocol, with modifications (Wang, et al. Stem Cell Reports, 2017, 9, 1221-1233, incorporated by reference). Briefly, iPSCs were transduced during plating with lentiviruses encoding TetO-mNgn2 and hUBC-M2rtTA to deliver doxycycline-responsive mouse Ngn2 cDNA. After transduction, cells were maintained in mTeSR Plus with 10 μM ROCKi for one day. The next day, media was changed to pre-differentiation media with 2 μg/mL doxycycline, as described in the Wang protocol. The media was changed daily. Two days later, the pre-differentiated cells were detached and re-plated in post-differentiation media with 2 μg/mL doxycycline at approximately 100,000 cells per cm². After the cells were re-plated, partial media changes not containing doxycycline occurred every 7 days.

Lentiviral Production and Titration. HEK293T cells were acquired from the American Tissue Collection Center (ATCC) and purchased through the Duke University Cell Culture Facility. The cells were maintained in DMEM High Glucose supplemented with 10% FBS and 1% penicillin-streptomycin and cultured at 37° C. with 5% CO₂.

For lentiviral production of the gRNA libraries, VP64-dCas9-VP64 and dCas9-KRAB, 4.5×10⁸ cells were transfected using the calcium phosphate precipitation method (Salmon, et al, Curr Protoc Hum Genet, 2007, Chap. 12, incorporated by reference) with 6 μg pMD2.G (Addgene #12259), 15 μg psPAX2 (Addgene #12260), and 20 μg of the transfer vector. The medium was exchanged 12-14 hours after transfection, and the viral supernatant was harvested 24 and 48 hours after this medium change. The viral supernatant was pooled and centrifuged at 600×g for 10 min, passed through a PVDF 0.45 μm filter (Millipore, SLHV033RB), and concentrated to 50× in 1×PBS using Lenti-X Concentrator (Clontech, 631232) in accordance with the manufacturer's protocol.

To produce lentivirus for gRNA validations or dCas9 effectors, 1.2×10⁶ cells were transfected in 6-well plates using Lipofectamine 3000 (Invitrogen, L3000008) according to the manufacturer's instructions with 200 ng pMD2.G (Addgene #12259), 600 ng psPAX2 (Addgene #12260), and 500 ng of the transfer vector. The medium was exchanged 6 hours after transfection, and the viral supernatant was harvested 24 and 48 hours after transfection. The viral supernatant was pooled and filtered through a 0.45 μm filter or centrifuged for 10 min at 600×g to remove cell debris, then concentrated to 50× in 1×PBS using Lenti-X Concentrator (Takara, 631232), in accordance with the manufacturer's protocol.

The titer of the lentiviral gRNA library pool was determined by transducing 3×10⁴ cells with serial dilutions of lentivirus and measuring the percent mCherry expression 4 days after transduction with a SH800 FACS Cell Sorter (Sony Biotechnology). All lentiviral titrations were performed in the cell lines used in the CRISPR screens.

RNA Isolation and Quantitative RT-PCR. Total RNA was isolated using RNeasy Plus (Qiagen, 74136) and QIAshredder kits (Qiagen, 79656) for VP64 and KRAB gRNA validations in SNRPN-2A-GFP iPSCs. Total RNA was isolated using Norgen Total RNA Purification Plus Micro kit (Norgen, 48500) for other experiments, including RNA sequencing.

Reverse transcription was carried out on 0.1-0.5 μg total RNA per sample in a 10 or 20 μL reaction using the SuperScript VILO Reverse Transcription Kit (Invitrogen, 11754). Ten nanograms of cDNA was used per PCR reaction with Perfecta SYBR Green Fastmix (Quanta BioSciences, 95072) using the CFX96 Real-Time PCR Detection System (Bio-Rad). All amplicon products were verified by melting curve analysis. Additionally, the qRT-PCR results are presented as fold change in RNA normalized to GAPDH expression.

To purify poly(A) RNA, total RNA was first isolated using RNeasy Plus (Qiagen) as described above. Poly(A) RNA was purified from 1 μg total RNA using RNA purification beads as part of the Truseq Stranded mRNA kit according to the manufacturer's protocol (Illumina). For each qRT-PCR reaction, 1 ng of poly(A)-enriched RNA was used.

RNA Sequencing andData Analysis. For RNA sequencing, total RNA isolated with the Norgen RNA Purification Plus Micro kit (Norgen, 48500) was submitted to Genewiz for total RNA sequencing. Genewiz verified quality of samples and libraries and performed DNAse treatment as necessary.

Sequencing reads were trimmed with Trimmomatic to remove adapters and filter on read quality. Each read was mapped to hg19 with STAR. DeepTools bamCoverage was used to generate RPKM-normalized bedgraph files, then bedGraphToBigWig was used to convert bedgraph to bigwig files for visualization in the genome browser. Additionally, read counts for each gene were created using the featureCounts function. Differential expression analysis was performed with DESeq2.

Plasmid Construction. The lentiviral VP64-dCas9-VP64 plasmid was generated by modifying Addgene #59791 to replace GFP with BSD blasticidin resistance. The Tet1c-dCas9 plasmid was generated by amplifying Tet1c from Addgene #108245, followed by Gibson assembly of Tet1c, dCas9, and BSD into a lentiviral expression backbone. The dCas9-KRAB plasmid is equivalent to Addgene #67620 but with BSD replacing GFP, and the LacZ gene is not present in the backbone. The Tet1c-Cas9-T2A-Thy1.1 plasmid for transfection was generated by replacing BFP in Addgene #167983 with mouse Thy1.1 (CD90).

The gRNA expression plasmid for the single gRNA screens was generated by modifying Addgene #83925 to contain an optimized gRNA scaffold, in which a puromycin resistance gene is incorporated in place of Bsr and an mCherry transgene is incorporated in place of GFP. For creating stable gRNA-expressing cell lines in WT and ΔPWS iPSCs for nucleofection of the Tet1c-Cas9 effector, we used a gRNA expression plasmid that was further modified by removing the mCherry transgene.

The gRNA expression plasmid for the dual gRNA screen was generated by further modification of the single gRNA expression plasmid to contain an additional gRNA cassette expressing sg-mat1 under control of the mU6 Pol III promoter with a modified gRNA scaffold described previously. Individual gRNAs were ordered as oligonucleotides (Integrated DNA Technologies) and cloned into the gRNA expression plasmids using BsmBI sites.

The SNRPN targeting vector was cloned by inserting approximately 700 bp homology arms (surrounding the SNRPN stop codon in exon 10), amplified by PCR from genomic DNA of RVR-iPS cells, and subsequently flanking a P2A-GFP sequence with a LoxP-puromycin resistance cassette.

gRNA Library Design and Cloning. For the full gRNA library, the gt-scan algorithm (O'Brien, et al, Bioinformatics, 2014, 30, 2673-2675, incorporated by reference) was used to identify all possible gRNAs within the chromosome 15q11-13 region and rank the gRNAs by off-target alignments to the human genome. DNase I hypersensitivity sites (DHSs) for H1 human embryonic stem cells (H1 hESCs) were downloaded from ENCODE (http://www.encodeproject.org). The high-density gRNA region spanned chr15:25,064,194-25,368,441 (hg19) and consisted of the top 30% of all gRNAs within this region ranked by off-target score. Outside of the high-density region and within chr15:23,692,325-26,425,399 (hg19), all gRNAs within DHS coordinates in H1 hESCs were selected for inclusion in the library. In total, 11,751 gRNAs were designed, including 531 non-targeting control gRNAs that were selected from a previous genome-wide CRISPRi gRNA library (Horibeck, et al, eLife, 2016, 5, incorporated by reference). For the gRNA sub-library used in the Tet1c-dCas9 screen, all hits (adjusted p-value<0.05) from the previous CRISPRa and CRISPRi screens were included, in addition to 50 non-targeting control gRNAs, for a total of 583 gRNAs. For both gRNA libraries, the oligonucleotide pool (Twist Bioscience) was amplified with PCR and cloned using Gibson assembly into the single or dual gRNA expression plasmid.

dCas9-KRAB and VP64-dCas9-VP64 SNRPN-2a-GFP Screens. Each screen was performed in triplicate with independent transductions. For each replicate, 24×10⁶ SNRPN-2A-GFP VP64-dCas9-VP64 (maternal) or dCas9-KRAB (paternal) iPSCs were detached from the plate using Accutase (Stemcell Tech, 7920) and transduced in suspension across five matrigel-coated 15-cm dishes in mTesR (Stemcell Tech 85850) supplemented with 10 μM ROCK inhibitor (Y-27632, Stemcell Tech, 72304). Cells were transduced at a multiplicity of infection (MOI) of 0.2 to obtain one gRNA per cell and ˜430-fold coverage of the gRNA library. The medium was changed to fresh mTesR without ROCK inhibitor 18-20 hours after transduction. Antibiotic selection was started 30 hours after transduction by adding 1 μg/mL puromycin (Sigma, P8833) directly to the plates without changing the medium. The cells were fed daily and passaged as necessary maintaining library coverage until harvest.

Cells were harvested for sorting 9 days after transduction of the gRNA library for all three screens. Cells were washed once with 1×PBS, detached using Accutase, filtered through a 30 μm CellTrics filter (Sysmex, 04-004-2326), and resuspended in fluorescence-activated cell sorting (FACS) Buffer (0.5% BSA (Sigma, A7906), 2 mM EDTA (Sigma. E7889) in PBS). Before sorting, an aliquot of 4.8×10⁸ cells was taken to represent a bulk unsorted population. The highest and lowest 10% of cells were sorted based on GFP expression and 4.8×10⁸ cells were sorted into each bin. Sorting was completed with a SH800 FACS Cell Sorter (Sony Biotechnology). After sorting, genomic DNA was harvested with the DNeasy Blood and Tissue Kit (Qiagen, 69506).

Tet1c-dCas9 and VP64-dCas9-VP64 SNRPN-2A-GFP Sub-Library Screens. The screen was performed in triplicate with independent transductions. For each replicate, 1.7×10⁶ matSNRPN-2A-GFP Tet1c-dCas9 or VP64-dCas9-VP64 iPSCs were detached using Accutase (Stemcell Tech, 7920) and transduced in suspension in a matrigel-coated 10-cm dish in mTesR (Stemcell Tech 85850) supplemented with 10 μM ROCK inhibitor (Y-27632, Stemcell Tech, 72304). Cells were transduced at a MOI of 0.2 to obtain one gRNA per cell and approximately 580-fold coverage of the gRNA sub-library. The medium was changed to fresh mTesR without ROCK inhibitor 18-20 hours after transduction. Antibiotic selection was started 30 hours after transduction by adding 1 μg/mL puromycin (Sigma, P8833) directly to the plates without changing the medium. The cells were fed daily and passaged as necessary maintaining library coverage until harvest.

Cells were harvested for sorting 14 days after transduction of the gRNA sub-library. Cells were washed once with 1×PBS, detached using Accutase, filtered through a 30 μm CellTrics filter (Sysmex, 04-004-2326) and resuspended in FACS Buffer (0.5% BSA (Sigma, A7906), 2 mM EDTA (Sigma, E7889) in PBS). Before sorting, an aliquot of 0.4×10⁸ cells was taken to represent a bulk unsorted population. The highest and lowest 10% of cells were sorted based on GFP expression, and 0.4×10⁶ cells were sorted into each bin. Sorting was done with a SH800 FACS Cell Sorter (Sony Biotechnology). After sorting, genomic DNA was harvested with the DNeasy Blood and Tissue Kit (Qiagen, 69506).

Genomic DNA Sequencing. The gRNA libraries were amplified from each gDNA sample across 100 μL PCR reactions using Q5 hot start polymerase (NEB, M0493) with 1 μg of gDNA per reaction. The PCR amplification was done according to the manufacturer's instructions, using 25 cycles at an annealing temperature of 60° C. with the following primers:

Fwd:
(SEQ ID NO: 1143)
5′-AATGATACGGCGACCACCGAGATCTACACAATTTCTTGGGTAG
TTTGCAGTT
Rev:
(SEQ ID NO: 1144)
5′-CAAGCAGAAGACGGCATACGAGAT-(6-bp index sequence)-
GACTCGGTGCCACTTTTTCAA

The amplified libraries were purified with Agencourt AMPure XP beads (Beckman Coulter, A63881) using double size selection of 0.65× and then 1× the original volume. Each sample was quantified after purification with the Qubit dsDNA High Sensitivity assay kit (Thermo Fisher, Q32854). Samples were pooled and sequenced on a MiSeq (Illumina) with 21-bp paired-end sequencing using the following custom read and index primers:

Read1:
(SEQ ID NO: 1145)
5′-GATTTCTTGGCTTTATATATCTTGTGGAAAGGACGAAACACCG
Index:
(SEQ ID NO: 1146)
5′-GCTAGTCCGTTATCAACTTGAAAAAGTGGCACCGAGTC
Read2:
(SEQ ID NO: 1147)
5′-GTTGATAACGGACTAGCCTTATTTAAACTTGCTATGCTGTTTC
CAGCATAGCTCTTAAAC

Data Processing and Enrichment Analysis. FASTQ files were aligned to custom indexes (generated from the bowtie2-build function) using Bowtie 2 (Langmead, B. & Salzberg, S. L. Nat. Methods 2012, 9, 357-359, incorporated by reference). Counts for each gRNA were extracted and used for further analysis. All enrichment analysis was done with R. Individual gRNA enrichment was determined using the DESeq2 (Love et al. Genome Biology 2014, 15, 550, incorporated by reference) package to compare between high and low, unsorted and low, or unsorted and high conditions for each screen.

gRNA Validations. The top enriched gRNAs from the screens were individually cloned into the appropriate gRNA expression vector as described previously. The gRNA validations were performed similarly as done with the screens using maternal or paternal SNRPN-2A-GFP lines stably expressing either dCas9-KRAB or VP64-dCas9-VP64, except the transductions were performed in 24-well plates and the virus was delivered at high MOI. For the validations of the dCas9-KRAB paternal screen, single gRNAs were tested per region. For the validations of the VP64-dCas9-VP64 maternal screen, pools of 3-4 gRNAs were tested per region in SNRPN-2A-GFP iPSCs. For validations of the Tet1c-dCas9 screen and all studies in WT and ΔPWS iPSCs, single gRNAs were tested except when otherwise specified. Cells were cultured on matrigel-coated 24-well plates in mTesR and harvested for flow cytometry or qRT-PCR 5-7 days after gRNA transduction for dCas9-KRAB and 9-14 days after gRNA transduction for VP64-dCas9-VP64 and Tetlc-dCas9.

Bisulphite Primer Design, Bisulphite Conversion, and Sequencing. Primer pairs for bisulphite sequencing were designed for the target region using Zymo Research Bisulfite Primer Seeker, with a maximum amplicon length of 400 bp. Each primer set was initially tested on WT and ΔPWS iPSC Tet1c-dCas9 stable lines (with no gRNA) with 400 ng input gDNA per conversion reaction. To validate primer pair on bisulphite-converted gDNA, we used bisulphite-converted product as input in PCR reactions and tested an annealing temperature gradient ranging from 55-82° C. for 30 cycles with Kapa Uracil+HotStart ReadyMix polymerase (Roche, 7959052001). Samples of each PCR product were run on a 2% agarose gel. Products displaying bands of the expected size without primer dimer or off-target were Sanger-sequenced (Genewiz) to verify that the correct region was amplified and that the expected proportion of T/C reads were present at each differentially methylated cytosine within the region. Of four tested primer pairs, two pairs were found to amplify the expected region within the PWS-IC specifically. We proceeded with the primer pair that produces a 400 bp amplicon covering 24 CpG sites within the PWS-IC.

For bisulphite sequencing of iPSCs and neurons, genomic DNA was extracted from cultured cells with the Qiagen DNEasy Blood and Tissue kit (Qiagen, 69504). gDNA was stored at −80° C. For the bisulphite conversion reaction 250 ng of gDNA was used. For bisulphite conversion and purification, Zymo EZ DNA Methylation Gold kit was used as instructed (Zymo, D5005). An aliquot (2 μL) of bisulphite-converted gDNA was used as input for PCR as described above, with an annealing temperature of 57° C. The PCR1 reactions were then cleaned with Ampure XP beads (Beckman, A63881) at a ratio of 1.8×. An aliquot ( 1/10^{th}) of PCR1 was used as input for PCR2 with Q5 polymerase. PCR2 added i5 and i7 barcodes and P5 and P7 overhangs for dual-index Illumina short read sequencing.

PCR2 products were purified with Ampure XP beads. Products were visualized via electrophoresis on an Agilent TapeStation 4200 and quantified with Qubit dsDNA HS assay kit (Invitrogen. Q32851) on a Qubit fluorometer (Invitrogen). Libraries were pooled and sequenced on an Illumina Miseq instrument with a Miseq v3 600 cycle kit (Illumina, MS-102-3003) with read lengths of 250×250.

Bisulphite Sequencing Data Analysis. From the raw read files, we used Trimmomatic (Bolger et al. Bioinformatics 2014, 30, 2114-2120, incorporated by reference) to trim reads using the following settings:

HEADCROP:25; ILLUMINACLIP:TruSeq3-PE-2.fa:2:30:10;
TRAILING:20; SLIDINGWINDOW:4:15; MINLEN:40

Bismark (Krueger and Andrews. Bioinformatics 2011, 27,1571-1572, incorporated by reference) was used to create a bisulphite-converted reference genome from the hg19 genome build. Trimmed, paired-end reads were aligned to the genome and analysed with Bismark version 0.22.3. The output coverage files were used to determine percentage of methylated cytosine for each CpG site in the amplicon.

HCR FlowRSH. For HCR FlowFISH, reagents and probe sets were ordered from Molecular Instruments. Buffer set formulations were for cells in suspension. Approximately 2 million cells per sample were detached from the plate with Accutase and processed according to the protocol as described in Reilly et al., except that probe hybridisation buffer, probe wash buffer, and amplification buffer were used from Molecular Instruments (Reilly, et al. Nature Genetics, 2021, 53,1166-1176, incorporated by reference). Probe sets were used at a 4 nM concentration. Fixation and permeabilization buffer was prepared fresh as 4% paraformaldehyde (Electron Microscopy Sciences, 15710) and 0.1% Tween-20 (Roche, 11332465001) in 1× divalent cation-free phosphate-buffered saline. After staining, cells were analysed on a Sony SH800Z cell sorter.

ATAC Sequencing. iPSCs were detached from the plate using Accutase, and viability and cell number were assessed with a Countess II cell counter (Invitrogen) and Trypan Blue (Invitrogen, T10282), 45,000 cells per sample were processed for ATAC-seq according to the Omni-ATAC protocol (Corces, et al. Nature Methods, 2017, 14, 959-962, incorporated by reference). Libraries were sequenced on an Illumina NovaSeq with 2×25 paired-end reads with a coverage of 50 million reads per sample.

Reads were trimmed with Trimmomatic (Bolger et al. Bioinformatics 2014, 30, 2114-2120, incorporated by reference) to remove adapter content and filter on quality score, then trimmed reads were aligned to hg19 using Bowtie2 (Langmead, B. & Salzberg, S. L. Nat. Methods 2012, 9, 357-359, incorporated by reference). Reads mapping to ENCODE blacklisted regions were removed, and duplicated reads were removed with Picard MarkDuplicates. Peaks were called using MACS2 (Zhang et al. Genome Biol. 2008, 9, R137, incorporated by reference). For visualization, bamCoverage was used to generate rpkm-normalized bigwig files from deduplicated bam files. Quality of each sample was assessed based on number of uniquely mapping reads after blacklist removal. A union peak set was generated from narrowPeak files of all samples. The count files for each sample were generated using the featureCounts function.

Differential peak analysis was conducted with DESeq2 on the feature count files with an adjusted p-value threshold of p<0.01 for differential peak analysis.

### Example 2

### Generation of SNRPN-2A-GFP Reporter Cell Lines for CRISPER Screening

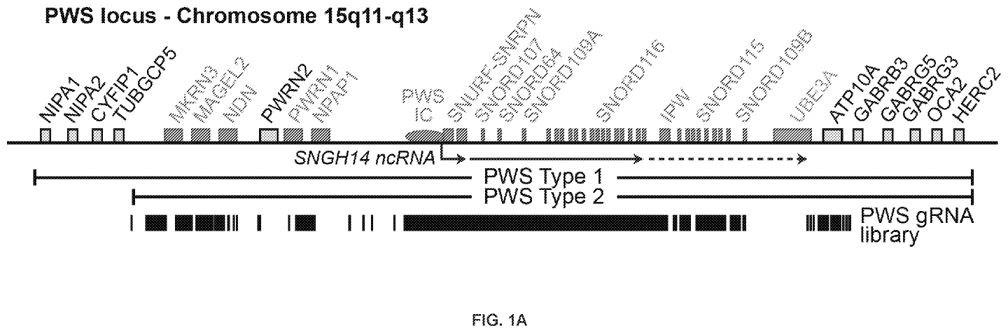

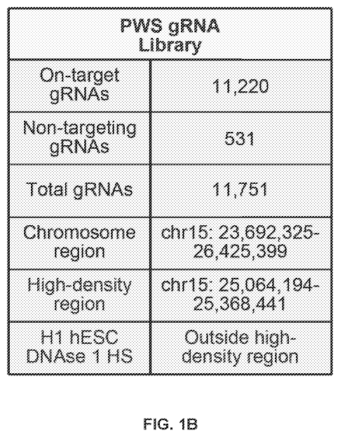

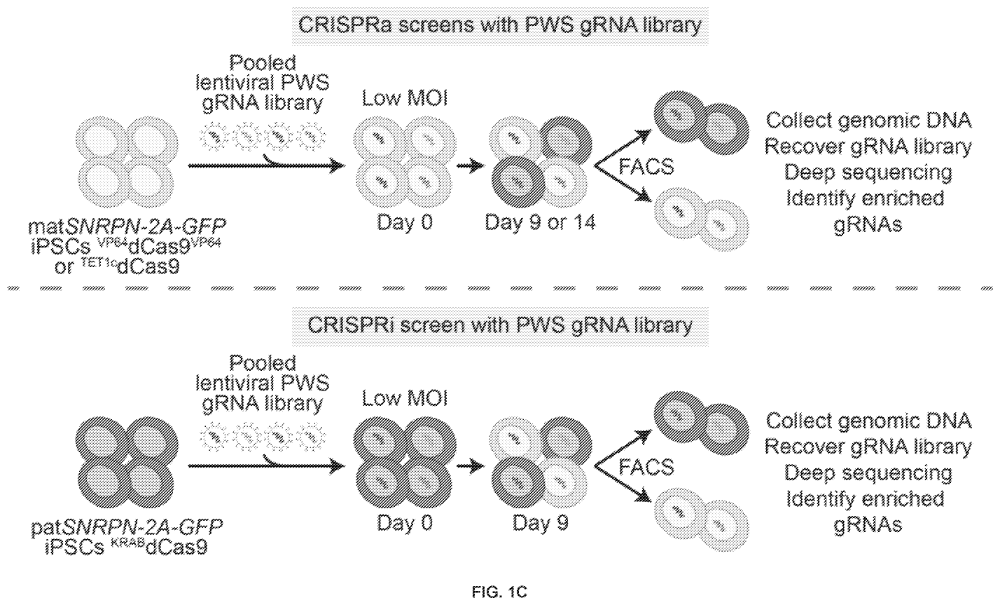

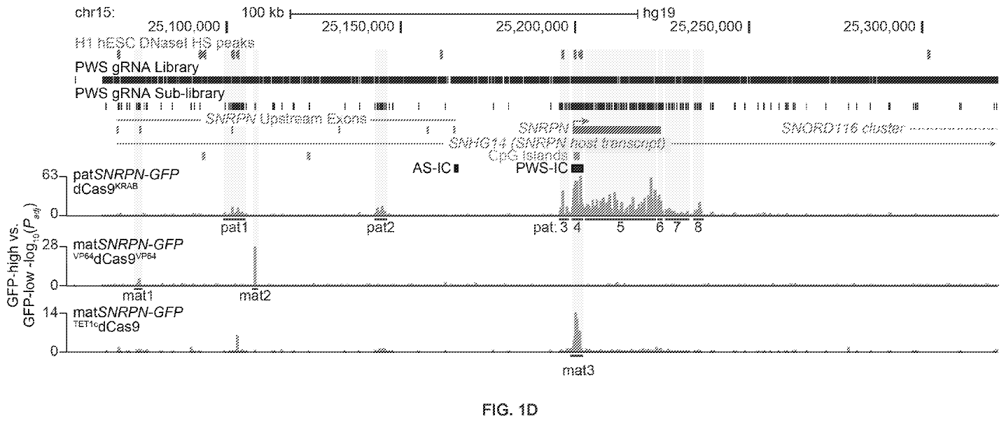

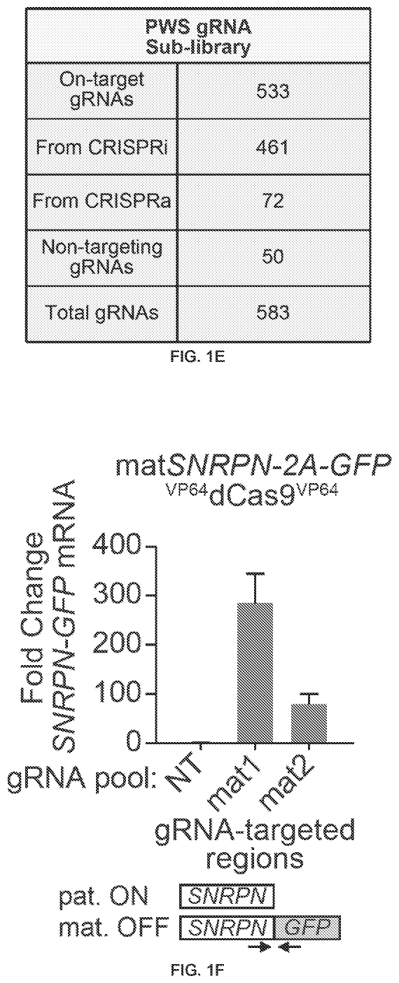

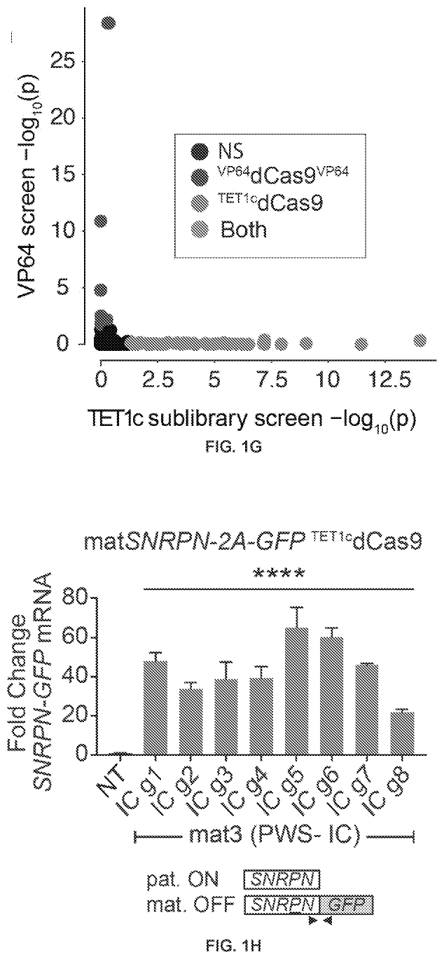

There are several imprinted genes within the chromosome 15q11-13 region, including the paternally expressed protein-coding genes MAGEL2, NDN, and SNURF-SNRPN, along with numerous noncoding RNAs (ncRNAs), including the snoRNA clusters SNORD115 and SNORD116 (FIG. 1A). Prader-Willi Syndrome (PWS) patient genotypes commonly consist of deletions within 15q11-13 that encompass several of the coding and noncoding genes, although a subset of genotypes emphasize the snoRNA clusters as having particular influence in the etiology of PWS. Further evidence suggests that SNURF-SNRPN and downstream ncRNAs, including SPA RNAs and snoRNAs, are processed from a single host transcript that initiates at the PWS imprinting center (PWS-IC). Given that imprinting within 15q11-13 may be orchestrated in part by the PWS-IC, which is in exon 1 of SNRPN and serves as the initiation of a host transcript that processes several genes implicated in PWS, SNRPN expression was chosen as a proxy for the imprinting status of the 15q11-13 locus.

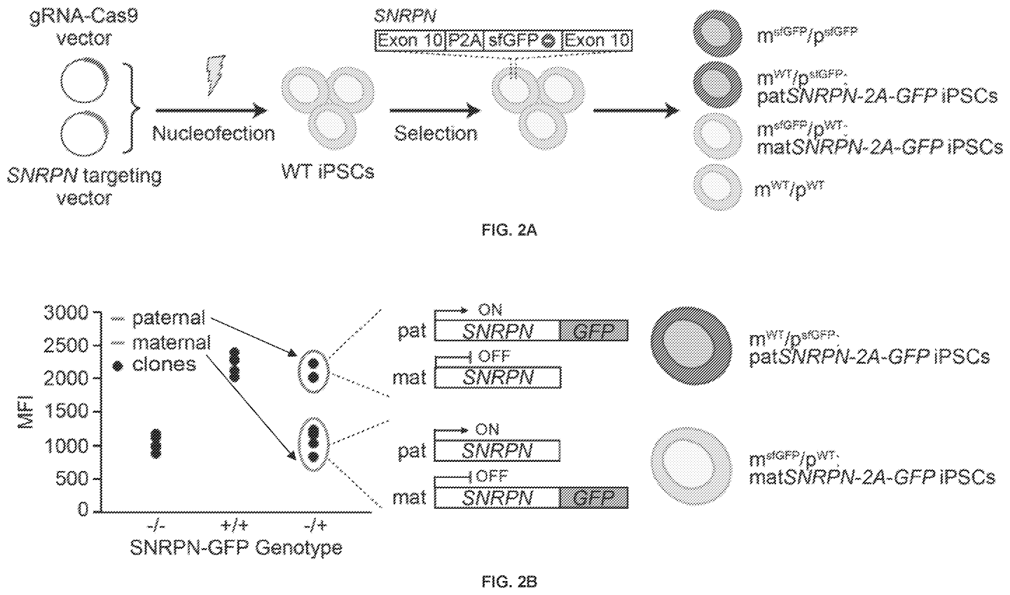

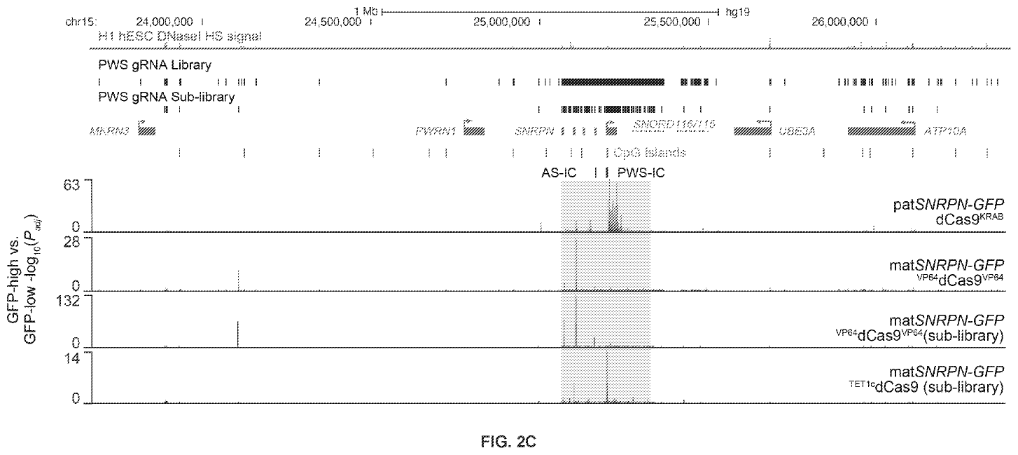

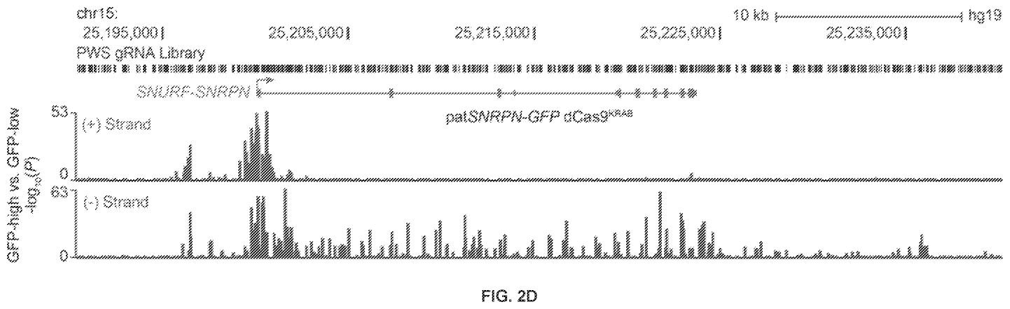

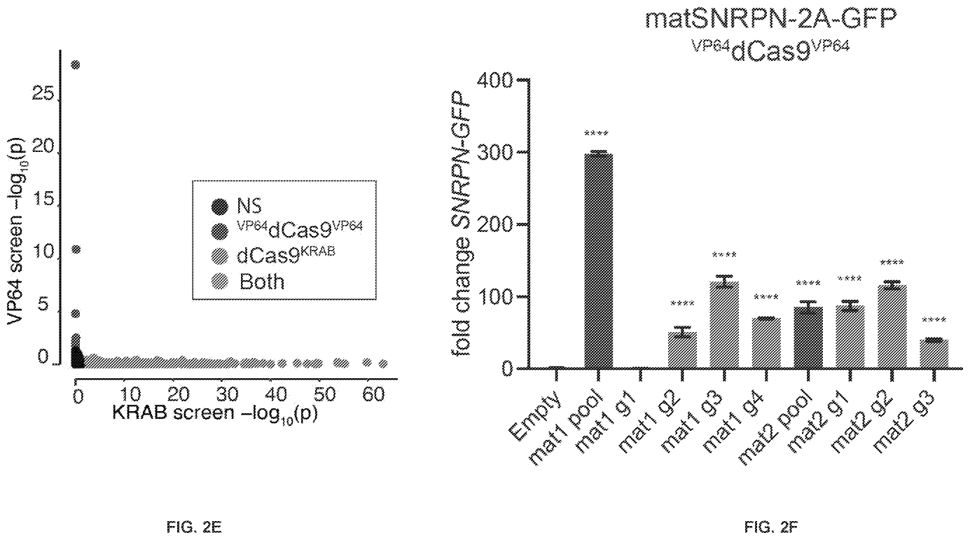

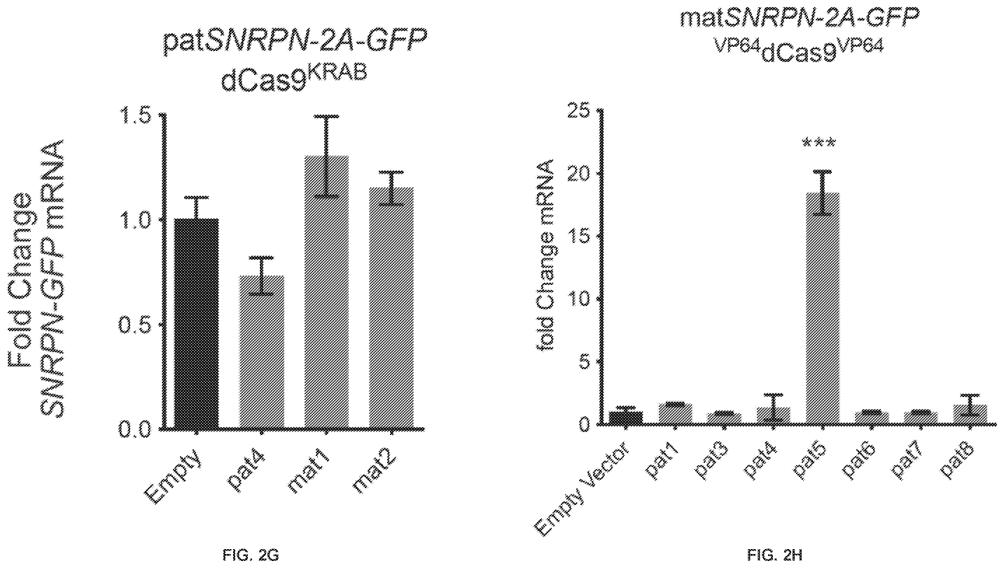

Through CRISPR/Cas9-based homologous recombination, the coding sequence of superfolder GFP (sfGFP) was inserted into exon 10 of SNRPN in a wild-type human iPSC line (FIG. 2A, FIG. 2B). A P2A skipping peptide was added between SNRPN and sfGFP to link the expression of the two proteins while avoiding disruption of SNRPN function, localization or stability that could result from direct fusion to GFP. Additionally, a wild-type iPSC line with two intact copies of 15q11-13 was used in order to isolate heterozygous clones with SNRPN-2A-GFP on either the maternal or paternal allele. Using this method, only the paternally tagged cells will be GFP-positive if imprinting is accurately maintained in the cell line (FIG. 2A). As expected, we derived clonal lines with either allele tagged, with the heterozygous clones uniquely displaying a bimodal distribution in GFP fluorescence (FIG. 2B). Two SNRPN-2A-GFP lines were selected that independently report on SNRPN expression from the paternal or maternal allele, respectively. These cell lines were used in subsequent CRISPR screens.

### Example 3

Identification of Allele-Specific Regulatory Elements with CRISPRa and CRISPRi Screens in SNRPN-2A-GFP hiPSCs

Parent-of-origin specific epigenetic marks within the 15q11-13 region are associated with allele-specific expression of PWS genes. Furthermore, putative cis-acting regulatory sequences have been identified within the PWS locus. CRISPR-based epigenetic editing was used to identify and reveal the function of regulatory regions that control allele-specific expression of PWS genes. Specifically, a gRNA library was designed within the 15q11-13 locus to screen for regulatory elements controlling expression of paternal or maternal SNRPN-2A-GFP (FIG. 1A, FIG. 1B, FIG. 2C).

CRISPR-based screening approaches to uncover regulatory elements found that regulatory elements were located in the proximity (i.e., within a megabase) of their target genes and were annotated with canonical markers of regulatory activity, such as DNase I hypersensitivity. However, regulatory elements may establish imprinting at early stages of differentiation and development such that the canonical signatures of the regulatory elements no longer exist. Thus, perturbation of the regulatory element function would require unbiased screening with gRNA libraries tiling the region. Consequently, the designed gRNA library consisted of a high-density region covering ˜300 kilobases (kb) centered at the PWS-IC and extending upstream to alternative SNRPN exons and downstream to the SNORD116 cluster. Additional gRNAs outside of the high-density region were designed to target putative regulatory elements throughout the remaining imprinted region based on DNase I hypersensitivity signal in human embryonic stem cells (Consortium, Nature. 2012, 489, 57-74, incorporated by reference). The full gRNA library consisted of 11.751 total gRNAs, including 531 scrambled non-targeting controls (FIG. 1B). Some gRNAs are shown in TABLE 5.

Two independent screens were performed in the paternal and maternal SNRPN-2A-GFP cell lines. A CRISPRi screen with the dCas9-KRAB repressor in paternally tagged SNRPN-2A-GFP cells (patSNRPN-2A-GFP) was performed to identify regions leading to repression of SNRPN transcription. The dCas9-KRAB repressor has been used for targeted gene repression and can function across a diversity of epigenetic contexts at promoters and distal regulatory elements. Similarly, a CRISPRa screen with the VP64-dCas9-VP64 activator in maternally tagged SNRPN-2A-GFP cells (matSNRPN-2A-GFP) was performed. The VP64 transactivation domain fused to both the N- and C-termini of dCas9 (VP64-dCas9-VP64) was used in the CRISPRa platform due to its reported broad activity across diverse chromatin contexts and its ability to initiate chromatin remodelling at the target site. In particular, the N- and C-termini double fusion significantly increases activation of endogenous genes compared to a single copy of VP64 across diverse loci and cell types. In both screens, the cells were transduced with the gRNA library at a multiplicity of infection (MOI) of 0.2 to ensure one gRNA per cell, cultured for nine days, and sorted via fluorescence-activation cell sorting (FACS) for the 10% highest and lowest GFP-expressing cells (FIG. 1C). Deep sequencing of gRNA abundance in each population followed by differential expression analysis was used to identify enriched or depleted gRNAs in GFP-high or GFP-low cells.

For the CRISPRi screen, the majority of significantly enriched or depleted gRNAs (gRNA “hits”) targeted the ˜300 kb high-density region (FIG. 1D, FIG. 2C). The CRISPRi screen of the paternal allele identified sites upstream of PWS-IC, within and downstream of the PWS-IC, and throughout the gene body of SNRPN (labelled pat1-pat8). The hits within and downstream of the SNRPN gene body had a strong DNA strand bias, with most hits located on the minus DNA strand and targeting the sense strand of the SNRPN gene (FIG. 2D), potentially indicating steric hindrance of gene transcription. Fewer hits were identified with the CRISPRa screen of the maternal allele. Two distinct clusters of gRNAs (labelled mat1 and mat2) were identified ˜100 kb upstream of the PWS-IC in the general region of annotated upstream SNRPN exons (FIG. 1D). There was no overlap in the gRNA hits between the maternal and paternal screens (FIG. 2E).

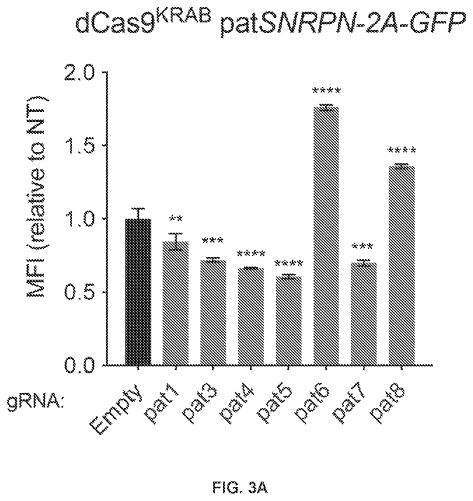

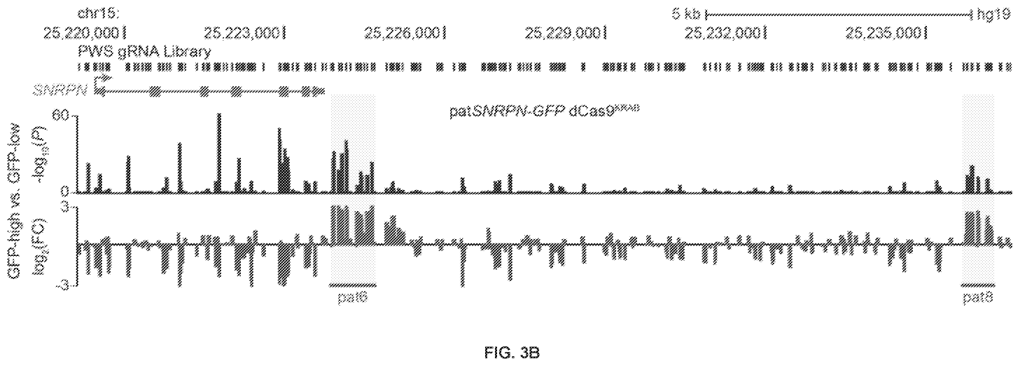

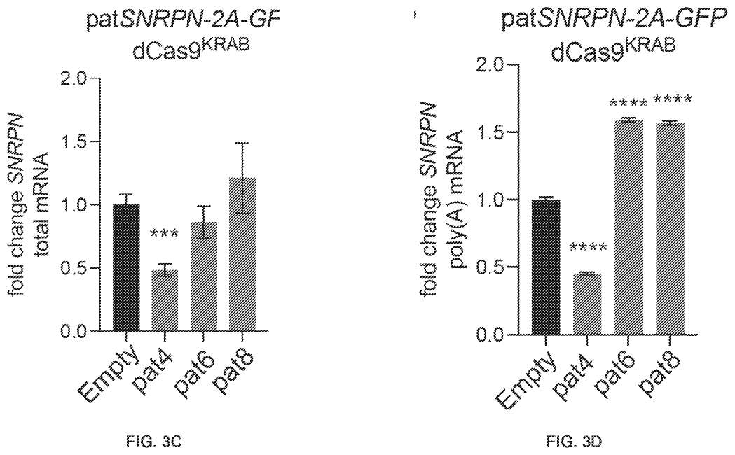

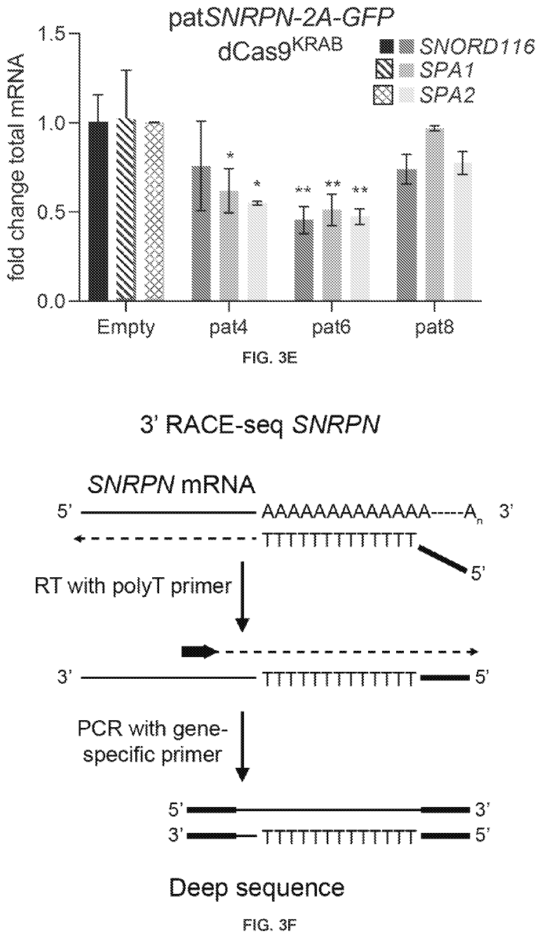

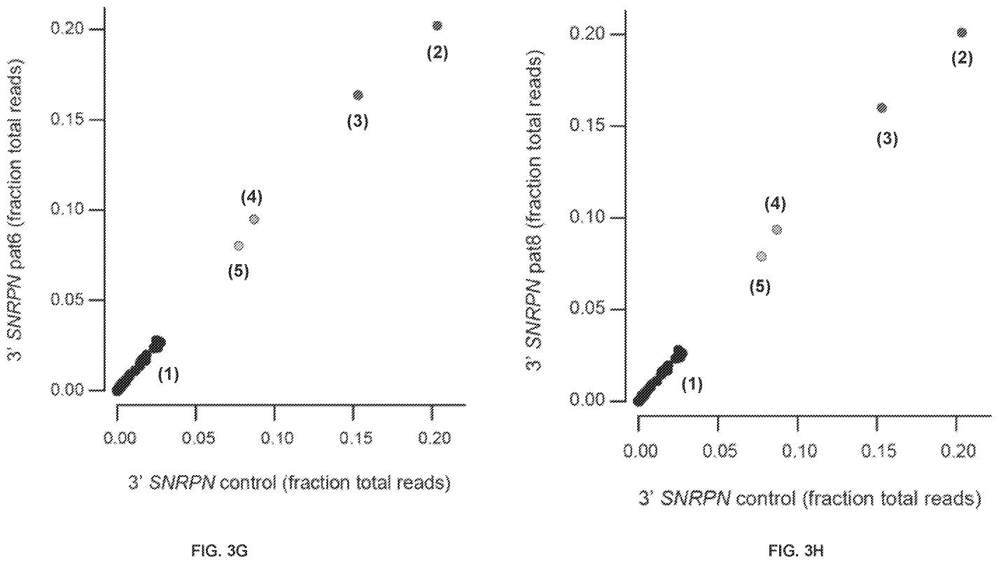

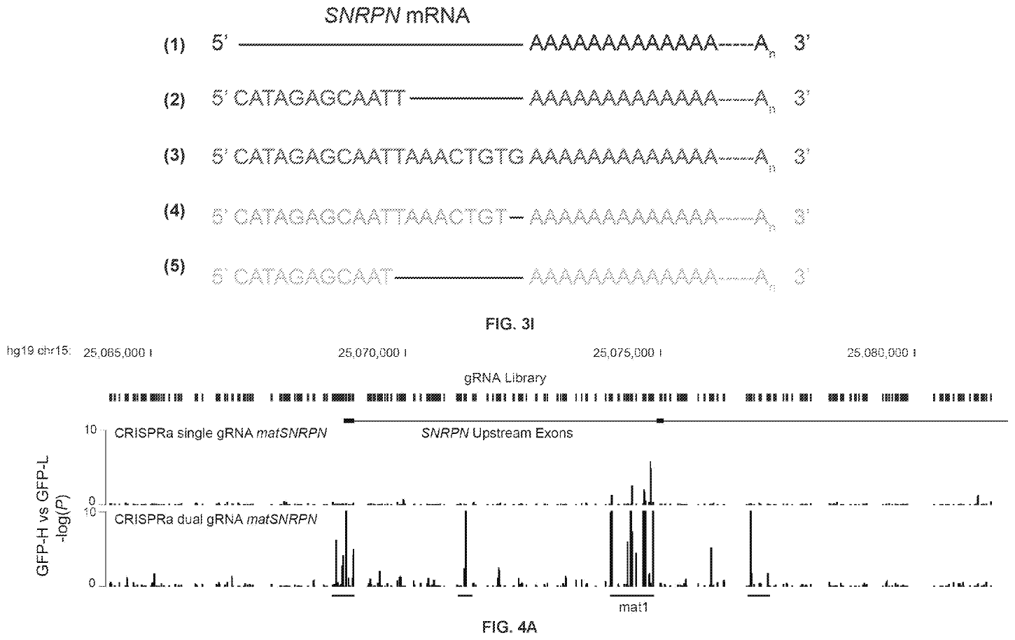

When evaluated individually, single gRNAs from the regions identified in the paternal dCas9-KRAB screen influenced GFP expression, as assessed by mean fluorescence intensity (MFI) of the GFP reporter (FIG. 3A). Rather than using single gRNAs for the initial CRISPRa validations, three or four gRNAs targeting the same region were pooled due to the reported synergistic activity of multiple gRNAs. Targeting both the mat1 and mat2 regions with VP64-dCas9-VP64 led to up-regulation of matSNRPN-2A-GFP as assessed by qRT-PCR (FIG. 1F, FIG. 2F). Individual gRNAs within each pool were validated and showed that the single best gRNAs for each region were mat1 g3 and mat2 g2, which were used for subsequent studies with VP64-dCas9-VP64 (FIG. 2F). Additionally, even though the pooled gRNAs within the mat1 region improved activation of SNRPN-GFP, the pooled gRNAs within the mat2 region appeared to have less activity on SNRPN-GFP than using mat2 g2 alone. This observation may have occurred due to the overlapping of the three mat2 gRNAs, which could hinder dCas9 binding if multiple gRNAs are present within the same cell. This was not the case with the four mat1 gRNAs.

To ensure that the lack of overlap between the maternal and paternal screens was not due to differences in sensitivity of the allele-specific GFP reporters, gRNA hits were individually tested in cells with the opposing reporter allele. Most gRNAs tested did not influence expression of the other allele (FIG. 2G, FIG. 2H). Region pat5, which overlaps the PWS-IC, exhibited a minor influence (˜20-fold) on maternal SNRPN expression with VP64-dCas9-VP64 (FIG. 2H). This level of activation was modest compared to the observed fold-change at the mat1 and mat2 regions, even with a single gRNA (FIG. 2F).

### Example 4

Activation of Paternal SNRPN-2A-GFP Through dCas9-KRAB Targeting

While most gRNA hits in the paternal screen were enriched in the GFP-low population, consistent with the expected repressive effect of dCas9-KRAB, several gRNAs within two regions, pat6 and pat8, increased the levels of measured GFP expression (FIG. 3A). These regions were both downstream of SNRPN exon 10, with pat6 directly adjacent to exon 10 and pat8 ˜10 kb further downstream (FIG. 3B). While both regions increased the MFI of the GFP reporter when targeted with dCas9-KRAB, they did not change the corresponding RNA expression measured by qRT-PCR on total RNA (FIG. 3C).

A recent study discovered that a weak polyadenylation (poly(A)) cleavage signal at the 3′ end of SNRPN enabled continued transcription of downstream ncRNAs, including SPA lncRNAs and snoRNAs. As shown herein, an up-regulation in polyadenylated SNRPN with pat6 and pat8 targeting was observed (FIG. 3D). Additionally, a down-regulation of some of the ncRNAs downstream of SNRPN, including SPA1, SPA2 and SNORD116, when targeting dCas9-KRAB to either the PWS-IC, pat6, or pat8 regions was observed (FIG. 3E). These results suggested that dCas9-KRAB targeting to the 3′ end of the SNRPN transcript may modulate polyadenylation of the host transcript and lead to changes in SNRPN protein expression without changes in transcription. Additionally, this data represents a unique application of CRISPR-based screens for alternative transcriptional termination sites.

A 3 RACE-sequencing was performed to record the distribution of UTR sequences in an unbiased fashion (FIG. 3F). There was a correlation in the abundance of SNRPN 3′ UTR sequence variants in cells treated with dCas9-KRAB and an empty gRNA vector or a gRNA targeting pat6 or pat8, indicating no change in the SNRPN 3′ UTR. The same four sequence variants were most abundantly enriched in all conditions (FIGS. 3G-3I). Therefore, targeting of dCas9-KRAB to pat6 and pat8 increased transcriptional termination and polyadenylation, but did so using the same termination sequences that function naturally in the absence of the dCas9-KRAB-mediated perturbation.

### Example 5

Additional gRNAs and Regulatory Elements Controlling Maternal SNRPN Expression Identified by a Dual gRNA Screen

Several studies have demonstrated enhanced CRISPRa activity with the delivery of multiple gRNAs functioning synergistically. Additionally, regulatory elements can function cooperatively to regulate gene expression. Therefore, simultaneous targeting of these sites may help reveal their activity. Consequently, an additional CRISPRa screen with VP64-dCas9-VP64 in matSNRPN-2A-GFP cells was performed with a dual gRNA vector, such that each cell received a pair of gRNAs. Because testing all pair-wise combinations of the original gRNA library would be too large to cover experimentally, one gRNA within the mat1 region (mat1 g3) was fixed and this fixed gRNA was paired with the 11,220 gRNA library. The screening protocol was performed identically to the original single gRNA CRISPRa screen.

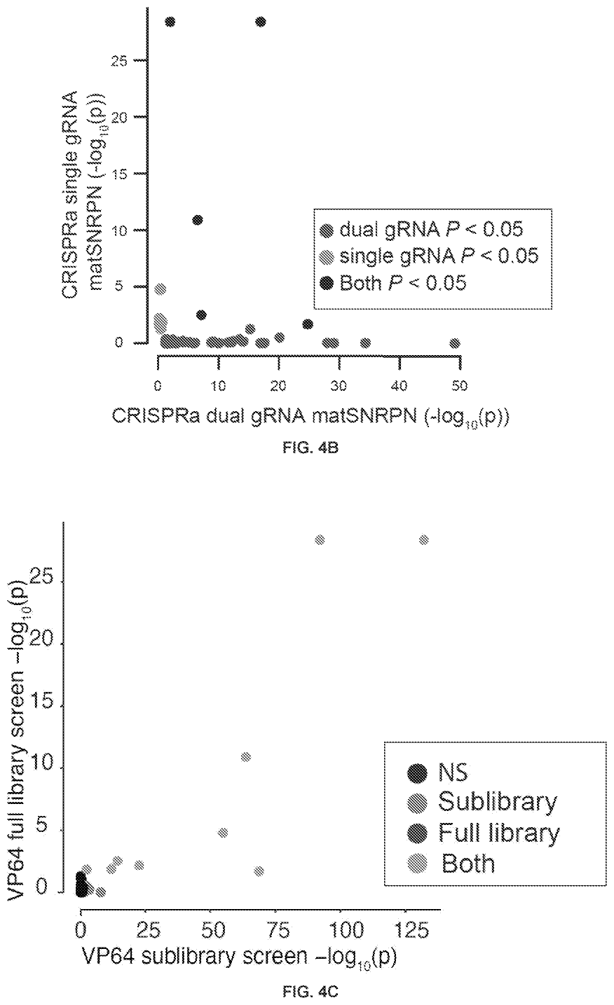

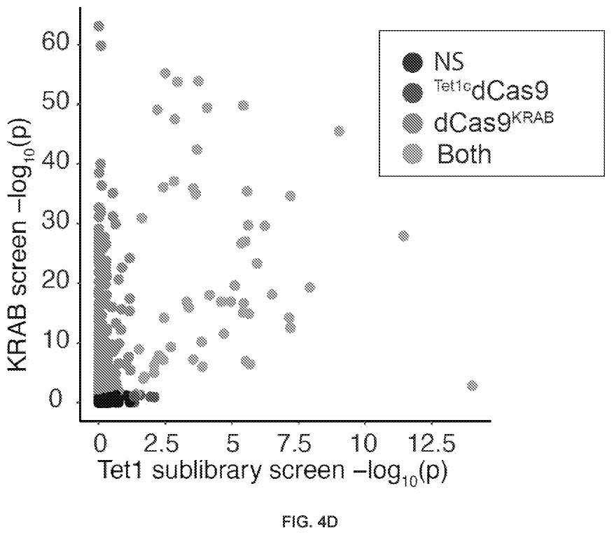

The dual gRNA screen revealed several additional gRNAs and regulatory regions that were not identified in the single gRNA screen, including a region ˜5 kb upstream from mat1 and adjacent to an annotated SNRPN exon (FIG. 4A). The most significantly enriched gRNAs in the single gRNA screen were also identified in the dual screen, with the addition of several more strongly enriched gRNAs unique to the dual screen (FIG. 46).

### Example 6

### Activation of Maternal SNRPN-2A-GFP Via Targeted Demethylation

Further studies were performed to assess whether the lack of overlap between the hits from the CRISPRa and CIRSPRi screens of the maternal and paternal alleles was related to differences in the epigenetic mechanisms regulating identified elements. VP64 activates gene expression through the recruitment of epigenetic modifiers and transcriptional machinery, often establishing a more accessible chromatin environment enriched in active histone marks, such as H3K27ac and H3K4me3. However, the influence of VP64 on other chromatin modifications linked to imprinting at the 15q11-13 locus, such as DNA methylation, may not be investigated by VP64. To directly assess the role of DNA methylation in the maintenance of the maternal imprint, dCas9 fused to the catalytic domain of Ten-eleven translocation methylcytosine dioxygenase 1 (Tet1c-dCas9) was used to catalyze DNA demethylation at the target locus. This fusion can demethylate DNA in a targeted manner and induce corresponding changes in gene expression.

A screen with Tell cdCas9 was performed similarly to the screen completed with VP64-dCas9-VP64, sorting cells based on expression of matSNRPN-2A-GFP (FIG. 1C, FIG. 1D). In this case, a gRNA sub-library was designed and consisted of all the hits from the three screens performed previously, totaling 583 gRNAs including 50 scrambled non-targeting controls (FIG. 1E). The sub-library was designed to enable small-scale screens that would facilitate the testing of several different dCas9-based epigenome editing effectors, including Tet1 cdCas9, to deconstruct the contribution of various chromatin marks at the regulatory regions identified in the initial CRISPRa and CRISPRi screens.

The sub-library screen with VP64-dCas9-VP64 recovered gRNAs within the same mat1 and mat2 regions as the screen with the full library, albeit with higher sensitivity (FIG. 2C, FIG. 4C), providing validation and adding confidence to the screening methods. The screen with Tet1c-dCas9 identified gRNA hits in the PWS-IC overlapping a CpG island within SNRPN exon 1 (mat3), which were not detected with VP64-dCas9-VP64 and largely overlapped with significant hits in the pat4 region identified in the dCas9-KRAB screen (FIG. 1G, FIG. 2C, FIG. 4D). We validated 8 individual gRNA hits in the PWS-IC, which showed robust activation of matSNRPN-GFP (FIG. 1H).

### Example 7

Activation of Maternal PWS Genes in Isogenic Wild-type and PWS iPSC Lines

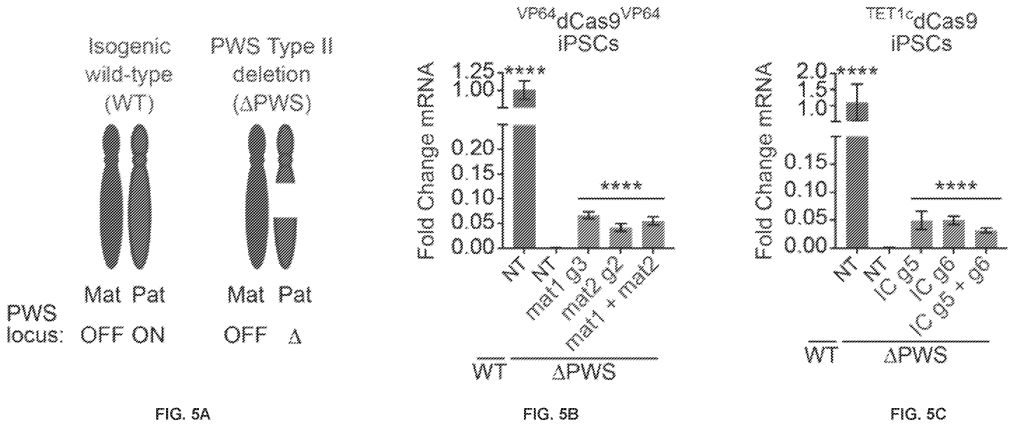

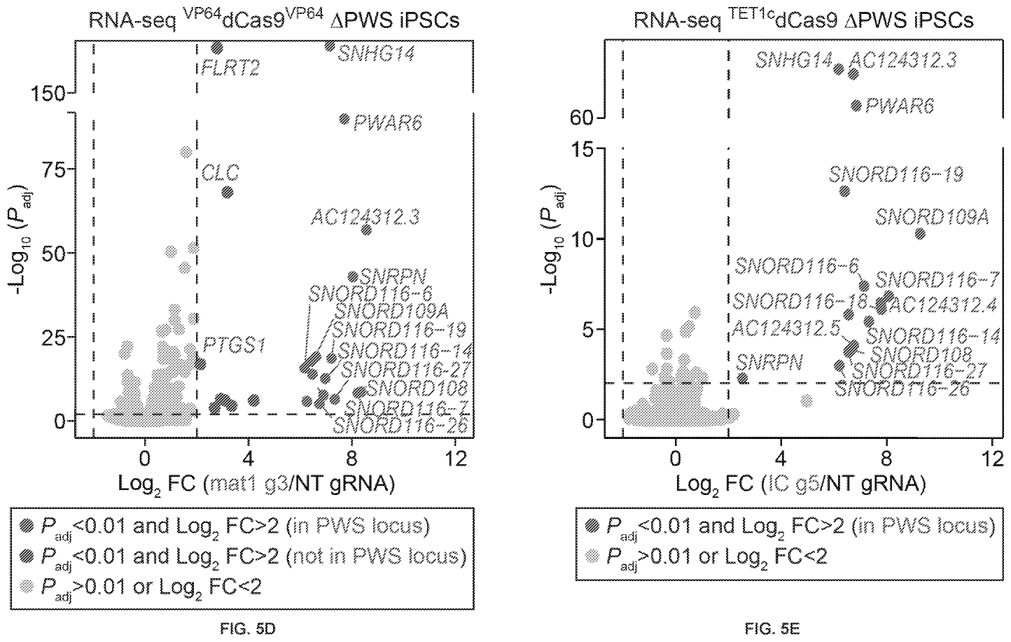

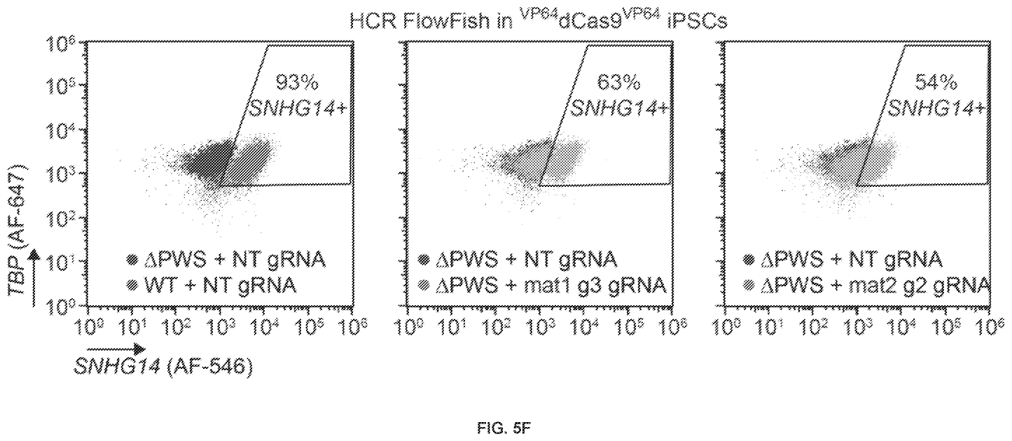

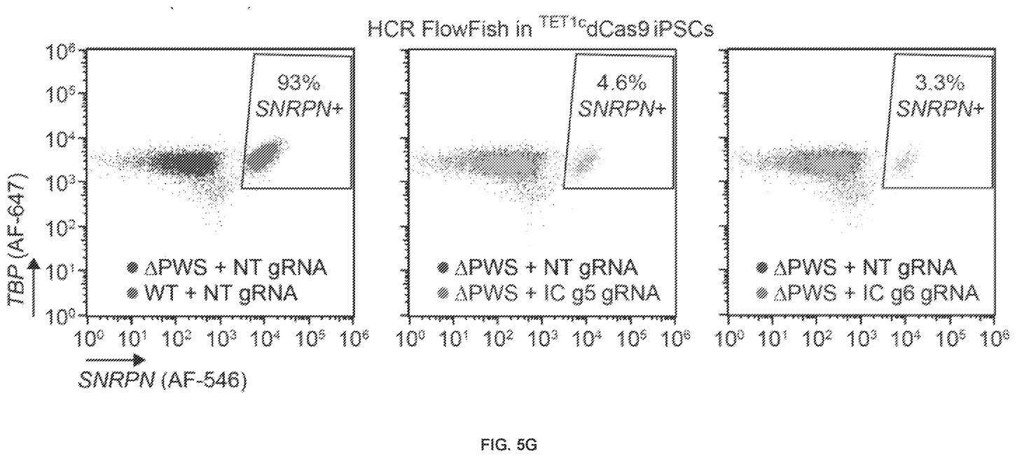

To assess how Tet1c-dCas9-mediated orVP64-dCas9-VP64-mediated maternal PWS gene activation compares to SNRPN expression from the paternal allele in the absence of any reporter construct, several effector-gRNA were tested combinations in isogenic wildtype (WT) iPSCs and iPSCs with a PWS Type II deletion introduced via Cas9 nuclease (FIG. 5A). As with screen validations, stable VP64-dCas9-VP64 and Tetlc-dCas9 cell lines were made via lentiviral transduction followed by antibiotic selection for the transgene cassette; then, these effector-expressing cell lines were transduced with individual gRNAs chosen from screen validations followed by antibiotic selection for the gRNA transgene cassette, and transcriptome-wide gene expression was assessed at 14 days post-transduction by total RNA-seq, as well as RT-qPCR for SNRPN and SNORD116.

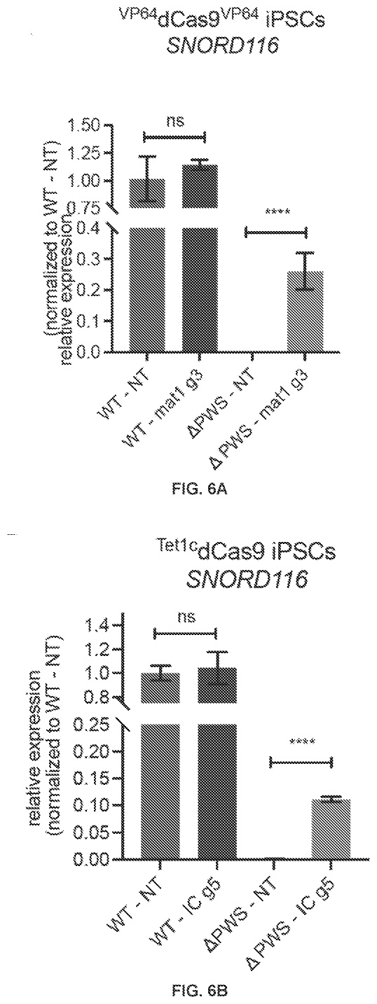

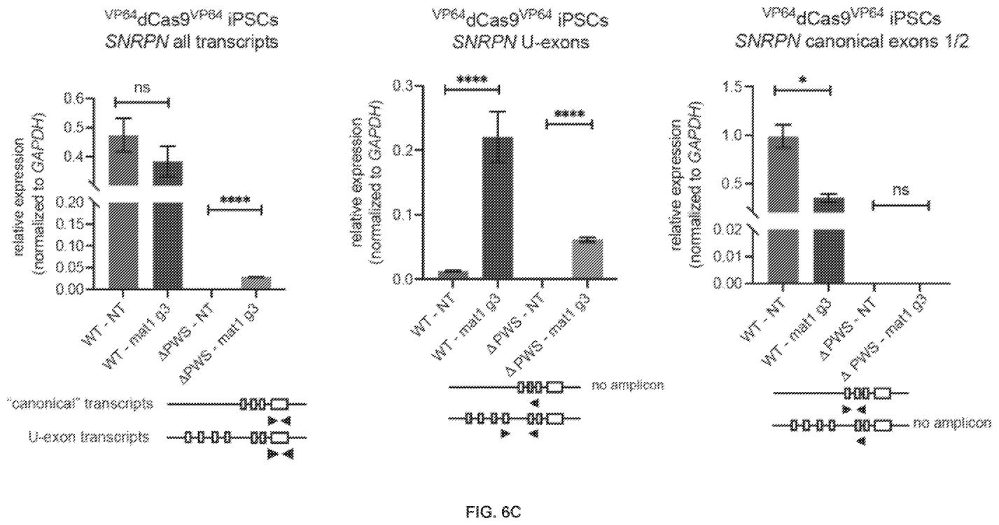

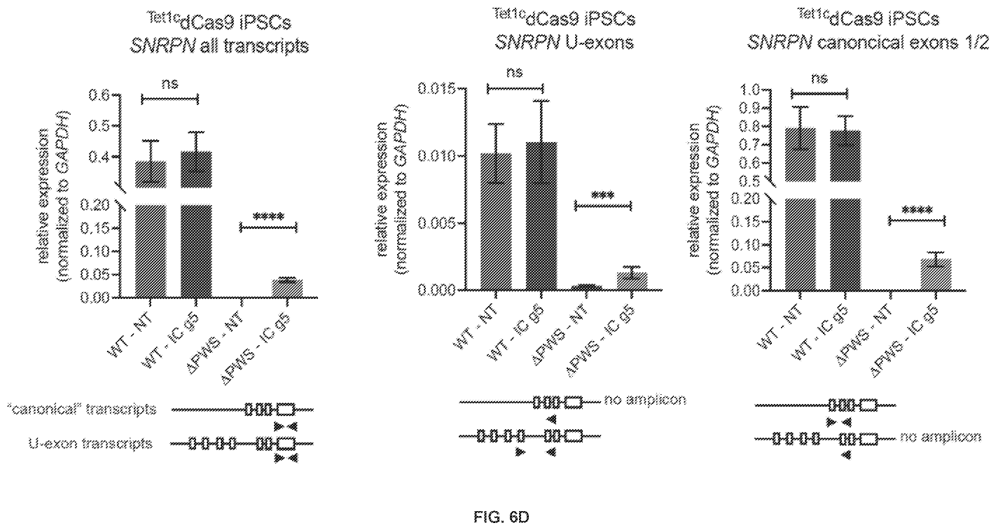

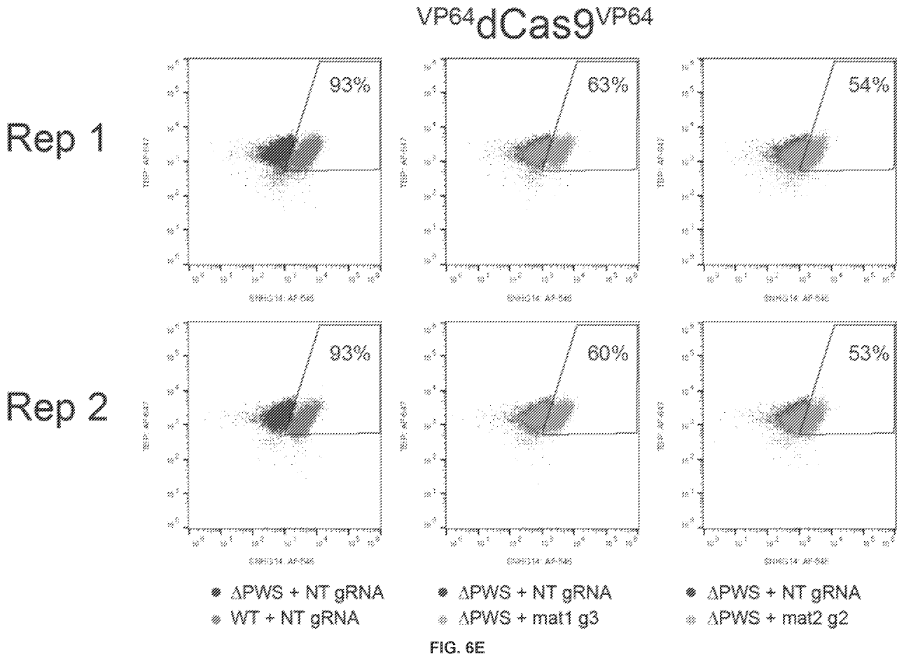

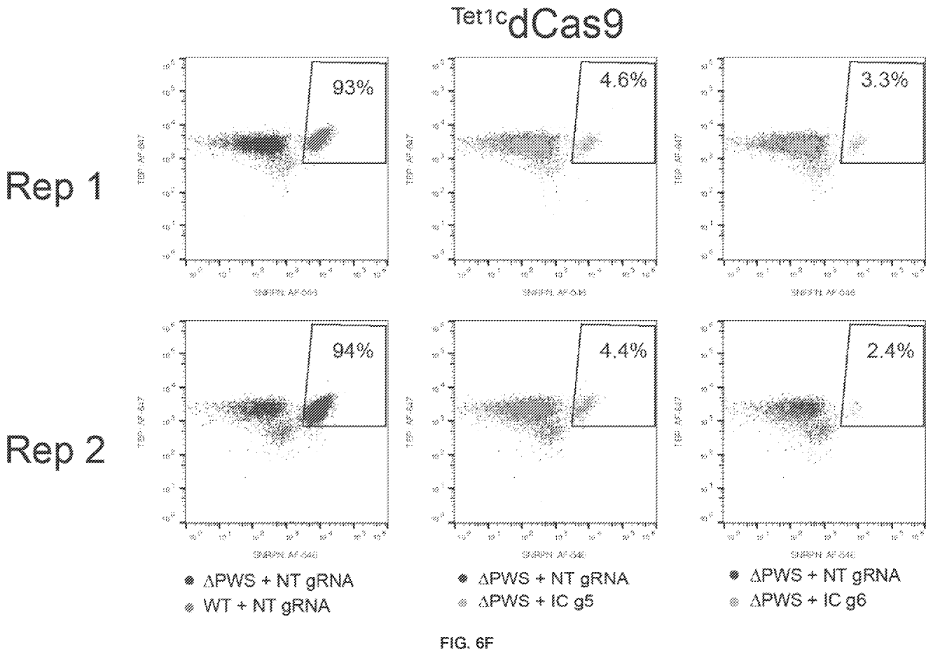

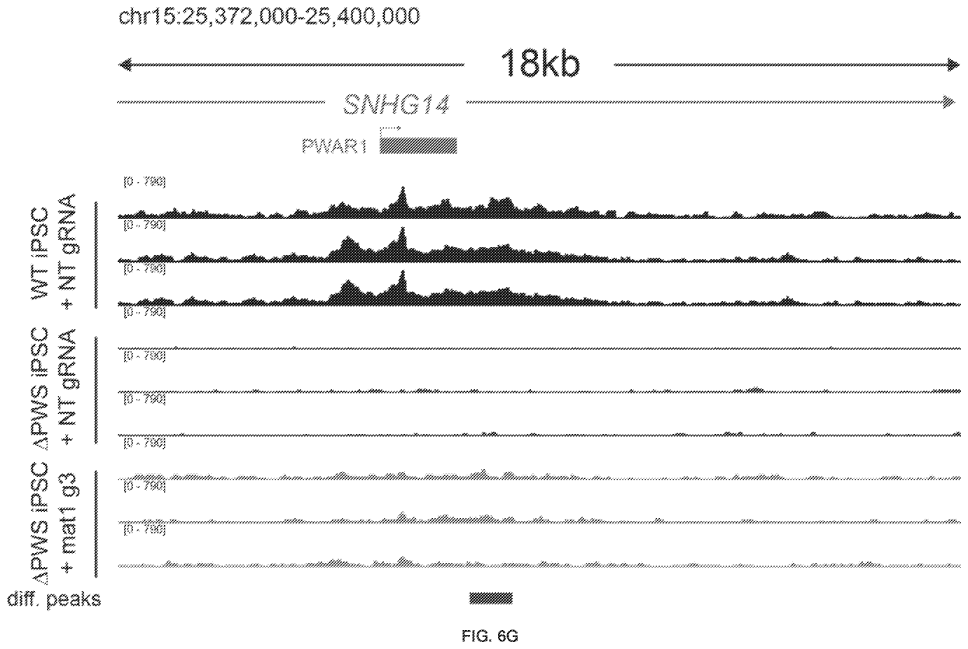

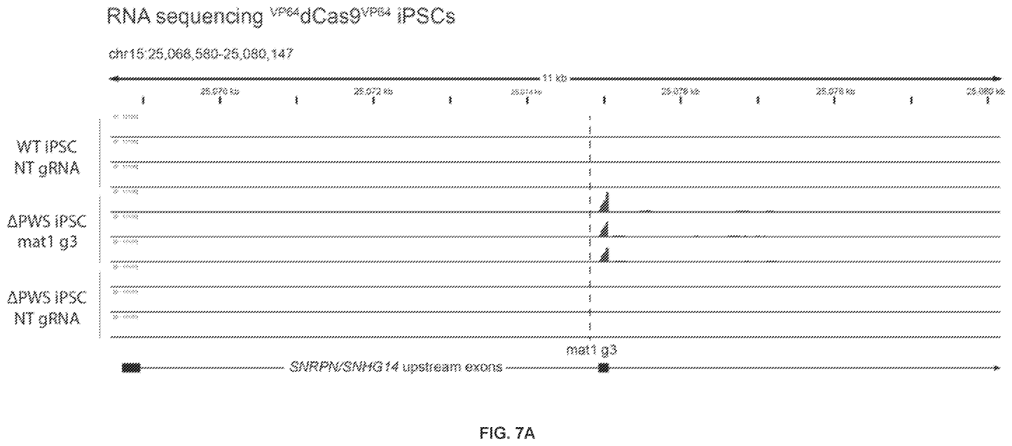

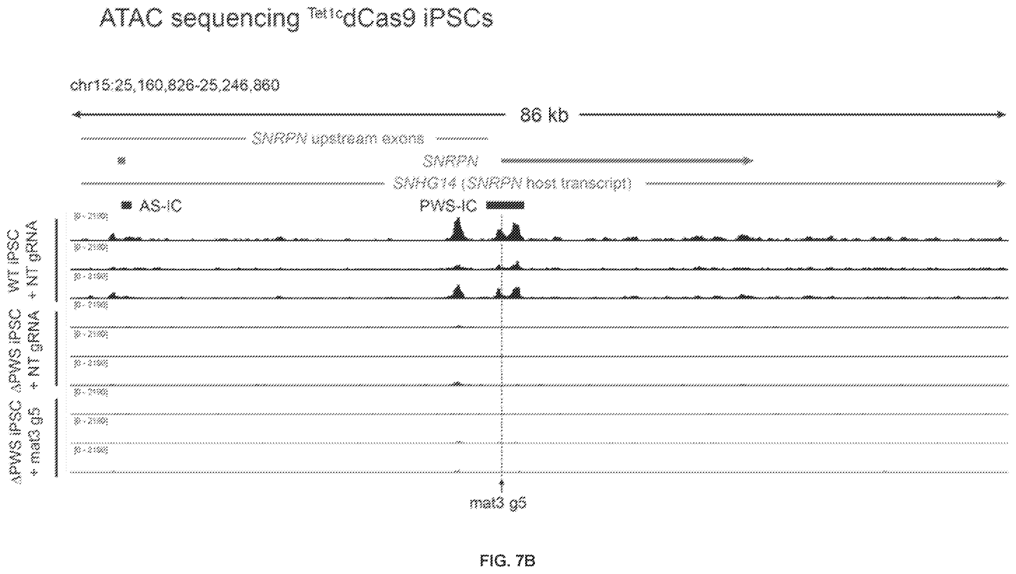

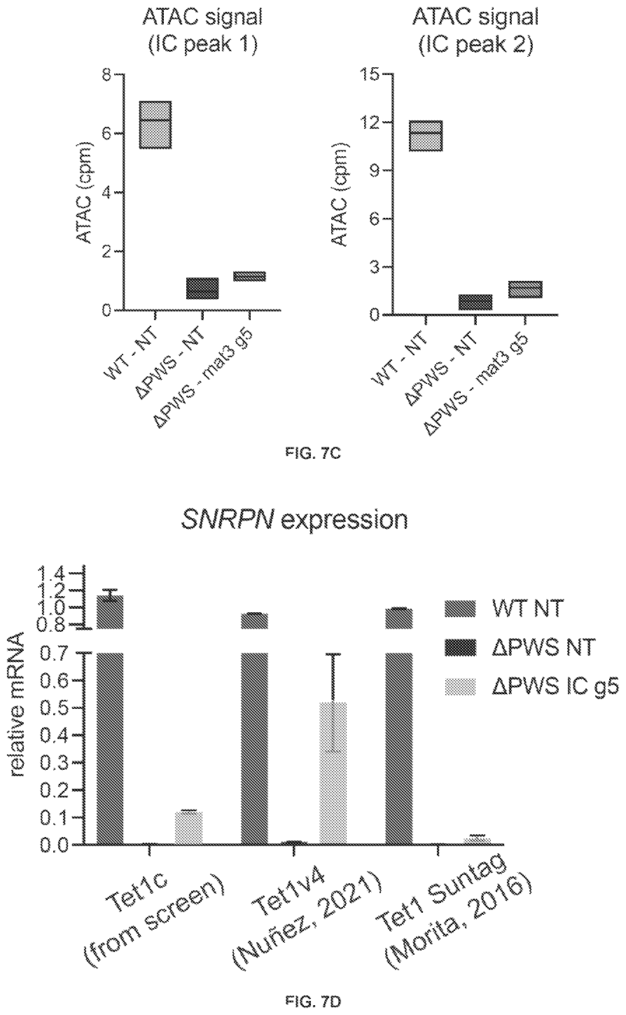

VP64-dCas9-VP64 and Tet1c-dCas9 plus a single gRNA specifically activated PWS transcripts downstream of the gRNA binding sites, including SNRPN, snoRNA 116 (SNORD116) transcripts. Additionally, the SNRPN and SNORD116/115 long host transcripts (AC124312 and SNHG14) displayed minimal off-target effects (FIGS. 5B-5E). In VP64-dCas9-VP64 ΔPWS iPSCs, 8 genes outside the PWS locus were significantly differentially expressed, although the changes in expression were lower than any PWS genes (FIG. 5D). It is possible that these expression changes outside the PWS locus may be consequences of upregulation of PWS genes, as several PWS lncRNAs, including IPW in particular, are known to regulate the expression of other genes in trans. However, there is no evidence that IPW or other PWS lncRNAs regulate the differentially-expressed genes in this study. While both VP64-dCas9-VP64 and Tet1cdCas9 robustly activated maternal SNRPN, expression was approximately 5-10% of wild-type as assessed by qPCR (FIG. 5B, FIG. 5C), indicating either incomplete reactivation of SNRPN or complete reactivation in only a subset of cells. Similarly, VP64-dCas9-VP64 and Tet1c-dCas9 activated downstream transcript SNORD116 to about 10-30% of wild-type expression, with VP64-dCas9-VP64 having a stronger effect (FIG. BA, FIG. 6B). VP64-dCas9-VP64 with mat1 g3 induced transcription upstream of the PWS IC but not at canonical exon 1 of SNRPN in both WT and ΔPWS iPSCs, whereas Tet1c-dCas9 with IC g5 induced transcription at SNRPN exon 1 (FIG. 6C, FIG. 6D), as assessed by qPCR of exon junctions specific to different subsets of transcripts. In support of the conclusion that VP64 activates transcript variants initiating upstream of the PWS-IC, RNA sequencing revealed an increase in reads in the exon immediately downstream of the mat1 g3 gRNA binding site, which is not highly expressed in WT iPSCs (FIG. 7A). Notably, SNRPN transcripts initiating at upstream exons are normally expressed in neurons but not in most other somatic cell types, suggesting that VP64-dCas9-VP64 with mat1 g3 may be recapitulating cell-type specific regulation. Additionally, VP64-dCas9-VP64 at the mat1 region appears to decrease transcripts containing exon 1 in Wr cells (FIG. 6C). Thus, transcription starting at upstream exons and proceeding through the canonical promoter region on the paternal allele may disrupt normal SNRPN transcription.

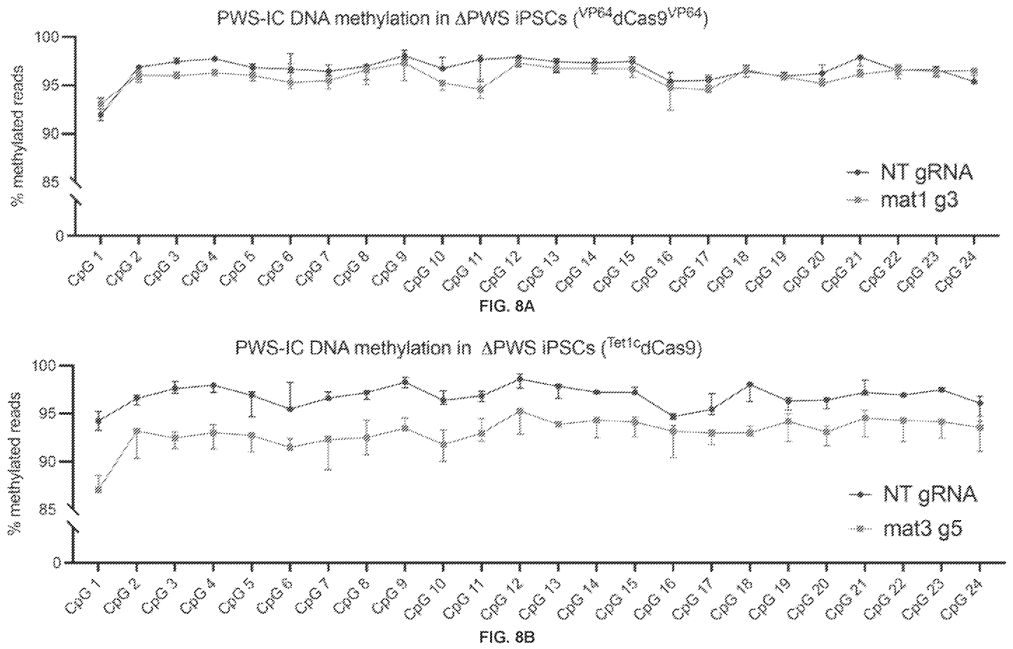

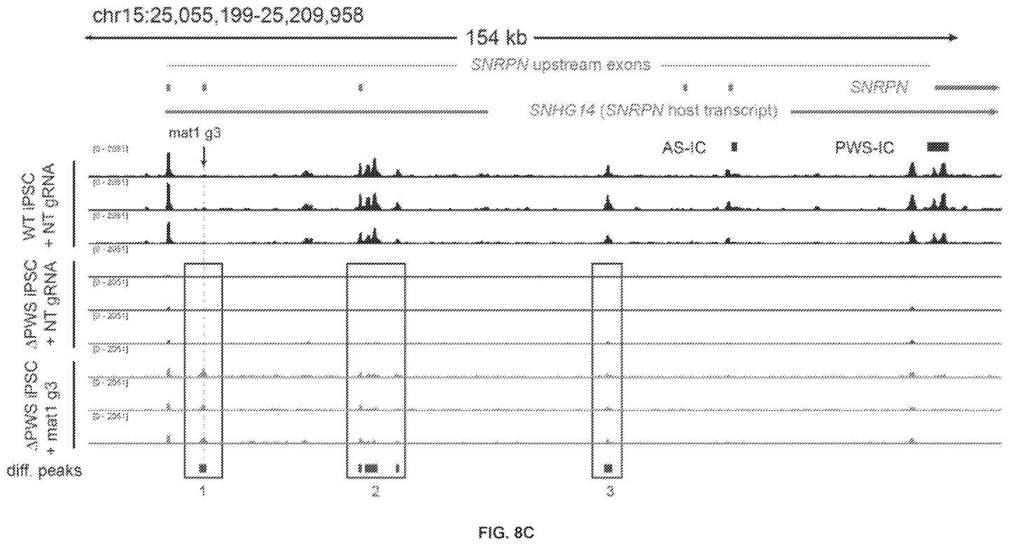

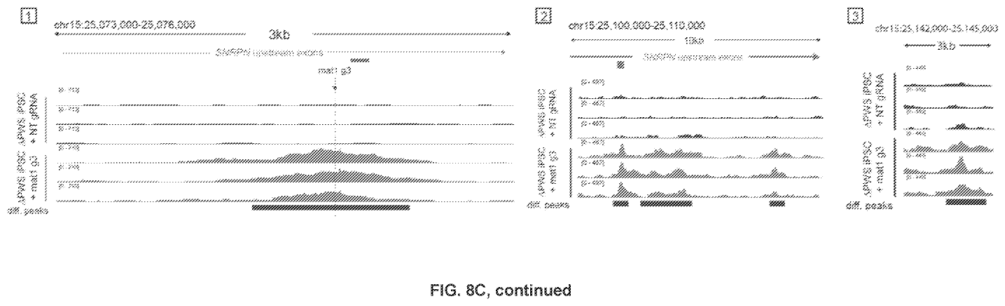

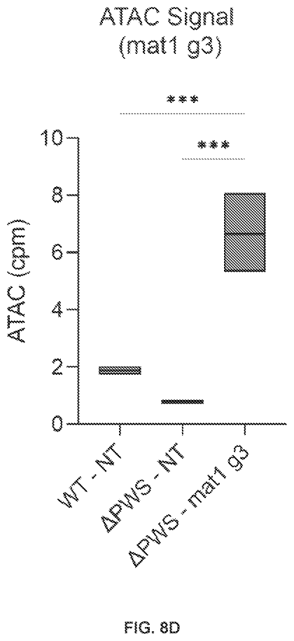

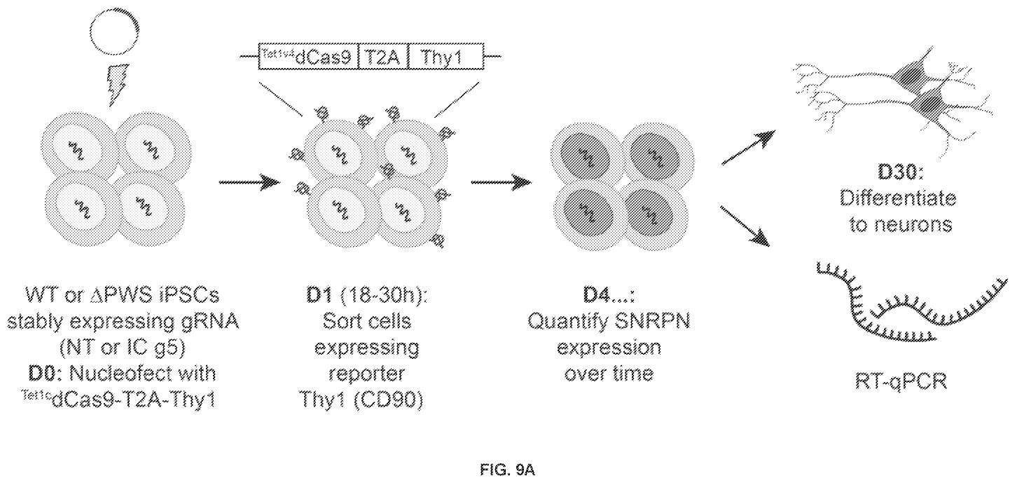

In order to identify the proportion of PWS cells that express maternal SNRPN. HCR-FlowFish was used to stain for SNHG14 transcript and SNRPN transcript variant 1 induced by VP64-dCas9-VP64 and Tetlc-Cas9, respectively. As before, cells were sequentially lentivirally transduced with separate dCas9-effector and gRNA transgenes and underwent antibiotic selection for both cassettes. Two different transcripts were measured because VP64-dCas9-VP64 and Tetlc-dCas9 preferentially upregulate different transcript variants. Whereas VP64-dCas9-VP64 with either mat1 g3 or mat2 g2 gRNAs modestly increased SNHG14 transcript to low levels in most cells, Tet1c-dCas9 with gRNAs IC g5 or IC g6 completely restored SNRPN transcripts to wild-type levels in only 5% (g5) or 3% (g6) of cells (FIG. 5F, FIG. 5G, FIG. 6E, FIG. 6F). This effect was further supported by DNA methylation analysis via bisulfite sequencing (FIG. 8A, FIG. 8B), which showed that Tet1c-dCas9 reduced DNA methylation levels at a CpG-rich region within the PWS IC by approximately 5%. Taken together, these findings indicate that demethylation of the PWS-IC on the silenced maternal locus can completely restore SNRPN transcription.

ATAC-sequencing was used to assess changes in chromatin accessibility in ΔPWS iPSCs in response to VP64-dCas9-VP64 or Tet1c-dCas9. There were 6 differential peaks (p_{adj}<0.01 and log₂(fold-change) >1) between ΔPWS iPSCs with VP64-dCas9-VP64+mat1 g3 gRNA and NT gRNA, all of which were contained in the PWS locus. Five of these peaks were upstream of SNRPN, within the SNRPN host transcript SNHG14 (FIG. 8C), and the sixth peak was in the PWAR1 gene, approximately 160 kb downstream of SNRPN and within the SNHG14 host transcript that transcribes through SNORD1181115 cluster (FIG. 6G). VP64-dCas9-VP64 binding created increases chromatin accessibility at the gRNA binding site, creating an ATAC peak not detected in WT cells (FIG. 8D). Downstream of the gRNA binding site, the chromatin accessibility landscape in ΔPWS iPSCs with VP64-dCas9-VP64+mat1 g3 gRNA resembled that of WT iPSCs, albeit with smaller peaks, likely because heterogeneity in the extent of VP64-induced chromatin remodelling and/or SNRPN activation in the cell population, as indicated by the HCR-FlowFISH data (FIG. 5F). Collectively, these unbiased and comprehensive profiling assays showed that VP64-dCas9-VP64 specifically facilitates transcription and increased chromatin accessibility throughout the PWS locus in a manner that recapitulates the paternal locus of WT cells, with few off-target effects.

There were no differential chromatin accessibility peaks detected in ΔPWS iPSCs with Tet1c-dCas9+IC g5 gRNA compared to NT gRNA (FIG. 7B), although there was a slight increase in ATAC signal at the PWS-IC (FIG. 7C). However, the FlowFISH and bisulphite sequencing showed that Tet1c-dCas9 demethylated the PWS IC and activated SNRPN expression in only 5% of the cells. Collectively, these results supported a model in which targeted activation with VP64 and demethylation by Tet1c are modulating the locus through distinct epigenetic mechanisms.

### Example 8

Transient Delivery of Tet1x-dCas9 to PWS iPSCs Induces Stable, Heritable Activation of PWS Genes

Deposition of DNA methylation at a promoter by dCas9-DNMT3 can induce stable silencing of target genes either alone or in combination with KRAB. Similarly, DNA demethylation via Tet1-dCas9 can activate genes with methylation-sensitive promoters. Because PWS genes are normally expressed from the paternal allele, the maternal allele may remain transcriptionally active, and demethylation of the PWS-IC at an initial time point by transient expression of Tet1-dCas9 may sufficient to stably activate the silenced PWS locus.

In an attempt to improve the efficiency of Tet1-mediated SNRPN activation, three different Tet1 constructs via lentiviral delivery were compared: Tet1c-dCas9, SunTag Tet1c, and Tet1v4 dCas9. SunTag Tet1c uses a recruitment strategy to recruit up to 5 copies of Tet1c to a single Cas9. Tet1v4, similar to the Tet1c-dCas9, was a direct fusion of Tet1c to the N-terminus of dCas9; however, Tet1v4 used the longer 80-amino acid XTEN80 linker, compared to the 49-amino acid linker between Tet1c and dCas9 in the original construct. The latter two Tet1 systems were cloned into a lentiviral backbone and modified with selectable markers to create stably transduced cell lines. It was found that the Tet1v4 configuration most strongly activated SNRPN in ΔPWS iPSCs (FIG. 7D). The Tet1v4 was cloned into a smaller plasmid for efficient transient transfection and the existing selectable marker was replaced with Thy1.1 (CD90), a surface protein that enables robust and sensitive antibody staining to sort cells that received the transgene.

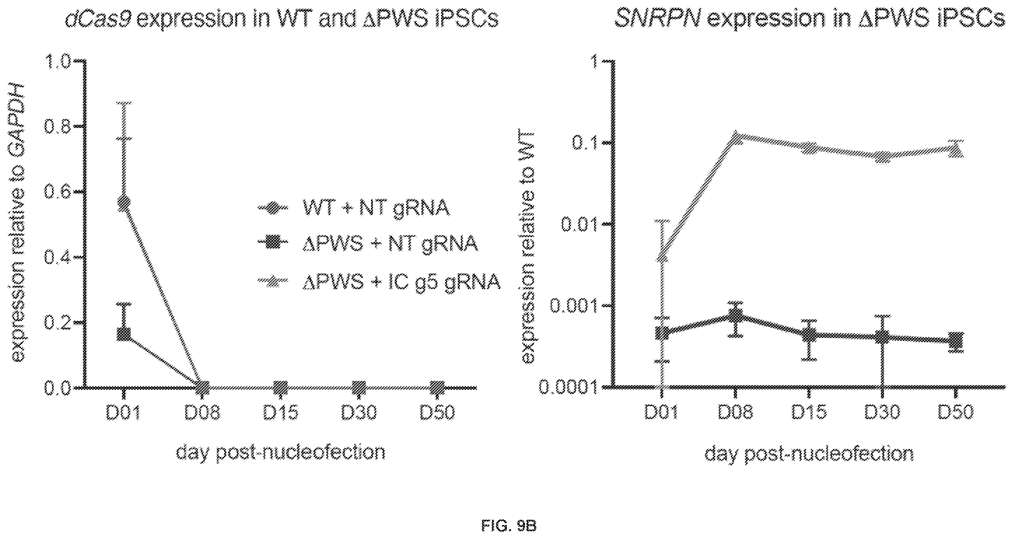

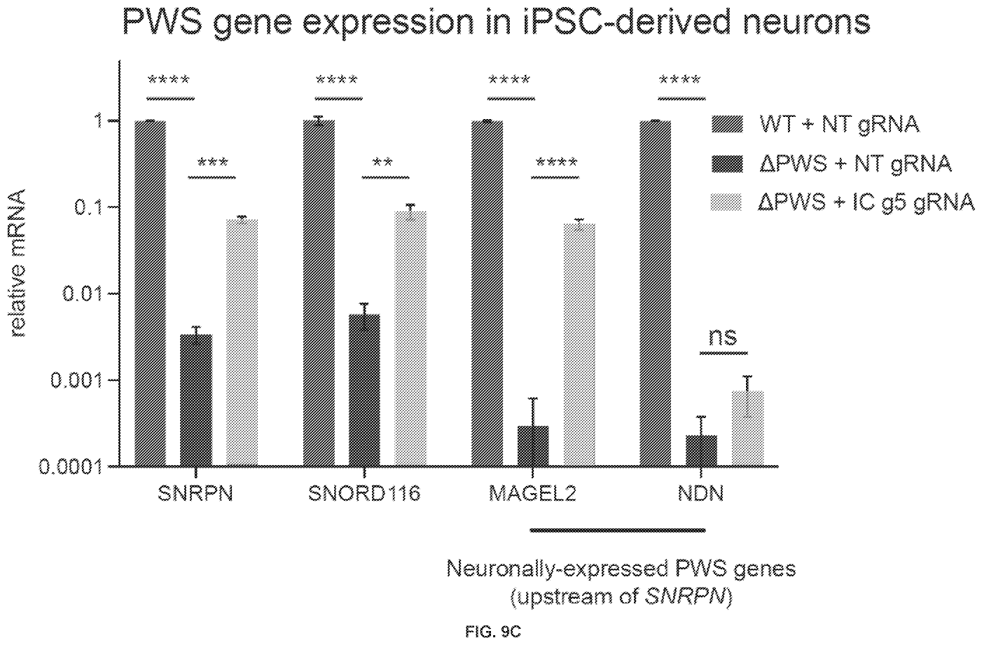

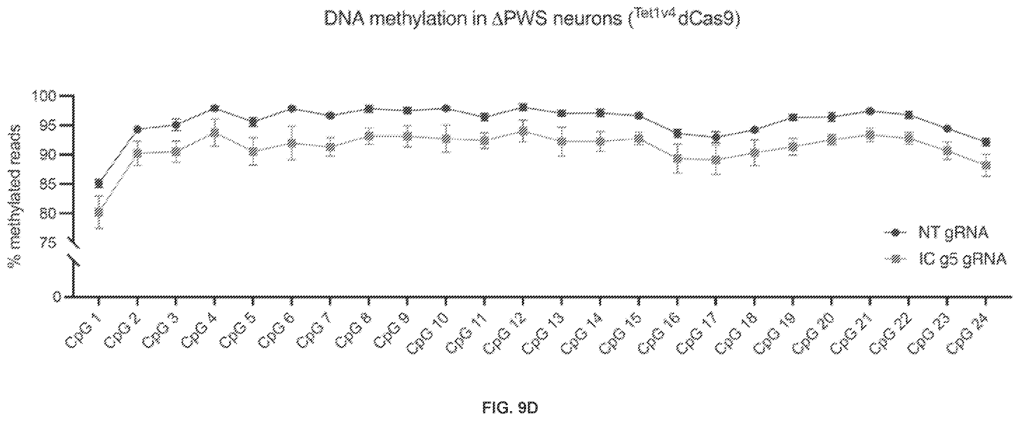

Nucleofection was used to deliver Tet1v4-dCas9-T2A-Thy1.1 (referred to here as Tet1v4-dCas9) plasmid to WT and PWS iPSCs stably expressing the gRNA (delivered via lentivirus) (FIG. 9A). The nucleofected cells were sorted at 2 days post-nucleofection on Thy1.1 reporter expression, which ensured that the assayed cells received both Tet1v4-dCas9 and gRNA. With this method, dCas9 expression in the cells was undetectable by day 8 post-nucleofection (FIG. 9B). However, SNRPN expression in the PWS iPSCs nucleofected with Tet1v4-dCas9 and IC g5 plasmids increased and remained stable through 7 weeks post nucleofection, indicating that transient expression of Tet1v4-dCas9 was sufficient to both stably and heritably reverse the silenced status of the PWS locus.

Despite perturbation in ΔPWS iPSCs, the PWS locus remained stably activated after differentiation to neurons. Approximately one-month post-nucleofection, iPSCs were differentiated to neurons via Ngn2 overexpression. At this late timepoint, dCas9 was no longer detectable in the cells. At 2-3 weeks post-differentiation, SNRPN expression was at a similar level in the iPSC-derived neurons compared to wild-type (FIG. 9C) as observed in the undifferentiated iPSCs (FIG. 6D). DNA methylation at the PWS-IC was approximately 5% lower in ΔPWS iPSC-derived neurons that had initially received Tet1v4-dCas9+IC g5 compared to NT gRNA (FIG. 9D). Furthermore, the PWS neurons that received Tet1-dCas9 and IC g5 were expressing MAGEL2 to approximately 10% of wild-type levels, which is a neuron-specific imprinted PWS transcript located 1.3 Mbp upstream of the PWS IC. NDN, another neuron-specific imprinted PWS gene located near MAGEL2, was approximately two-fold upregulated, but still well below wild-type expression levels (FIG. 9C).

### Example 9

### Discussion

In this study, CRISPR-based epigenetic screens at the 15q11-13 locus identified regulatory elements controlling expression of the SNRPN host transcript implicated in PWS. Targeting these regulatory elements with dCas9-based epigenetic editors lead to robust changes in gene expression of several candidate PWS-associated genes. This work provides compositions and methods for a therapeutic strategy for PWS by reactivating maternal gene expression at the 15q11-13 locus through targeted dCas9-based epigenetic editing.

Because the PWS locus is imprinted, fluorescent allele-specific SNRPN reporter lines were generated to enable independent analysis of maternal or paternal SNRPN gene expression. dCas9-KRAB repressed paternal SNRPN when placed throughout the promoter and gene body, except at two regions at the 3′ region of the gene that increased overall SNRPN expression. Consistent with the idea that dCas9-KRAB at the 3′ end of the gene alters polyadenylation, an increase in polyadenylated SNRPN as well as a decrease in downstream transcripts SPA1, SPA2, and SNORD116 was observed. Additionally, VP64-dCas9-VP64 less effectively activated maternal SNRPN when targeted to the canonical promoter, the PWS-IC. DNA methylation may impede activation, which could limit the effectiveness of VP64 at the methylated SNRPN promoter. However, two regions located within SNRPN upstream exons were identified that allow for upregulation of alternative SNRPN transcripts with VP64-dCas9-VP64. Targeting VP64-dCas9-VP64 to the mat1 region upstream of the SNRPN promoter activated maternal SNRPN in a DNA-methylation independent manner. In contrast, Tet1c-Cas9-mediated targeted DNA demethylation of the PWS-IC activated maternal SNRPN. Different epigenome or transcriptional modifiers may function at different regulatory elements, depending on the underlying epigenetic landscape. Initiating transcription of upstream SNRPN exons may be important for brain-specific effects, as suggested by a case of a PWS patient with an unusual deletion in the SNRPN upstream exons. Furthermore, as detailed herein, VP64-dCas9-VP84 upregulated SNORD116 more strongly than Tetlc-dCas9, possibly due to its location within the SNRPN upstream exons, which may more effectively upregulate transcripts such as SNHG14 that continue through SNRPN and into downstream PWS genes.

Transient delivery of Tet1c-Cas9 to ΔPWS iPSCs resulted in stable activation of SNRPN that persisted through neuronal differentiation. Furthermore, the imprinted gene MAGEL2 was expressed in these neurons post-differentiation, indicating that DNA methylation maintained the maternal PWS imprint. The PWS-IC is known to regulate expression of upstream neuronal transcripts. While DNA methylation-induced stable repression of a target gene has been demonstrated in several studies, removal of DNA methylation via transient delivery of a targeted DNA demethylase has not been previously explored at an endogenous imprinted locus.

Although cells that expressed both the gRNA and Tet1c-Cas9 cassettes after nucleofection were initially sorted, only a portion of that cell population expressed matSNRPN. Similarly, with lentiviral delivery of the TET1 construct and gRNA and drug selection for cells expressing both cassettes, only a subset of the cells expressed matSNRPN. There are several possible explanations for these observations. Firstly, Tet1c-Cas9 and/or gRNA expression levels may determine the efficiency of activation of the target gene, with some minimum expression threshold for successfully demethylating the PWS-IC. Therefore, variation in expression between cells may be the source of stochastic locus activation. However, lower cell viability and efficiency by nucleofection, possibly due to the large size of the Tet1c-Cas9 plasmid, and low event sort rates may decrease overall purity of the sorted population. Alternately, as iPSC populations comprise a heterogeneous cell population, it is possible that Tet1-mediated DNA demethylation was sufficient to induce SNRPN expression in a particular subset of cells, but that some cells may require perturbation of additional chromatin marks and/or transcription factors to enable a more transcriptionally permissive environment. This work builds upon other studies that have used targeted DNA demethylation editing to restore silenced gene expression in stem cells and neurons, including BDNF, FMR1, MECP2, and the imprinted Dlk1-Dio3 locus. These studies show that targeting a dCas9-TET1 fusion to the silenced gene's methylated promoter is sufficient to activate the silenced gene. In this work, there was no activation of maternal PWS genes with Tetlc-dCas9 directly in iPSC-derived neurons (data not shown). Similarly, it has been established that direct DNA methylation editing of MECP2 in neurons was only partially successful, but recruitment of the transcriptional repressor CTCF to CTCF anchor sites near the target gene improved MECP2 activation, indicating that additional barriers to transcriptional activation of these genes may be present in neurons. It is also possible that similar, further modifications to chromatin may help activate the silenced PWS locus in neurons compared to in stem cells.

Given that transient expression of Tet1c-dCas9 stably and heritably activated PWS gene expression, this work established the possibility of a one-time treatment for PWS. However, non-viral delivery of large dCas9-based effectors may be a challenge in vivo. Additionally, because PWS is a complex disorder that affects the pituitary-hypothalamic axis and manifests with several co-morbidities including sleep disturbances, improvement of symptoms may benefit from targeting multiple cell and tissue types in vivo.

Additional studies on the phenotypic effects of rescuing PWS gene expression in animal models or patient-derived organoids will be completed to further examine the potential of DNA methylation editing as a treatment for PWS. PWS patient-derived hypothalamic organoids have an impaired leptin response; these organoids may be used as a model for examining the effects of dCas9-based epigenome editing to restore PWS gene expression in a disease-relevant cell type. Further work will include direct epigenome editing of neurons to restore PWS gene expression.

The foregoing description of the specific aspects will so fully reveal the general nature of the invention that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects but should be defined only in accordance with the following claims and their equivalents.

All publications, patents, patent applications, and/or other documents cited in this application are incorporated by reference in their entirety for all purposes to the same extent as if each individual publication, patent, patent application, and/or other document were individually indicated to be incorporated by reference for all purposes.

For reasons of completeness, various aspects of the invention are set out in the following numbered clauses:

Clause 1. A method of stably activating a gene or gene product within the imprinted 15q11-13 locus in a subject having Prader Willi Syndrome (PWS) or Prader-Willi-like disorder, the method comprising non-virally administering to the subject a DNA targeting system that targets a target region in the imprinted 15q11-13 locus, the DNA targeting system comprising: a Cas protein or a fusion protein, wherein the fusion protein comprises two heterologous polypeptide domains, wherein the first polypeptide domain comprises a DNA-binding protein and wherein the second polypeptide domain has an activity selected from transcription activation activity, transcription repression activity, transcription release factor activity, histone modification activity, nuclease activity, nucleic acid association activity, methylase activity, demethylase activity, acetylation activity, and deacetylation activity, wherein the Cas protein or fusion protein is targeted to the target region in the imprinted 15q11-13 locus.

Clause 2. The method of clause 1, wherein at least one component of the DNA targeting system is transiently expressed in a cell from the subject or transiently delivered to a cell from the subject.

Clause 3. The method of any one of clauses 1-2, wherein expression of a gene within the imprinted 15q11-13 locus is maintained in a cell from the subject for at least 10, at least 15, at least 20, at least 25, at least 28, at least 30, at least 35, at least 40, at least 45, at least 48, at least 50, or at least 55 days post-administration.

Clause 4. The method of any one of clauses 1-3, wherein the DNA-binding protein comprises a Cas protein, a zinc finger protein, or a transcription activator-like effector (TALE) protein.

Clause 5. The method of any one of clauses 1-4, wherein the DNA-binding protein comprises a Cas protein and the DNA targeting system further comprises one or more guide RNAs (gRNA) that binds to the target region in the imprinted 15q11-13 locus.

Clause 6. The method of any one of clauses 1-5, wherein the Cas protein comprises a Cas9 protein.

Clause 7. The method of any one of clauses 1-6, wherein the second polypeptide domain comprises VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swm1/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

Clause 8. The method of any one of clauses 1-6, wherein the second polypeptide domain comprises KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARIDIB/PLU-1, JARID1C/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hdal. Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

Clause 9. The method of any one of clauses 1-8, wherein the second polypeptide domain comprises Tet1c or Tet1v4.

Clause 10. The method of any one of clauses 1-9, wherein the second polypeptide domain comprises the amino acid sequence of SEQ ID NO: 1139 or SEQ ID NO: 1166, or is encoded by a polynucleotide comprising the sequence of SEQ ID NO: 1138 or SEQ ID NO: 1167.

Clause 11. The method of any one of clauses 1-8, wherein the fusion protein comprises VP64-dCas9-VP64, dCas9-KRAB, Tet1c-dCas9, or Tet1v4-dCas9.

Clause 12. The method of any one of clauses 1-11, wherein the fusion protein comprises the amino acid sequence of SEQ ID NO: 1168 or SEQ ID NO: 1169, or is encoded by a polynucleotide comprising the sequence of SEQ ID NO: 1169 or SEQ ID NO: 1171.

Clause 13. The method of any one of clauses 1-12, wherein the target region in the imprinted 15q11-13 PWS-associated locus is on the maternal copy.

Clause 14. The method of any one of clauses 1-12, wherein the target region in the imprinted 15q11-13 PWS-associated locus is on the paternal copy.

Clause 15. The method of any one of clauses 1-14, wherein the expression of a gene or gene product within the imprinted 15q11-13 locus is increased.

Clause 16. The method of any one of clauses 1-15, wherein the gene within the imprinted 15q11-13 locus comprises SNRPN, MAGEL2, MKRN3, NDN, C150RF2, SNURF-SNRPN, SNHG14, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@, 115HG, SNORD115-1 to 48, SNORD109B, SNG14, or a snoRNA in the SNORD116 cluster, or a combination thereof.

Clause 17. The method of clause 16, wherein the gene within the imprinted 15q11-13 locus comprises SNRPN, SNORD116, MAGEL2, SNORD115. SPA1, and/or SPA2.

Clause 18. The method of clause 17, wherein the expression of MAGEL2 or its products is increased.

Clause 19. The method of clause 17, wherein the expression of SNORD116 or its products is increased.

Clause 20. The method of clause 17, wherein the expression of the SNRPN gene or its products is increased.

Clause 21. The method of clause 20, wherein expression of the SNRPN gene is maintained in a cell from the subject for at least 10, at least 15, at least 20, at least 25, at least 26, at least 30, at least 35, at least 40, at least 45, at least 48, at least 50, or at least 55 days post-administration.

Clause 22. The method of any one of clauses 1-21, wherein the gRNA is encoded by a polynucleotide comprising a sequence selected from SEQ ID NOs: 1148-1156 or binds to a polynucleotide comprising a sequence selected from SEQ ID NOs: 1148-1156 or comprises a sequence selected from SEQ ID NOs: 1157-1165.

Clause 23. The method of any one of clauses 5-22, wherein the DNA targeting system comprises two or more gRNAs.

Clause 24. The method of any one of clauses 1-23, wherein the subject is administered a vector comprising a polynucleotide encoding the DNA targeting system.

Clause 25. The method of clause 24, wherein the vector is a plasmid or a synthetic vector.

Clause 26. The method of clause 24, wherein the vector comprises RNA.

Clause 27. The method of clause 24, wherein the vector comprises ribonucleoprotein (RNP).

Clause 28. The method of any one of clauses 24-27, wherein the vector is a vector within a nanoparticle.

Clause 29. The method of clause 28, wherein the nanoparticle is a lipid nanoparticle or a polymeric nanoparticle.

Clause 30. A DNA targeting system that targets the imprinted 15q11-13 locus, the DNA targeting system comprising: (a) a Cas9 fusion protein, wherein the fusion protein comprises two heterologous polypeptide domains, wherein the first polypeptide domain comprises a Cas protein and the second polypeptide domain comprises Tet1, Tet1c, or Tet1v4; and (b) one or more guide RNAs (gRNA) that bind to a target region in the imprinted 15q11-13 locus.

Clause 31. The DNA targeting system of clause 30, for use in stably activating expression of a gene or gene product within the imprinted 15q11-13 locus in a subject having Prader Willi Syndrome (PWS) or Prader-Willi-like disorder.

Clause 32. An isolated polynucleotide sequence encoding the DNA targeting system of clause 30 or 31.

Clause 33. A vector comprising the isolated polynucleotide sequence of clause 32.

Clause 34. A nanoparticle comprising the DNA targeting system of clause 30 or 31, or the isolated polynucleotide sequence of clause 32, or the vector of clause 33, or a combination thereof.

Clause 35. The nanoparticle of clause 34, wherein the nanoparticle is a lipid nanoparticle or a polymeric nanoparticle.

Clause 36. A pharmaceutical composition comprising the DNA targeting system of clause 30 or 31, or the isolated polynucleotide sequence of clause 32, or the vector of clause 33, or the nanoparticle of clause 34 or 35, or a combination thereof.

SEQUENCES

| | SEQ ID | |
|---|---|---|
| gRNA name | NO | gRNA sequence |
| PWS_G.3511 | 1 | ctaagcagtggactaaggat |
| PWS_G.3549 | 2 | ctactttagattctattgta |
| PWS_G.4662 | 3 | gcagctcagaaagtgatgca |
| PWS_G.4738 | 4 | gccaggttactgattcatag |
| PWS_G.4862 | 5 | gogcactgcagcgcagacca |
| PWS_G.5117 | 6 | ggaaccagtcagaacaggtg |
| PWS_G.5706 | 7 | ggtcggcttcaggagggaag |
| PWS_G.6131 | 8 | gtggataggtgcgttcaagg |
| PWS_G.6318 | 9 | gtttcaggcctggactgggt |
| PWS_G.8738 | 10 | acagttcagggcatgagata |
| PWS_G.9414 | 11 | cagttcagggcatgagataa |
| PWS_G.10218 | 12 | gggcatgagataagggcagt |

SEQ ID NO: 13
NGG
SEQ ID NO: 14
NGA
SEQ ID NO: 15
NGAN
SEQ ID NO: 16
NGNG
SEQ ID NO: 17
NNAGAAW (W = A or T)
SEQ ID NO: 18
NAAR (R = A or G)
SEQ ID NO: 19
NNGRR (R = A or G; N can be any nucleotide residue, such as, any of A, G, C, or T)
SEQ ID NO: 20
NNGRRN (R = A or G; N can be any nucleotide residue, such as, any of A, G, C, or T)
SEQ ID NO: 21
NNGRRT (R = A or G; N can be any nucleotide residue, such as, any of A, G, C, or T)
SEQ ID NO: 22
NNGRRV (R = A or G; N can be any nucleotide residue, such as, any of A, G, C, or T)
SEQ ID NO: 23
codon optimized polynucleotide encoding S. pyogenes Cas9
atggataaaa agtacagcat cgggctggac atcggtacaa actcagtggg gtgggccgtg
attacggacg agtacaaggt accctccaaa aaatttaaag tgctgggtaa cacggacaga
cactctataa agaaaaatct tattggagcc ttgctgttcg actcaggcga gacagccgaa
gccacaaggt tgaagcggac cgccaggagg cggtatacca ggagaaagaa ccgcatatgc
tacctgcaag aaatcttcag taacgagatg gcaaaggttg acgatagctt tttccatcgc
ctggaagaat cctttcttgt tgaggaagac aagaagcacg aacggcaccc catctttggc
aatattgtcg acgaagtggc atatcacgaa aagtacccga ctatctacca cctcaggaag
aagctggtgg actctaccga taaggcggac ctcagactta tttatttggc actcgcccac
atgattaaat ttagaggaca tttcttgatc gagggcgacc tgaacccgga caacagtgac
gtcgataagc tgttcatcca acttgtgcag acctacaatc aactgttcga agaaaaccct
ataaatgctt caggagtcga cgctaaagca atcctgtccg cgcgcctctc aaaatctaga
agacttgaga atctgattgc tcagttgccc ggggaaaaga aaaatggatt gtttggcaac
ctgatcgccc tcagtctcgg actgacccca aatttcaaaa gtaacttcga cctggccgaa
gacgctaagc tccagctgtc caaggacaca tacgatgacg acctcgacaa tctgctggcc
cagattgggg atcagtacgc cgatctcttt ttggcagcaa agaacctgtc cgacgccatc
ctgttgagcg atatcttgag agtgaacacc gaaattacta aagcacccct tagcgcatct
atgatcaagc ggtacgacga gcatcatcag gatctgaccc tgctgaaggc tcttgtgagg
caacagctcc ccgaaaaata caaggaaatc ttctttgacc agagcaaaaa cggctacgct
ggctatatag atggtggggc cagtcaggag gaattctata aattcatcaa gcccattctc
gagaaaatgg acggcacaga ggagttgctg gtcaaactta acagggagga cctgctgcgg
aagcagcgga cctttgacaa cgggtctatc ccccaccaga ttcatctggg cgaactgcac
gcaatcctga ggaggcagga ggatttttat ccttttctta aagataaccg cgagaaaata
gaaaagattc ttacattcag gatcccgtac tacgtgggac ctctcgcccg gggcaattca
cggtttgcct ggatgacaag gaagtcagag gagactatta caccttggaa cttcgaagaa
gtggtggaca agggtgcatc tgcccagtct ttcatcgagc ggatgacaaa ttttgacaag
aacctcccta atgagaaggt gctgcccaaa cattctctgc tctacgagta ctttaccgtc
tacaatgaac tgactaaagt caagtacgtc accgagggaa tgaggaagcc ggcattcctt
agtggagaac agaagaaggc gattgtagac ctgttgttca agaccaacag gaaggtgact
gtgaagcaac ttaaagaaga ctactttaag aagatcgaat gttttgacag tgtggaaatt
tcaggggttg aagaccgctt caatgcgtca ttggggactt accatgatct tctcaagatc
ataaaggaca aagacttcct ggacaacgaa gaaaatgagg atattctcga agacatcgtc
ctcaccctga ccctgttcga agacagggaa atgatagaag agcgcttgaa aacctatgcc
cacctcttcg acgataaagt tatgaagcag ctgaagcgca ggagatacac aggatgggga
agattgtcaa ggaagctgat caatggaatt agggataaac agagtggcaa gaccatactg
gatttcctca aatctgatgg cttcgccaat aggaacttca tgcaactgat tcacgatgac
tctcttacct tcaaggagga cattcaaaag gctcaggtga gcgggcaggg agactccctt
catgaacaca tcgcgaattt ggcaggttcc cccgctatta aaaagggcat ccttcaaact
gtcaaggtgg tggatgaatt ggtcaaggta atgggcagac ataagccaga aaatattgtg
atcgagatgg cccgcgaaaa ccagaccaca cagaagggcc agaaaaatag tagagagcgg
atgaagagga tcgaggaggg catcaaagag ctgggatctc agattctcaa agaacacccc
gtagaaaaca cacagctgca gaacgaaaaa ttgtacttgt actatctgca gaacggcaga
gacatgtacg tcgaccaaga acttgatatt aatagactgt ccgactatga cgtagaccat
atcgtgcccc agtccttcct gaaggacgac tccattgata acaaagtctt gacaagaagc
gacaagaaca ggggtaaaag tgataatgtg cctagcgagg aggtggtgaa aaaaatgaag
aactactggc gacagctgct taatgcaaag ctcattacac aacggaagtt cgataatctg
acgaaagcag agagaggtgg cttgtctgag ttggacaagg cagggtttat taagcggcag
ctggtggaaa ctaggcagat cacaaagcac gtggcgcaga ttttggacag ccggatgaac
acaaaatacg acgaaaatga taaactgata cgagaggtca aagttatcac gctgaaaagc
aagctggtgt ccgattttcg gaaagacttc cagttctaca aagttcgcga gattaataac
taccatcatg ctcacgatgc gtacctgaac gctgttgtcg ggaccgcctt gataaagaag
tacccaaagc tggaatccga gttcgtatac ggggattaca aagtgtacga tgtgaggaaa
atgatagcca agtccgagca ggagattgga aaggccacag ctaagtactt cttttattct
aacatcatga atttttttaa gacggaaatt accctggcca acggagagat cagaaagcgg
ccccttatag agacaaatgg tgaaacaggt gaaatcgtct gggataaggg cagggatttc
gctactgtga ggaaggtgct gagtatgcca caggtaaata tcgtgaaaaa aaccgaagta
cagaccggag gattttccaa ggaaagcatt ttgcctaaaa gaaactcaga caagctcatc
gcccgcaaga aagattggga ccctaagaaa tacgggggat ttgactcacc caccgtagcc
tattctgtgc tggtggtagc taaggtggaa aaaggaaagt ctaagaagct gaagtccgtg
aaggaactct tgggaatcac tatcatggaa agatcatcct ttgaaaagaa ccctatcgat
ttcctggagg ctaagggtta caaggaggtc aagaaagacc tcatcattaa actgccaaaa
tactctctct tcgagctgga aaatggcagg aagagaatgt tggccagcgc cggagagctg
caaaagggaa acgagcttgc tctgccctcc aaatatgtta attttctcta tctcgcttcc
cactatgaaa agctgaaagg gtctcccgaa gataacgagc agaagcagct gttcgtcgaa
cagcacaagc actatctgga tgaaataatc gaacaaataa gcgagttcag caaaagggtt
atcctggcgg atgctaattt ggacaaagta ctgtctgctt ataacaagca ccgggataag
cctattaggg aacaagccga gaatataatt cacctcttta cactcacgaa tctcggagcc
cccgccgcct tcaaatactt tgatacgact atcgaccgga aacggtatac cagtaccaaa
gaggtcctcg atgccaccct catccaccag tcaattactg gcctgtacga
aacacggatcgacctctctc aactggggg cgactag
SEQ ID NO: 24
Amino acid sequence of codon optimized polynucleotide encoding S. pyogenes Cas9
MDKKYSIGLDIGTNSVGWAVITDEYKVPSKKFKVLGNTDRHSIKKNLIGALLFDSGETAEATRLKRTA
RRRYTRRKNRICYLQEIFSNEMAKVDDSFFHRLEESFLVEEDKKHERHPIFGNIVDEVAYHEKYPTIY
HLRKKLVDSTDKADLRLIYLALAHMIKFRGHFLIEGDLNPDNSDVDKLFIQLVQTYNQLFEENPINAS
GVDAKAILSARLSKSRRLENLIAQLPGEKKNGLFGNLIALSLGLTPNEKSNEDLAEDAKLQLSKDTYD
DDLDNLLAQIGDQYADLFLAAKNISDAILLSDILRVNTEITKAPLSASMIKRYDEHHQDLTLLKALVR
QQLPEKYKEIFFDQSKNGYAGYIDGGASQEEFYKFIKPILEKMDGTEELLVKLNREDLLRKQRTEDNG
SIPHQIHLGELHAILRRQEDFYPFLKDNREKIEKILTFRIPYYVGPLARGNSRFAWMTRKSEETITPW
NFEEVVDKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRENASLGTYHDLLKIIKDKDELDNEEN
EDILEDIVLTLTLFEDREMIEERLKTYAHLEDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVKVVDELV
KVMGRHKPENIVIEMARENQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGKSDNVPSEEVVKKMKNYWR
QLLNAKLITQRKEDNITKAERGGLSELDKAGFIKRQLVETRQITKHVAQILDSRMNTKYDENDKLIRE
VKVITLKSKLVSDERKDFQFYKVREINNYHHAHDAYLNAVVGTALIKKYPKLESEFVYGDYKVYDVRK
MIAKSEQEIGKATAKYFFYSNIMNFFKTEITLANGEIRKRPLIETNGETGEIVWDKGRDFATVRKVLS
MPQVNIVKKTEVQTGGFSKESILPKRNSDKLIARKKDWDPKKYGGEDSPTVAYSVLVVAKVEKGKSKK
LKSVKELLGITIMERSSFEKNPIDFLEAKGYKEVKKDLIIKLPKYSLFELENGRKRMLASAGELQKGN
ELALPSKYVNFLYLASHYEKLKGSPEDNEQKQLFVEQHKHYLDEIIEQISEFSKRVILADANLDKVLS
AYNKHRDKPIREQAENIIHLFTLTNLGAPAAFKYFDTTIDRKRYTSTKEVLDATLIHQSITGLYETRI
DLSQLGGD
SEQ ID NO: 25
codon optimized nucleic acid sequences encoding S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
tccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cgggggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 26
codon optimized nucleic acid sequences encoding S. aureus Cas9
atgaagcgga actacatcct gggcctggac atcggcatca ccagcgtggg ctacggcatc
atcgactacg agacacggga cgtgatcgat gccggcgtgc ggctgttcaa agaggccaac
gtggaaaaca acgagggcag gcggagcaag agaggcgcca gaaggctgaa gcggcggagg
cggcatagaa tccagagagt gaagaagctg ctgttcgact acaacctgct gaccgaccac
agcgagctga gcggcatcaa cccctacgag gccagagtga agggcctgag ccagaagctg
agcgaggaag agttctctgc cgccctgctg cacctggcca agagaagagg cgtgcacaac
gtgaacgagg tggaagagga caccggcaac gagctgtcca ccaaagagca gatcagccgg
aacagcaagg ccctggaaga gaaatacgtg gccgaactgc agctggaacg gctgaagaaa
gacggcgaag tgcggggcag catcaacaga ttcaagacca gcgactacgt gaaagaagcc
aaacagctgc tgaaggtgca gaaggcctac caccagctgg accagagctt catcgacacc
tacatcgacc tgctggaaac ccggcggacc tactatgagg gacctggcga gggcagcccc
ttcggctgga aggacatcaa agaatggtac gagatgctga tgggccactg cacctacttc
cccgaggaac tgcggagcgt gaagtacgcc tacaacgccg acctgtacaa cgccctgaac
gacctgaaca atctcgtgat caccagggac gagaacgaga agctggaata ttacgagaag
ttccagatca tcgagaacgt gttcaagcag aagaagaagc ccaccctgaa gcagatcgcc
aaagaaatcc tcgtgaacga agaggatatt aagggctaca gagtgaccag caccggcaag
cccgagttca ccaacctgaa ggtgtaccac gacatcaagg acattaccgc ccggaaagag
attattgaga acgccgagct gctggatcag attgccaaga tcctgaccat ctaccagagc
agcgaggaca tccaggaaga actgaccaat ctgaactccg agctgaccca ggaagagatc
gagcagatct ctaatctgaa gggctatacc ggcacccaca acctgagcct gaaggccatc
aacctgatcc tggacgagct gtggcacacc aacgacaacc agatcgctat cttcaaccgg
ctgaagctgg tgcccaagaa ggtggacctg tcccagcaga aagagatccc caccaccctg
gtggacgact tcatcctgag ccccgtcgtg aagagaagct tcatccagag catcaaagtg
atcaacgcca tcatcaagaa gtacggcctg cccaacgaca tcattatcga gctggcccgc
gagaagaact ccaaggacgc ccagaaaatg atcaacgaga tgcagaagcg gaaccggcag
accaacgagc ggatcgagga aatcatccgg accaccggca aagagaacgc caagtacctg
atcgagaaga tcaagctgca cgacatgcag gaaggcaagt gcctgtacag cctggaagcc
atccctctgg aagatctgct gaacaacccc ttcaactatg aggtggacca catcatcccc
agaagcgtgt ccttcgacaa cagcttcaac aacaaggtgc tcgtgaagca ggaagaaaac
agcaagaagg gcaaccggac cccattccag tacctgagca gcagcgacag caagatcagc
tacgaaacct tcaagaagca catcctgaat ctggccaagg gcaagggcag aatcagcaag
accaagaaag agtatctgct ggaagaacgg gacatcaaca ggttctccgt gcagaaagac
ttcatcaacc ggaacctggt ggataccaga tacgccacca gaggcctgat gaacctgctg
cggagctact tcagagtgaa caacctggac gtgaaagtga agtccatcaa tggcggcttc
accagctttc tgcggcggaa gtggaagttt aagaaagagc ggaacaaggg gtacaagcac
cacgccgagg acgccctgat cattgccaac gccgatttca tcttcaaaga gtggaagaaa
ctggacaagg ccaaaaaagt gatggaaaac cagatgttcg aggaaaagca ggccgagagc
atgcccgaga tcgaaaccga gcaggagtac aaagagatct tcatcacccc ccaccagatc
aagcacatta aggacttcaa ggactacaag tacagccacc gggtggacaa gaagcctaat
agagagctga ttaacgacac cctgtactcc acccggaagg acgacaaggg caacaccctg
atcgtgaaca atctgaacgg cctgtacgac aaggacaatg acaagctgaa aaagctgatc
aacaagagcc ccgaaaagct gctgatgtac caccacgacc cccagaccta ccagaaactg
aagctgatta tggaacagta cggcgacgag aagaatcccc tgtacaagta ctacgaggaa
accgggaact acctgaccaa gtactccaaa aaggacaacg gccccgtgat caagaagatt
aagtattacg gcaacaaact gaacgcccat ctggacatca ccgacgacta ccccaacagc
agaaacaagg tcgtgaagct gtccctgaag ccctacagat tcgacgtgta cctggacaat
ggcgtgtaca agttcgtgac cgtgaagaat ctggatgtga tcaaaaaaga aaactactac
gaagtgaata gcaagtgcta tgaggaagct aagaagctga agaagatcag caaccaggcc
gagtttatcg cctccttcta caacaacgat ctgatcaaga tcaacggcga gctgtataga
gtgatcggcg tgaacaacga cctgctgaac cggatcgaag tgaacatgat cgacatcacc
taccgcgagt acctggaaaa catgaacgac aagaggcccc ccaggatcat taagacaatc
gcctccaaga cccagagcat taagaagtac agcacagaca ttctgggcaa cctgtatgaa
gtgaaatcta agaagcaccc tcagatcatc aaaaagggc
SEQ ID NO: 27
codon optimized nucleic acid sequences encoding S. aureus Cas9
atgaagcgca actacatcct cggactggac atcggcatta cctccgtggg atacggcatc
atcgattacg aaactaggga tytgatcgac gctggagtca ggctgttcaa agaggcgaac
gtggagaaca acgaggggcg gcgctcaaag aggggggccc gccggctgaa gcgccgccgc
agacatagaa tccagcgcgt gaagaagctg ctgttcgact acaaccttct gaccgaccac
tccgaacttt ccggcatcaa cccatatgag gctagagtga agggattgtc ccaaaagctg
tccgaggaag agttctccgc cgcgttgctc cacctcgcca agcgcagggg agtgcacaat
gtgaacgaag tggaagaaga taccggaaac gagctgtcca ccaaggagca gatcagccgg
aactccaagg ccctggaaga gaaatacgtg gcggaactgc aactggagcg gctgaagaaa
gacggagaag tgcgcggctc gatcaaccgc ttcaagacct cggactacgt gaaggaggcc
aagcagctcc tgaaagtgca aaaggcctat caccaacttg accagtcctt tatcgatacc
tacatcgatc tgctcgagac tcggcggact tactacgagg gtccagggga gggctcccca
tttggttgga aggatattaa ggagtggtac gaaatgctga tgggacactg cacatacttc
cctgaggagc tgcggagcgt gaaatacgca tacaacgcag acctgtacaa cgcgctgaac
gacctgaaca atctcgtgat cacccgggac gagaacgaaa agctcgagta ttacgaaaag
ttccagatta ttgagaacgt gttcaaacag aagaagaagc cgacactgaa gcagattgcc
aaggaaatcc tcgtgaacga agaggacatc aagggctatc gagtgacctc aacgggaaag
ccggagttca ccaatctgaa ggtctaccac gacatcaaag acattaccgc ccggaaggag
atcattgaga acgcggagct gttggaccag attgcgaaga ttctgaccat ctaccaatcc
tccgaggata ttcaggaaga actcaccaac ctcaacagcg aactgaccca ggaggagata
gagcaaatct ccaacctgaa gggctacacc ggaactcata acctgagcct gaaggccatc
aacttgatcc tggacgagct gtggcacacc aacgataacc agatcgctat tttcaatcgg
ctgaagctgg tccccaagaa agtggacctc tcacaacaaa aggagatccc tactaccctt
gtggacgatt tcattctgtc ccccgtggtc aagagaagct tcatacagtc aatcaaagtg
atcaatgcca ttatcaagaa atacggtctg cccaacgaca ttatcattga gctcgcccgc
gagaagaact cgaaggacgc ccagaagatg attaacgaaa tgcagaagag gaaccgacag
actaacgaac ggatcgaaga aatcatccgg accaccggga aggaaaacgc gaagtacctg
atcgaaaaga tcaagctcca tgacatgcag gaaggaaagt gtctgtactc gctggaggcc
attccgctgg aggacttgct gaacaaccct tttaactacg aagtggatca tatcattccg
aggagcgtgt cattcgacaa ttccttcaac aacaaggtcc tcgtgaagca ggaggaaaac
tcgaagaagg gaaaccgcac gccgttccag tacctgagca gcagcgactc caagatttcc
tacgaaacct tcaagaagca catcctcaac ctggcaaagg ggaagggtcg catctccaag
accaagaagg aatatctgct ggaagaaaga gacatcaaca gattctccgt gcaaaaggac
ttcatcaacc gcaacctcgt ggatactaga tacgctactc ggggtctgat gaacctcctg
agaagctact ttagagtgaa caatctggac gtgaaggtca agtcgattaa cggaggtttc
acctccttcc tgcggcgcaa gtggaagttc aagaaggaac ggaacaaggg ctacaagcac
cacgccgagg acgccctgat cattgccaac gccgacttca tcttcaaaga atggaagaaa
cttgacaagg ctaagaaggt catggaaaac cagatgttcg aagaaaagca ggccgagtct
atgcctgaaa tcgagactga acaggagtac aaggaaatct ttattacgcc acaccagatc
aaacacatca aggatttcaa ggattacaag tactcacatc gcgtggacaa aaagccgaac
agggaactga tcaacgacac cctctactcc acccggaagg atgacaaagg gaataccctc
atcgtcaaca accttaacgg cctgtacgac aaggacaacg ataagctgaa gaagctcatt
aacaagtcgc ccgaaaagtt gctgatgtac caccacgacc ctcagactta ccagaagctc
aagctgatca tggagcagta tggggacgag aaaaacccgt tgtacaagta ctacgaagaa
actgggaatt atctgactaa gtactccaag aaagataacg gccccgtgat taagaagatt
aagtactacg gcaacaagct gaacgcccat ctggacatca ccgatgacta ccctaattcc
cgcaacaagg tcgtcaagct gagcctcaag ccctaccggt ttgatgtgta ccttgacaat
ggagtgtaca agttcgtgac tgtgaagaac cttgacgtga tcaagaagga gaactactac
gaagtcaact ccaagtgcta cgaggaagca aagaagttga agaagatctc gaaccaggcc
gagttcattg cctccttcta taacaacgac ctgattaaga tcaacggcga actgtaccgc
gtcattggcg tgaacaacga tctcctgaac cgcatcgaag tgaacatgat cgacatcact
taccgggaat acctggagaa tatgaacgac aagcgcccgc cccggatcat taagactatc
gcctcaaaga cccagtcgat caagaagtac agcaccgaca tcctgggcaa cctgtacgag
gtcaaatcga agaagcaccc ccagatcatc aagaaggga
SEQ ID NO: 28
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccagagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaggcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggggccacgaaaaaggccggccaggcaaaaaagaaaaag
SEQ ID NO: 29
codon optimized nucleic acid sequences encoding S. aureus Cas9
accggtgcca ccatgtaccc atacgatgtt ccagattacg cttcgccgaa gaaaaagcgc
aaggtcgaag cgtccatgaa aaggaactac attctggggc tggacatcgg gattacaagc
gtggggtatg ggattattga ctatgaaaca agggacgtga tcgacgcagg cgtcagactg
ttcaaggagg ccaacgtgga aaacaatgag ggacggagaa gcaagagggg agccaggcgc
ctgaaacgac ggagaaggca cagaatccag agggtgaaga aactgctgtt cgattacaac
ctgctgaccg accattctga gctgagtgga attaatcctt atgaagccag ggtgaaaggc
ctgagtcaga agctgtcaga ggaagagttt tccgcagctc tgctgcacct ggctaagcgc
cgaggagtgc ataacgtcaa tgaggtggaa gaggacaccg gcaacgagct gtctacaaag
gaacagatct cacgcaatag caaagctctg gaagagaagt atgtcgcaga gctgcagctg
gaacggctga agaaagatgg cgaggtgaga gggtcaatta ataggttcaa gacaagcgac
tacgtcaaag aagccaagca gctgctgaaa gtgcagaagg cttaccacca gctggatcag
agcttcatcg atacttatat cgacctgctg gagactcgga gaacctacta tgagggacca
ggagaaggga gccccttcgg atggaaagac atcaaggaat ggtacgagat gctgatggga
cattgcacct attttccaga agagctgaga agcgtcaagt acgcttataa cgcagatct
tacaacgccc tgaatgacct gaacaacctg gtcatcacca gggatgaaaa cgagaaactg
gaatactatg agaagttcca gatcatcgaa aacgtgttta agcagaagaa aaagcctaca
ctgaaacaga ttgctaagga gatcctggtc aacgaagagg acatcaaggg ctaccgggtg
acaagcactg gaaaaccaga gttcaccaat ctgaaagtgt atcacgatat taaggacatc
acagcacgga aagaaatcat tgagaacgcc gaactgctgg atcagattgc taagatcctg
actatctacc agagctccga ggacatccag gaagagctga ctaacctgaa cagcgagctg
acccaggaag agatcgaaca gattagtaat ctgaaggggt acaccggaac acacaacctg
tccctgaaag ctatcaatct gattctggat gagctgtggc atacaaacga caatcagatt
gcaatcttta accggctgaa gctggtccca aaaaaggtgg acctgagtca gcagaaagag
atcccaacca cactggtgga cgatttcatt ctgtcacccg tggtcaagcg gagcttcatc
cagagcatca aagtgatcaa cgccatcatc aagaagtacg gcctgcccaa tgatatcatt
atcgagctgg ctagggagaa gaacagcaag gacgcacaga agatgatcaa tgagatgcag
aaacgaaacc ggcagaccaa tgaacgcatt gaagagatta tccgaactac cgggaaagag
aacgcaaagt acctgattga aaaaatcaag ctgcacgata tgcaggaggg aaagtgtctg
tattctctgg aggccatccc cctggaggac ctgctgaaca atccattcaa ctacgaggtc
gatcatatta tccccagaag cgtgtccttc gacaattcct ttaacaacaa ggtgctggtc
aagcaggaag agaactctaa aaagggcaat aggactcctt tccagtacct gtctagttca
gattccaaga tctcttacga aacctttaaa aagcacattc tgaatctggc caaaggaaag
ggccgcatca gcaagaccaa aaaggagtac ctgctggaag agcgggacat Caacagattc
tccgtccaga aggattttat taaccggaat ctggtggaca caagatacgc tactcgcggc
ctgatgaatc tgctgcgatc ctatttccgg gtgaacaatc tggatgtgaa agtcaagtcc
atcaacggcg ggttcacatc ttttctgagg cgcaaatgga agtttaaaaa ggagcgcaac
aaagggtaca agcaccatgc cgaagatgct ctgattatcg caaatgccga cttcatcttt
aaggagtgga aaaagctgga caaagccaag aaagtgatgg agaaccagat gttcgaagag
aagcaggccg aatctatgcc cgaaatcgag acagaacagg agtacaagga gattttcatc
actcctcacc agatcaagca tatcaaggat ttcaaggact acaagtactc tcaccgggtg
gataaaaagc ccaacagaga gctgatcaat gacaccctgt atagtacaag aaaagacgat
aaggggaata ccctgattgt gaacaatctg aacggactgt acgacaaaga taatgacaag
ctgaaaaagc tgatcaacaa aagtcccgag aagctgctga tgtaccacca tgatcctcag
acatatcaga aactgaagct gattatggag cagtacggcg acgagaagaa cccactgtat
aagtactatg aagagactgg gaactacctg accaagtata gcaaaaagga taatggcccc
gtgatcaaga agatcaagta ctatgggaac aagctgaatg cccatctgga catcacagac
gattacccta acagtcgcaa caaggtggtc aagctgtcac tgaagccata cagattcgat
gtctatctgg acaacggcgt gtataaattt gtgactgtca agaatctgga tgtcatcaaa
aaggagaact actatgaagt gaatagcaag tgctacgaag aggctaaaaa gctgaaaaag
attagcaacc aggcagagtt catcgcctcc ttttacaaca acgacctgat taagatcaat
ggcgaactgt atagggtcat cggggtgaac aatgatctgc tgaaccgcat tgaagtgaat
atgattgaca tcacttaccg agagtatctg gaaaacatga atgataagcg cccccctcga
attatcaaaa caattgcctc taagactcag agtatcaaaa agtactcaac cgacattctg
ggaaacctgt atgaggtgaa gagcaaaaag caccctcaga ttatcaaaaa gggctaagaa
ttc
SEQ ID NO: 30
Amino acid sequence of codon optimized nucleic acid sequences encoding S. aureus Cas9
MKRNYILGLDIGITSVGYGIIDYETRDVIDAGVRLFKEANVENNEGRRSKRGARRLKRRRRHRIQRVK
KLLEDYNLLTDHSELSGINPYEARVKGLSQKLSEEEFSAALLHLAKRRGVHNVNEVEEDTGNELSTKE
QISRNSKALEEKYVAELQLERLKKDGEVRGSINREKTSDYVKEAKQLLKVQKAYHQLDQSFIDTYIDL
LETRRTYYEGPGEGSPFGWKDIKEWYEMLMGHCTYFPEELRSVKYAYNADLYNALNDINNLVITRDEN
EKLEYYEKFQIIENVEKQKKKPTLKQIAKEILVNEEDIKGYRVTSTGKPEFTNLKVYHDIKDITARKE
IIENAELLDQIAKILTIYQSSEDIQEELTNINSELTQEEIEQISNLKGYTGTHNLSLKAINLILDELW
HTNDNQIAIFNRLKLVPKKVDLSQQKEIPTTLVDDFILSPVVKRSFIQSIKVINAIIKKYGLPNDIII
ELAREKNSKDAQKMINEMQKRNRQTNERIEEIIRTTGKENAKYLIEKIKLHDMQEGKCLYSLEAIPLE
DLLNNPFNYEVDHIIPRSVSFDNSFNNKVLVKQEENSKKGNRTPFQYLSSSDSKISYETFKKHILNLA
KGKGRISKTKKEYLLEERDINRFSVQKDFINRNLVDTRYATRGLMNLLRSYFRVNNLDVKVKSINGGF
TSFLRRKWKFKKERNKGYKHHAEDALIIANADFIFKEWKKLDKAKKVMENQMFEEKQAESMPEIETEQ
EYKEIFITPHQIKHIKDEKDYKYSHRVDKKPNRELINDTLYSTRKDDKGNTLIVNNINGLYDKDNDKL
KKLINKSPEKLLMYHHDPQTYQKLKLIMEQYGDEKNPLYKYYEETGNYLTKYSKKDNGPVIKKIKYYG
NKLNAHLDITDDYPNSRNKVVKLSLKPYRFDVYLDNGVYKFVTVKNLDVIKKENYYEVNSKCYEEAKK
LKKISNQAEFIASFYNNDLIKINGELYRVIGVNNDLLNRIEVNMIDITYREYLENMNDKRPPRIIKTI
ASKTQSIKKYSTDILGNLYEVKSKKHPQIIKKG
SEQ ID NO: 31
D10A mutant of S. aureus Cas9
atgaaaagga actacattct ggggctggcc atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagagaac
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 32
N580A mutant of S. aureus Cas9
atgaaaagga actacattct ggggctggac atcgggatta caagcgtggg gtatgggatt
attgactatg aaacaaggga cgtgatcgac gcaggcgtca gactgttcaa ggaggccaac
gtggaaaaca atgagggacg gagaagcaag aggggagcca ggcgcctgaa acgacggaga
aggcacagaa tccagagggt gaagaaactg ctgttcgatt acaacctgct gaccgaccat
tctgagctga gtggaattaa tccttatgaa gccagggtga aaggcctgag tcagaagctg
tcagaggaag agttttccgc agctctgctg cacctggcta agcgccgagg agtgcataac
gtcaatgagg tggaagagga caccggcaac gagctgtcta caaaggaaca gatctcacgc
aatagcaaag ctctggaaga gaagtatgtc gcagagctgc agctggaacg gctgaagaaa
gatggcgagg tgagagggtc aattaatagg ttcaagacaa gcgactacgt caaagaagcc
aagcagctgc tgaaagtgca gaaggcttac caccagctgg atcagagctt catcgatact
tatatcgacc tgctggagac tcggagaacc tactatgagg gaccaggaga agggagcccc
ttcggatgga aagacatcaa ggaatggtac gagatgctga tgggacattg cacctatttt
ccagaagagc tgagaagcgt caagtacgct tataacgcag atctgtacaa cgccctgaat
gacctgaaca acctggtcat caccagggat gaaaacgaga aactggaata ctatgagaag
ttccagatca tcgaaaacgt gtttaagcag aagaaaaagc ctacactgaa acagattgct
aaggagatcc tggtcaacga agaggacatc aagggctacc gggtgacaag cactggaaaa
ccagagttca ccaatctgaa agtgtatcac gatattaagg acatcacagc acggaaagaa
atcattgaga acgccgaact gctggatcag attgctaaga tcctgactat ctaccagagc
tccgaggaca tccaggaaga gctgactaac ctgaacagcg agctgaccca ggaagagatc
gaacagatta gtaatctgaa ggggtacacc ggaacacaca acctgtccct gaaagctatc
aatctgattc tggatgagct gtggcataca aacgacaatc agattgcaat ctttaaccgg
ctgaagctgg tcccaaaaaa ggtggacctg agtcagcaga aagagatccc aaccacactg
gtggacgatt tcattctgtc acccgtggtc aagcggagct tcatccagag catcaaagtg
atcaacgcca tcatcaagaa gtacggcctg cccaatgata tcattatcga gctggctagg
gagaagaaca gcaaggacgc acagaagatg atcaatgaga tgcagaaacg aaaccggcag
accaatgaac gcattgaaga gattatccga actaccggga aagagaacgc aaagtacctg
attgaaaaaa tcaagctgca cgatatgcag gagggaaagt gtctgtattc tctggaggcc
atccccctgg aggacctgct gaacaatcca ttcaactacg aggtcgatca tattatcccc
agaagcgtgt ccttcgacaa ttcctttaac aacaaggtgc tggtcaagca ggaagaggcc
tctaaaaagg gcaataggac tcctttccag tacctgtcta gttcagattc caagatctct
tacgaaacct ttaaaaagca cattctgaat ctggccaaag gaaagggccg catcagcaag
accaaaaagg agtacctgct ggaagagcgg gacatcaaca gattctccgt ccagaaggat
tttattaacc ggaatctggt ggacacaaga tacgctactc gcggcctgat gaatctgctg
cgatcctatt tccgggtgaa caatctggat gtgaaagtca agtccatcaa cggcgggttc
acatcttttc tgaggcgcaa atggaagttt aaaaaggagc gcaacaaagg gtacaagcac
catgccgaag atgctctgat tatcgcaaat gccgacttca tctttaagga gtggaaaaag
ctggacaaag ccaagaaagt gatggagaac cagatgttcg aagagaagca ggccgaatct
atgcccgaaa tcgagacaga acaggagtac aaggagattt tcatcactcc tcaccagatc
aagcatatca aggatttcaa ggactacaag tactctcacc gggtggataa aaagcccaac
agagagctga tcaatgacac cctgtatagt acaagaaaag acgataaggg gaataccctg
attgtgaaca atctgaacgg actgtacgac aaagataatg acaagctgaa aaagctgatc
aacaaaagtc ccgagaagct gctgatgtac caccatgatc ctcagacata tcagaaactg
aagctgatta tggagcagta cggcgacgag aagaacccac tgtataagta ctatgaagag
actgggaact acctgaccaa gtatagcaaa aaggataatg gccccgtgat caagaagatc
aagtactatg ggaacaagct gaatgcccat ctggacatca cagacgatta ccctaacagt
cgcaacaagg tggtcaagct gtcactgaag ccatacagat tcgatgtcta tctggacaac
ggcgtgtata aatttgtgac tgtcaagaat ctggatgtca tcaaaaagga gaactactat
gaagtgaata gcaagtgcta cgaagaggct aaaaagctga aaaagattag caaccaggca
gagttcatcg cctcctttta caacaacgac ctgattaaga tcaatggcga actgtatagg
gtcatcgggg tgaacaatga tctgctgaac cgcattgaag tgaatatgat tgacatcact
taccgagagt atctggaaaa catgaatgat aagcgccccc ctcgaattat caaaacaatt
gcctctaaga ctcagagtat caaaaagtac tcaaccgaca ttctgggaaa cctgtatgag
gtgaagagca aaaagcaccc tcagattatc aaaaagggc
SEQ ID NO: 33
NGGNG
SEQ ID NO: 34
codon optimized nucleic acid sequences encoding S. aureus Cas9
atggccccaaagaagaagcggaaggtcggtatccacggagtcccagcagccaagcggaactacatcct
gggcctggacatcggcatcaccagcgtgggctacggcatcatcgactacgagacacgggacgtgatcg
atgccggcgtgcggctgttcaaagaggccaacgtggaaaacaacgagggcaggcggagcaagagaggc
gccagaaggctgaagcggcggaggcggcatagaatccagagagtgaagaagctgctgttcgactacaa
cctgctgaccgaccacagcgagctgagcggcatcaacccctacgaggccagagtgaagggcctgagcc
agaagctgagcgaggaagagttctctgccgccctgctgcacctggccaagagaagaggcgtgcacaac
gtgaacgaggtggaagaggacaccggcaacgagctgtccaccaaagagcagatcagccggaacagcaa
ggccctggaagagaaatacgtggccgaactgcagctggaacggctgaagaaagacggcgaagtgcggg
gcagcatcaacagattcaagaccagcgactacgtgaaagaagccaaacagctgctgaaggtgcagaag
gcctaccaccagctggaccagagcttcatcgacacctacatcgacctgctggaaacccggcggaccta
ctatgagggacctggcgagggcagccccttcggctggaaggacatcaaagaatggtacgagatgctga
tgggccactgcacctacttccccgaggaactgcggagcgtgaagtacgcctacaacgccgacctgtac
aacgccctgaacgacctgaacaatctcgtgatcaccagggacgagaacgagaagctggaatattacga
gaagttccagatcatcgagaacgtgttcaagcagaagaagaagcccaccctgaagcagatcgccaaag
aaatcctcgtgaacgaagaggatattaagggctacagagtgaccagcaccggcaagcccgagttcacc
aacctgaaggtgtaccacgacatcaaggacattaccgcccggaaagagattattgagaacgccgagct
gctggatcagattgccaagatcctgaccatctaccagagcagcgaggacatccaggaagaactgacca
atctgaactccgagctgacccaggaagagatcgagcagatctctaatctgaagggctataccggcacc
cacaacctgagcctgaaggccatcaacctgatcctggacgagctgtggcacaccaacgacaaccagat
cgctatcttcaaccggctgaagctggtgcccaagaaggtggacctgtcccagcagaaagagatcccca
ccaccctggtggacgacttcatcctgagccccgtcgtgaagagaagcttcatccagagcatcaaagtg
atcaacgccatcatcaagaagtacggcctgcccaacgacatcattatcgagctggcccgcgagaagaa
ctccaaggacgcccagaaaatgatcaacgagatgcagaagcggaaccggcagaccaacgagcggatcg
aggaaatcatccggaccaccggcaaagagaacgccaagtacctgatcgagaagatcaagctgcacgac
atgcaggaaggcaagtgcctgtacagcctggaagccatccctctggaagatctgctgaacaacccctt
caactatgaggtggaccacatcatccccagaagcgtgtccttcgacaacagcttcaacaacaaggtgc
tcgtgaagcaggaagaaaacagcaagaagggcaaccggaccccattccagtacctgagcagcagcgac
agcaagatcagctacgaaaccttcaagaagcacatcctgaatctggccaagggcaagggcagaatcag
caagaccaagaaagagtatctgctggaagaacgggacatcaacaggttctccgtgcagaaagacttca
tcaaccggaacctggtggataccagatacgccaccagaggcctgatgaacctgctgcggagctacttc
agagtgaacaacctggacgtgaaagtgaagtccatcaatggcggcttcaccagctttctgcggcggaa
gtggaagtttaagaaagagcggaacaaggggtacaagcaccacgccgaggacgccctgatcattgcca
acgccgatttcatcttcaaagagtggaagaaactggacaaggccaaaaaagtgatggaaaaccagatg
ttcgaggaaaagcaggccgagagcatgcccgagatcgaaaccgagcaggagtacaaagagatcttcat
caccccccaccagatcaagcacattaaggacttcaaggactacaagtacagccaccgggtggacaaga
agcctaatagagagctgattaacgacaccctgtactccacccggaaggacgacaagggcaacaccctg
atcgtgaacaatctgaacggcctgtacgacaaggacaatgacaagctgaaaaagctgatcaacaagag
ccccgaaaagctgctgatgtaccaccacgacccccagacctaccagaaactgaagctgattatggaac
agtacggcgacgagaagaatcccctgtacaagtactacgaggaaaccgggaactacctgaccaagtac
tccaaaaaggacaacggccccgtgatcaagaagattaagtattacggcaacaaactgaacgcccatct
ggacatcaccgacgactaccccaacagcagaaacaaggtcgtgaagctgtccctgaagccctacagat
tcgacgtgtacctggacaatggcgtgtacaagttcgtgaccgtgaagaatctggatgtgatcaaaaaa
gaaaactactacgaagtgaatagcaagtgctatgaggaagctaagaagctgaagaagatcagcaacca
ggccgagtttatcgcctccttctacaacaacgatctgatcaagatcaacggcgagctgtatagagtga
tcggcgtgaacaacgacctgctgaaccggatcgaagtgaacatgatcgacatcacctaccgcgagtac
ctggaaaacatgaacgacaagaggccccccaggatcattaagacaatcgcctccaagacccagagcat
taagaagtacagcacagacattctgggcaacctgtatgaagtgaaatctaagaagcaccctcagatca
tcaaaaagggcaaaaggccggcggccacgaaaaaggccggccaggcaaaaaagaaaaag
SEQ ID NO: 35
codon optimized nucleic acid sequences encoding S. aureus Cas9
aagcggaactacatcctgggcctggacatcggcatcaccagcgtgggctacggcatcatcgactacga
gacacgggacgtgatcgatgccggcgtgcggctgttcaaagaggccaacgtggaaaacaacgagggca
ggcggagcaagagaggcgccagaaggctgaagcggcggaggcggcatagaatccagagagtgaagaag
ctgctgttcgactacaacctgctgaccgaccacagcgagctgagcggcatcaacccctacgaggccag
agtgaagggcctgagccagaagctgagcgaggaagagttctctgccgccctgctgcacctggccaaga
gaagaggcgtgcacaacgtgaacgaggtggaagaggacaccggcaacgagctgtccaccaaagagcag
atcagccggaacagcaaggccctggaagagaaatacgtggccgaactgcagctggaacggctgaagaa
agacggcgaagtgcggggcagcatcaacagattcaagaccagcgactacgtgaaagaagccaaacagc
tgctgaaggtgcagaaggcctaccaccagctggaccagagcttcatcgacacctacatcgacctgctg
gaaacccggcggacctactatgagggacctggcgagggcagccccttcggctggaaggacatcaaaga
atggtacgagatgctgatgggccactgcacctacttccccgaggaactgcggagcgtgaagtacgcct
acaacgccgacctgtacaacgccctgaacgacctgaacaatctcgtgatcaccagggacgagaacgag
aagctggaatattacgagaagttccagatcatcgagaacgtgttcaagcagaagaagaagcccaccct
gaagcagatcgccaaagaaatcctcgtgaacgaagaggatattaagggctacagagtgaccagcaccg
gcaagcccgagttcaccaacctgaaggtgtaccacgacatcaaggacattaccgcccggaaagagatt
attgagaacgccgagctgctggatcagattgccaagatcctgaccatctaccagagcagcgaggacat
ccaggaagaactgaccaatctgaactccgagctgacccaggaagagatcgagcagatctctaatctga
agggctataccggcacccacaacctgagcctgaaggccatcaacctgatcctggacgagctgtggcac
accaacgacaaccagatcgctatcttcaaccggctgaagctggtgcccaagaaggtggacctgtccca
gcagaaagagatccccaccaccctggtggacgacttcatcctgagccccgtcgtgaagagaagcttca
tccagagcatcaaagtgatcaacgccatcatcaagaagtacggcctgcccaacgacatcattatcgag
ctggcccgcgagaagaactccaaggacgcccagaaaatgatcaacgagatgcagaagcggaaccggca
gaccaacgagcggatcgaggaaatcatccggaccaccggcaaagagaacgccaagtacctgatcgaga
agatcaagctgcacgacatgcaggaaggcaagtgcctgtacagcctggaagccatccctctggaagat
ctgctgaacaaccccttcaactatgaggtggaccacatcatccccagaagcgtgtccttcgacaacag
cttcaacaacaaggtgctcgtgaagcaggaagaaaacagcaagaagggcaaccggaccccattccagt
acctgagcagcagcgacagcaagatcagctacgaaaccttcaagaagcacatcctgaatctggccaag
ggcaagggcagaatcagcaagaccaagaaagagtatctgctggaagaacgggacatcaacaggttctc
cgtgcagaaagacttcatcaaccggaacctggtggataccagatacgccaccagaggcctgatgaacc
tgctgcggagctacttcagagtgaacaacctggacgtgaaagtgaagtccatcaatggcggcttcacc
agctttctgcggcggaagtggaagtttaagaaagagcggaacaaggggtacaagcaccacgccgagga
cgccctgatcattgccaacgccgatttcatcttcaaagagtggaagaaactggacaaggccaaaaaag
tgatggaaaaccagatgttcgaggaaaagcaggccgagagcatgcccgagatcgaaaccgagcaggag
tacaaagagatcttcatcaccccccaccagatcaagcacattaaggacttcaaggactacaagtacag
ccaccgggtggacaagaagcctaatagagagctgattaacgacaccctgtactccacccggaaggacg
acaagggcaacaccctgatcgtgaacaatctgaacggcctgtacgacaaggacaatgacaagctgaaa
aagctgatcaacaagagccccgaaaagctgctgatgtaccaccacgacccccagacctaccagaaact
gaagctgattatggaacagtacggcgacgagaagaatcccctgtacaagtactacgaggaaaccggga
actacctgaccaagtactccaaaaaggacaacggccccgtgatcaagaagattaagtattacggcaac
aaactgaacgcccatctggacatcaccgacgactaccccaacagcagaaacaaggtcgtgaagctgtc
cctgaagccctacagattcgacgtgtacctggacaatggcgtgtacaagttcgtgaccgtgaagaatc
tggatgtgatcaaaaaagaaaactactacgaagtgaatagcaagtgctatgaggaagctaagaagctg
aagaagatcagcaaccaggccgagtttatcgcctccttctacaacaacgatctgatcaagatcaacgg
cgagctgtatagagtgatcggcgtgaacaacgacctgctgaaccggatcgaagtgaacatgatcgaca
tcacctaccgcgagtacctggaaaacatgaacgacaagaggccccccaggatcattaagacaatcgcc
tccaagacccagagcattaagaagtacagcacagacattctgggcaacctgtatgaagtgaaatctaa
gaagcaccctcagatcatcaaaaagggc
SEQ ID NO: 36
Amino acid sequence of codon optimized nucleic acid sequences encoding S. aureus Cas9
KRNYILGLDIGITSVGYGIIDYETRDVIDAGVRLFKEANVENNEGRRSKRGARRLKRRRRHRIQRVKK
LLFDYNLLTDHSELSGINPYEARVKGLSQKLSEEEFSAALLHLAKRRGVHNVNEVEEDTGNELSTKEQ
ISRNSKALEEKYVAELQLERLKKDGEVRGSINRFKTSDYVKEAKQLLKVQKAYHQLDQSFIDTYIDLL
ETRRTYYEGPGEGSPFGWKDIKEWYEMLMGHCTYFPEELRSVKYAYNADLYNALNDLNNLVITRDENE
KLEYYEKFQIIENVFKQKKKPTLKQIAKEILVNEEDIKGYRVTSTGKPEFTNLKVYHDIKDITARKEI
IENAELLDQIAKILTIYQSSEDIQEELTNINSELTQEEIEQISNLKGYTGTHNLSLKAINLILDELWH
TNDNQIAIFNRLKLVPKKVDLSQQKEIPTTLVDDFILSPVVKRSFIQSIKVINAIIKKYGLPNDIIIE
LAREKNSKDAQKMINEMQKRNRQTNERIEEIIRTTGKENAKYLIEKIKLHDMQEGKCLYSLEAIPLED
LLNNPFNYEVDHIIPRSVSFDNSFNNKVLVKQEENSKKGNRTPFQYLSSSDSKISYETFKKHILNLAK
GKGRISKTKKEYLLEERDINRFSVQKDFINRNLVDTRYATRGLMNLLRSYFRVNNLDVKVKSINGGFT
SFLRRKWKFKKERNKGYKHHAEDALIIANADFIFKEWKKLDKAKKVMENQMFEEKQAESMPEIETEQE
YKEIFITPHQIKHIKDEKDYKYSHRVDKKPNRELINDTLYSTRKDDKGNTLIVNNINGLYDKDNDKLK
KLINKSPEKLLMYHHDPQTYQKLKLIMEQYGDEKNPLYKYYEETGNYLTKYSKKDNGPVIKKIKYYGN
KINAHLDITDDYPNSRNKVVKLSLKPYRFDVYLDNGVYKFVTVKNLDVIKKENYYEVNSKCYEEAKKL
KKISNQAEFIASFYNNDLIKINGELYRVIGVNNDLLNRIEVNMIDITYREYLENMNDKRPPRIIKTIA
SKTQSIKKYSTDILGNLYEVKSKKHPQIIKKG
SEQ ID NO: 37
Vector (pDO242) encoding codon optimized nucleic acid sequences encoding S. aureus
Cas9
ctaaattgtaagcgttaatattttgttaaaattcgcgttaaatttttgttaaatcagctcatttttta
accaataggccgaaatcggcaaaatcccttataaatcaaaagaatagaccgagatagggttgagtgtt
gttccagtttggaacaagagtccactattaaagaacgtggactccaacgtcaaagggcgaaaaaccgt
ctatcagggcgatggcccactacgtgaaccatcaccctaatcaagttttttggggtcgaggtgccgta
aagcactaaatcggaaccctaaagggagcccccgatttagagcttgacggggaaagccggcgaacgtg
gcgagaaaggaagggaagaaagcgaaaggagcgggcgctagggcgctggcaagtgtagcggtcacgct
gcgcgtaaccaccacacccgccgcgcttaatgcgccgctacagggcgcgtcccattcgccattcaggc
tgcgcaactgttgggaagggcgatcggtgcgggcctcttcgctattacgccagctggcgaaaggggga
tgtgctgcaaggcgattaagttgggtaacgccagggttttcccagtcacgacgttgtaaaacgacggc
cagtgagcgcgcgtaatacgactcactatagggcgaattgggtacCtttaattctagtactatgcaTg
cgttgacattgattattgactagttattaatagtaatcaattacggggtcattagttcatagcccata
tatggagttccgcgttacataacttacggtaaatggcccgcctggctgaccgcccaacgacccccgcc
cattgacgtcaataatgacgtatgttcccatagtaacgccaatagggactttccattgacgtcaatgg
gtggagtatttacggtaaactgcccacttggcagtacatcaagtgtatcatatgccaagtacgccccc
tattgacgtcaatgacggtaaatggcccgcctggcattatgcccagtacatgaccttatgggactttc
ctacttggcagtacatctacgtattagtcatcgctattaccatggtgatgcggttttggcagtacatc
aatgggcgtggatagcggtttgactcacggggatttccaagtctccaccccattgacgtcaatgggag
tttgttttggcaccaaaatcaacgggactttccaaaatgtcgtaacaactccgccccattgacgcaaa
tgggcggtaggcgtgtacggtgggaggtctatataagcagagctctctggctaactaccggtgccacc
ATGAAAAGGAACTACATTCTGGGGCTGGACATCGGGATTACAAGCGTGGGGTATGGGATTATTGACTA
TGAAACAAGGGACGTGATCGACGCAGGCGTCAGACTGTTCAAGGAGGCCAACGTGGAAAACAATGAGG
GACGGAGAAGCAAGAGGGGAGCCAGGCGCCTGAAACGACGGAGAAGGCACAGAATCCAGAGGGTGAAG
AAACTGCTGTTCGATTACAACCTGCTGACCGACCATTCTGAGCTGAGTGGAATTAATCCTTATGAAGC
CAGGGTGAAAGGCCTGAGTCAGAAGCTGTCAGAGGAAGAGTTTTCCGCAGCTCTGCTGCACCTGGCTA
AGCGCCGAGGAGTGCATAACGTCAATGAGGTGGAAGAGGACACCGGCAACGAGCTGTCTACAAAGGAA
CAGATCTCACGCAATAGCAAAGCTCTGGAAGAGAAGTATGTCGCAGAGCTGCAGCTGGAACGGCTGAA
GAAAGATGGCGAGGTGAGAGGGTCAATTAATAGGTTCAAGACAAGCGACTACGTCAAAGAAGCCAAGC
AGCTGCTGAAAGTGCAGAAGGCTTACCACCAGCTGGATCAGAGCTTCATCGATACTTATATCGACCTG
CTGGAGACTCGGAGAACCTACTATGAGGGACCAGGAGAAGGGAGCCCCTTCGGATGGAAAGACATCAA
GGAATGGTACGAGATGCTGATGGGACATTGCACCTATTTTCCAGAAGAGCTGAGAAGCGTCAAGTACG
CTTATAACGCAGATCTGTACAACGCCCTGAATGACCTGAACAACCTGGTCATCACCAGGGATGAAAAC
GAGAAACTGGAATACTATGAGAAGTTCCAGATCATCGAAAACGTGTTTAAGCAGAAGAAAAAGCCTAC
ACTGAAACAGATTGCTAAGGAGATCCTGGTCAACGAAGAGGACATCAAGGGCTACCGGGTGACAAGCA
CTGGAAAACCAGAGTTCACCAATCTGAAAGTGTATCACGATATTAAGGACATCACAGCACGGAAAGAA
ATCATTGAGAACGCCGAACTGCTGGATCAGATTGCTAAGATCCTGACTATCTACCAGAGCTCCGAGGA
CATCCAGGAAGAGCTGACTAACCTGAACAGCGAGCTGACCCAGGAAGAGATCGAACAGATTAGTAATC
TGAAGGGGTACACCGGAACACACAACCTGTCCCTGAAAGCTATCAATCTGATTCTGGATGAGCTGTGG
CATACAAACGACAATCAGATTGCAATCTTTAACCGGCTGAAGCTGGTCCCAAAAAAGGTGGACCTGAG
TCAGCAGAAAGAGATCCCAACCACACTGGTGGACGATTTCATTCTGTCACCCGTGGTCAAGCGGAGCT
TCATCCAGAGCATCAAAGTGATCAACGCCATCATCAAGAAGTACGGCCTGCCCAATGATATCATTATC
GAGCTGGCTAGGGAGAAGAACAGCAAGGACGCACAGAAGATGATCAATGAGATGCAGAAACGAAACCG
GCAGACCAATGAACGCATTGAAGAGATTATCCGAACTACCGGGAAAGAGAACGCAAAGTACCTGATTG
AAAAAATCAAGCTGCACGATATGCAGGAGGGAAAGTGTCTGTATTCTCTGGAGGCCATCCCCCTGGAG
GACCTGCTGAACAATCCATTCAACTACGAGGTCGATCATATTATCCCCAGAAGCGTGTCCTTCGACAA
TTCCTTTAACAACAAGGTGCTGGTCAAGCAGGAAGAGAACTCTAAAAAGGGCAATAGGACTCCTTTCC
AGTACCTGTCTAGTTCAGATTCCAAGATCTCTTACGAAACCTTTAAAAAGCACATTCTGAATCTGGCC
AAAGGAAAGGGCCGCATCAGCAAGACCAAAAAGGAGTACCTGCTGGAAGAGCGGGACATCAACAGATT
CTCCGTCCAGAAGGATTTTATTAACCGGAATCTGGTGGACACAAGATACGCTACTCGCGGCCTGATGA
ATCTGCTGCGATCCTATTTCCGGGTGAACAATCTGGATGTGAAAGTCAAGTCCATCAACGGCGGGTTC
ACATCTTTTCTGAGGCGCAAATGGAAGTTTAAAAAGGAGCGCAACAAAGGGTACAAGCACCATGCCGA
AGATGCTCTGATTATCGCAAATGCCGACTTCATCTTTAAGGAGTGGAAAAAGCTGGACAAAGCCAAGA
AAGTGATGGAGAACCAGATGTTCGAAGAGAAGCAGGCCGAATCTATGCCCGAAATCGAGACAGAACAG
GAGTACAAGGAGATTTTCATCACTCCTCACCAGATCAAGCATATCAAGGATTTCAAGGACTACAAGTA
CTCTCACCGGGTGGATAAAAAGCCCAACAGAGAGCTGATCAATGACACCCTGTATAGTACAAGAAAAG
ACGATAAGGGGAATACCCTGATTGTGAACAATCTGAACGGACTGTACGACAAAGATAATGACAAGCTG
AAAAAGCTGATCAACAAAAGTCCCGAGAAGCTGCTGATGTACCACCATGATCCTCAGACATATCAGAA
ACTGAAGCTGATTATGGAGCAGTACGGCGACGAGAAGAACCCACTGTATAAGTACTATGAAGAGACTG
GGAACTACCTGACCAAGTATAGCAAAAAGGATAATGGCCCCGTGATCAAGAAGATCAAGTACTATGGG
AACAAGCTGAATGCCCATCTGGACATCACAGACGATTACCCTAACAGTCGCAACAAGGTGGTCAAGCT
GTCACTGAAGCCATACAGATTCGATGTCTATCTGGACAACGGCGTGTATAAATTTGTGACTGTCAAGA
ATCTGGATGTCATCAAAAAGGAGAACTACTATGAAGTGAATAGCAAGTGCTACGAAGAGGCTAAAAAG
CTGAAAAAGATTAGCAACCAGGCAGAGTTCATCGCCTCCTTTTACAACAACGACCTGATTAAGATCAA
TGGCGAACTGTATAGGGTCATCGGGGTGAACAATGATCTGCTGAACCGCATTGAAGTGAATATGATTG
ACATCACTTACCGAGAGTATCTGGAAAACATGAATGATAAGCGCCCCCCTCGAATTATCAAAACAATT
GCCTCTAAGACTCAGAGTATCAAAAAGTACTCAACCGACATTCTGGGAAACCTGTATGAGGTGAAGAG
CAAAAAGCACCCTCAGATTATCAAAAAGGGCagcggaggcaagcgtcctgctgctactaagaaagctg
gtcaagctaagaaaaagaaaggatcctacccatacgatgttccagattacgcttaagaattcctagag
ctcgctgatcagcctcgactgtgccttctagttgccagccatctgttgtttgcccctcccccgtgcct
tccttgaccctggaaggtgccactcccactgtcctttcctaataaaatgaggaaattgcatcgcattg
tctgagtaggtgtcattctattctggggggtygggtggggcaggacagcaagggggaggattgggaag
agaatagcaggcatgctggggaggtagcggccgcCCgcggtggagctccagcttttgttccctttagt
gagggttaattgcgcgcttggcgtaatcatggtcatagctgtttcctgtgtgaaattgttatccgctc
acaattccacacaacatacgagccggaagcataaagtgtaaagcctggggtgcctaatgagtgagcta
actcacattaattgcgttgcgctcactgcccgctttccagtcgggaaacctgtcgtgccagctgcatt
aatgaatcggccaacgcgcggggagaggcggtttgcgtattgggcgctcttccgcttcctcgctcact
gactcgctgcgctcggtcgttcggctgcggcgagcggtatcagctcactcaaaggcggtaatacggtt
atccacagaatcaggggataacgcaggaaagaacatgtgagcaaaaggccagcaaaaggccaggaacc
gtaaaaaggccgcgttgctggcgtttttccataggctccgcccccctgacgagcatcacaaaaatcga
cgctcaagtcagaggtggcgaaacccgacaggactataaagataccaggcgtttccccctggaagctc
cctcgtgcgctctcctgttccgaccctgccgcttaccggatacctgtccgcctttctcccttcgggaa
gcgtggcgctttctcatagctcacgctgtaggtatctcagttcggtgtaggtcgttcgctccaagctg
ggctgtgtgcacgaaccccccgttcagcccgaccgctgcgccttatccggtaactatcgtcttgagtc
caacccggtaagacacgacttatcgccactggcagcagccactggtaacaggattagcagagcgaggt
atgtaggcggtgctacagagttcttgaagtggtggcctaactacggctacactagaaggacagtattt
ggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctc
aagaagatcctttgatcttttctacggggtctgacgctcagtggaacgaaaactcacgttaagggatt
ttggtcatgagattatcaaaaaggatcttcacctagatccttttaaattaaaaatgaagttttaaatc
aatctaaagtatatatgagtaaacttggtctgacagttaccaatgcttaatcagtgaggcacctatct
cagcgatctgtctatttcgttcatccatagttgcctgactccccgtcgtgtagataactacgatacgg
gagggcttaccatctggccccagtgctgcaatgataccgcgagacccacgctcaccggctccagattt
atcagcaataaaccagccagccggaagggccgagcgcagaagtggtcctgcaactttatccgcctcca
tccagtctattaattgttgccgggaagctagagtaagtagttcgccagttaatagtttgcgcaacgtt
gttgccattgctacaggcatcgtggtgtcacgctcgtcgtttggtatggcttcattcagctccggttc
ccaacgatcaaggcgagttacatgatcccccatgttgtgcaaaaaagcggttagctccttcggtcctc
cgatcgttgtcagaagtaagttggccgcagtgttatcactcatggttatggcagcactgcataattct
cttactgtcatgccatccgtaagatgcttttctgtgactggtgagtactcaaccaagtcattctgaga
atagtgtatgcggcgaccgagttgctcttgcccggcgtcaatacgggataataccgcgccacatagca
gaactttaaaagtgctcatcattggaaaacgttcttcggggcgaaaactctcaaggatcttaccgctg
ttgagatccagttcgatgtaacccactcgtgcacccaactgatcttcagcatcttttactttcaccag
cgtttctgggtgagcaaaaacaggaaggcaaaatgccgcaaaaaagggaataagggcgacacggaaat
gttgaatactcatactcttcctttttcaatattattgaagcatttatcagggttattgtctcatgagc
ggatacatatttgaatgtatttagaaaaataaacaaataggggttccgcgcacatttccccgaaaagt
gccac
SEQ ID NO: 38
Fwd primer
5′-aatgatacggcgaccaccgagatctacacaatttcttgggtagtttgcagtt
SEQ ID NO: 39
Reverse primer
5′-caagcagaagacggcatacgagat-(6-bp index sequence)-
actcggtgccactttttcaa
SEQ ID NO: 40
Read1
5′-gatttcttggctttatatatcttgtggaaaggacgaaacaccg
SEQ ID NO: 41
Index
5′-gctagtccgttatcaacttgaaaaagtggcaccgagtc
SEQ ID NO: 42
Read2
5′-gttgataacggactagccttattttaacttgctatttctagctctaaaac
SEQ ID NO: 43
VP64-dCas9-VP64 protein
RADALDDFDLDMLGSDALDDEDLDMLGSDALDDEDLDMLGSDALDDEDLDMVNPKKKRKVGRGMDKKY
SIGLAIGTNSVGWAVITDEYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIVDEVAYHEKYPTIYHLRKK
LVDSTDKADLRLIYLALAHMIKERGHFLIEGDLNPDNSDVDKLFIQLVQTYNQLFEENPINASGVDAK
AILSARLSKSRRLENLIAQLPGEKKNGLEGNLIALSLGLTPNEKSNFDLAEDAKLQLSKDTYDDDLDN
LLAQIGDQYADLFLAAKNLSDAILLSDILRVNTEITKAPLSASMIKRYDEHHQDITLLKALVRQQLPE
KYKEIFFDQSKNGYAGYIDGGASQEEFYKFIKPILEKMDGTEELLVKLNREDLLRKQRTEDNGSIPHQ
IHLGELHAILRRQEDFYPFLKDNREKIEKILTFRIPYYVGPLARGNSRFAWMTRKSEETITPWNFEEV
VDKGASAQSFIERMTNFDKNLPNEKVLPKHSLLYEYFTVYNELTKVKYVTEGMRKPAFLSGEQKKAIV
DLLFKTNRKVTVKQLKEDYFKKIECFDSVEISGVEDRENASLGTYHDLLKIIKDKDFLDNEENEDILE
DIVLTLTLFEDREMIEERLKTYAHLEDDKVMKQLKRRRYTGWGRLSRKLINGIRDKQSGKTILDFLKS
DGFANRNFMQLIHDDSLTFKEDIQKAQVSGQGDSLHEHIANLAGSPAIKKGILQTVKVVDELVKVMGR
HKPENIVIEMARENQTTQKGQKNSRERMKRIEEGIKELGSQILKEHPVENTQLQNEKLYLYYLQNGRD
MYVDQELDINRLSDYDVDAIVPQSFLKDDSIDNKVLTRSDKNRGKSDNVPSEEVVKKMKNYWRQLLNA
KLITQRKEDNITKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVRKMIAKS
EQEIGKATAKYFFYSNIMNFFKTEITLANGEIRKRPLIETNGETGEIVWDKGRDFATVRKVLSMPQVN
IVKKTEVQTGGFSKESILPKRNSDKLIARKKDWDPKKYGGFDSPTVAYSVLVVAKVEKGKSKKLKSVK
ELLGITIMERSSFEKNPIDFLEAKGYKEVKKDLIIKLPKYSLFELENGRKRMLASAGELQKGNELALP
SKYVNFLYLASHYEKLKGSPEDNEQKQLFVEQHKHYLDEIIEQISEFSKRVILADANLDKVLSAYNKH
RDKPIREQAENIIHLFTLINLGAPAAFKYFDTTIDRKRYTSTKEVLDATLIHQSITGLYETRIDLSQL
GGDSRADPKKKRKVASRADALDDEDLDMLGSDALDDEDLDMLGSDALDDEDLDMLGSDALDDEDLDML
I
SEQ ID NO: 44
VP64-dCas9-VP64 DNA
cgggctgacgcattggacgattttgatctggatatgctgggaagtgacgccctcgatgattttgacct
tgacatgcttggttcggatgcccttgatgactttgacctcgacatgctcggcagtgacgcccttgatg
atttcgacctggacatggttaaccccaagaagaagaggaaggtgggccgcggaatggacaagaagtac
tccattgggctcgccatcggcacaaacagcgtcggctgggccgtcattacggacgagtacaaggtgcc
gagcaaaaaattcaaagttctgggcaataccgatcgccacagcataaagaagaacctcattggcgccc
tcctgttcgactccggggaaaccgccgaagccacgcggctcaaaagaacagcacggcgcagatatacc
cgcagaaagaatcggatctgctacctgcaggagatctttagtaatgagatggctaaggtggatgactc
tttcttccataggctggaggagtcctttttggtggaggaggataaaaagcacgagcgccacccaatct
ttggcaatatcgtggacgaggtggcgtaccatgaaaagtacccaaccatatatcatctgaggaagaag
cttgtagacagtactgataaggctgacttgcggttgatctatctcgcgctggcgcatatgatcaaatt
tcggggacacttcctcatcgagggggacctgaacccagacaacagcgatgtcgacaaactctttatcc
aactggttcagacttacaatcagcttttcgaagagaacccgatcaacgcatccggagttgacgccaaa
gcaatcctgagcgctaggctgtccaaatcccggcggctcgaaaacctcatcgcacagctccctgggga
gaagaagaacggcctgtttggtaatcttatcgccctgtcactcgggctgacccccaactttaaatcta
acttcgacctggccgaagatgccaagcttcaactgagcaaagacacctacgatgatgatctcgacaat
ctgctggcccagatcggcgaccagtacgcagacctttttttggcggcaaagaacctgtcagacgccat
tctgctgagtgatattctgcgagtgaacacggagatcaccaaagctccgctgagcgctagtatgatca
agcgctatgatgagcaccaccaagacttgactttgctgaaggcccttgtcagacagcaactgcctgag
aagtacaaggaaattttcttcgatcagtctaaaaatggctacgccggatacattgacggcggagcaag
ccaggaggaattttacaaatttattaagcccatcttggaaaaaatggacggcaccgaggagctgctgg
taaagcttaacagagaagatctgttgcgcaaacagcgcactttcgacaatggaagcatcccccaccag
attcacctgggcgaactgcacgctatcctcaggcggcaagaggatttctacccctttttgaaagataa
cagggaaaagattgagaaaatcctcacatttcggataccctactatgtaggccccctcgcccggggaa
attccagattcgcgtggatgactcgcaaatcagaagagaccatcactccctggaacttcgaggaagtc
gtggataagggggcctctgcccagtccttcatcgaaaggatgactaactttgataaaaatctgcctaa
cgaaaaggtgcttcctaaacactctctgctgtacgagtacttcacagtttataacgagctcaccaagg
tcaaatacgtcacagaagggatgagaaagccagcattcctgtctggagagcagaagaaagctatcgtg
gacctcctcttcaagacgaaccggaaagttaccgtgaaacagctcaaagaagactatttcaaaaagat
tgaatgtttcgactctgttgaaatcagcggagtggaggatcgcttcaacgcatccctgggaacgtatc
acgatctcctgaaaatcattaaagacaaggacttcctggacaatgaggagaacgaggacattcttgag
gacattgtcctcacccttacgttgtttgaagatagggagatgattgaagaacgcttgaaaacttacgc
tcatctcttcgacgacaaagtcatgaaacagctcaagaggcgccgatatacaggatgggggcggctgt
caagaaaactgatcaatgggatccgagacaagcagagtggaaagacaatcctggattttcttaagtcc
gatggatttgccaaccggaacttcatgcagttgatccatgatgactctctcacctttaaggaggacat
ccagaaagcacaagtttctggccagggggacagtcttcacgagcacatcgctaatcttgcaggtagcc
cagctatcaaaaagggaatactgcagaccgttaaggtcgtggatgaactcgtcaaagtaatgggaagg
cataagcccgagaatatcgttatcgagatggcccgagagaaccaaactacccagaagggacagaagaa
cagtagggaaaggatgaagaggattgaagagggtataaaagaactggggtcccaaatccttaaggaac
acccagttgaaaacacccagcttcagaatgagaagctctacctgtactacctgcagaacggcagggac
atgtacgtggatcaggaactggacatcaatcggctctccgactacgacgtggatgccatcgtgcccca
gtcttttctcaaagatgattctattgataataaagtgttgacaagatccgataaaaatagagggaaga
gtgataacgtcccctcagaagaagttgtcaagaaaatgaaaaattattggcggcagctgctgaacgcc
aaactgatcacacaacggaagttcgataatctgactaaggctgaacgaggtggcctgtctgagttgga
taaagccggcttcatcaaaaggcagcttgttgagacacgccagatcaccaagcacgtggcccaaattc
tcgattcacgcatgaacaccaagtacgatgaaaatgacaaactgattcgagaggtgaaagttattact
ctgaagtctaagctggtctcagatttcagaaaggactttcagttttataaggtgagagagatcaacaa
ttaccaccatgcgcatgatgcctacctgaatgcagtggtaggcactgcacttatcaaaaaatatccca
agcttgaatctgaatttgtttacggagactataaagtgtacgatgttaggaaaatgatcgcaaagtct
gagcaggaaataggcaaggccaccgctaagtacttcttttacagcaatattatgaattttttcaagac
cgagattacactggccaatggagagattcggaagcgaccacttatcgaaacaaacggagaaacaggag
aaatcgtgtgggacaagggtagggatttcgcgacagtccggaaggtcctgtccatgccgcaggtgaac
atcgttaaaaagaccgaagtacagaccggaggcttctccaaggaaagtatcctcccgaaaaggaacag
cgacaagctgatcgcacgcaaaaaagattgggaccccaagaaatacggcggattcgattctcctacag
tcgcttacagtgtactggttgtggccaaagtggagaaagggaagtctaaaaaactcaaaagcgtcaag
gaactgctgggcatcacaatcatggagcgatcaagcttcgaaaaaaaccccatcgactttctcgaggc
gaaaggatataaagaggtcaaaaaagacctcatcattaagcttcccaagtactctctctttgagcttg
aaaacggccggaaacgaatgctcgctagtgcgggcgagctgcagaaaggtaacgagctggcactgccc
tctaaatacgttaatttcttgtatctggccagccactatgaaaagctcaaagggtctcccgaagataa
tgagcagaagcagctgttcgtggaacaacacaaacactaccttgatgagatcatcgagcaaataagcg
aattctccaaaagagtgatcctcgccgacgctaacctcgataaggtgctttctgcttacaataagcac
agggataagcccatcagggagcaggcagaaaacattatccacttgtttactctgaccaacttgggcgc
gcctgcagccttcaagtacttcgacaccaccatagacagaaagcggtacacctctacaaaggaggtcc
tggacgccacactgattcatcagtcaattacggggctctatgaaacaagaatcgacctctctcagctc
ggtggagacagcagggctgaccccaagaagaagaggaaggtggctagccgcgccgacgcgctggacga
tttcgatctcgacatgctgggttctgatgccctcgatgactttgacctggatatgttgggaagcgacg
cattggatgactttgatctggacatgctcggctccgatgctctggacgatttcgatctcgatatgtta
atc
SEQ ID NO: 45
Human p300 (with L553M mutation) protein
MAENVVEPGPPSAKRPKLSSPALSASASDGTDFGSLEDLEHDLPDELINSTELGLINGGDINQLQTSL
GMVQDAASKHKQLSELLRSGSSPNLNMGVGGPGQVMASQAQQSSPGLGLINSMVKSPMTQAGLTSPNM
GMGTSGPNQGPTQSTGMMNSPVNQPAMGMNTGMNAGMNPGMLAAGNGQGIMPNQVMNGSIGAGRGRQN
MQYPNPGMGSAGNLLTEPLQQGSPQMGGQTGLRGPQPLKMGMMNNPNPYGSPYTQNPGQQIGASGLGL
QIQTKTVLSNNLSPFAMDKKAVPGGGMPNMGQQPAPQVQQPGLVTPVAQGMGSGAHTADPEKRKLIQQ
QLVLLLHAHKCQRREQANGEVRQCNLPHCRTMKNVLNHMTHCQSGKSCQVAHCASSRQIISHWKNCTR
HDCPVCLPLKNAGDKRNQQPILTGAPVGLGNPSSLGVGQQSAPNLSTVSQIDPSSIERAYAALGLPYQ
VNQMPTQPQVQAKNQQNQQPGQSPQGMRPMSNMSASPMGVNGGVGVQTPSLLSDSMLHSAINSQNPMM
SENASVPSMGPMPTAAQPSTTGIRKQWHEDITQDLRNHLVHKLVQAIFPTPDPAALKDRRMENLVAYA
RKVEGDMYESANNRAEYYHLLAEKIYKIQKELEEKRRTRLQKQNMLPNAAGMVPVSMNPGPNMGQPQP
GMTSNGPLPDPSMIRGSVPNQMMPRITPQSGLNQFGQMSMAQPPIVPRQTPPLQHHGQLAQPGALNPP
MGYGPRMQQPSNQGQFLPQTQFPSQGMNVTNIPLAPSSGQAPVSQAQMSSSSCPVNSPIMPPGSQGSH
IHCPQLPQPALHQNSPSPVPSRTPTPHHTPPSIGAQQPPATTIPAPVPTPPAMPPGPQSQALHPPPRQ
TPTPPTTQLPQQVQPSLPAAPSADQPQQQPRSQQSTAASVPTPTAPLLPPQPATPLSQPAVSIEGQVS
NPPSTSSTEVNSQAIAEKQPSQEVKMEAKMEVDQPEPADTQPEDISESKVEDCKMESTETEERSTELK
TEIKEEEDQPSTSATQSSPAPGQSKKKIFKPEELRQALMPTLEALYRQDPESLPFRQPVDPQLLGIPD
YFDIVKSPMDLSTIKRKLDTGQYQEPWQYVDDIWLMENNAWLYNRKTSRVYKYCSKLSEVFEQEIDPV
MQSLGYCCGRKLEFSPQTLCCYGKQLCTIPRDATYYSYQNRYHFCEKCFNEIQGESVSLGDDPSQPQT
TINKEQFSKRKNDTLDPELFVECTECGRKMHQICVLHHEIIWPAGFVCDGCLKKSARTRKENKESAKR
LPSTRLGTFLENRVNDFLRRQNHPESGEVTVRVVHASDKTVEVKPGMKARFVDSGEMAESFPYRTKAL
FAFEEIDGVDLCFFGMHVQEYGSDCPPPNQRRVYISYLDSVHFFRPKCLRTAVYHEILIGYLEYVKKL
GYTTGHIWACPPSEGDDYIFHCHPPDQKIPKPKRLQEWYKKMLDKAVSERIVHDYKDIFKQATEDRLT
SAKELPYFEGDFWPNVLEESIKELEQEEEERKREENTSNESTDVTKGDSKNAKKKNNKKTSKNKSSLS
RGNKKKPGMPNVSNDLSQKLYATMEKHKEVFFVIRLIAGPAANSLPPIVDPDPLIPCDLMDGRDAFLT
LARDKHLEFSSLRRAQWSTMCMIVELHTQSQDREVYTCNECKHHVETRWHCTVCEDYDLCITCYNTKN
HDHKMEKLGLGLDDESNNQQAAATQSPGDSRRLSIQRCIQSLVHACQCRNANCSLPSCQKMKRVVQHT
KGCKRKINGGCPICKQLIALCCYHAKHCQENKCPVPFCLNIKQKLRQQQLQHRLQQAQMLRRRMASMQ
RTGVVGQQQGLPSPTPATPTTPTGQQPTTPQTPQPTSQPQPTPPNSMPPYLPRTQAAGPVSQGKAAGQ
VTPPTPPQTAQPPLPGPPPAAVEMAMQIQRAAETQRQMAHVQIFQRPIQHQMPPMTPMAPMGMNPPPM
TRGPSGHLEPGMGPTGMQQQPPWSQGGLPQPQQLQSGMPRPAMMSVAQHGQPLNMAPQPGLGQVGISP
LKPGTVSQQALQNLLRTLRSPSSPLQQQQVISILHANPQLLAAFIKQRAAKYANSNPQPIPGQPGMPQ
GQPGLQPPTMPGQQGVHSNPAMQNMNPMQAGVQRAGLPQQQPQQQLQPPMGGMSPQAQQMNMNHNTMP
SQFRDILRRQQMMQQQQQQGAGPGIGPGMANHNQFQQPQGVGYPPQQQQRMQHHMQQMQQGNMGQIGQ
LPQALGAEAGASLQAYQQRLLQQQMGSPVQPNPMSPQQHMLPNQAQSPHLQGQQIPNSLSNQVRSPQP
VPSPRPQSQPPHSSPSPRMQPQPSPHHVSPQTSSPHPGLVAAQANPMEQGHFASPDQNSMLSQLASNP
GMANLHGASATDLGLSTDNSDLNSNLSQSTLDIH
SEQ ID NO: 46
Human p300 Core Effector protein (aa 1048-1664 of SEQ ID NO: 134)
IFKPEELRQALMPTLEALYRQDPESLPFRQPVDPQLLGIPDYFDIVKSPMDLSTIKRKLDTGQYQEPW
QYVDDIWLMFNNAWLYNRKTSRVYKYCSKLSEVFEQEIDPVMQSLGYCCGRKLEFSPQTLCCYGKQLC
TIPRDATYYSYQNRYHFCEKCFNEIQGESVSLGDDPSQPQTTINKEQFSKRKNDTLDPELFVECTECG
RKMHQICVLHHEIIWPAGFVCDGCLKKSARTRKENKFSAKRLPSTRLGTFLENRVNDFLRRQNHPESG
EVTVRVVHASDKTVEVKPGMKARFVDSGEMAESFPYRTKALFAFEEIDGVDLCFFGMHVQEYGSDCPP
PNQRRVYISYLDSVHFFRPKCLRTAVYHEILIGYLEYVKKLGYTTGHIWACPPSEGDDYIFHCHPPDQ
KIPKPKRLQEWYKKMLDKAVSERIVHDYKDIFKCATEDRLTSAKELPYFEGDFWPNVLEESIKELEQE
EEERKREENTSNESTDVTKGDSKNAKKKNNKKTSKNKSSLSRGNKKKPGMPNVSNDLSQKLYATMEKH
KEVFFVIRLIAGPAANSLPPIVDPDPLIPCDLMDGRDAFLTLARDKHLEFSSLRRAQWSTMCMLVELH
TQSQD
SEQ ID NO: 87
Polynucleotide sequence encoding Streptococcus pyogenes dCas9-KRAB
atggactacaaagaccatgacggtgattataaagatcatgacatcgattacaaggatgacga
tgacaagatggcccccaagaagaagaggaaggtgggccgcggaatggacaagaagtactcca
ttgggctcgccatcggcacaaacagcgtcggctgggccgtcattacggacgagtacaaggtg
ccgagcaaaaaattcaaagttctgggcaataccgatcgccacagcataaagaagaacctcat
tggcgccctcctgttcgactccggggaaaccgccgaagccacgcggctcaaaagaacagcac
ggcgcagatatacccgcagaaagaatcggatctgctacctgcaggagatctttagtaatgag
atggctaaggtggatgactctttcttccataggctggaggagtcctttttggtggaggagga
taaaaagcacgagcgccacccaatctttggcaatatcgtggacgaggtggcgtaccatgaaa
agtacccaaccatatatcatctgaggaagaagcttgtagacagtactgataaggctgacttg
cggttgatctatctcgcgctggcgcatatgatcaaatttcggggacacttcctcatcgaggg
ggacctgaacccagacaacagcgatgtcgacaaactctttatccaactggttcagacttaca
atcagcttttcgaagagaacccgatcaacgcatccggagttgacgccaaagcaatcctgagc
gctaggctgtccaaatcccggcggctcgaaaacctcatcgcacagctccctggggagaagaa
gaacggcctgtttggtaatcttatcgccctgtcactcgggctgacccccaactttaaatcta
acttcgacctggccgaagatgccaagcttcaactgagcaaagacacctacgatgatgatctc
gacaatctgctggcccagatcggcgaccagtacgcagacctttttttggcggcaaagaacct
gtcagacgccattctgctgagtgatattctgcgagtgaacacggagatcaccaaagctccgc
tgagcgctagtatgatcaagcgctatgatgagcaccaccaagacttgactttgctgaaggcc
cttgtcagacagcaactgcctgagaagtacaaggaaattttcttcgatcagtctaaaaatgg
ctacgccggatacattgacggcggagcaagccaggaggaattttacaaatttattaagccca
tcttggaaaaaatggacggcaccgaggagctgctggtaaagcttaacagagaagatctgttg
cgcaaacagcgcactttcgacaatggaagcatcccccaccagattcacctgggcgaactgca
cgctatcctcaggcggcaagaggatttctacccctttttgaaagataacagggaaaagattg
agaaaatcctcacatttcggataccctactatgtaggccccctcgcccggggaaattccaga
ttcgcgtggatgactcgcaaatcagaagagaccatcactccctggaacttcgaggaagtcgt
ggataagggggcctctgcccagtccttcatcgaaaggatgactaactttgataaaaatctgc
ctaacgaaaaggtgcttcctaaacactctctgctgtacgagtacttcacagtttataacgag
ctcaccaaggtcaaatacgtcacagaagggatgagaaagccagcattcctgtctggagagca
gaagaaagctatcgtggacctcctcttcaagacgaaccggaaagttaccgtgaaacagctca
aagaagactatttcaaaaagattgaatgtttcgactctgttgaaatcagcggagtggaggat
cgcttcaacgcatccctgggaacgtatcacgatctcctgaaaatcattaaagacaaggactt
cctggacaatgaggagaacgaggacattcttgaggacattgtcctcacccttacgttgtttg
aagatagggagatgattgaagaacgcttgaaaacttacgctcatctcttcgacgacaaagtc
atgaaacagctcaagaggcgccgatatacaggatgggggcggctgtcaagaaaactgatcaa
tgggatccgagacaagcagagtggaaagacaatcctggattttcttaagtccgatggatttg
ccaaccggaacttcatgcagttgatccatgatgactctctcacctttaaggaggacatccag
aaagcacaagtttctggccagggggacagtcttcacgagcacatcgctaatcttgcaggtag
cccagctatcaaaaagggaatactgcagaccgttaaggtcgtggatgaactcgtcaaagtaa
tgggaaggcataagcccgagaatatcgttatcgagatggcccgagagaaccaaactacccag
aagggacagaagaacagtagggaaaggatgaagaggattgaagagggtataaaagaactggg
gtcccaaatccttaaggaacacccagttgaaaacacccagcttcagaatgagaagctctacc
tgtactacctgcagaacggcagggacatgtacgtggatcaggaactggacatcaatcggctc
tccgactacgacgtggatgccatcgtgccccagtcttttctcaaagatgattctattgataa
taaagtgttgacaagatccgataaaaatagagggaagagtgataacgtcccctcagaagaag
ttgtcaagaaaatgaaaaattattggcggcagctgctgaacgccaaactgatcacacaacgg
aagttcgataatctgactaaggctgaacgaggtggcctgtctgagttggataaagccggctt
catcaaaaggcagcttgttgagacacgccagatcaccaagcacgtggcccaaattctcgatt
cacgcatgaacaccaagtacgatgaaaatgacaaactgattcgagaggtgaaagttattact
ctgaagtctaagctggtctcagatttcagaaaggactttcagttttataaggtgagagagat
caacaattaccaccatgcgcatgatgcctacctgaatgcagtggtaggcactgcacttatca
aaaaatatcccaagcttgaatctgaatttgtttacggagactataaagtgtacgatgttagg
aaaatgatcgcaaagtctgagcaggaaataggcaaggccaccgctaagtacttcttttacag
caatattatgaattttttcaagaccgagattacactggccaatggagagattcggaagcgac
cacttatcgaaacaaacggagaaacaggagaaatcgtgtgggacaagggtagggatttcgcg
acagtccggaaggtcctgtccatgccgcaggtgaacatcgttaaaaagaccgaagtacagac
cggaggcttctccaaggaaagtatcctcccgaaaaggaacagcgacaagctgatcgcacgca
aaaaagattgggaccccaagaaatacggcggattcgattctcctacagtcgcttacagtgta
ctggttgtggccaaagtggagaaagggaagtctaaaaaactcaaaagcgtcaaggaactgct
gggcatcacaatcatggagcgatcaagcttcgaaaaaaaccccatcgactttctcgaggcga
aaggatataaagaggtcaaaaaagacctcatcattaagcttcccaagtactctctctttgag
cttgaaaacggccggaaacgaatgctcgctagtgcgggcgagctgcagaaaggtaacgagct
ggcactgccctctaaatacgttaatttcttgtatctggccagccactatgaaaagctcaaag
ggtctcccgaagataatgagcagaagcagctgttcgtggaacaacacaaacactaccttgat
gagatcatcgagcaaataagcgaattctccaaaagagtgatcctcgccgacgctaacctcga
taaggtgctttctgcttacaataagcacagggataagcccatcagggagcaggcagaaaaca
ttatccacttgtttactctgaccaacttgggcgcgcctgcagccttcaagtacttcgacacc
accatagacagaaagcggtacacctctacaaaggaggtcctggacgccacactgattcatca
gtcaattacggggctctatgaaacaagaatcgacctctctcagctcggtggagacagcaggg
ctgaccccaagaagaagaggaaggtggctagcgatgctaagtcactgactgcctggtcccgg
acactggtgaccttcaaggatgtgtttgtggacttcaccagggaggagtggaagctgctgga
cactgctcagcagatcctgtacagaaatgtgatgctggagaactataagaacctggtttcct
tgggttatcagcttactaagccagatgtgatcctccggttggagaagggagaagagccctgg
ctggtggagagagaaattcaccaagagacccatcctgattcagagactgcatttgaaatcaa
atcatcagttccgaaaaagaaacgcaaagtttga
SEQ ID NO: 88
Polypeptide sequence of Streptococcus pyogenes dCas9-KRAB protein
MDYKDHDGDYKDHDIDYKDDDDKMAPKKKRKVGRGMDKKYSIGLAIGTNSVGWAVITDEYKV
PSKKFKVLGNTDRHSIKKNLIGALLEDSGETAEATRLKRTARRRYTRRKNRICYLQEIESNE
MAKVDDSFFHRLEESFLVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPINASGVDAKAILS
ARLSKSRRLENLIAQLPGEKKNGLFGNLIALSLGLTPNEKSNFDLAEDAKLQLSKDTYDDDL
DNLLAQIGDQYADLFLAAKNLSDAILLSDILRVNTEITKAPLSASMIKRYDEHHQDLTLLKA
LVRQQLPEKYKEIFFDQSKNGYAGYIDGGASQEEFYKFIKPILEKMDGTEELLVKLNREDLL
RKQRTFDNGSIPHQIHLGELHAILRRQEDFYPFLKDNREKIEKILTFRIPYYVGPLARGNSR
FAWMIRKSEETITPWNFEEVVDKGASAQSFIERMTNEDKNIPNEKVLPKHSLLYEYETVYNE
LTKVKYVTEGMRKPAFLSGEQKKAIVDLLFKTNRKVTVKQLKEDYFKKIECFDSVEISGVED
RENASLGTYHDLLKIIKDKDELDNEENEDILEDIVLILTLFEDREMIEERLKTYAHLEDDKV
MKQLKRRRYTGWGRLSRKLINGIRDKQSGKTILDFLKSDGFANRNEMQLIHDDSLTFKEDIQ
KAQVSGQGDSLHEHIANLAGSPAIKKGILQTVKVVDELVKVMGRHKPENIVIEMARENQTTQ
KGQKNSRERMKRIEEGIKELGSQILKEHPVENTQLQNEKLYLYYLQNGRDMYVDQELDINRL
SDYDVDAIVPQSFLKDDSIDNKVLTRSDKNRGKSDNVPSEEVVKKMKNYWRQLLNAKLITQR
KEDNLTKAERGGLSELDKAGFIKRQLVETRQITKHVAQILDSRMNTKYDENDKLIREVKVIT
LKSKLVSDFRKDFQFYKVREINNYHHAHDAYLNAVVGTALIKKYPKLESEFVYGDYKVYDVR
KMIAKSEQEIGKATAKYFFYSNIMNFFKTEITLANGEIRKRPLIEINGETGEIVWDKGRDFA
TVRKVLSMPQVNIVKKTEVQTGGESKESILPKRNSDKLIARKKDWDPKKYGGFDSPTVAYSV
LVVAKVEKGKSKKLKSVKELLGITIMERSSFEKNPIDFLEAKGYKEVKKDLIIKLPKYSLFE
LENGRKRMLASAGELQKGNELALPSKYVNFLYLASHYEKLKGSPEDNEQKQLFVEQHKHYLD
EIIEQISEFSKRVILADANLDKVLSAYNKHRDKPIREQAENIIHLETLTNLGAPAAFKYEDT
TIDRKRYTSTKEVLDATLIHQSITGLYETRIDLSQLGGDSRADPKKKRKVASDAKSLTAWSR
TLVTFKDVFVDFTREEWKLLDTAQQILYRNVMLENYKNLVSLGYQLTKPDVILRLEKGEEPW
LVEREIHQETHPDSETAFEIKSSVPKKKRKV
SEQ ID NO: 1138
Polynucleotide sequence of Tet1CD (also referred to as Tet1c)
CTGCCCACCTGCAGCTGTCTTGATCGAGTTATACAAAAAGACAAAGGCCCATATTATACACA
CCTTGGGGCAGGACCAAGTGTTGCTGCTGTCAGGGAAATCATGGAGAATAGGTATGGTCAAA
AAGGAAACGCAATAAGGATAGAAATAGTAGTGTACACCGGTAAAGAAGGGAAAAGCTCTCAT
GGGTGTCCAATTGCTAAGTGGGTTTTAAGAAGAAGCAGTGATGAAGAAAAAGTTCTTTGTTT
GGTCCGGCAGCGTACAGGCCACCACTGTCCAACTGCTGTGATGGTGGTGCTCATCATGGTGT
GGGATGGCATCCCTCTTCCAATGGCCGACCGGCTATACACAGAGCTCACAGAGAATCTAAAG
TCATACAATGGGCACCCTACCGACAGAAGATGCACCCTCAATGAAAATCGTACCTGTACATG
TCAAGGAATTGATCCAGAGACTTGTGGAGCTTCATTCTCTTTTGGCTGTTCATGGAGTATGT
ACTTTAATGGCTGTAAGTTTGGTAGAAGCCCAAGCCCCAGAAGATTTAGAATTGATCCAAGC
TCTCCCTTACATGAAAAAAACCTTGAAGATAACTTACAGAGTTTGGCTACACGATTAGCTCC
AATTTATAAGCAGTATGCTCCAGTAGCTTACCAAAATCAGGTGGAATATGAAAATGTTGCCC
GAGAATGTCGGCTTGGCAGCAAGGAAGGTCGACCCTTCTCTGGGGTCACTGCTTGCCTGGAC
TTCTGTGCTCATCCCCACAGGGACATTCACAACATGAATAATGGAAGCACTGTGGTTTGTAC
CTTAACTCGAGAAGATAACCGCTCTTTGGGTGTTATTCCTCAAGATGAGCAGCTCCATGTGC
TACCTCTTTATAAGCTTTCAGACACAGATGAGTTTGGCTCCAAGGAAGGAATGGAAGCCAAG
ATCAAATCTGGGGCCATCGAGGTCCTGGCACCCCGCCGCAAAAAAAGAACGTGTTTCACTCA
GCCTGTTCCCCGTTCTGGAAAGAAGAGGGCTGCGATGATGACAGAGGTTCTTGCACATAAGA
TAAGGGCAGTGGAAAAGAAACCTATTCCCCGAATCAAGCGGAAGAATAACTCAACAACAACA
AACAACAGTAAGCCTTCGTCACTGCCAACCTTAGGGAGTAACACTGAGACCGTGCAACCTGA
AGTAAAAAGTGAAACCGAACCCCATTTTATCTTAAAAAGTTCAGACAACACTAAAACTTATT
CGCTGATGCCATCCGCTCCTCACCCAGTGAAAGAGGCATCTCCAGGCTTCTCCTGGTCCCCG
AAGACTGCTTCAGCCACACCAGCTCCACTGAAGAATGACGCAACAGCCTCATGCGGGTTTTC
AGAAAGAAGCAGCACTCCCCACTGTACGATGCCTTCGGGAAGACTCAGTGGTGCCAATGCTG
CAGCTGCTGATGGCCCTGGCATTTCACAGCTTGGCGAAGTGGCTCCTCTCCCCACCCTGTCT
GCTCCTGTGATGGAGCCCCTCATTAATTCTGAGCCTTCCACTGGTGTGACTGAGCCGCTAAC
GCCTCATCAGCCAAACCACCAGCCCTCCTTCCTCACCTCTCCTCAAGACCTTGCCTCTTCTC
CAATGGAAGAAGATGAGCAGCATTCTGAAGCAGATGAGCCTCCATCAGACGAACCCCTATCT
GATGACCCCCTGTCACCTGCTGAGGAGAAATTGCCCCACATTGATGAGTATTGGTCAGACAG
TGAGCACATCTTTTTGGATGCAAATATTGGTGGGGTGGCCATCGCACCTGCTCACGGCTCGG
TTTTGATTGAGTGTGCCCGGCGAGAGCTGCACGCTACCACTCCTGTTGAGCACCCCAACCGT
AATCATCCAACCCGCCTCTCCCTTGTCTTTTACCAGCACAAAAACCTAAATAAGCCCCAACA
TGGTTTTGAACTAAACAAGATTAAGTTTGAGGCTAAAGAAGCTAAGAATAAGAAAATGAAGG
CCTCAGAGCAAAAAGACCAGGCAGCTAATGAAGGTCCAGAACAGTCCTCTGAAGTAAATGAA
TTGAACCAAATTCCTTCTCATAAAGCATTAACATTAACCCATGACAATGTTGTCACCGTGTC
CCCTTATGCTCTCACACACGTTGCGGGGCCCTATAACCATTGGGTC
SEQ ID NO: 1139
Polypeptide sequence of Tet1CD (also referred to as Tet1c)
LPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLK
SYNGHPTDRRCTINENRTCTCQGIDPETCGASFSFGCSWSMYENGCKFGRSPSPRRFRIDPS
SPLHEKNLEDNIQSLATRLAPIYKQYAPVAYQNQVEYENVARECRLGSKEGRPESGVTACLD
FCAHPHRDIHNMNNGSTVVCTLTREDNRSLGVIPQDEQLHVLPLYKLSDTDEFGSKEGMEAK
IKSGAIEVLAPRRKKRTCFTQPVPRSGKKRAAMMTEVLAHKIRAVEKKPIPRIKRKNNSTTT
NNSKPSSLPTLGSNTETVQPEVKSETEPHFILKSSDNTKTYSLMPSAPHPVKEASPGFSWSP
KTASATPAPLKNDATASCGFSERSSTPHCTMPSGRLSGANAAAADGPGISQLGEVAPLPTLS
APVMEPLINSEPSTGVTEPLTPHQPNHQPSFLTSPQDLASSPMEEDEQHSEADEPPSDEPLS
DDPLSPAEEKLPHIDEYWSDSEHIFLDANIGGVAIAPAHGSVLIECARRELHATTPVEHPNR
NHPTRLSLVFYQHKNLNKPQHGFELNKIKFEAKEAKNKKMKASEQKDQAANEGPEQSSEVNE
LNQIPSHKALTLTHDNVVTVSPYALTHVAGPYNHWV
SEQ ID NO: 1140
DNA sequence of the gRNA constant region
gtttaagagctatgctggaaacagcatagcaagtttaaataaggctagtccgttatcaactt
gaaaaagtggcaccgagtcggtgc
SEQ ID NO: 1141
RNA sequence of the gRNA constant region
guuuaagagcuaugcuggaaacagcauagcaaguuuaaauaaggcuaguccguuaucaacuu
gaaaaaguggcaccgagucggugc
SEQ ID NO: 1142
RNA sequence of the full gRNA, including SEQ ID NO: 588 (underlined)
Ggaaccagucagaacaggugguuuaagagcuaugcuggaaacagcauagcaaguuuaaauaa
ggcuaguccguuaucaacuugaaaaaguggcaccgagucggugc
SEQ ID NO: 1143
Forward primer
5′-AATGATACGGCGACCACCGAGATCTACACAATTTCTTGGGTAGTTTGCAGTT
SEQ ID NO: 1144
Reverse primer
5′-CAAGCAGAAGACGGCATACGAGAT-(NNNNNN)-GACTCGGTGCCACTTTTTCAA
SEQ ID NO: 1145
Read1
5′-GATTTCTTGGCTTTATATATCTTGTGGAAAGGACGAAACACCG
SEQ ID NO: 1146
Index
5′-GCTAGTCCGTTATCAACTTGAAAAAGTGGCACCGAGTC
SEQ ID NO: 1147
Read2
5′-
GTTGATAACGGACTAGCCTTATTTAAACTTGCTATGCTGTTTCCAGCATAGCTOTTAAAC
SEQ ID NOS: 1148-1165
See TABLE 5.
SEQ ID NO: 1166
Tet1v4 polypetide sequence
LPTCSCLDRVIQKDKGPYYTHLGAGPSVAAVREIMENRYGQKGNAIRIEIVVYTGKEGKSSH
GCPIAKWVLRRSSDEEKVLCLVRQRTGHHCPTAVMVVLIMVWDGIPLPMADRLYTELTENLK
SYNGHPTDRRCTINENRTCTCQGIDPETCGASESFGCSWSMYFNGCKEGRSPSPRRFRIDPS
SPLHEKNLEDNLQSLATRLAPIYKQYAPVAYQNQVEYENVARECRLGSKEGRPFSGVTACLD
FCAHPHRDIHNMNNGSTVVCTLTREDNRSLGVIPQDEQLHVLPLYKLSDTDEFGSKEGMEAK
IKSGAIEVLAPRRKKRTCFTQPVPRSGKKRAAMMTEVLAHKIRAVEKKPIPRIKRKNNSTTT
NNSKPSSLPTLGSNTETVQPEVKSETEPHFILKSSDNTKTYSLMPSAPHPVKEASPGESWSP
KTASATPAPLKNDATASCGFSERSSTPHCTMPSGRLSGANAAAADGPGISQLGEVAPLPTLS
APVMEPLINSEPSTGVTEPLTPHQPNHQPSELTSPQDLASSPMEEDEQHSEADEPPSDEPLS
DDPLSPAEEKLPHIDEYWSDSEHIFLDANIGGVAIAPAHGSVLIECARRELHATTPVEHPNR
NHPTRLSLVFYQHKNLNKPQHGFELNKIKFEAKEAKNKKMKASEQKDQAANEGPEQSSEVNE
LNQIPSHKALTLTHDNVVTVSPYALTHVAGPYNHWV
SEQ ID NO: 1167
Tet1v4 DNA sequence
ctgcccacctgcagctgtcttgatcgagttatacaaaaagacaaaggcccatattatacaca
ccttggggcaggaccaagtgttgctgctgtcagggaaatcatggagaataggtatggtcaaa
aaggaaacgcaataaggatagaaatagtagtgtacaccggtaaagaagggaaaagctctcat
gggtgtccaattgctaagtgggttttaagaagaagcagtgatgaagaaaaagttctttgttt
ggtccggcagcgtacaggccaccactgtccaactgctgtgatggtggtgctcatcatggtgt
gggatggcatccctcttccaatggccgaccggctatacacagagctcacagagaatctaaag
tcatacaatgggcaccctaccgacagaagatgcaccctcaatgaaaatcgtacctgtacatg
tcaaggaattgatccagagacttgtggagcttcattctcttttggctgttcatggagtatgt
actttaatggctgtaagtttggtagaagcccaagccccagaagatttagaattgatccaagc
tctcccttacatgaaaaaaaccttgaagataacttacagagtttggctacacgattagctcc
aatttataagcagtatgctccagtagcttaccaaaatcaggtggaatatgaaaatgttgccc
gagaatgtcggcttggcagcaaggaaggtcgacccttctctggggtcactgcttgcctggac
ttctgtgctcatccccacagggacattcacaacatgaataatggaagcactgtggtttgtac
cttaactcgagaagataaccgctctttgggtgttattcctcaagatgagcagctccatgtgc
tacctctttataagctttcagacacagatgagtttggctccaaggaaggaatggaagccaag
atcaaatctggggccatcgaggtcctggcaccccgccgcaaaaaaagaacgtgtttcactca
gcctgttccccgttctggaaagaagagggctgcgatgatgacagaggttcttgcacataaga
taagggcagtggaaaagaaacctattccccgaatcaagcggaagaataactcaacaacaaca
aacaacagtaagccttcgtcactgccaaccttagggagtaacactgagaccgtgcaacctga
agtaaaaagtgaaaccgaaccccattttatcttaaaaagttcagacaacactaaaacttatt
cgctgatgccatccgctcctcacccagtgaaagaggcatctccaggcttctcctggtccccg
aagactgcttcagccacaccagctccactgaagaatgacgcaacagcctcatgcgggttttc
agaaagaagcagcactccccactgtacgatgccttcgggaagactcagtggtgccaatgctg
cagctgctgatggccctggcatttcacagcttggcgaagtggctcctctccccaccctgtct
gctcctgtgatggagcccctcattaattctgagccttccactggtgtgactgagccgctaac
gcctcatcagccaaaccaccagccctccttcctcacctctcctcaagaccttgcctcttctc
caatggaagaagatgagcagcattctgaagcagatgagcctccatcagacgaacccctatct
gatgaccccctgtcacctgctgaggagaaattgccccacattgatgagtattggtcagacag
tgagcacatctttttggatgcaaatattggtggggtggccatcgcacctgctcacggctcgg
ttttgattgagtgtgcccggcgagagctgcacgctaccactcctgttgagcaccccaaccgt
aatcatccaacccgcctctcccttgtcttttaccagcacaaaaacctaaataagccccaaca
tggttttgaactaaacaagattaagtttgaggctaaagaagctaagaataagaaaatgaagg
cctcagagcaaaaagaccaggcagctaatgaaggtccagaacagtcctctgaagtaaatgaa
ttgaaccaaattccttctcataaagcattaacattaacccatgacaatgttgtcaccgtgtc
cccttatgctctcacacacgttgcggggccctataaccattgggtc
SEQ ID NO: 1168
Tet1c-dCas9 polypeptide sequence (with two NLS sequences; Tet1c in bold; dCas9 in
italics)
MVPKKKRKVGGGGSGSLPTCSCLDRVIQKDKGPYYTHLGAGPSVAAVREIMENRYGQKGNAI
RIEIVVYTGKEGKSSHGCPIAKWVLRRSSDEEKVLCLVRQRTGHHCPTAVMVVLIMVWDGIP
LPMADRLYTELTENLKSYNGHPTDRRCTLNENRTCTCQGIDPETCGASFSFGCSWSMYENGC
KFGRSPSPRRFRIDPSSPLHEKNLEDNLQSLATRLAPIYKQYAPVAYQNQVEYENVARECRL
GSKEGRPFSGVTACLDFCAHPERDIHNMNNGSTVVCTLTREDNRSLGVIPQDEQLHVLPLYK
LSDTDEFGSKEGMEAKIKSGAIEVLAPRRKKRTCFTQPVPRSGKKRAAMMTEVLAHKIRAVE
KKPIPRIKRKNNSTTTNNSKPSSLPTLGSNTETVQPEVKSETEPHFILKSSDNTKTYSLMPS
APHPVKEASPGFSWSPKTASATPAPLKNDATASCGFSERSSTPHCTMPSGRLSGANAAAADG
PGISQLGEVAPLPTLSAPVMEPLINSEPSTGVTEPLTPHQPNHQPSFLTSPQDLASSPMEED
EQHSEADEPPSDEPLSDDPLSPAEEKLPHIDEYWSDSEHIFLDANIGGVAIAPAHGSVLIEC
ARRELHATTPVEHPNRNHPTRLSLVFYQHKNLNKPQHGFELNKIKFEAKEAKNKKMKASEQK
DQAANEGPEQSSEVNELNQIPSHKALTLTHDNVVTVSPYALTHVAGPYNHWVGRPFGGGGSM
DYKDHDGDYKDHDIDYKDDDDKMAPKKKRKVGIHGVPAADKKYSIGLAIGINSVGWAVITDE
YKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDK
ADERLIYLALAHMIKERGHFLIEGDLNPDNSDVDKLFIQLVQTYNQLFEENPINASGVDAKA
ILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTD
LKALVRQQLPEKYKEIFFDQSKNGYAGYIDGGASQEEFYKFIKPILEKMDGTEELLVKLNRE
DLLRKQRTEDNGSIPHQIHLGELHAILRRQEDFYPFLKDNREKIEKILTFRIPYYVGPLARG
NSRFAWMTRKSEETITPWNFEEVVDKGASAQSFIERMINEDKNLPNEKVLPKHSLLYEYETV
YNELTKVKYVTEGMRKPAFLSGEQKKAIVDLLEKTNRKVTVKQLKEDYFKKIECFDSVEISG
VEDRFNASLGTYHDLLKIIKDKDELDNEENEDILEDIVLTLILFEDREMIEERLKTYAHLED
DKVMKQLKRRRYTGWGRLSRKLINGIRDKQSGKTILDFLKSDGFANRNEMQLIHDDSLTEKE
DIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDI
NRLSDYDVDAIVPQSELKDDSIDNKVLTRSDKNRGKSDNVPSEEVVKKMKNYWRQLLNAKLI
TQRKFDNLTKAERGGLSELDKAGFIKRQLVETRQITKHVAQILDSRMNTKYDENDKLIREVK
VITLKSKLVSDERKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIEINGETGEIVWDKGR
DEATVRKVLSMPQVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLFVEQHKH
YIDEIIEQISEFSKRVILADANLDKVLSAYNKHRDKPIREQAENIIHLFTLINLGAPAAFKY
FDTTIDRKRYTSTKEVIDATLIHQSITGLYETRIDLSQLGGDKRPAATKKAGQAKKKKGGDF
GAS
SEQ ID NO: 1169
Tet1c-dCas9 DNA sequence (with two NLS sequences; Tet1c in bold; dCas9 in italics)
atggtgCCAAAAAAGAAGAGAAAGGTAggcggagGCGGGAGCGGATCCCTGCCCACCTGCAG
CTGTCTTGATCGAGTTATACAAAAAGACAAAGGCCCATATTATACACACCTTGGGGCAGGAC
CAAGTGTTGCTGCTGTCAGGGAAATCATGGAGAATAGGTATGGTCAAAAAGGAAACGCAATA
AGGATAGAAATAGTAGTGTACACCGGTAAAGAAGGGAAAAGCTCTCATGGGTGTCCAATTGC
TAAGTGGGTTTTAAGAAGAAGCAGTGATGAAGAAAAAGTTCTTTGTTTGGTCCGGCAGCGTA
CAGGCCACCACTGTCCAACTGCTGTGATGGTGGTGCTCATCATGGTGTGGGATGGCATCCCT
CTTCCAATGGCCGACCGGCTATACACAGAGCTCACAGAGAATCTAAAGTCATACAATGGGCA
CCCTACCGACAGAAGATGCACCCTCAATGAAAATCGTACCTGTACATGTCAAGGAATTGATC
CAGAGACTTGTGGAGCTTCATTCTCTTTTGGCTGTTCATGGAGTATGTACTTTAATGGCTGT
AAGTTTGGTAGAAGCCCAAGCCCCAGAAGATTTAGAATTGATCCAAGCTCTCCCTTACATGA
AAAAAACCTTGAAGATAACTTACAGAGTTTGGCTACACGATTAGCTCCAATTTATAAGCAGT
ATGCTCCAGTAGCTTACCAAAATCAGGTGGAATATGAAAATGTTGCCCGAGAATGTCGGCTT
GGCAGCAAGGAAGGTCGACCCTTCTCTGGGGTCACTGCTTGCCTGGACTTCTGTGCTCATCC
CCACAGGGACATTCACAACATGAATAATGGAAGCACTGTGGTTTGTACCTTAACTCGAGAAG
ATAACCGCTCTTTGGGTGTTATTCCTCAAGATGAGCAGCTCCATGTGCTACCTCTTTATAAG
CTTTCAGACACAGATGAGTTTGGCTCCAAGGAAGGAATGGAAGCCAAGATCAAATCTGGGGC
CATCGAGGTCCTGGCACCCCGCCGCAAAAAAAGAACGTGTTTCACTCAGCCTGTTCCCCGTT
CTGGAAAGAAGAGGGCTGCGATGATGACAGAGGTTCTTGCACATAAGATAAGGGCAGTGGAA
AAGAAACCTATTCCCCGAATCAAGCGGAAGAATAACTCAACAACAACAAACAACAGTAAGCC
TTCGTCACTGCCAACCTTAGGGAGTAACACTGAGACCGTGCAACCTGAAGTAAAAAGTGAAA
CCGAACCCCATTTTATCTTAAAAAGTTCAGACAACACTAAAACTTATTCGCTGATGCCATCC
GCTCCTCACCCAGTGAAAGAGGCATCTCCAGGCTTCTCCTGGTCCCCGAAGACTGCTTCAGC
CACACCAGCTCCACTGAAGAATGACGCAACAGCCTCATGCGGGTTTTCAGAAAGAAGCAGCA
CTCCCCACTGTACGATGCCTTCGGGAAGACTCAGTGGTGCCAATGCTGCAGCTGCTGATGGC
CCTGGCATTTCACAGCTTGGCGAAGTGGCTCCTCTCCCCACCCTGTCTGCTCCTGTGATGGA
GCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGAT
GAGCAGCATTCTGAAGCAGATGAGCCTCCATCAGACGAACCCCTATCTGATGACCCCCTGTC
ACCTGCTGAGGAGAAATTGCCCCACATTGATGAGTATTGGTCAGACAGTGAGCACATCTTTT
TGGATGCAAATATTGGTGGGGTGGCCATCGCACCTGCTCACGGCTCGGTTTTGATTGAGTGT
GCCCGGCGAGAGCTGCACGCTACCACTCCTGTTGAGCACCCCAACCGTAATCATCCAACCCG
CCTCTCCCTTGTCTTTTACCAGCACAAAAACCTAAATAAGCCCCAACATGGTTTTGAACTAA
ACAAGATTAAGTTTGAGGCTAAAGAAGCTAAGAATAAGAAAATGAAGGCCTCAGAGCAAAAA
GACCAGGCAGCTAATGAAGGTCCAGAACAGTCCTCTGAAGTAAATGAATTGAACCAAATTCC
TTCTCATAAAGCATTAACATTAACCCATGACAATGTTGTCACCGTGTCCCCTTATGCTCTCA
CACACGTTGCGGGGCCCTATAAccattgggtcggccggccattcggtggaggtggctCCATG
GACTATAAGGACCACGACGGAGACTACAAGGATCATGATATTGATTACAAAGACGATGACGA
TAAGATGGCCCCAAAGAAGAAGCGGAAGGTCGGTATCCACGGAGTCCCAGCAGCCGACAAGA
AGTACAGCATCGGCCTGGCCATCGGCACCAACTCTGTGGGCTGGGCCGTGATCACCGACGAG
TACAAGGTGCCCAGCAAGAAATTCAAGGTGCTGGGCAACACCGACCGGCACAGCATCAAGAA
GAACCTGATCGGAGCCCTGCTGTTCGACAGCGGCGAAACAGCCGAGGCCACCCGGCTGAAGA
GAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGT
GGAAGAGGATAAGAAGCACGAGCGGCACCCCATCTTCGGCAACATCGTGGACGAGGTGGCCT
ACCACGAGAAGTACCCCACCATCTACCACCTGAGAAAGAAACTGGTGGACAGCACCGACAAG
GCCGACCTGCGGCTGATCTATCTGGCCCTGGCCCACATGATCAAGTTCCGGGGCCACTTCCT
GATCGAGGGCGACCTGAACCCCGACAACAGCGACGTGGACAAGCTGTTCATCCAGCTGGTGC
AGACCTACAACCAGCTGTTCGAGGAAAACCCCATCAACGCCAGCGGCGTGGACGCCAAGGCC
ATCCTGTCTGCCAGACTGAGCAAGAGCAGACGGCTGGAAAATCTGATCGCCCAGCTGCCCGG
CGAGAAGAAGAATGGCCTGTTCGGAAACCTGATTGCCCTGAGCCTGGGCCTGACCCCCAACT
TCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGCCGC
CAAGAACCTGTCCGACGCCATCCTGCTGAGCGACATCCTGAGAGTGAACACCGAGATCACCA
AGGCCCCCCTGAGCGCCTCTATGATCAAGAGATACGACGAGCACCACCAGGACCTGACCCTG
CTGAAAGCTCTCGTGCGGCAGCAGCTGCCTGAGAAGTACAAAGAGATTTTCTTCGACCAGAG
CAAGAACGGCTACGCCGGCTACATTGACGGCGGAGCCAGCCAGGAAGAGTTCTACAAGTTCA
TCAAGCCCATCCTGGAAAAGATGGACGGCACCGAGGAACTGCTCGTGAAGCTGAACAGAGAG
GACCTGCTGCGGAAGCAGCGGACCTTCGACAACGGCAGCATCCCCCACCAGATCCACCTGGG
AGAGCTGCACGCCATTCTGCGGCGGCAGGAAGATTTTTACCCATTCCTGAAGGACAACCGGG
AAAAGATCGAGAAGATCCTGACCTTCCGCATCCCCTACTACGTGGGCCCTCTGGCCAGGGGA
AACAGCAGATTCGCCTGGATGACCAGAAAGAGCGAGGAAACCATCACCCCCTGGAACTTCGA
GGAAGTGGTGGACAAGGGCGCTTCCGCCCAGAGCTTCATCGAGCGGATGACCAACTTCGATA
AGAACCTGCCCAACGAGAAGGTGCTGCCCAAGCACAGCCTGCTGTACGAGTACTTCACCGTG
TATAACGAGCTGACCAAAGTGAAATACGTGACCGAGGGAATGAGAAAGCCCGCCTTCCTGAG
CGGCGAGCAGAAAAAGGCCATCGTGGACCTGCTGTTCAAGACCAACCGGAAAGTGACCGTGA
AGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGA
CAAGGACTTCCTGGACAATGAGGAAAACGAGGACATTCTGGAAGATATCGTGCTGACCCTGA
CACTGTTTGAGGACAGAGAGATGATCGAGGAACGGCTGAAAACCTATGCCCACCTGTTCGAC
GACAAAGTGATGAAGCAGCTGAAGCGGCGGAGATACACCGGCTGGGGCAGGCTGAGCCGGAA
GCTGATCAACGGCATCCGGGACAAGCAGTCCGGCAAGACAATCCTGGATTTCCTGAAGTCCG
ACGGCTTCGCCAACAGAAACTTCATGCAGCTGATCCACGACGACAGCCTGACCTTTAAAGAG
GACATCCAGAAAGCCCAGGTGTCCGGCCAGGGCGATAGCCTGCACGAGCACATTGCCAATCT
GGCCGGCAGCCCCGCCATTAAGAAGGGCATCCTGCAGACAGTGAAGGTGGTGGACGAGCTCG
TGAAAGTGATGGGCCGGCACAAGCCCGAGAACATCGTGATCGAAATGGCCAGAGAGAACCAG
ACCACCCAGAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAGAACGAGA
AGCTGTACCTGTACTACCTGCAGAATGGGCGGGATATGTACGTGGACCAGGAACTGGACATC
AACCGGCTGTCCGACTACGATGTGGACGCCATCGTGCCTCAGAGCTTTCTGAAGGACGACTC
CATCGACAACAAGGTGCTGACCAGAAGCGACAAGAACCGGGGCAAGAGCGACAACGTGCCCT
CCGAAGAGGTCGTGAAGAAGATGAAGAACTACTGGCGGCAGCTGCTGAACGCCAAGCTGATT
ACCCAGAGAAAGTTCGACAATCTGACCAAGGCCGAGAGAGGCGGCCTGAGCGAACTGGATAA
GGCCGGCTTCATCAAGAGACAGCTGGTGGAAACCCGGCAGATCACAAAGCACGTGGCACAGA
TCCTGGACTCCCGGATGAACACTAAGTACGACGAGAATGACAAGCTGATCCGGGAAGTGAAA
GTGATCACCCTGAAGTCCAAGCTGGTGTCCGATTTCCGGAAGGATTTCCAGTTTTACAAAGT
GCGCGAGATCAACAACTACCACCACGCCCACGACGCCTACCTGAACGCCGTCGTGGGAACCG
CCCTGATCAAAAAGTACCCTAAGCTGGAAAGCGAGTTCGTGTACGGCGACTACAAGGTGTAC
GACGTGCGGAAGATGATCGCCAAGAGCGAGCAGGAAATCGGCAAGGCTACCGCCAAGTACTT
CTTCTACAGCAACATCATGAACTTTTTCAAGACCGAGATTACCCTGGCCAACGGCGAGATCC
GGAAGCGGCCTCTGATCGAGACAAACGGCGAAACCGGGGAGATCGTGTGGGATAAGGGCCGG
GATTTTGCCACCGTGCGGAAAGTGCTGAGCATGCCCCAAGTGAATATCGTGAAAAAGACCGA
GGTGCAGACAGGCGGCTTCAGCAAAGAGTCTATCCTGCCCAAGAGGAACAGCGATAAGCTGA
TCGCCAGAAAGAAGGACTGGGACCCTAAGAAGTACGGCGGCTTCGACAGCCCCACCGTGGCC
TATTCTGTGCTGGTGGTGGCCAAAGTGGAAAAGGGCAAGTCCAAGAAACTGAAGAGTGTGAA
AGAGCTGCTGGGGATCACCATCATGGAAAGAAGCAGCTTCGAGAAGAATCCCATCGACTTTC
TGGAAGCCAAGGGCTACAAAGAAGTGAAAAAGGACCTGATCATCAAGCTGCCTAAGTACTCC
CTGTTCGAGCTGGAAAACGGCCGGAAGAGAATGCTGGCCTCTGCCGGCGAACTGCAGAAGGG
AAACGAACTGGCCCTGCCCTCCAAATATGTGAACTTCCTGTACCTGGCCAGCCACTATGAGA
AGCTGAAGGGCTCCCCCGAGGATAATGAGCAGAAACAGCTGTTTGTGGAACAGCACAAGCAC
TACCTGGACGAGATCATCGAGCAGATCAGCGAGTTCTCCAAGAGAGTGATCCTGGCCGACGC
TAATCTGGACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTTCAAGTAC
TTTGACACCACCATCGACCGGAAGAGGTACACCAGCACCAAAGAGGTGCTGGACGCCACCCT
GATCCACCAGAGCATCACCGGCCTGTACGAGACACGGATCGACCTGTCTCAGCTGGGAGGCG
ACAAAAGGCCGGCGGCCACGAAAAAGGccggacaggccaaaaagaaaaagGGCggagatttc
ggcGctagC
SEQ ID NO: 1170
Tet1v4-dCas9 polypeptide sequence (with three NLS sequences; Tet1v4 in bold; dCas9 in
italics)
MALPTCSCLDRVIQKDKGPYYTHLGAGPSVAAVREIMENRYGQKGNAIRIEIVVYTGKEGKS
SHGCPIAKWVLRRSSDEEKVLCLVRQRTGHHCPTAVMVVLIMVWDGIPLPMADRLYTELTEN
LKSYNGHPTDRRCTLNENRTCTCQGIDPETCGASFSFGCSWSMYENGCKFGRSPSPRRFRID
PSSPLHEKNLEDNLQSLATRLAPIYKQYAPVAYQNQVEYENVARECRLGSKEGRPFSGVTAC
LDFCAHPHRDIHNMNNGSTVVCTLTREDNRSLGVIPQDEQLHVLPLYKLSDTDEFGSKEGME
AKIKSGAIEVLAPRRKKRTCFTQPVPRSGKKRAAMMTEVLAHKIRAVEKKPIPRIKRKNNST
TTNNSKPSSLPTLGSNTETVQPEVKSETEPHFILKSSDNTKTYSLMPSAPHPVKEASPGFSW
SPKTASATPAPLKNDATASCGFSERSSTPHCTMPSGRLSGANAAAADGPGISQLGEVAPLPT
LSAPVMEPLINSEPSTGVTEPLTPHQPNHQPSFLTSPQDLASSPMEEDEQHSEADEPPSDEP
LSDDPLSPAEEKLPHIDEYWSDSEHIFLDANIGGVAIAPAHGSVLIECARRELHATTPVEHP
NRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWVGGPSSGAPPPSGGSPAGSPTSTEE
GTSESATPESGPGTSTEPSEGSAPGSPAGSPTSTEEGTSTEPSEGSAPGTSTEPSEMDKKYS
IGLAIGTNSVGWAVITDEYKVPSKKFKVLGNTDRHSIKKNLIGALLFDSGETAEATRLKRTA
RRRYTRRKNRICYLQEIFSNEMAKVDDSFFHRLEESELVEEDKKHERHPIFGNIVDEVAYHE
KYPTIYHLRKKLVDSTDKADLRLIYLALAHMIKERGHFLIEGDLNPDNSDVDKLFIQLVQTY
NQLFEENPINASGVDAKAILSARLSKSRRLENLIAQLPGEKKNGLEGNLIALSLGLTPNEKS
NEDLAEDAKLQLSKDTYDDDLDNILAQIGDQYADLFLAAKNLSDAILLSDIERVNTEITKAP
LSASMIKRYDEHHQDITLLKALVRQQLPEKYKEIFFDQSKNGYAGYIDGGASQEEFYKFIKP
ILEKMDGTEELLVKINREDLLRKQRTEDNGSIPHQIHLGELHAILRRQEDEYPELKDNREKI
EKILTFRIPYYVGPLARGNSRFAWMTRKSEETITPWNFEEVVDKGASAQSFIERMINEDKNL
PNEKVLPKHSLLYEYFTVYNELTKVKYVTEGMRKPAFLSGEQKKAIVDLLEKTNRKVTVKQL
KEDYFKKIECFDSVEISGVEDRENASLGTYHDLLKIIKDKDFLDNEENEDILEDIVLILTLF
EDREMIEERLKTYAHLEDDKVMKQLKRRRYTGWGRLSRKLINGIRDKQSGKTILDELKSDGF
ANRNEMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNVPSEE
VVKKMKNYWRQLLNAKLITQRKEDNLTKAERGGLSELDKAGFIKRQLVETRQITKHVAQILD
SRMNTKYDENDKLIREVKVITLKSKLVSDERKDFQFYKVREINNYHHAHDAYLNAVVGTALI
KKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGESKESILPKRNSDKLIAR
KKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAEN
IIHLFTLTNLGAPAAFKYEDTTIDRKRYTSTKEVLDATLIHQSITGLYETRIDLSQLGGDGG
GGSPKKKRKVDPKKKRKVDPKKKRKVGGSG
SEQ ID NO: 1171
Tet1v4-dCas9 DNA sequence (with three NLS sequences; Tet1v4 in bold; dCas9 in italics)
atggccctgcccacctgcagctgtcttgatcgagttatacaaaaagacaaaggcccatatta
tacacaccttggggcaggaccaagtgttgctgctgtcagggaaatcatggagaataggtatg
gtcaaaaaggaaacgcaataaggatagaaatagtagtgtacaccggtaaagaagggaaaagc
tctcatgggtgtccaattgctaagtgggttttaagaagaagcagtgatgaagaaaaagttct
ttgtttggtccggcagcgtacaggccaccactgtccaactgctgtgatggtggtgctcatca
tggtgtgggatggcatccctcttccaatggccgaccggctatacacagagctcacagagaat
ctaaagtcatacaatgggcaccctaccgacagaagatgcaccctcaatgaaaatcgtacctg
tacatgtcaaggaattgatccagagacttgtggagcttcattctcttttggctgttcatgga
gtatgtactttaatggctgtaagtttggtagaagcccaagccccagaagatttagaattgat
ccaagctctcccttacatgaaaaaaaccttgaagataacttacagagtttggctacacgatt
agctccaatttataagcagtatgctccagtagcttaccaaaatcaggtggaatatgaaaatg
ttgcccgagaatgtcggcttggcagcaaggaaggtcgacccttctctggggtcactgcttgc
ctggacttctgtgctcatccccacagggacattcacaacatgaataatggaagcactgtggt
ttgtaccttaactcgagaagataaccgctctttgggtgttattcctcaagatgagcagctcc
atgtgctacctctttataagctttcagacacagatgagtttggctccaaggaaggaatggaa
gccaagatcaaatctggggccatcgaggtcctggcaccccgccgcaaaaaaagaacgtgttt
cactcagcctgttccccgttctggaaagaagagggctgcgatgatgacagaggttcttgcac
ataagataagggcagtggaaaagaaacctattccccgaatcaagcggaagaataactcaaca
acaacaaacaacagtaagccttcgtcactgccaaccttagggagtaacactgagaccgtgca
acctgaagtaaaaagtgaaaccgaaccccattttatcttaaaaagttcagacaacactaaaa
cttattcgctgatgccatccgctcctcacccagtgaaagaggcatctccaggcttctcctgg
tccccgaagactgcttcagccacaccagctccactgaagaatgacgcaacagcctcatgcgg
gttttcagaaagaagcagcactccccactgtacgatgccttcgggaagactcagtggtgcca
atgctgcagctgctgatggccctggcatttcacagcttggcgaagtggctcctctccccacc
ctgtctgctcctgtgatggagcccctcattaattctgagccttccactggtgtgactgagcc
gctaacgcctcatcagccaaaccaccagccctccttcctcacctctcctcaagaccttgcct
cttctccaatggaagaagatgagcagcattctgaagcagatgagcctccatcagacgaaccc
ctatctgatgaccccctgtcacctgctgaggagaaattgccccacattgatgagtattggtc
agacagtgagcacatctttttggatgcaaatattggtggggtggccatcgcacctgctcacg
gctcggttttgattgagtgtgcccggcgagagctgcacgctaccactcctgttgagcacccc
aaccgtaatcatccaacccgcctctcccttgtcttttaccagcacaaaaacctaaataagcc
ccaacatggttttgaactaaacaagattaagtttgaggctaaagaagctaagaataagaaaa
tgaaggcctcagagcaaaaagaccaggcagctaatgaaggtccagaacagtcctctgaagta
aatgaattgaaccaaattccttctcataaagcattaacattaacccatgacaatgttgtcac
cgtgtccccttatgctctcacacacgttgcggggccctataaccattgggtcggagggccga
gctctggcgcacccccaccaagtggagggtctcctgccgggtccccaacatctactgaagaa
ggcaccagcgaatccgcaacgcccgagtcaggccctggtacctccacagaaccatctgaagg
tagtgcgcctggttccccagctggaagccctacttccaccgaagaaggcacgtcaaccgaac
caagtgaaggatctgcccctgggaccagcactgaaccatctgagatggacaagaagtattct
atcggactggccatcgggactaatagcgtcgggtgggccgtgatcactgacgagtacaaggt
gccctctaagaagttcaaggtgctcgggaacaccgaccggcattccatcaagaaaaatctga
tcggagctctcctctttgattcaggggagaccgctgaagcaacccgcctcaagcggactgct
agacggcggtacaccaggaggaagaaccggatttgttaccttcaagagatattctccaacga
aatggcaaaggtcgacgacagcttcttccataggctggaagaatcattcctcgtggaagagg
ataagaagcatgaacggcatcccatcttcggtaatatcgtcgacgaggtggcctatcacgag
aaatacccaaccatctaccatcttcgcaaaaagctggtggactcaaccgacaaggcagacct
ccggcttatctacctggccctggcccacatgatcaagttcagaggccacttcctgatcgagg
gcgacctcaatcctgacaatagcgatgtggataaactgttcatccagctggtgcagacttac
aaccagctctttgaagagaaccccatcaatgcaagcggagtcgatgccaaggccattctgtc
agcccggctgtcaaagagccgcagacttgagaatcttatcgctcagctgccgggtgaaaaga
aaaatggactgttcgggaacctgattgctctttcacttgggctgactcccaatttcaagtct
aatttcgacctggcagaggatgccaagctgcaactgtccaaggacacctatgatgacgatct
cgacaacctcctggcccagatcggtgaccaatacgccgaccttttccttgctgctaagaatc
tttctgacgccatcctgctgtctgacattctccgcgtgaacactgaaatcaccaaggcccct
ctttcagcttcaatgattaagcggtatgatgagcaccaccaggacctgaccctgcttaaggc
actcgtccggcagcagcttccggagaagtacaaggaaatcttctttgaccagtcaaagaatg
gatacgccggctacatcgacggaggtgcctcccaagaggaattttataagtttatcaaacct
atccttgagaagatggacggcaccgaagagctcctcgtgaaactgaatcgggaggatctgct
gcggaagcagcgcactttcgacaatgggagcattccccaccagatccatcttggggagcttc
acgccatccttcggcgccaagaggacttctacccctttcttaaggacaacagggagaagatt
gagaaaattctcactttccgcatcccctactacgtgggacccctcgccagaggaaatagccg
gtttgcttggatgaccagaaagtcagaagaaactatcactccctggaacttcgaagaggtgg
tggacaagggagccagcgctcagtcattcatcgaacggatgactaacttcgataagaacctc
cccaatgagaaggtcctgccgaaacattccctgctctacgagtactttaccgtgtacaacga
gctgaccaaggtgaaatatgtcaccgaagggatgaggaagcccgcattcctgtcaggcgaac
aaaagaaggcaattgtggaccttctgttcaagaccaatagaaaggtgaccgtgaagcagctg
aaggaggactatttcaagaaaattgaatgcttcgactctgtggagattagcggggtcgaaga
tcggttcaacgcaagcctgggtacctaccatgatctgcttaagatcatcaaggacaaggatt
ttctggacaatgaggagaacgaggacatccttgaggacattgtcctgactctcactctgttc
gaggaccgggaaatgatcgaggagaggcttaagacctacgcccatctgttcgacgataaagt
gatgaagcaacttaaacggagaagatataccggatggggacgccttagccgcaaactcatca
acggaatccgggacaaacagagcggaaagaccattcttgatttccttaaaagcgacggattc
gctaatcgcaacttcatgcaacttatccatgatgattccctgacctttaaggaggacatcca
gaaggcccaagtgtctggacaaggtgactcactgcacgagcatatcgcaaatctggctggtt
cacccgctattaagaagggtattctccagaccgtgaaagtcgtggacgagctggtcaaggtg
atgggtcgccataaaccagagaacattgtcatcgagatggccagggaaaaccagactaccca
gaagggacagaagaacagcagggagcggatgaaaagaattgaggaagggattaaggagctcg
ggtcacagatccttaaagagcacccggtggaaaacacccagcttcagaatgagaagctctat
ctgtactaccttcaaaatggacgcgatatgtatgtggaccaagagcttgatatcaacaggct
ctcagactacgacgtggacgccatcgtccctcagagcttcctcaaagacgactcaattgaca
ataaggtgctgactcgctcagacaagaaccggggaaagtcagataacgtgccctcagaggaa
gtcgtgaaaaagatgaagaactattggcgccagcttctgaacgcaaagctgatcactcagcg
gaagttcgacaatctcactaaggctgagaggggcggactgagcgaactggacaaagcaggat
tcattaaacggcaacttgtggagactcggcagattactaaacatgtcgcccaaatccttgac
tcacgcatgaataccaagtacgacgaaaacgacaaacttatccgcgaggtgaaggtgattac
cctgaagtccaagctggtcagcgatttcagaaaggactttcaattctacaaagtgcgggaga
tcaataactatcatcatgctcatgacgcatatctgaatgccgtggtgggaaccgccctgatc
aagaagtacccaaagctggaaagcgagttcgtgtacggagactacaaggtctacgacgtgcg
caagatgattgccaaatctgagcaggagatcggaaaggccaccgcaaagtacttcttctaca
gcaacatcatgaatttcttcaagaccgaaatcacccttgcaaacggtgagatccggaagagg
ccgctcatcgagactaatggggagactggcgaaatcgtgtgggacaagggcagagatttcgc
taccgtgcgcaaagtgctttctatgcctcaagtgaacatcgtgaagaaaaccgaggtgcaaa
ccggaggcttttctaaggaatcaatcctccccaagcgcaactccgacaagctcattgcaagg
aagaaggattgggaccctaagaagtacggcggattcgattcaccaactgtggcttattctgt
cctggtcgtggctaaggtggaaaaaggaaagtctaagaagctcaagagcgtgaaggaactgc
tgggtatcaccattatggagcgcagctccttcgagaagaacccaattgactttctcgaagcc
aaaggttacaaggaagtcaagaaggaccttatcatcaagctcccaaagtatagcctgttcga
actggagaatgggcggaagcggatgctcgcctccgctggcgaacttcagaagggtaatgagc
tggctctcccctccaagtacgtgaatttcctctaccttgcaagccattacgagaagctgaag
gggagccccgaggacaacgagcaaaagcaactgtttgtggagcagcataagcattatctgga
cgagatcattgagcagatttccgagttttctaaacgcgtcattctcgctgatgccaacctcg
ataaagtccttagcgcatacaataagcacagagacaaaccaattcgggagcaggctgagaat
atcatccacctgttcaccctcaccaatcttggtgcccctgccgcattcaagtacttcgacac
caccatcgaccggaaacgctatacctccaccaaagaagtgctggacgccaccctcatccacc
agagcatcaccggactttacgaaactcggattgacctctcacagctcggaggggatggtggc
ggaggctcgccaaaaaagaagagaaaggtagacccaaagaaaaaacgaaaagtagatccgaa
aaagaagaggaaggtgggaggtagtggg
SEQ ID NO: 1172
Tet1c-dCas9-Thy1.1 polypeptide sequence (SV40 NLS-Tet1 catalytic domain-dCas9-
nucleoplasmin NLS-T2A-mouse Thy1.1 reporter)
MVPKKKRKVGGGGSGSIPTCSCLDRVIQKDKGPYYTHLGAGPSVAAVREIMENRYGQKGNAI
RIEIVVYTGKEGKSSHGCPIAKWVLRRSSDEEKVLCLVRQRTGHHCPTAVMVVLIMVWDGIP
LPMADRLYTELTENLKSYNGHPTDRRCTINENRTCTCQGIDPETCGASFSEGCSWSMYENGC
KFGRSPSPRRFRIDPSSPLHEKNLEDNLQSLATRLAPIYKQYAPVAYQNQVEYENVARECRL
GSKEGRPFSGVTACLDFCAHPHRDIHNMNNGSTVVCTLTREDNRSLGVIPQDEQLHVLPLYK
LSDTDEFGSKEGMEAKIKSGAIEVLAPRRKKRTCFTQPVPRSGKKRAAMMTEVLAHKIRAVE
KKPIPRIKRKNNSTTTNNSKPSSLPTLGSNTETVQPEVKSETEPHFILKSSDNTKTYSIMPS
APHPVKEASPGFSWSPKTASATPAPLKNDATASCGFSERSSTPHCTMPSGRLSGANAAAADG
PGISQLGEVAPLPTLSAPVMEPLINSEPSTGVTEPLTPHQPNHQPSELTSPQDLASSPMEED
EQHSEADEPPSDEPLSDDPLSPAEEKLPHIDEYWSDSEHIFLDANIGGVAIAPAHGSVLIEC
ARRELHATTPVEHPNRNHPTRLSLVFYQHKNLNKPQHGFELNKIKFEAKEAKNKKMKASEQK
DQAANEGPEQSSEVNELNQIPSHKALTLTHDNVVTVSPYALTHVAGPYNHWVGRPFGGGGSM
DYKDHDGDYKDHDIDYKDDDDKMAPKKKRKVGIHGVPAADKKYSIGLAIGTNSVGWAVITDE
YKVPSKKFKVLGNTDRHSIKKNLIGALLFDSGETAEATRLKRTARRRYTRRKNRICYLQEIF
SNEMAKVDDSFFHRLEESFLVEEDKKHERHPIFGNIVDEVAYHEKYPTIYHLRKKLVDSTDK
ADLRLIYLALAHMIKFRGHFLIEGDLNPDNSDVDKLFIQLVQTYNQLFEENPINASGVDAKA
ILSARLSKSRRLENLIAQLPGEKKNGLFGNLIALSLGLTPNFKSNEDLAEDAKLQLSKDTYD
DDLDNLLAQIGDQYADLFLAAKNLSDAILLSDILRVNTEITKAPLSASMIKRYDEHHQDLTL
LKALVRQQLPEKYKEIFFDQSKNGYAGYIDGGASQEEFYKFIKPILEKMDGTEELLVKINRE
DLLRKQRTFDNGSIPHQIHLGELHAILRRQEDFYPELKDNREKIEKILTFRIPYYVGPLARG
NSRFAWMTRKSEETITPWNFEEVVDKGASAQSFIERMINEDKNLPNEKVLPKHSLLYEYFTV
YNELTKVKYVTEGMRKPAFLSGEQKKAIVDLLFKTNRKVTVKQLKEDYFKKIECFDSVEISG
VEDRENASLGTYHDLLKIIKDKDELDNEENEDILEDIVLTLTLFEDREMIEERLKTYAHLED
DKVMKQLKRRRYTGWGRISRKLINGIRDKQSGKTILDELKSDGFANRNFMQLIHDDSLTFKE
DIQKAQVSGQGDSLHEHIANLAGSPAIKKGILQTVKVVDELVKVMGRHKPENIVIEMARENQ
TTQKGQKNSRERMKRIEEGIKELGSQILKEHPVENTQLQNEKLYLYYLQNGRDMYVDQELDI
NRLSDYDVDAIVPQSELKDDSIDNKVLTRSDKNRGKSDNVPSEEVVKKMKNYWRQLLNAKLI
TQRKEDNLTKAERGGLSELDKAGFIKRQLVETRQITKHVAQILDSRMNTKYDENDKLIREVK
VITLKSKLVSDERKDEQFYKVREINNYHHAHDAYLNAVVGTALIKKYPKLESEFVYGDYKVY
DVRKMIAKSEQEIGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDPKKYGGEDSPTVA
YSVLVVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLFVEQHKH
YLDEIIEQISEFSKRVILADANLDKVLSAYNKHRDKPIREQAENIIHLFTLINLGAPAAFKY
FDTTIDRKRYTSTKEVLDATLIHQSITGLYETRIDLSQLGGDKRPAATKKAGQAKKKKGGDF
GASEGRGSLLTCGDVEENPGPGTMNPAISVALLLSVLQVSRGQKVTSLTACLVNQNLRLDCR
HENNTKDNSIQHEFSLTREKRKHVISGTLGIPEHTYRSRVTLSNQPYIKVLTLANFTTKDEG
DYFCELRVSGANPMSSNKSISVYRDKLVKCGGISLLVQNTSWMLLLLLSLSLLQALDFISL*
SEQ ID NO: 1173
Tet1c-dCas9-Thy1.1 DNA sequence (SV40 NLS-Tet1 catalytic domain-dCas9-
nucleoplasmin NLS-T2A-mouse Thy1.1 reporter)
atggtgCCAAAAAAGAAGAGAAAGGTAggcggagGCGGGAGCGGATCCCTGCCCACCTGCAG
CTGTCTTGATCGAGTTATACAAAAAGACAAAGGCCCATATTATACACACCTTGGGGCAGGAC
CAAGTGTTGCTGCTGTCAGGGAAATCATGGAGAATAGGTATGGTCAAAAAGGAAACGCAATA
AGGATAGAAATAGTAGTGTACACCGGTAAAGAAGGGAAAAGCTCTCATGGGTGTCCAATTGC
TAAGTGGGTTTTAAGAAGAAGCAGTGATGAAGAAAAAGTTCTTTGTTTGGTCCGGCAGCGTA
CAGGCCACCACTGTCCAACTGCTGTGATGGTGGTGCTCATCATGGTGTGGGATGGCATCCCT
CTTCCAATGGCCGACCGGCTATACACAGAGCTCACAGAGAATCTAAAGTCATACAATGGGCA
CCCTACCGACAGAAGATGCACCCTCAATGAAAATCGTACCTGTACATGTCAAGGAATTGATC
CAGAGACTTGTGGAGCTTCATTCTCTTTTGGCTGTTCATGGAGTATGTACTTTAATGGCTGT
AAGTTTGGTAGAAGCCCAAGCCCCAGAAGATTTAGAATTGATCCAAGCTCTCCCTTACATGA
AAAAAACCTTGAAGATAACTTACAGAGTTTGGCTACACGATTAGCTCCAATTTATAAGCAGT
ATGCTCCAGTAGCTTACCAAAATCAGGTGGAATATGAAAATGTTGCCCGAGAATGTCGGCTT
GGCAGCAAGGAAGGTCGACCCTTCTCTGGGGTCACTGCTTGCCTGGACTTCTGTGCTCATCC
CCACAGGGACATTCACAACATGAATAATGGAAGCACTGTGGTTTGTACCTTAACTCGAGAAG
ATAACCGCTCTTTGGGTGTTATTCCTCAAGATGAGCAGCTCCATGTGCTACCTCTTTATAAG
CTTTCAGACACAGATGAGTTTGGCTCCAAGGAAGGAATGGAAGCCAAGATCAAATCTGGGGC
CATCGAGGTCCTGGCACCCCGCCGCAAAAAAAGAACGTGTTTCACTCAGCCTGTTCCCCGTT
CTGGAAAGAAGAGGGCTGCGATGATGACAGAGGTTCTTGCACATAAGATAAGGGCAGTGGAA
AAGAAACCTATTCCCCGAATCAAGCGGAAGAATAACTCAACAACAACAAACAACAGTAAGCC
TTCGTCACTGCCAACCTTAGGGAGTAACACTGAGACCGTGCAACCTGAAGTAAAAAGTGAAA
CCGAACCCCATTTTATCTTAAAAAGTTCAGACAACACTAAAACTTATTCGCTGATGCCATCC
GCTCCTCACCCAGTGAAAGAGGCATCTCCAGGCTTCTCCTGGTCCCCGAAGACTGCTTCAGC
CACACCAGCTCCACTGAAGAATGACGCAACAGCCTCATGCGGGTTTTCAGAAAGAAGCAGCA
CTCCCCACTGTACGATGCCTTCGGGAAGACTCAGTGGTGCCAATGCTGCAGCTGCTGATGGC
CCTGGCATTTCACAGCTTGGCGAAGTGGCTCCTCTCCCCACCCTGTCTGCTCCTGTGATGGA
GCCCCTCATTAATTCTGAGCCTTCCACTGGTGTGACTGAGCCGCTAACGCCTCATCAGCCAA
ACCACCAGCCCTCCTTCCTCACCTCTCCTCAAGACCTTGCCTCTTCTCCAATGGAAGAAGAT
GAGCAGCATTCTGAAGCAGATGAGCCTCCATCAGACGAACCCCTATCTGATGACCCCCTGTC
ACCTGCTGAGGAGAAATTGCCCCACATTGATGAGTATTGGTCAGACAGTGAGCACATCTTTT
TGGATGCAAATATTGGTGGGGTGGCCATCGCACCTGCTCACGGCTCGGTTTTGATTGAGTGT
GCCCGGCGAGAGCTGCACGCTACCACTCCTGTTGAGCACCCCAACCGTAATCATCCAACCCG
CCTCTCCCTTGTCTTTTACCAGCACAAAAACCTAAATAAGCCCCAACATGGTTTTGAACTAA
ACAAGATTAAGTTTGAGGCTAAAGAAGCTAAGAATAAGAAAATGAAGGCCTCAGAGCAAAAA
GACCAGGCAGCTAATGAAGGTCCAGAACAGTCCTCTGAAGTAAATGAATTGAACCAAATTCC
TTCTCATAAAGCATTAACATTAACCCATGACAATGTTGTCACCGTGTCCCCTTATGCTCTCA
CACACGTTGCGGGGCCCTATAAccattgggtcggccggccattcggtggaggtggctCCATG
GACTATAAGGACCACGACGGAGACTACAAGGATCATGATATTGATTACAAAGACGATGACGA
TAAGATGGCCCCAAAGAAGAAGCGGAAGGTCGGTATCCACGGAGTCCCAGCAGCCGACAAGA
AGTACAGCATCGGCCTGGCCATCGGCACCAACTCTGTGGGCTGGGCCGTGATCACCGACGAG
TACAAGGTGCCCAGCAAGAAATTCAAGGTGCTGGGCAACACCGACCGGCACAGCATCAAGAA
GAACCTGATCGGAGCCCTGCTGTTCGACAGCGGCGAAACAGCCGAGGCCACCCGGCTGAAGA
GAACCGCCAGAAGAAGATACACCAGACGGAAGAACCGGATCTGCTATCTGCAAGAGATCTTC
AGCAACGAGATGGCCAAGGTGGACGACAGCTTCTTCCACAGACTGGAAGAGTCCTTCCTGGT
GGAAGAGGATAAGAAGCACGAGCGGCACCCCATCTTCGGCAACATCGTGGACGAGGTGGCCT
ACCACGAGAAGTACCCCACCATCTACCACCTGAGAAAGAAACTGGTGGACAGCACCGACAAG
GCCGACCTGCGGCTGATCTATCTGGCCCTGGCCCACATGATCAAGTTCCGGGGCCACTTCCT
GATCGAGGGCGACCTGAACCCCGACAACAGCGACGTGGACAAGCTGTTCATCCAGCTGGTGC
AGACCTACAACCAGCTGTTCGAGGAAAACCCCATCAACGCCAGCGGCGTGGACGCCAAGGCC
ATCCTGTCTGCCAGACTGAGCAAGAGCAGACGGCTGGAAAATCTGATCGCCCAGCTGCCCGG
CGAGAAGAAGAATGGCCTGTTCGGAAACCTGATTGCCCTGAGCCTGGGCCTGACCCCCAACT
TCAAGAGCAACTTCGACCTGGCCGAGGATGCCAAACTGCAGCTGAGCAAGGACACCTACGAC
GACGACCTGGACAACCTGCTGGCCCAGATCGGCGACCAGTACGCCGACCTGTTTCTGGCCGC
CAAGAACCTGTCCGACGCCATCCTGCTGAGCGACATCCTGAGAGTGAACACCGAGATCACCA
AGGCCCCCCTGAGCGCCTCTATGATCAAGAGATACGACGAGCACCACCAGGACCTGACCCTG
CTGAAAGCTCTCGTGCGGCAGCAGCTGCCTGAGAAGTACAAAGAGATTTTCTTCGACCAGAG
CAAGAACGGCTACGCCGGCTACATTGACGGCGGAGCCAGCCAGGAAGAGTTCTACAAGTTCA
TCAAGCCCATCCTGGAAAAGATGGACGGCACCGAGGAACTGCTCGTGAAGCTGAACAGAGAG
GACCTGCTGCGGAAGCAGCGGACCTTCGACAACGGCAGCATCCCCCACCAGATCCACCTGGG
AGAGCTGCACGCCATTCTGCGGCGGCAGGAAGATTTTTACCCATTCCTGAAGGACAACCGGG
AAAAGATCGAGAAGATCCTGACCTTCCGCATCCCCTACTACGTGGGCCCTCTGGCCAGGGGA
AACAGCAGATTCGCCTGGATGACCAGAAAGAGCGAGGAAACCATCACCCCCTGGAACTTCGA
GGAAGTGGTGGACAAGGGCGCTTCCGCCCAGAGCTTCATCGAGCGGATGACCAACTTCGATA
AGAACCTGCCCAACGAGAAGGTGCTGCCCAAGCACAGCCTGCTGTACGAGTACTTCACCGTG
TATAACGAGCTGACCAAAGTGAAATACGTGACCGAGGGAATGAGAAAGCCCGCCTTCCTGAG
CGGCGAGCAGAAAAAGGCCATCGTGGACCTGCTGTTCAAGACCAACCGGAAAGTGACCGTGA
AGCAGCTGAAAGAGGACTACTTCAAGAAAATCGAGTGCTTCGACTCCGTGGAAATCTCCGGC
GTGGAAGATCGGTTCAACGCCTCCCTGGGCACATACCACGATCTGCTGAAAATTATCAAGGA
CAAGGACTTCCTGGACAATGAGGAAAACGAGGACATTCTGGAAGATATCGTGCTGACCCTGA
CACTGTTTGAGGACAGAGAGATGATCGAGGAACGGCTGAAAACCTATGCCCACCTGTTCGAC
GACAAAGTGATGAAGCAGCTGAAGCGGCGGAGATACACCGGCTGGGGCAGGCTGAGCCGGAA
GCTGATCAACGGCATCCGGGACAAGCAGTCCGGCAAGACAATCCTGGATTTCCTGAAGTCCG
ACGGCTTCGCCAACAGAAACTTCATGCAGCTGATCCACGACGACAGCCTGACCTTTAAAGAG
GACATCCAGAAAGCCCAGGTGTCCGGCCAGGGCGATAGCCTGCACGAGCACATTGCCAATCT
GGCCGGCAGCCCCGCCATTAAGAAGGGCATCCTGCAGACAGTGAAGGTGGTGGACGAGCTCG
TGAAAGTGATGGGCCGGCACAAGCCCGAGAACATCGTGATCGAAATGGCCAGAGAGAACCAG
ACCACCCAGAAGGGACAGAAGAACAGCCGCGAGAGAATGAAGCGGATCGAAGAGGGCATCAA
AGAGCTGGGCAGCCAGATCCTGAAAGAACACCCCGTGGAAAACACCCAGCTGCAGAACGAGA
AGCTGTACCTGTACTACCTGCAGAATGGGGGGGATATGTACGTGGACCAGGAACTGGACATC
AACCGGCTGTCCGACTACGATGTGGACGCCATCGTGCCTCAGAGCTTTCTGAAGGACGACTC
CATCGACAACAAGGTGCTGACCAGAAGCGACAAGAACCGGGGCAAGAGCGACAACGTGCCCT
CCGAAGAGGTCGTGAAGAAGATGAAGAACTACTGGGGGCAGCTGCTGAACGCCAAGCTGATT
ACCCAGAGAAAGTTCGACAATCTGACCAAGGCCGAGAGAGGCGGCCTGAGCGAACTGGATAA
GGCCGGCTTCATCAAGAGACAGCTGGTGGAAACCCGGCAGATCACAAAGCACGTGGCACAGA
TCCTGGACTCCCGGATGAACACTAAGTACGACGAGAATGACAAGCTGATCCGGGAAGTGAAA
GTGATCACCCTGAAGTCCAAGCTGGTGTCCGATTTCCGGAAGGATTTCCAGTTTTACAAAGT
GCGCGAGATCAACAACTACCACCACGCCCACGACGCCTACCTGAACGCCGTCGTGGGAACCG
CCCTGATCAAAAAGTACCCTAAGCTGGAAAGCGAGTTCGTGTACGGCGACTACAAGGTGTAC
GACGTGCGGAAGATGATCGCCAAGAGCGAGCAGGAAATCGGCAAGGCTACCGCCAAGTACTT
CTTCTACAGCAACATCATGAACTTTTTCAAGACCGAGATTACCCTGGCCAACGGCGAGATCC
GGAAGCGGCCTCTGATCGAGACAAACGGCGAAACCGGGGAGATCGTGTGGGATAAGGGCCGG
GATTTTGCCACCGTGCGGAAAGTGCTGAGCATGCCCCAAGTGAATATCGTGAAAAAGACCGA
GGTGCAGACAGGCGGCTTCAGCAAAGAGTCTATCCTGCCCAAGAGGAACAGCGATAAGCTGA
TCGCCAGAAAGAAGGACTGGGACCCTAAGAAGTACGGCGGCTTCGACAGCCCCACCGTGGCC
TATTCTGTGCTGGTGGTGGCCAAAGTGGAAAAGGGCAAGTCCAAGAAACTGAAGAGTGTGAA
AGAGCTGCTGGGGATCACCATCATGGAAAGAAGCAGCTTCGAGAAGAATCCCATCGACTTTC
TGGAAGCCAAGGGCTACAAAGAAGTGAAAAAGGACCTGATCATCAAGCTGCCTAAGTACTCC
CTGTTCGAGCTGGAAAACGGCCGGAAGAGAATGCTGGCCTCTGCCGGCGAACTGCAGAAGGG
AAACGAACTGGCCCTGCCCTCCAAATATGTGAACTTCCTGTACCTGGCCAGCCACTATGAGA
AGCTGAAGGGCTCCCCCGAGGATAATGAGCAGAAACAGCTGTTTGTGGAACAGCACAAGCAC
TACCTGGACGAGATCATCGAGCAGATCAGCGAGTTCTCCAAGAGAGTGATCCTGGCCGACGC
TAATCTGGACAAAGTGCTGTCCGCCTACAACAAGCACCGGGATAAGCCCATCAGAGAGCAGG
CCGAGAATATCATCCACCTGTTTACCCTGACCAATCTGGGAGCCCCTGCCGCCTTCAAGTAC
TTTGACACCACCATCGACCGGAAGAGGTACACCAGCACCAAAGAGGTGCTGGACGCCACCCT
GATCCACCAGAGCATCACCGGCCTGTACGAGACACGGATCGACCTGTCTCAGCTGGGAGGCG
ACAAAAGGCCGGCGGCCACGAAAAAGGccggacaggccaaaaagaaaaagGGCggagatttc
ggcGctagCGAGGGCAGAGGAAGTCTTCTAACATGCGGTGACGTGGAGGAGAATCCCGGCCC
TGgtacCatgaacccagccatcagcgtcgctctcctgctctcagtcttgcaggtgtcccgag
ggcagaaggtgaccagcctgacagcctgcctggtgaaccaaaaccttcgcctggactgccgc
catgagaataacaccaaggataactccatccagcatgagttcagcctgacccgagagaagag
gaagcacgtgctctcaggcacccttgggatacccgagcacacgtaccgctcccgcgtcaccc
tctccaaccagccctatatcaaggtccttaccctagccaacttcaccaccaaggatgagggc
gactacttttgtgagcttcgcgtttcgggcgcgaatcccatgagctccaataaaagtatcag
tgtgtatagagacaagctggtcaagtgtggcggcataagcctgctggttcagaacacatcct
ggatgctgctgctgctgctttccctctccctcctccaagccctggacttcatttctctgtga
SEQ ID NO: 1174
Tet1v4-dCas9-Thy1.1 polypeptide sequence ((Tet1v4-dCas9-Thy1.1; modified slightly from
construct previously published as Tet1v4 in Nunez et al. Cell 2021, 184, 2503-2519)
MALPTCSCLDRVIQKDKGPYYTHLGAGPSVAAVREIMENRYGQKGNAIRIEIVVYTGKEGKS
SHGCPIAKWVLRRSSDEEKVLCLVRQRTGHHCPTAVMVVLIMVWDGIPLPMADRLYTELTEN
LKSYNGHPTDRRCTLNENRTCTCQGIDPETCGASESFGCSWSMYENGCKEGRSPSPRRFRID
PSSPLHEKNLEDNLQSLATRLAPIYKQYAPVAYQNQVEYENVARECRLGSKEGRPFSGVTAC
LDFCAHPHRDIHNMNNGSTVVCTLTREDNRSLGVIPQDEQLHVLPLYKLSDTDEFGSKEGME
AKIKSGAIEVLAPRRKKRTCFTQPVPRSGKKRAAMMTEVLAHKIRAVEKKPIPRIKRKNNST
TTNNSKPSSLPTLGSNTETVQPEVKSETEPHFILKSSDNTKTYSLMPSAPHPVKEASPGFSW
SPKTASATPAPLKNDATASCGFSERSSTPHCTMPSGRLSGANAAAADGPGISQLGEVAPLPT
LSAPVMEPLINSEPSTGVTEPLTPHQPNHQPSFLTSPQDLASSPMEEDEQHSEADEPPSDEP
LSDDPLSPAREKLPHIDEYWSDSEHIFLDANIGGVAIAPAHGSVLIECARRELHATTPVEHP
NRNHPTRLSLVFYQHKNLNKPQHGFELNKIKFEAKEAKNKKMKASEQKDQAANEGPEQSSEV
NELNQIPSHKALTLTHDNVVTVSPYALTHVAGPYNHWVGGPSSGAPPPSGGSPAGSPTSTEE
GTSESATPESGPGTSTEPSEGSAPGSPAGSPISTEEGTSTEPSEGSAPGTSTEPSEMDKKYS
IGLAIGTNSVGWAVITDEYKVPSKKFKVIGNTDRHSIKKNLIGALLEDSGETAEATRLKRTA
RRRYTRRKNRICYLQEIFSNEMAKVDDSFFHRLEESFLVEEDKKHERHPIFGNIVDEVAYHE
KYPTIYHLRKKLVDSTDKADLRLIYLALAHMIKFRGHFLIEGDLNPDNSDVDKLFIQLVQTY
NQLFEENPINASGVDAKAILSARLSKSRRLENLIAQLPGEKKNGLEGNLIALSLGLTPNEKS
NEDLAEDAKLQLSKDTYDDDLDNLLAQIGDQYADLFLAAKNLSDAILLSDILRVNTEITKAP
LSASMIKRYDEHHQDLTILKALVRQQLPEKYKEIFFDQSKNGYAGYIDGGASQEEFYKFIKP
ILEKMDGTEELLVKINREDLLRKQRTEDNGSIPHQIHLGELHAILRRQEDFYPFLKDNREKI
EKILTFRIPYYVGPLARGNSRFAWMTRKSEETITPWNFEEVVDKGASAQSFIERMINEDKNL
PNEKVLPKHSLLYEYFTVYNELTKVKYVTEGMRKPAFLSGEQKKAIVDLLEKTNRKVTVKQL
KEDYFKKIECFDSVEISGVEDRENASLGTYHDLLKIIKDKDELDNEENEDILEDIVLTLTLE
EDREMIEERLKTYAHLEDDKVMKQLKRRRYTGWGRLSRKLINGIRDKQSGKTILDELKSDGF
ANRNFMQLIHDDSLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHPVENTQLQNEKLY
LYYLQNGRDMYVDQELDINRLSDYDVDAIVPQSFLKDDSIDNKVLTRSDKNRGKSDNVPSEE
VVKKMKNYWRQLLNAKLITQRKEDNLTKAERGGLSELDKAGFIKRQLVETRQITKHVAQILD
SRMNTKYDENDKLIREVKVITLKSKLVSDFRKDFQFYKVREINNYHHAHDAYLNAVVGTALI
KKYPKLESEFVYGDYKVYDVRKMIAKSEQEIGKATAKYFFYSNIMNFFKTEITLANGEIRKR
PLIETNGETGEIVWDKGRDFATVRKVLSMPQVNIVKKTEVQTGGFSKESILPKRNSDKLIAR
KKDWDPKKYGGFDSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVNFLYLASHYEKLK
GSPEDNEQKQLFVEQHKHYLDEIIEQISEFSKRVILADANLDKVLSAYNKHRDKPIREQAEN
IIHLETLINIGAPAAFKYEDTTIDRKRYTSTKEVLDATLIHQSITGLYETRIDLSQLGGDGG
GGSPKKKRKVDPKKKRKVDPKKKRKVGGSGATNFSLLKQAGDVEENPGPMNPAISVALLISV
LQVSRGQKVTSLTACLVNQNLRLDCRHENNTKDNSIQHEFSLTREKRKHVLSGTIGIPEHTY
RSRVTLSNQPYIKVLTLANFTTKDEGDYFCELRVSGANPMSSNKSISVYRDKLVKCGGISLL
VQNTSWMLLLLLSLSLLQALDFISL*
SEQ ID NO: 1175
Tet1v4-dCas9-Thy1.1 DNA sequence (Tet1v4-dCas9-Thy1.1; modified slightly from
construct previously published as Tet1v4 in Nunez et al. Cell 2021, 184, 2503-2519)
CTGCCCACctgcagctgtcttgatcgagttatacaaaaagacaaaggcccatattatacaca
ccttggggcaggaccaagtgttgctgctgtcagggaaatcatggagaataggtatggtcaaa
aaggaaacgcaataaggatagaaatagtagtgtacaccggtaaagaagggaaaagctctcat
gggtgtccaattgctaagtgggttttaagaagaagcagtgatgaagaaaaagttctttgttt
ggtccggcagcgtacaggccaccactgtccaactgctgtgatggtggtgctcatcatggtgt
gggatggcatccctcttccaatggccgaccggctatacacagagctcacagagaatctaaag
tcatacaatgggcaccctaccgacagaagatgcaccctcaatgaaaatcgtacctgtacatg
tcaaggaattgatccagagacttgtggagcttcattctcttttggctgttcatggagtatgt
actttaatggctgtaagtttggtagaagcccaagccccagaagatttagaattgatccaagc
tctcccttacatgaaaaaaaccttgaagataacttacagagtttggctacacgattagctcc
aatttataagcagtatgctccagtagcttaccaaaatcaggtggaatatgaaaatgttgccc
gagaatgtcggcttggcagcaaggaaggtcgacccttctctggggtcactgcttgcctggac
ttctgtgctcatccccacagggacattcacaacatgaataatggaagcactgtggtttgtac
cttaactcgagaagataaccgctctttgggtgttattcctcaagatgagcagctccatgtgc
tacctctttataagctttcagacacagatgagtttggctccaaggaaggaatggaagccaag
atcaaatctggggccatcgaggtcctggcaccccgccgcaaaaaaagaacgtgtttcactca
gcctgttccccgttctggaaagaagagggctgcgatgatgacagaggttcttgcacataaga
taagggcagtggaaaagaaacctattccccgaatcaagcggaagaataactcaacaacaaca
aacaacagtaagccttcgtcactgccaaccttagggagtaacactgagaccgtgcaacctga
agtaaaaagtgaaaccgaaccccattttatcttaaaaagttcagacaacactaaaacttatt
cgctgatgccatccgctcctcacccagtgaaagaggcatctccaggcttctcctggtccccg
aagactgcttcagccacaccagctccactgaagaatgacgcaacagcctcatgcgggttttc
agaaagaagcagcactccccactgtacgatgccttcgggaagactcagtggtgccaatgctg
cagctgctgatggccctggcatttcacagcttggcgaagtggctcctctccccaccctgtct
gctcctgtgatggagcccctcattaattctgagccttccactggtgtgactgagccgctaac
gcctcatcagccaaaccaccagccctccttcctcacctctcctcaagaccttgcctcttctc
caatggaagaagatgagcagcattctgaagcagatgagcctccatcagacgaacccctatct
gatgaccccctgtcacctgctgaggagaaattgccccacattgatgagtattggtcagacag
tgagcacatctttttggatgcaaatattggtggggtggccatcgcacctgctcacggctcgg
ttttgattgagtgtgcccggcgagagctgcacgctaccactcctgttgagcaccccaaccgt
aatcatccaacccgcctctcccttgtcttttaccagcacaaaaacctaaataagccccaaca
tggttttgaactaaacaagattaagtttgaggctaaagaagctaagaataagaaaatgaagg
cctcagagcaaaaagaccaggcagctaatgaaggtccagaacagtcctctgaagtaaatgaa
ttgaaccaaattccttctcataaagcattaacattaacccatgacaatgttgtcaccgtgtc
cccttatgctctcacacacgttgcggggccctataaccattgggtcggagggccgagctctg
gcgcacccccaccaagtggagggtctcctgccgggtccccaacatctactgaagaaggcacc
agcgaatccgcaacgcccgagtcaggccctggtacctccacagaaccatctgaaggtagtgc
gcctggttccccagctggaagccctacttccaccgaagaaggcacgtcaaccgaaccaagtg
aaggatctgcccctgggaccagcactgaaccatctgagatggacaagaagtattctatcgga
ctggccatcgggactaatagcgtcgggtgggccgtgatcactgacgagtacaaggtgccctc
taagaagttcaaggtgctcgggaacaccgaccggcattccatcaagaaaaatctgatcggag
ctctcctctttgattcaggggagaccgctgaagcaacccgcctcaagcggactgctagacgg
cggtacaccaggaggaagaaccggatttgttaccttcaagagatattctccaacgaaatggc
aaaggtcgacgacagcttcttccataggctggaagaatcattcctcgtggaagaggataaga
agcatgaacggcatcccatcttcggtaatatcgtcgacgaggtggcctatcacgagaaatac
ccaaccatctaccatcttcgcaaaaagctggtggactcaaccgacaaggcagacctccggct
tatctacctggccctggcccacatgatcaagttcagaggccacttcctgatcgagggcgacc
tcaatcctgacaatagcgatgtggataaactgttcatccagctggtgcagacttacaaccag
ctctttgaagagaaccccatcaatgcaagcggagtcgatgccaaggccattctgtcagcccg
gctgtcaaagagccgcagacttgagaatcttatcgctcagctgccgggtgaaaagaaaaatg
gactgttcgggaacctgattgctctttcacttgggctgactcccaatttcaagtctaatttc
gacctggcagaggatgccaagctgcaactgtccaaggacacctatgatgacgatctcgacaa
cctcctggcccagatcggtgaccaatacgccgaccttttccttgctgctaagaatctttctg
acgccatcctgctgtctgacattctccgcgtgaacactgaaatcaccaaggcccctctttca
gcttcaatgattaagcggtatgatgagcaccaccaggacctgaccctgcttaaggcactcgt
ccggcagcagcttccggagaagtacaaggaaatcttctttgaccagtcaaagaatggatacg
ccggctacatcgacggaggtgcctcccaagaggaattttataagtttatcaaacctatcctt
gagaagatggacggcaccgaagagctcctcgtgaaactgaatcgggaggatctgctgcggaa
gcagcgcactttcgacaatgggagcattccccaccagatccatcttggggagcttcacgcca
tccttcggcgccaagaggacttctacccctttcttaaggacaacagggagaagattgagaaa
attctcactttccgcatcccctactacgtgggacccctcgccagaggaaatagccggtttgc
ttggatgaccagaaagtcagaagaaactatcactccctggaacttcgaagaggtggtggaca
agggagccagcgctcagtcattcatcgaacggatgactaacttcgataagaacctccccaat
gagaaggtcctgccgaaacattccctgctctacgagtactttaccgtgtacaacgagctgac
caaggtgaaatatgtcaccgaagggatgaggaagcccgcattcctgtcaggcgaacaaaaga
aggcaattgtggaccttctgttcaagaccaatagaaaggtgaccgtgaagcagctgaaggag
gactatttcaagaaaattgaatgcttcgactctgtggagattagcggggtcgaagatcggtt
caacgcaagcctgggtacctaccatgatctgcttaagatcatcaaggacaaggattttctgg
acaatgaggagaacgaggacatccttgaggacattgtcctgactctcactctgttcgaggac
cgggaaatgatcgaggagaggcttaagacctacgcccatctgttcgacgataaagtgatgaa
gcaacttaaacggagaagatataccggatggggacgccttagccgcaaactcatcaacggaa
tccgggacaaacagagcggaaagaccattcttgatttccttaaaagcgacggattcgctaat
cgcaacttcatgcaacttatccatgatgattccctgacctttaaggaggacatccagaaggc
ccaagtgtctggacaaggtgactcactgcacgagcatatcgcaaatctggctggttcacccg
ctattaagaagggtattctccagaccgtgaaagtcgtggacgagctggtcaaggtgatgggt
cgccataaaccagagaacattgtcatcgagatggccagggaaaaccagactacccagaaggg
acagaagaacagcagggagcggatgaaaagaattgaggaagggattaaggagctcgggtcac
agatccttaaagagcacccggtggaaaacacccagcttcagaatgagaagctctatctgtac
taccttcaaaatggacgcgatatgtatgtggaccaagagcttgatatcaacaggctctcaga
ctacgacgtggacgccatcgtccctcagagcttcctcaaagacgactcaattgacaataagg
tgctgactcgctcagacaagaaccggggaaagtcagataacgtgccctcagaggaagtcgtg
aaaaagatgaagaactattggcgccagcttctgaacgcaaagctgatcactcagcggaagtt
cgacaatctcactaaggctgagaggggcggactgagcgaactggacaaagcaggattcatta
aacggcaacttgtggagactcggcagattactaaacatgtcgcccaaatccttgactcacgc
atgaataccaagtacgacgaaaacgacaaacttatccgcgaggtgaaggtgattaccctgaa
gtccaagctggtcagcgatttcagaaaggactttcaattctacaaagtgcgggagatcaata
actatcatcatgctcatgacgcatatctgaatgccgtggtgggaaccgccctgatcaagaag
tacccaaagctggaaagcgagttcgtgtacggagactacaaggtctacgacgtgcgcaagat
gattgccaaatctgagcaggagatcggaaaggccaccgcaaagtacttcttctacagcaaca
tcatgaatttcttcaagaccgaaatcacccttgcaaacggtgagatccggaagaggccgctc
atcgagactaatggggagactggcgaaatcgtgtgggacaagggcagagatttcgctaccgt
gcgcaaagtgctttctatgcctcaagtgaacatcgtgaagaaaaccgaggtgcaaaccggag
gcttttctaaggaatcaatcctccccaagcgcaactccgacaagctcattgcaaggaagaag
gattgggaccctaagaagtacggcggattcgattcaccaactgtggcttattctgtcctggt
cgtggctaaggtggaaaaaggaaagtctaagaagctcaagagcgtgaaggaactgctgggta
tcaccattatggagcgcagctccttcgagaagaacccaattgactttctcgaagccaaaggt
tacaaggaagtcaagaaggaccttatcatcaagctcccaaagtatagcctgttcgaactgga
gaatgggcggaagcggatgctcgcctccgctggcgaacttcagaagggtaatgagctggctc
tcccctccaagtacgtgaatttcctctaccttgcaagccattacgagaagctgaaggggagc
cccgaggacaacgagcaaaagcaactgtttgtggagcagcataagcattatctggacgagat
cattgagcagatttccgagttttctaaacgcgtcattctcgctgatgccaacctcgataaag
tccttagcgcatacaataagcacagagacaaaccaattcgggagcaggctgagaatatcatc
cacctgttcaccctcaccaatcttggtgcccctgccgcattcaagtacttcgacaccaccat
cgaccggaaacgctatacctccaccaaagaagtgctggacgccaccctcatccaccagagca
tcaccggactttacgaaactcggattgacctctcacagctcggaggggatggtggcggaggc
tcgccaaaaaagaagagaaaggtagacccaaagaaaaaacgaaaagtagatccgaaaaagaa
gaggaaggtgggaggtagtggggctactaacttcagcctgctgaagcaggctggagacgtgg
aggagaaccctggacctATGAACCCAGCCATCAGcgtcgctctcctgctctcagtcttgcag
gtgtcccgagggcagaaggtgaccagcctgacagcctgcctggtgaaccaaaaccttcgcct
ggactgccgccatgagaataacaccaaggataactccatccagcatgagttcagcctgaccc
gagagaagaggaagcacgtgctctcaggcacccttgggatacccgagcacacgtaccgctcc
cgcgtcaccctctccaaccagccctatatcaaggtccttaccctagccaacttcaccaccaa
ggatgagggcgactacttttgtgagcttcgcgtttcgggcgcgaatcccatgagctccaata
aaagtatcagtgtgtatagagacaagctggtcaagtgtggcggcataagcctgctggttcag
aacacatcctggatgctgctgctgctgctttccctctccctcctccaagcCCTGGACTTCAT
TTCTCTGTGA
SEQ ID NO: 1176
Polypeptide sequence of KRAB protein
RTLVTFKDVFVDFTREEWKLLDTAQQILYRNVMLENYKNIVSLGYQLTKPDVILRLEKGEEP
WLV
SEQ ID NO: 1177
Polynucleotide sequence for KRAB
cggacactggtgaccttcaaggatgtgtttgtggacttcaccagggaggagtggaagctgct
ggacactgctcagcagatcctgtacagaaatgtgatgctggagaactataagaacctggttt
ccttgggttatcagcttactaagccagatgtgatcctccggttggagaagggagaagagccc
tggctggtg

1. A method of stably activating a gene or gene product within the imprinted 15q11-13 locus in a subject having Prader Willi Syndrome (PWS) or Prader-Willi-like disorder, the method comprising non-virally administering to the subject a DNA targeting system that targets a target region in the imprinted 15q11-13 locus, the DNA targeting system comprising:
a Cas protein or a fusion protein, wherein the fusion protein comprises two heterologous polypeptide domains, wherein the first polypeptide domain comprises a DNA-binding protein and wherein the second polypeptide domain has an activity selected from transcription activation activity, transcription repression activity, transcription release factor activity, histone modification activity, nuclease activity, nucleic acid association activity, methylase activity, demethylase activity, acetylation activity, and deacetylation activity,
wherein the Cas protein or fusion protein is targeted to the target region in the imprinted 15q11-13 locus.

2. The method of claim 1, wherein at least one component of the DNA targeting system is transiently expressed in a cell from the subject or transiently delivered to a cell from the subject.

3. The method of any one of claims 1-2, wherein expression of a gene within the imprinted 15q1-13 locus is maintained in a cell from the subject for at least 10, at least 15, at least 20, at least 25, at least 26, at least 30, at least 35, at least 40, at least 45, at least 48, at least 50, or at least 55 days post-administration.

4. The method of any one of claims 1-3, wherein the DNA-binding protein comprises a Cas protein, a zinc finger protein, or a transcription activator-like effector (TALE) protein.

5. The method of any one of claims 1-4, wherein the DNA-binding protein comprises a Cas protein and the DNA targeting system further comprises one or more guide RNAs (gRNA) that binds to the target region in the imprinted 15q11-13 locus.

6. The method of any one of claims 1-5, wherein the Cas protein comprises a Cas9 protein.

7. The method of any one of claims 1-6, wherein the second polypeptide domain comprises VP64, VP16; GAL4; p65 subdomain (NFkB); KMT2 family transcriptional activators: hSET1A, hSET1B, MLL1 to 5, ASH1, and homologs (Trx, Trr, Ash1); KMT3 family: SYMD2, NSD1; KMT4 family: DOT1L and homologs; KDM1: LSD1/BHC110 and homologs (SpLsd1/Swml/Saf110, Su(var)3-3); KDM3 family: JHDM2a/b; KDM4 family: JMJD2A/JHDM3A, JMJD2B, JMJD2C/GASC1, JMJD2D, and homologs (Rph1); KDM6 family: UTX, JMJD3, VP64-p65-Rta (VPR); synergistic action mediator (SAM); p300; VP160; VP64-dCas9-BFP-VP64; KAT2 family: hGCN5, PCAF, and homologs (dGCN5/PCAF, Gcn5; KAT3 family: CBP, p300 and homologs (dCBP/NEJ); KAT4: TAF1 and homologs (dTAF1); KAT5: TIP60/PLIP, and homologs; KAT6: MOZ/MYST3, MORF/MYST4, and homologs (Mst2, Sas3, CG1894); KAT7: HBO1/MYST2, and homologs (CHM, Mst2); KAT8: HMOF/MYST1, and homologs (dMOF, CG1894, Sas2, Mst2); KAT13 family: SRC1, ACTR, P160, CLOCK, and homologs; AID/Apobed deaminase family: AID; TET dioxygenase family: TET1; DEMETER glycosylase family: DME, DML1, DML2, or ROS1.

8. The method of any one of claims 1-6, wherein the second polypeptide domain comprises KRAB, Mad mSIN3 interaction domain (SID); the ERF repressor domain (ERD); KMT1 family: SUV39H1, SUV39H2, G9A, ESET/SETBD1, and homologs (Cir4, Su(var)3-9); KMT5 family: Pr-SET7/8, SUV4-20H1, and homologs (PR-set7, Suv4-20, and Set9); KMT6: EZH2, KMT8: RIZ1, KDM4 family: JMJD2A/JHDM3A, JMJD2B, JMJ2D2C/GASC1, JMJD2D, and homologs (Rph1); KDM5 family JARID1A/RBP2, JARID1B/PLU-1, JARID1C/SMCX, JARID1D/SMCY, and homologs (Lid, Jhn2, Jmj2); HDAC1, HDAC2, HDAC3, HDAC8, and its homologs (Rpd3, Hos1, Cir6); HDAC4, HDAC5, HDAC7, HDAC9, and its homologs (Hda1, Cir3); SIRT1, SIRT2, and its homologs (Sir2, Hst1, Hst2, Hst3, and Hst4); HDAC11, DNMT1, DNMT3a/3b, MET1, DRM3, and homologs, ZMET2, CMT1, CMT2, Laminin A, Laminin B, or CTCF.

9. The method of any one of claims 1-8, wherein the second polypeptide domain comprises Tet1c or Tet1v4.

10. The method of any one of claims 1-9, wherein the second polypeptide domain comprises the amino acid sequence of SEQ ID NO: 1139 or SEQ ID NO: 1166, or is encoded by a polynucleotide comprising the sequence of SEQ ID NO: 1138 or SEQ ID NO: 1167.

11. The method of any one of claims 1-8, wherein the fusion protein comprises VP64-dCas9-VP64, dCas9-KRAB, Tet1c-dCas9, or Tet1v4-dCas9.

12. The method of any one of claims 1-11, wherein the fusion protein comprises the amino acid sequence of SEQ ID NO: 1168 or SEQ ID NO: 1169, or is encoded by a polynucleotide comprising the sequence of SEQ ID NO: 1169 or SEQ ID NO: 1171.

13. The method of any one of claims 1-12, wherein the target region in the imprinted 15q11-13 PWS-associated locus is on the maternal copy.

14. The method of any one of claims 1-12, wherein the target region in the imprinted 15q11-13 PWS-associated locus is on the paternal copy.

15. The method of any one of claims 1-14, wherein the expression of a gene or gene product within the imprinted 15q11-13 locus is increased.

16. The method of any one of claims 1-15, wherein the gene within the imprinted 15q11-13 locus comprises SNRPN, MAGEL2, MKRN3, NDN, C15ORF2, SNURF-SNRPN, SNHG14, SNORD107, SNORD64, SNORD109A, SNORD116, SNORD116@, SPA1, SPA2, 116HG, SNORD116-1 to 30, Sno-Inc RNA 1 to 5, IPW, SNORD115, SNORD115@M, 115HG, SNORD115-1 to 48, SNORD109B, SNG14, or a snoRNA in the SNORD116 cluster, or a combination thereof.

17. The method of claim 16, wherein the gene within the imprinted 15q11-13 locus comprises SNRPN, SNORD116, MAGEL2, SNORD115, SPA1, and/or SPA2.

18. The method of claim 17, wherein the expression of MAGEL2 or its products is increased.

19. The method of claim 17, wherein the expression of SNORD116 or its products is increased.

20. The method of claim 17, wherein the expression of the SNRPN gene or its products is increased.

21. The method of claim 20, wherein expression of the SNRPN gene is maintained in a cell from the subject for at least 10, at least 15, at least 20, at least 25, at least 26, at least 30, at least 35, at least 40, at least 45, at least 48, at least 50, or at least 55 days post-administration.

22. The method of any one of claims 1-21, wherein the gRNA is encoded by a polynucleotide comprising a sequence selected from SEQ ID NOs: 1148-1156 or binds to a polynucleotide comprising a sequence selected from SEQ ID NOs: 1148-1156 or comprises a sequence selected from SEQ ID NOs: 1157-1165.

23. The method of any one of claims 5-22, wherein the DNA targeting system comprises two or more gRNAs.

24. The method of any one of claims 1-23, wherein the subject is administered a vector comprising a polynucleotide encoding the DNA targeting system.

25. The method of claim 24, wherein the vector is a plasmid or a synthetic vector.

26. The method of claim 24, wherein the vector comprises RNA.

27. The method of claim 24, wherein the vector comprises ribonucleoprotein (RNP).

28. The method of any one of claims 24-27, wherein the vector is a vector within a nanoparticle.

29. The method of claim 28, wherein the nanoparticle is a lipid nanoparticle or a polymeric nanoparticle.

30. A DNA targeting system that targets the imprinted 15q11-13 locus, the DNA targeting system comprising:
(a) a Cas9 fusion protein, wherein the fusion protein comprises two heterologous polypeptide domains, wherein the first polypeptide domain comprises a Cas protein and the second polypeptide domain comprises Tet1, Tet1c, or Tet1v4; and
(b) one or more guide RNAs (gRNA) that bind to a target region in the imprinted 15q11-13 locus.

31. The DNA targeting system of claim 30, for use in stably activating expression of a gene or gene product within the imprinted 15q11-13 locus in a subject having Prader Willi Syndrome (PWS) or Prader-Willi-like disorder.

32. An isolated polynucleotide sequence encoding the DNA targeting system of claim 30 or 31.

33. A vector comprising the isolated polynucleotide sequence of claim 32.

34. A nanoparticle comprising the DNA targeting system of claim 30 or 31, or the isolated polynucleotide sequence of claim 32, or the vector of claim 33, or a combination thereof.

35. The nanoparticle of claim 34, wherein the nanoparticle is a lipid nanoparticle or a polymeric nanoparticle.

36. A pharmaceutical composition comprising the DNA targeting system of claim 30 or 31, or the isolated polynucleotide sequence of claim 32, or the vector of claim 33, or the nanoparticle of claim 34 or 35, or a combination thereof.
